SEQUENCE LISTING (57 sequences)

SEQ ID NO: 1 - protein, 447 aa, Streptomyces collinus
  REGION 1..447: note = Ccr
MTVKDILDAIQSKDATSADFAALQLPESYRAITVHKDETEMFAGLETRDKDPRKSIHLDEVPVPELGPGEALVAVMASSVNYNSVWTSIFEPVSTFAFLERYGKLSPLTKRHDLPYHIIGSDLAGVVLRTGPGVNAWQPGDEVVAHCLSVELESPDGHDDTMLDPEQRIWGFETNFGGLAEIALVKTNQLMPKPKHLTWEEAAAPGLVNSTAYRQLVSRNGAAMKQGDNVLIWGASGGLGSYATQFALAGGANPICVVSSPQKAEICRSMGAEAIIDRNAEGYKFWKDEHTQDPKEWKRFGKRIRELTGGEDIDIVFEHPGRETFGASVYVTRKGGTITTCASTSGYMHEYDNRYLWMSLKRIIGSHFANYREAYEANRLIAKGKIHPTLSKTYSLEETGQAAYDVHRNLHQGKVGVLCLAPEEGLGVRDAEMRAQHIDAINRFRNV

SEQ ID NO: 2 - protein, 397 aa, Treponema denticola
  REGION 1..397: note = FabV
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 3 - protein, 262 aa, Escherichia coli
  REGION 1..262: note = FabI
MGFLSGKRILVTGVASKLSIAYGIAQAMHREGAELAFTYQNDKLKGRVEEFAAQLGSDIVLQCDVAEDASIDTMFAELGKVWPKFDGFVHSIGFAPGDQLDGDYVNAVTREGFKIAHDISSYSFVAMAKACRSMLNPGSALLTLSYLGAERAIPNYNVMGLAKASLEANVRYMANAMGPEGVRVNAISAGPIRTLAASGIKDFRKMLAHCEAVTPIRRTVTIEDVGNSAAFLCSDLSAGISGEVVHVDGGFSIAAMNELELK

SEQ ID NO: 4 - protein, 383 aa, Megasphaera elsdenii
  REGION 1..383: note = short-chain acyl-CoA dehydrogenase
MDFNLTDIQQDFLKLAHDFGEKKLAPTVTERDHKGIYDKELIDELLSLGITGAYFEEKYGGSGDDGGDVLSYILAVEELAKYDAGVAITLSATVSLCANPIWQFGTEAQKEKFLVPLVEGTKLGAFGLTEPNAGTDASGQQTIATKNDDGTYTLNGSKIFITNGGAADIYIVFAMTDKSKGNHGITAFILEDGTPGFTYGKKEDKMGIHTSQTMELVFQDVKVPAENMLGEEGKGFKIAMMTLDGGRIGVAAQALGIAEAALADAVEYSKQRVQFGKPLCKFQSISFKLADMKMQIEAARNLVYKAACKKQEGKPFTVDAAIAKRVASDVAMRVTTEAVQIFGGYGYSEEYPVARHMRDAKITQIYEGTNEVQLMVTGGALLR

SEQ ID NO: 5 - protein, 383 aa, Acidaminococcus fermentans
  REGION 1..383: note = butyryl-CoA dehydrogenase
MDFNLTEDQQMIKDMAAEFAEKFLAPTVEERDKAHIWDRKLIDKMGEAGFCGICFPEEYGGMGLDVLSYILAVEELSKVDDGTGITLSANVSLCATPIYMFGTEEQKQKYLAPIAEGTHVGAFGLTEPSAGTDASAQQTTAVLKGDKYILNGSKIFITNGKEADTYVVFAMTDKSQGVHGISAFILEKGMPGFRFGKIEDKMGGHTSITAELIFEDCEVPKENLLGKEGEGFKIAMETLDGGRIGVAAQALGIAEGALAAAVKYSKEREQFGRSISKFQALQFMMADMATKIEAARYLVYHAAMLKNEGKPYSEAAAMAKCFASDVAMEVTTDAVQIFGGYGYTVDYPAERYMRNAKITQIYEGTNQVMRIVTSRALLRDKKK

SEQ ID NO: 6 - protein, 136 aa, Escherichia coli
  REGION 1..136: note = MenI
MIWKRKITLEALNAMGEGNMVGFLDIRFEHIGDDTLEATMPVDSRTKQPFGLLHGGASVVLAESIGSVAGYLCTEGEQKVVGLEINANHVRSAREGRVRGVCKPLHLGSRHQVWQIEIFDEKGRLCCSSRLTTAIL

SEQ ID NO: 7 - protein, 136 aa, Shigella flexneri
  REGION 1..136: note = MenI
MIWKRKITLEALNAMGEGNMVGFLDIRFEHIGDDTLEATMPVDSRTKQPFGLLHGGASVVLAESIGSVAGYLCTEGEQKVVGLEINANHVRSAREGRVRGICKPLHLGSRHQVWQIEIFDEKGRLCCSSRLTTAIL

SEQ ID NO: 8 - protein, 286 aa, Escherichia coli
  REGION 1..286: note = TesB
MSQALKNLLTLLNLEKIEEGLFRGQSEDLGLRQVFGGQVVGQALYAAKETVPEERLVHSFHSYFLRPGDSKKPIIYDVETLRDGNSFSARRVAAIQNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 9 - protein, 542 aa, Cupriavidus necator
  REGION 1..542: note = YdiF (Pct)
MKVITAREAAALVQDGWTVASAGFVGAGHAEAVTEALEQRFLQSGLPRDLTLVYSAGQGDRGARGVNHFGNAGMTASIVGGHWRSATRLATLAMAEQCEGYNLPQGVLTHLYRAIAGGKPGVMTKIGLHTFVDPRTAQDARYHGGAVNERARQAIAEGKACWVDAVDFRGDEYLFYPSFPIHCALIRCTAADARGNLSTHREAFHHELLAMAQAAHNSGGIVIAQVESLVDHHEILQAIHVPGILVDYVVVCDNPANHQMTFAESYNPAYVTPWQGEAAVAEAEAAPVAAGPLDARTIVQRRAVMELARRAPRVVNLGVGMPAAVGMLAHQAGLDGFTLTVEAGPIGGTPADGLSFGASAYPEAVVDQPAQFDFYEGGGIDLAILGLAELDGHGNVNVSKFGEGEGASIAGVGGFINITQSARAVVFMGTLTAGGLEVRAGDGGLQIVREGRVKKIVPEVSHLSFNGPYVASLGIPVLYITERAVFEMRAGADGEARLTLVEIAPGVDLQRDVLDQCSTPIAVAQDLREMDARLFQAGPLHL

SEQ ID NO: 10 - protein, 447 aa, Syntrophomonas wolfei
  REGION 1..447: note = Swol_1932
  source 1..447: note = subsp. Wolfei
MYQKLLEEYKSKLVTADEAAKQVKSGDWVEYGFGINCARDFDEALAKRKDELEDVKIRCDIGAYQHFTAEVDPDNKHFTWNSWHVAGHDRKFINKNLFYIPMKFHENPMMTRKDCVPTNVAVIQCTAMDKHGYFNFGGSSVNCCAMMETARVTILEVNEKMPRCLGGNQECLHISQVDYIIQSKNEPIATIGSAEPSPVEIAMAQHIIERLYDGNCIQLGIGGTPNAVGSMVAASDLKDLGVHTEMYVDAYLLMAKAGKITGARKSIDKYKQVYSFAMGSQELYDYIDDNPGLASYSVDYTNNPWVVAQIDDFVSINACIEVDLYGQVCAESVGTRHISGTGGQLDFVEGAYKSKNGQSFICLPSTIEIKGEVTSRIKPILTPGAIVTDPRTATHMMVTEFGIATLKGRSTWERAEELIKIAHPDFQDELVKEAQKMNIWRKSNKIG

SEQ ID NO: 11 - protein, 446 aa, Syntrophomonas wolfei
  REGION 1..446: note = Swol_0436
  source 1..446: note = subsp. Wolfei
MNYVNEYRKKLVSADEAVKVVNSGDWIDYGSMCGQVVMLDEALARRKEELKDVKIWTLLTLRRPRVMDVDPEEESFVWHSWHLSAMDRKIASERAVYYSPIRYSELPRYVREHIEPLAVAMLQVAPMDKHGYFNFGPQNSHTRAVCERARIVIVEVNNQLPRCLGGYEESIHISEVDYIVEGNNPPLPQLPDAASSEVDKKAAELIIEEMKDECCVQLGIGGMPNAVGKMIAESDLKDLGCHTEMLVDAYVHMYEAGRLTGRKKNIDPGKMVYDFAVGSQRLYDFIDNNPLCASYPVNYTNRPSIAALNDNLITINNAIEVDLYGQVCAESVGTRIISGTGGQLDFVLAAYESRGGKSFVCLPSCSEREGKLKSKIVPTLKPGAIVTDPRSVAHYIVTEYGKFAMKGMSVWQRAEGLINLAHPQLRDELIQAAQAQGIWRRSNKIS

SEQ ID NO: 12 - protein, 726 aa, Syntrophomonas wolfei
  REGION 1..726: note = Q9Y1N2
  source 1..726: note = subsp. Wolfei
MRQNYSTKYKKMGKLSFLTNPASVAVIGASPNAGKVGNTVVTNIKESGYTGKVYPINPTATEILGYKTYKSVLDVPDSIDVVIVVIPSKAVLAAAKECAQKKVKSLVVITAGFKEIGGEGVQMEQDLTKICKDAGIRLVGPNCLGIVTPNLNCTFASAKPSKGSIAFLSQSGAMLTSILDWALTNGIGFSNFISLGNKADVDEVDLIMEVAEDPNTDIILLYLESIVDGRKFLEQIPTCVHKKPVIILKSGTSAAGAAAASSHTGALAGNDIAFDLAFEKAGVLRAATMSDLFDLGRLFVSHRLPKGDNFVIVTNAGGPGIVTTDAFETYHVGMAALSDKTKEALAKVLPGEASVKNPVDIVGDAPPKRYEDALEICFKEPPETVAGAVILVTPQGQTKPCEVAELCTRMYAKYPDRLVVSAFMGGLTMQEPSKILNNAKMPVFPFPEPAIHATGAVLKYRKIKNRKTLAEKKVEVFKVDNERIKKIIAGARADGRTVLLSHETSEIFTLYGVNAPKTKLATNEAEAATFAKEVTFPVVMKIVSPQIIHKSDCGGVKLNIKTEAEATAAFKEIMANAAKNGPKGAVLKGVEIQQMVDFSKYQKTTEMIVGVNRDPTWGPMIMVGQGGIYANYIKDVAFDLAYKYDREDAEAQLKKTKIYEILNGVRGQPRSDIKGLLDTMVKLAQLVNDFSEITELDMNPLLVFEEQKEGKNPGIAAVDVKITLSH

SEQ ID NO: 13 - protein, 696 aa, Chloroflexus aurantiacus
  REGION 1..696: note = Caur_3920
MLEAIFSPQSVAVIGASPDPARLGHRVLKNILDHGYQGNIYPIHPRATEVLGLCAYPSVLDVPNPIDLAVVVIPPQHVIAAVDECGRKGIKGLVVITAGFKEVGGAGRELERELLATVRRYGMRMIGPNSLGIIDTISKLNASFANAMPLPGNIALMSQSGAICTAILDWSHQQGIGFSRFVSLGNKTDVDEVALLEAWNRDEHSRVILAYLEAIDDGPGFIRVAREVTKTTPVVAIKSGTTAAGTRAASSHTGSLAGSEAAYETAFAQSGILRARTMNELFDLALVFAYQPMIRGNRVAIVTNAGGPGIIATDAVERSGLAMAEFTPETIRHLQETLPPNANVFNPIDVIGDATPDRYAVAIKAALADPNVDGLIILFTPQAVSEPLTTANLIIDLVKGSDKPVVTSFMGGASIEKAVQALNAARIPNYLFPERAVQSLAAMYRQSLWQRRPTPTYRTFSVDRERVARLFASVREAGRVELGEVEAREVIEAYGMRLPKSRLAQTPDEAVAIANEFGYPVVMKISSPDILHKSDIGGVRVGLSDASAVRDAFELIEYRARKYLPSARIWGVLVQEMVRKGREVLVGVSRDPQFGPLIAVGMGGIYVEVLKDVAFRLAPLSVEEVQEQIRSIRAYPLLRGVRGEAPADIAAIEETVLRVSQLVTDFPEIVEMDINPLVVHHEGEGAVVLDARIILN

SEQ ID NO: 14 - protein, 713 aa, Entamoeba histolytica
  REGION 1..713: note = EHI_178960
MQFEPLFNPKSVAVIGASDRKESVGYAVMNNMIKGGYKGNLYPVGRKPELFGKKCYAKIGQIEEKVDLAVIAIPAKFVPGVCIECGEAGVKGLIIITAGFAEAGEEGKKMCIEIQATCQKYNMRMIGPNCLGIINPRDGVNASFASVMPEAGGVAFISQSGALCTAILDWAANQHVGFSYFVSIGSSIDTDYADLFEFFAKDPKVTSILMYIESIKDAKKFVLRAREFAADKPIILLKAGKSSEGAAAAMSHTGSLAGNDAVYDAVFDRCGCIRVDSICDLWDCAHVLATQNIPQNNRLCIITNAGGPGVISTDRLVSVHGHLAKLSESTMNELNAFLSPFWSHSNPVDVLGDATAGVYQKTLDIVIKDPQIDGVVVVLTPQAMTDPVAVAKSLVEHGPYQKPVLASWMGQSEVEAGVKILEEGKIPNFETPERAVTAFGYIMRHPDIAAKLKEIPKYLDVQVDYEGAKKLIADVVADGRTTFTEYEGKMMFSKYGIPIKGMAKASTEDEAVAEAMKIGTPVVMKILSPDIMHKTDVGGVKVKLTTEEEIRKAYRDIMTSVKEKKPEARIHGVLLEKMVGFKYECIIGCKKDPLFGPVIVFGMGGVTVELYKDTNIALPPIGLQEADRLIDGTKISKLLRGYRGMPACDVEGLKKILVQFSKMIMDFPEISEVDINPLAVSYEEFLVLDAKIVLDKNMIGKEVPKYSHLVIQP

SEQ ID NO: 15 - protein, 301 aa, Clostridium acetobutylicum
  REGION 1..301: note = Ptb
MIKSFNEIIMKVKSKEMKKVAVAVAQDEPVLEAVRDAKKNGIADAILVGDHDEIVSIALKIGMDVNDFEIVNEPNVKKAALKAVELVSTGKADMVMKGLVNTATFLRSVLNKEVGLRTGKTMSHVAVFETEKFDRLLFLTDVAFNTYPELKEKIDIVNNSVKVAHAIGIENPKVAPICAVEVINPKMPSTLDAAMLSKMSDRGQIKGCVVDGPLALDIALSEEAAHHKGVTGEVAGKADIFLMPNIETGNVMYKTLTYTTDSKNGGILVGTSAPVVLTSRADSHETKMNSIALAALVAGNK

SEQ ID NO: 16 - protein, 355 aa, Clostridium acetobutylicum
  REGION 1..355: note = Buk
MYRLLIINPGSTSTKIGIYDDEKEIFEKTLRHSAEEIEKYNTIFDQFQFRKNVILDALKEANIEVSSLNAVVGRGGLLKPIVSGTYAVNQKMLEDLKVGVQGQHASNLGGIIANEIAKEINVPAYIVDPVVVDELDEVSRISGMADIPRKSIFHALNQKAVARRYAKEVGKKYEDLNLIVVHMGGGTSVGTHKDGRVIEVNNTLDGEGPFSPERSGGVPIGDLVRLCFSNKYTYEEVMKKINGKGGVVSYLNTIDFKAVVDKALEGDKKCALIYEAFTFQVAKEIGKCSTVLKGNVDAIILTGGIAYNEHVCNAIEDRVKFIAPVVRYGGEDELLALAEGGLRVLRGEEKAKEYK

SEQ ID NO: 17 - protein, 267 aa, Meiothermus ruber
  REGION 1..267: note = Short-chain-enoyl-CoA hydratase
MAQTFELELPEFEYLTYRVEDGMGIVTISRPQALNALNQDLLIELASVTEVITQDPEVKVAIFTGEGKAFVAGADIAQIASLQDVFAAREFAIMGQSVFNEIAALPVPSIAAINGYALGGGLELALACDLRVASRNAKLGLPEVGLGIIPGFGGTQRLPRLVGRGRALDLIFTGRHVPAEEALGLGLVNRVGEDALQTAKELAAAILKNGPVALALAKEAVGRGENLDLQEALEVEADLFGLACATQDMREGTRAFLEKRPAQFKGE

SEQ ID NO: 18 - protein, 651 aa, Metallosphaera sedula
  REGION 1..651: note = Short-chain-enoyl-CoA hydratase
MKVTVIGSGVMGHGIAELAAIAGNEVWMNDISTEILQQAMERIKWSLSKLRESGSLKEGVEQVLARIHPETDQAQALKGSDFVIEAVKEDLELKRTIFRNAEAHASPSAVLATNTSSLPISEIASVLKSPQRVVGMHFFNPPVLMPLVEIVRGKDTSDEVVKTTAEMAKSMNKETIVVKDVPGFFVNRVLLRIMEAGCYLVEKGIASIQEVDSSAIEELGFPMGVFLLADYTGLDIGYSVWKAVTARGFKAFPCSSTEKLVSQGKLGVKSGSGYYQYPSPGKFVRPTLPSTSKKLGRYLISPAVNEVSYLLREGIVGKDDAEKGCVLGLGLPKGILSYADEIGIDVVVNTLEEMRQTSGMDHYSPDPLLLSMVKEGKLGRKSGQGFHTYAHEEAKYSTIVVRVEPPLAWIVLNRPTRYNAINGDMIREINQALDSLEEREDVRVIAITGQGRVFSAGADVTEFGSLTPVKAMIASRKFHEVFMKIQFLTKPVIAVINGLALGGGMELALSADFRVASKTAEMGQPEINLGLIPGGGGTQRLSRLSGRKGLELVLTGRRVKAEEAYRLGIVEFLAEPEELESEVRKLANAIAEKSPLAVASAKLAYKLGEETHIWTGTSLEASLFGLLFSTKDFEEGVRAFLEKRKPNFRGE

SEQ ID NO: 19 - protein, 261 aa, Clostridium acetobutylicum
  REGION 1..261: note = Short-chain-enoyl-CoA hydratase
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 20 - protein, 658 aa, Ferroglobus placidus
  REGION 1..658: note = 3-hydroxybutyryl-CoA dehydratase
MEVKNIAVLGAGAMGHAIAELAAVSGFNVKIRDIKEEILKNAMERIKQSLEKDFKKGRLKEDPQAVLSRITATLDLKEAVEDADMIIEAIPEIMDLKKQVFAECEEYCREDTIIATNTSSLSITELSKALKKPERFIGLHFFNPPKVMRLVEIVWGEKTSEETVKITEEVARKMNRVIIHVRKDVPGFVVNRIFVTMANEAAWALEKGEGSVEEIDSAVKYRMGLPMGLFELHDLLGGGCIDVSYHVLEYFRQTLGESYRPAPPFERLFKAGHLGKKSGKGFYDWSEGKSNEVPLRAGANFDLLRLIAPAVNEAAWLIEKEVATAEEIDLGVIHGLNYPRGLLRMADDIGIDKIVEKLEKLYEEYGEERYKVNPVLQKMVEENKLGRKTGEGFYKYGRGLYEFVKVEKPKENVAVIKLNRPQRANALNETFLKELEDALEWLEEDENVRCIVITGEGRNFCAGADIAVFAEGKVERMFEFSQLGQKVFNKIEKLSKPVIAAVNGAAMGGGFELALACDLRVVSSRAILALPELNLGIFPGWGGTQRLALAVGVSKAKQLIFMRENIDAKTAYDLGIANYIAEENEFWDKVMEVAEKIAEGPPLAYKFTKRVMFHGIKPELEASLFMESAAGGDIVLSDDVAEGIQAFSYRRKPNFKGR

SEQ ID NO: 21 - protein, 290 aa, Rattus norvegicus
  REGION 1..290: note = Enoyl-CoA hydratase
MAALRALLPRACNSLLSPVRCPEFRRFASGANFQYIITEKKGKNSSVGLIQLNRPKALNALCNGLIEELNQALETFEEDPAVGAIVLTGGEKAFAAGADIKEMQNRTFQDCYSGKFLSHWDHITRIKKPVIAAVNGYALGGGCELAMMCDIIYAGEKAQFGQPEILLGTIPGAGGTQRLTRAVGKSLAMEMVLTGDRISAQDAKQAGLVSKIFPVETLVEEAIQCAEKIANNSKIIVAMAKESVNAAFEMTLTEGNKLEKKLFYSTFATDDRREGMSAFVEKRKANFKDH

SEQ ID NO: 22 - protein, 133 aa, Photobacterium profundum
  REGION 1..133: note = Palmitoyl-CoA hydrolase
MSKIYHHPVQIYYEDTDHSGVVYHPNFLKYFERAREHVIDSDKLATLWNDHGLGFAVYKANMIFQDGVEFAEICDIRTSFTLDGKYKTLWRQEVWRPGASRAAVIGDIEMVCLDKEKRLQPVPADILASMMAD

SEQ ID NO: 23 - protein, 286 aa, Escherichia coli; identical to SEQ ID NO: 8
  REGION 1..286: note = TesB

SEQ ID NO: 24 - protein, 132 aa, Escherichia coli
  REGION 1..132: note = YciA
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 25 - protein, 258 aa, Bacillus subtilis
  REGION 1..258: note = FabI
MNFSLEGRNIVVMGVANKRSIAWGIARSLHEAGARLIFTYAGERLEKSVHELAGTLDRNDSIILPCDVTNDAEIETCFASIKEQVGVIHGIAHCIAFANKEELVGEYLNTNRDGFLLAHNISSYSLTAVVKAARPMMTEGGSIVTLTYLGGELVMPNYNVMGVAKASLDASVKYLAADLGKENIRVNSISAGPIRTLSAKGISDFNSILKDIEERAPLRRTTTPEEVGDTAAFLFSDMSRGITGENLHVDSGFHITAR

SEQ ID NO: 26 - protein, 250 aa, Bacillus subtilis
  REGION 1..250: note = FabL
MEQNKCALVTGSSRGVGKAAAIRLAENGYNIVINYARSKKAALETAEEIEKLGVKVLVVKANVGQPAKIKEMFQQIDETFGRLDVFVNNAASGVLRPVMELEETHWDWTMNINAKALLFCAQEAAKLMEKNGGGHIVSISSLGSIRYLENYTTVGVSKAALEALTRYLAVELSPKQIIVNAVSGGAIDTDALKHFPNREDLLEDARQNTPAGRMVEIKDMVDTVEFLVSSKADMIRGQTIIVDGGRSLLV

SEQ ID NO: 27 - protein, 256 aa, Staphylococcus aureus
  REGION 1..256: note = FabI
MLNLENKTYVIMGIANKRSIAFGVAKVLDQLGAKLVFTYRKERSRKELEKLLEQLNQPEAHLYQIDVQSDEEVINGFEQIGKDVGNIDGVYHSIAFANMEDLRGRFSETSREGFLLAQDISSYSLTIVAHEAKKLMPEGGSIVATTYLGGEFAVQNYNVMGVAKASLEANVKYLALDLGPDNIRVNAISASPIRTLSAKGVGGFNTILKEIEERAPLKRNVDQVEVGKTAAYLLSDLSSGVTGENIHVDSGFHAIK

SEQ ID NO: 28 - protein, 313 aa, Porphyromonas gingivalis
  REGION 1..313: note = FabK
MNRICELLGIEHPIISGGMVWCSGWKLASAVSNCGGLGLIGAGSMHPDNLEHHIRSCKAATDKPFGVNVPLLYPEMDKIMEIIMREHVPVVVTSAGSPKVWTAKLKAAGSKVIHVVSSATFARKSEAAGVDAIVAEGFEAGGHNGREETTTLCLIPEVVDAVNIPVVAAGGIASGRAVAAALALGADAVQVGTRFALSEESSAHEDFKAHCRRSVEGDTMLSLKAVSPTRLLKNKFYQDVFAAEQRGASVEELRELLGRGRAKQGIFEGDLHEGELEIGQAVSQISHAETVAEIMVDLVDGYKRSLAGMPTEI

SEQ ID NO: 29 - protein, 324 aa, Streptococcus pneumoniae
  REGION 1..324: note = FabK
MKTRITELLKIDYPIFQGGMAWVADGDLAGAVSKAGGLGIIGGGNAPKEVVKANIDKIKSLTDKPFGVNIMLLSPFVEDIVDLVIEEGVKVVTTGAGNPSKYMERFHEAGIIVIPVVPSVALAKRMEKIGADAVIAEGMEAGGHIGKLTTMTLVRQVATAISIPVIAAGGIADGEGAAAGFMLGAEAVQVGTRFVVAKESNAHPNYKEKILKARDIDTTISAQHFGHAVRAIKNQLTRDFELAEKDAFKQEDPDLEIFEQMGAGALAKAVVHGDVDGGSVMAGQIAGLVSKEETAEEILKDLYYGAAKKIQEEASRWTGVVRND

SEQ ID NO: 30 - protein, 380 aa, Saccharomyces cerevisiae
  REGION 1..380: note = ETR1
MLPTFKRYMSSSAHQIPKHFKSLIYSTHEVEDCTKVLSVKNYTPKQDLSQSIVLKTLAFPINPSDINQLQGVYPSRPEKTYDYSTDEPAAIAGNEGVFEVVSLPSGSSKGDLKLGDRVIPLQANQGTWSNYRVFSSSSDLIKVNDLDLFSAATVSVNGCTGFQLVSDYIDWNSNGNEWIIQNAGTSSVSKIVTQVAKAKGIKTLSVIRDRDNFDEVAKVLEDKYGATKVISESQNNDKTFAKEVLSKILGENARVRLALNSVGGKSSASIARKLENNALMLTYGGMSKQPVTLPTSLHIFKGLTSKGYWVTEKNKKNPQSKIDTISDFIKMYNYGHIISPRDEIETLTWNTNTTTDEQLLELVKKGITGKGKKKMVVLEW

SEQ ID NO: 31 - protein, 397 aa, Burkholderia mallei
  REGION 1..397: note = FabV
MIIKPRVRGFICVTTHPAGCAASVREQIAYVARRGPIERGPKKVLVIGASTGYGLAARIAAAFGVGAATLGVFFERAPADAKPGTAGWYNSAAFHDEAAARGLQATSVNGDAFSDEIKHKTIDAIRRDLGQVDLVVYSVAAPRRTHPKTGVTHQSTLKPIGHAVRLRGIDTDNEAIKETLLQPATPDEIADTVAVMGGEDWRMWIDALDAAGVLADGAKTTAFTYLGEQVTHDIYWNGSIGEAKKDLDRTVLALRGKLAARGGDARVSVLKAVVTQASSAIPMMPLYLSLLFKVMKARGTHEGCIEQVDGLLRDSLYSAQPHVDAEGRLRADRLELDPAVQARVLELWDQVTDDNLYTLTDFAGYKAEFLRLFGFGIDGVDYDAPVEPNVRIPNLIE

SEQ ID NO: 32 - protein, 398 aa, Pseudomonas aeruginosa
  REGION 1..398: note = FabV
MIIKPRVRGFICVTTHPAGCEANVKQQIDYVEAKGPVVNGPKKVLVIGSSTGYGLAARITAAFGSGADTLGVFFERPGSESKPGTAGWYNSAAFEKFAHEKGLYARSINGDAFSDEVKRLTIETIKRDLGKVDLVVYSLAAPRRTHPKSGEVFSSTLKPIGKSVSFRGLDTDKEVIKDVVLEAASDQEVADTVAVMGGEDWQMWIDALLEADVLADGAKTTAFTYLGEKITHDIYWNGSIGAAKKDLDQKVLGIRDKLAPLGGDARVSVLKAVVTQASSAIPMMPLYLSLLFKVMKEQGTHEGCIEQVDGLYRESLYGAEPRLDEEGRLRADYKELQPEVQSRVEELWDKVTNENLYELTDFAGYKSEFLNLFGFEVAGVDYEQDVNPDVQIANLIQA

SEQ ID NO: 33 - protein, 401 aa, Syntrophomonas wolfei
  REGION 1..401: note = FabV
  source 1..401: note = subsp. Wolfei
MIIKPKIRGFICTTTHPVGCEANVKEQIAYTKAQGPIKNAPKRVLVVGSSSGYGLSSRIAAAFGGGAATIGVFFEKPGTDKKPGTAGFYNAAAFDKLAHEAGLYAKSLNGDAFSNEAKQKAIELIKQDLGQIDLVVYSLASPVRKMPDTGELVRSALKPIGETYTSTAVDTNKDVIIEASVEPATEQEIADTVTVMGGQDWELWIQALEEAGVLAEGCKTVAYSYIGTELTWPIYWDGALGRAKMDLDRAATALNEKLAAKGGTANVAVLKSVVTQASSAIPVMPLYIAMVFKKMREQGVHEGCMEQIYRMFSQRLYKEDGSAPEVDDHNRLRLDDWELRDDIQQHCRDLWPQITTENLRELTDYDMYKEEFIKLFGFGIEGIDYDADVNPEVEFDVIDIE

SEQ ID NO: 34 - protein, 265 aa, Pseudomonas aeruginosa
  REGION 1..265: note = FabI
MGFLTGKRALIVGVASKLSIASGIAAAMHREGAELAFTYQNDKLRGRVEEFASGWGSRPELCFPCDVADDSQIEAVFAALGKHWDGLDIIVHSVGFAPGDQLDGDFTAVTTREGFRIAHDISAYSFIALAKAGREMMKGRNGSLLTLSYLGAERTMPNYNVMGMAKASLEAGVRYLAGSLGAEGTRVNAVSAGPIRTLAASGIKSFRKMLAANERQTPLRRNVTIEEVGNAGAFLCSDLASGISGEILYVDGGFNTTAMGPLDDD

SEQ ID NO: 35 - protein, 263 aa, Burkholderia pseudomallei
  REGION 1..263: note = FabI
MGFLDGKRILLTGLLSNRSIAYGIAKACKREGAELAFTYVGDRFKDRITEFAAEFGSELVFPCDVADDAQIDALFASLKTHWDSLDGLVHSIGFAPREAIAGDFLDGLTRENFRIAHDISAYSFPALAKAALPMLSDDASLLTLSYLGAERAIPNYNTMGLAKAALEASVRYLAVSLGAKGVRVNAISAGPIKTLAASGIKSFGKILDFVESNSPLKRNVTIEQVGNAGAFLLSDLASGVTAEVMHVDSGFNAVVGGMAGLEE

SEQ ID NO: 36 - DNA, 591 bp, Escherichia coli
  misc_feature 1..591: note = paaY
  source 1..591: mol_type = genomic DNA
atgccaatttatcagatagacggtctgactccggttgtgccagaagagagttttgtccatccgacagcggtattgatcggcgatgttattctcggcaagggcgtttacgttgggccaaatgccagcctgcgtggcgattttggtcgtatcgtggtgaaagatggcgcgaacattcaggataattgcgttatgcacggttttcccgagcaggatactgttgtaggagaagatggacatattggtcatagcgctatccttcacggctgcattatccgccgcaatgcattagtgggaatgaacgcggtagtgatggacggtgcggtgattggcgagaacagcattgttggtgcatccgcatttgtgaaagccaaagcagaaatgccagctaattacctgattgtcggcagcccggcgaaagcgattcgtgaactcagtgagcaggagttggcatggaaaaagcagggtacgcatgagtaccaggtgctggtgacacgctgtaagcagacgttacatcaagtcgagccattgcgggaaattgaacctggcaggaaacgcctggtatttgatgagaatctgcgaccgaaacagtaa

SEQ ID NO: 37 - DNA, 423 bp, Escherichia coli
  misc_feature 1..423: note = paaI
  source 1..423: mol_type = genomic DNA
atgagtcataaggcctggcaaaatgcccatgcaatgtatgagaacgatgcctgcgccaaagcgcttggcatcgacattatctcaatggatgaaggctttgctgtagtgaccatgaccgtcactgcacaaatgcttaacggtcatcaaagttgccacggcgggcagctattttcactggctgatactgcctttgcctacgcctgcaatagccaggggctggcagccgtcgcttctgcctgcacgattgattttttgcgtccaggctttgccggagacaccttaactgctactgcgcaggtacgtcatcagggcaagcaaaccggtgtttacgacatcgaaattgttaaccaacaacaaaaaacggttgcgctgtttcgcggtaaatctcaccgcatcggcggcaccattacaggagaagcctga

SEQ ID NO: 38 - DNA, 627 bp, Escherichia coli
  misc_feature 1..627: note = tesA
  source 1..627: mol_type = genomic DNA
atgatgaacttcaacaatgttttccgctggcatttgcccttcctgttcctggtcctgttaaccttccgtgccgccgcagcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgcctgcaaactatggtcgccgttataatgaagcctttagcgccatttaccccaaactcgccaaagagtttgatgttccgctgctgcccttttttatggaagaggtctacctcaagccacaatggatgcaggatgacggtattcatcccaaccgcgacgcccagccgtttattgccgactggatggcgaagcagttgcagcctttagtaaatcatgactcataa

SEQ ID NO: 39 - DNA, 399 bp, Escherichia coli
  misc_feature 1..399: note = yciA
  source 1..399: mol_type = genomic DNA
atgtctacaacacataacgtccctcagggcgatcttgttttacgtactttagccatgcccgccgataccaatgccaatggtgacatctttggtggttggttaatgtcacaaatggatattggcggcgctattctggcaaaagaaattgcccacggtcgcgtagtgactgtgcgggttgaaggaatgactttcttacggccggttgcggtcggcgatgtggtgtgctgctatgcacgctgtgtccagaaagggacgacatcggtcagcattaatattgaagtgtgggtgaaaaaagtagcgtctgaaccaattgggcaacgctataaagcgacagaagcattatttaagtatgtcgcggttgatcctgaaggaaaacctcgcgccttacctgttgagtaa

SEQ ID NO: 40 - DNA, 414 bp, Escherichia coli
  misc_feature 1..414: note = entH
  source 1..414: mol_type = genomic DNA
atgatctggaaacgccatttaacgctcgacgaactgaacgccaccagcgataacacaatggtggcgcatctgggaattgtgtatacccgtctgggcgatgatgtgctggaagccgaaatgccggttgatacccgtactcatcagccgttcggtttactacatggcggcgcgtcggcggcgctggcggaaacgctgggatcgatggccggatttatgatgacccgcgacggacagtgtgtggtaggcacagaacttaatgcaacacaccatcgcccggtgtctgagggaaaggtacgcggcgtctgccagccgctgcatcttggtcggcaaaatcagagctgggaaatcgtcgttttcgatgaacaggggcggcgttgctgcacttgtcggctgggtacggcagttttgggatga

SEQ ID NO: 41 - DNA, 861 bp, Escherichia coli
  misc_feature 1..861: note = tesB
  source 1..861: mol_type = genomic DNA
atgagtcaggcgctaaaaaatttactgacattgttaaatctggaaaaaattgaggaaggactctttcgcggccagagtgaagatttaggtttacgccaggtgtttggcggccaggtcgtgggtcaggccttgtatgctgcaaaagagaccgtccctgaagagcggctggtacattcgtttcacagctactttcttcgccctggcgatagtaagaagccgattatttatgatgtcgaaacgctgcgtgacggtaacagcttcagcgcccgccgggttgctgctattcaaaacggcaaaccgattttttatatgactgcctctttccaggcaccagaagcgggtttcgaacatcaaaaaacaatgccgtccgcgccagcgcctgatggcctcccttcggaaacgcaaatcgcccaatcgctggcgcacctgctgccgccagtgctgaaagataaattcatctgcgatcgtccgctggaagtccgtccggtggagtttcataacccactgaaaggtcacgtcgcagaaccacatcgtcaggtgtggatccgcgcaaatggtagcgtgccggatgacctgcgcgttcatcagtatctgctcggttacgcttctgatcttaacttcctgccggtagctctacagccgcacggcatcggttttctcgaaccggggattcagattgccaccattgaccattccatgtggttccatcgcccgtttaatttgaatgaatggctgctgtatagcgtggagagcacctcggcgtccagcgcacgtggctttgtgcgcggtgagttttatacccaagacggcgtactggttgcctcgaccgttcaggaaggggtgatgcgtaatcacaattaa

SEQ ID NO: 42 - DNA, 399 bp, Escherichia coli
  misc_feature 1..399: note = fadM
  source 1..399: mol_type = genomic DNA
atgcaaacacaaatcaaagttcgtggatatcatctcgacgtttaccagcacgtcaacaacgcccgctaccttgaatttctcgaagaagcccgctgggatgggttggaaaatagcgacagttttcagtggatgacggcccataacatcgccttcgtcgtggtcaatatcaatattaactatcgtcgcccagcggtattaagtgacctgttaactattaccagtcagttgcagcaattaaacggtaaaagcggcatcttaagccaggtcattacactggagccggaagggcaggtggtagcggatgcgcttattacgtttgtttgtattgatcttaaaacgcagaaagcattagctctggaaggggaattgcgcgaaaagctggagcagatggttaagtaa

SEQ ID NO: 43 - DNA, 411 bp, Escherichia coli
  misc_feature 1..411: note = menI
  source 1..411: mol_type = genomic DNA
atgatatggaaacggaaaatcaccctggaagcactgaatgctatgggtgaaggaaacatggtggggttcctggatattcgctttgaacatattggtgatgacacccttgaagcgacaatgccagtagactcgcggacaaagcagcctttcgggttgctgcatggaggagcatccgtggtactggccgaaagtatcggttccgttgccggttatttatgtaccgaaggtgagcaaaaagtggttggtctggaaatcaatgctaaccacgtccgctcggcacgagaagggcgggtgcgcggcgtatgcaaaccgttgcatctcggttcgcgtcaccaggtctggcagattgaaatcttcgatgagaaagggcgtttgtgctgttcgtcacgattgacgaccgccattttgtga

SEQ ID NO: 44 - protein, 629 aa, Syntrophomonas wolfei
  REGION 1..629: note = Tneu_0420
  source 1..629: note = subsp. Wolfei
MSAEFVEVYRKSLEDPIGFWEKQAERLYWRERWEKTYDDSNPPFYRWFVGGKTNISYNALDRHVKGGRANKAALIWVSADGATRVLRYWDLYREVNRFAVLLKSLGVERGDRVAIYMPMIPEAMVAMLAVNRIGAVHTVVFSGFGPQALAERIKDAEAKVVITADGMRRRGRVIPLKPTVDEALKIVGNDIFTVVYKHTGVEVPMKQGRDLWWQEEIAKIPPNTYIEPEWVPGEAPLFILYTSGTTGKPKGILHLHGQYMVWIWYAFNHLTGAERDFREDIVFFSTADIGWISGHHYGVHGPLLNGLTVLWYEDAPDYPHPGIWWEIADTYKVTHMLFSPTAIRLLMKYGDEWPRRYKLDSIMALYPTGEVLNEEAYNWMRREVCRGRPDCQIADIWGQTETACFVTAPGSMNLGGFRYKYGSVGMPYPTLNLQILDDDGKPLPPGAKGHVVAKPPLPPAFLHTLWRDPERYVKSYWSRFPGYYYTGDLGYIDQDGHLHIMGRSDDVIKVAGHRLSTREVEDILTSHPAVAEAAVVGVPDEVRGEVLGVFVVPKQGMKITEEEVVKHLRNSLGPVAVIGKVAILDKLPKTRTGKVMRRVLRAMATGQPVGDLSTLEDEEALEELRKKLG

SEQ ID NO: 45 - protein, 542 aa, Syntrophomonas wolfei
  REGION 1..542: note = SAR11_0248
  source 1..542: note = subsp. Wolfei
MSHYDTNLDKNEANYVPLSPLTFLERTKDIYPNYEALVYESRSYTWEEVYKRCVKFASALDKLGVKTGDTVSIMAFNTPEIFEAHYSIPMVGAVINAINTRLDPNTISYILQHSDAKVLIVDRQFHDVIEKALKNVKNKITIIDIDDQDIDTSSFKRIGELEYESFLNTGNENYEWKKPKDEWEAISLGYTSGTTGNPKGVVYHHRGSYLMATGSVTAWNMPNKLNFLCVVPMFHCNGWCYPWTLAMLHARVICLRNIDVKKMFELIDKYEVTHFGGAPIVLNMIVNAPKEDQKALKRKVNVLTAGAPPPSIIFEKMENLGFEVMHVYGLTETYGHMLQCAWNDDWNSLEKDKKNEIKARQGVRYPNTEGAIVMDPETMKPVPKDGKTMGEIMIRGNIVMKGYYKDKEATDKSMAGGWFHSGDLAVTHPDGYIKIQDRSKDIIISGGENISSIEIENAIAKHPSVSLAAVVAKPDEKWGETPCAFVELIKDKPATEKEIIDFCRETLAGFKLPKSVIFCDLPKTSTGKIQKFELRKKVKELS

SEQ ID NO: 46 - protein, 542 aa, Ruegeria pomeroyi
  REGION 1..542: note = SPO0677
MTGSTIYDQHLDRTPANFTPLSPLSYIERTAAVYPDYPSVVYGDRRYTWAETYTRCRRLASALAGRGLGKGDTVSIIAANIPEMYEAHFGVPMAGAVLNAINTRLDAPIIAFILSHAEARVLIVDPEFSEVVRDALAQIDRPDLLVVDIEDPSFAGGAPVGTLSYDALLAEGDPDFDWSLPGDEWDAIALNYTSGTTGNPKGVVYHHRGAALNATSNILTWGMPQHAVYLWTLPMFHCNGWCFPWTMAANAGVSVCLRAVRDEPIYRAFREEKVTHFCGAPIVLNMLANAPDHMKDFDHQIKVMTAGAPPPAAVIEKMEAMGVDVTHVYGLTETYGPSVVCAWKEEWDGRPGAERAALKVRQGVRYVALSGLMVADPETLEPVPADGETMGEIFMQGNIVMKGYLKNPDATDRAFRGGWFASGDLGVMHPDGYIALKDRSKDIIISGGENISSVEVEDVLYKHPAVMEAAVVARPDEKWGETPCAFVELKPGQSVEAADLIAHCRANMAHFKAPKTVVFGELPKTSTGKIQKFLLRERARAL

SEQ ID NO: 47 - protein, 539 aa, Ruegeria pomeroyi
  REGION 1..539: note = SPO2045
MLGQMMYQPLLISSLIDHAARYHGEAQIWSVSTEGGVEETNWAGIADNARRLGSVLTDAGLAPQSRVATLAWNNRRHLEIYYGVSGAGFVLHTINPRLFPEQLVYILNHAEDRILFFDATFLPLVEGIRPHLTTVERLVLMGPRDEAAAARIEGLEFYDEFVATGDAGFDWPDLDERTASSLCYTSGTTGNPKGVLYSHRSTVLHSFGSNTRDCIGFSARDVVMPVVPMFHVNAWGTPYACAMSGSCMVLPGPDLHGEALVGLIDRYRVTIALGVPTIWQGLLATARAKGSTLESLTRTVIGGAACPPSMIAEFRDRYGVDTVHAWGMSEMSPLGTTNQPLAKHGALPIEAQHKLRENQGRPPYGVELKIVDDDGNTLPNDGQTQGDLMVRGHWVLDSYFQLQDQPILSDGWFATGDVATLDRDGYMTIRDRSKDIIKSGGEWISSVELENIAVAHPKLATAAVIGVPHPKWDERPLLVAVKAEGETPDEAELLAFFDGKIAKWQVPDRVVFVEALPLNATGKVLKRTLREQFRDVLTG

SEQ ID NO: 48 - protein, 542 aa, Ruegeria lacuscaerulensis
  REGION 1..542: note = SL1157_1815
MATHNIYQRDLDKCPANFTALSPLSYIERTAAVYPDYPAVVYGDRRYSWAQTYARCRRLASALAARGVGTGDTVSIIAANIPEMYEAHFGVPMAGAVLNAINTRLDAPIIAFILNHAESKVLLVDPEFSGVVKQALEQVDHDILVIDIEDPSFDGGEKLGALTYDDLLAEGDPEFDWSLPADEWDAITLNYTSGTTGNPKGVVYHHRGAALNATSNILTWGMPQHSVYLWTLPMFHCNGWCFPWTMAANAGTSVCLRAVRDAPIYRAFRDEKVTHFCGAPIVLNMLANAPDHLKDFGHEIKVMTAGAPPPAAVIAAMEGMGIEVTHVYGLTETYGPSVVCAWKDEWNDKPAEERAALKVRQGVKYVALSGLMVADPETLEPVPADGETMGEIFMQGNIVMKGYLKNPEATRKAFRGGWFASGDLGVMHPDGYIALKDRSKDIIISGGENISSVEVEDILYKHPAVMEAAVVAKPDEKWGETPCAFVELKPGAEATEADLIAFCRDHMAHFKAPKTVVFGPLPKTSTGKIQKFKLREQARALG

SEQ ID NO: 49 - protein, 539 aa, Ruegeria lacuscaerulensis
  REGION 1..539: note = SL1157_2728
MLGQMMTQPLLISSLIDHAARYHGQTEIVSVETDGTVTRTNWGEIAANARRMGSALTKLGLQPQDRIGTLAWNNRRHLEIYYAASGAGFVCHTINPRLFPEQLVYIINHAQDRVLFFDATFLPLVAAIRDQLTEVKHFVLMGPRNEDALQQIPGLEFYDELIETGDTDFEWPVFDENTASSLCYTSGTTGHPKGVLYSHRSTVLHSFASNTRDVIGYSAMDVVMPVVPMFHVNAWGSPYGCAMSGAQMVLPGPDLHGEALVNLIDTYGVTLAMGVPTIWQGLLAHAAKCGTKLESLERTVIGGAACPPSMIATFREKYGVDTVHAWGMSEMSPLGTANIPLAKHRKLPIEEQHKLRENQGRPPFGVELKIVDDDGNDLPHDGVTQGDLMVRGHWVLDSYFQLKDQELLQDGWFATGDVATLDPDGYMTIRDRSKDIIKSGGEWISSVELENIAVAHPKLATAAVIGVPHPKWDERPLLVAVKAEGEDPSEAELLEFFDGKIAKWQVPDKVVFVDALPLNATGKVLKRKLRDEFKDALTG

SEQ ID NO: 50 - protein, 540 aa, Pseudomonas aeruginosa
  REGION 1..540: note = PA4198
MSIFEQGLAPAAVNHIALTPLSFIERTASVYPHYPAVIHGNIRRDWAQTYQRCRRLASALAGRGIGQGDTVAVMLPNIPEMLEAHYGVPMIGAVLNTLNVRLDAEAIAFMLQHGEAKVLITDYEFHEVIHAAIGMLDHPPLVVDVNDPEYGEGRPVGELDYEALLAEGDPQFAWEWPRDEWQAISLNYTSGTTGNPKGVVYHHRGAYLNALGNQMTWSMGQHPVYLWTLPMFHCNGWCYPWTVTALAGVHVCLRRVDPQKVLTLIREHQVSHLCGAPIVLNALINMPDSAKAAIDHPVHAMVAGAAPPAKVIGAVEEMGIRVTHVYGLTEVYGPVTVCAWHGEWDDLPLERRAAIKSRQGVRYPTLEGVMVADPKTLEPVPRDGQSIGEIFMRGNTVMKGYLKNPSATEEAFAGGWFHTGDLAVCHPDGYIEIRDRLKDIIISGGENISTIELEGVLYRHPAVLEAAVVARPDEKWGETPCAFITLKSDHQGLAESEIVAFCREHLAAFKIPRTVVFSELPKTSTGKIQKYVLREWAAAL

SEQ ID NO: 51 - protein, 589 aa, Burkholderia thailandensis
  REGION 1..589: note = BTH_I2141
MLLTGKLADIVSPRRAGRSKPPARRRAVCSRPYRTNTSGRRKTMGKPLLGQMMDMPLLVSSLIAHAARHAGDVEIVSRRVEGDIHRYTYRDCEARSKRLAQALTRLGVGVGDRIGTLAWNGYRHVEAYYGISGMGAVCHTINPRLFPEQIAYIVNHAEDRYVLFDLTFAPLVDQLAPQCPNVKGWIAMTDDAHLPRGATPYLCYETLVGAQDGDYAWPLLDERQASSLCYTSGTTGHPKGALYSHRSTVLHAYGAALPDAMGLSSRDAALPVVPMFHVNAWGLPYTAALTGTKLVLPGKDLDGKSLYELIESERVTFSAGVPTVWLGLLAYMREAGVRFSTLDRTVIGGSACPPSMLETFEDVYDVRVIHAWGMTELSPLGTLSKLNWAQSQRSVDEQRRLLEKQGRVIYGIDMRIVGDDGRELPWDGVAFGDLQVCGPWVIDRYFGIDASPLVDGWFPTGDVATIDADGFLQITDRSKDVIKSGGEWISSIDVENVAVAHPAVAEAACIACAHPKWTERPLLVVVKRAGMDVTRGELLAFYDGKVAKWWIPDDVVFVDALPHTATGKLQKLKLREQFRDHVLPTAVDA

SEQ ID NO: 52 - protein, 501 aa, Streptomyces sp.
  REGION 1..501: note = UbiD-like decarboxylase
MYVQMVFVKEVPVQHPSDLREHIEALEKLGDLDRVHDEVDWNLEAAAQTRYSTEHHLPAPLFENVAGVAEGFRLLGAPAALSSDPSRPYARVALSVGLRPEATGREVVEHLVAARHRPGVPPVAVAAEAAPVKANVLLGDEADLNRFPVPFVHEGDGNRYANTYGVIIAQTPDGSWTNWSIARIMMIDGKHMTGLVMHPQHIAQVWQQWADLGKPMPYALVQGGDPAIPYVGGIPIGDGVAESAYIGALIGRPLEVVKAELSDLMVPAGAEIVIEGHLSVQRDGVEGPFGEFAGYIPRETSLQPVYTVEAITHRDAPIWPLVAEGKPTDDFHTVTGIGEAAGALDAIREAGLPAASAWAPLSAASHWLVVTAPGNWRELLPGVSEEQYARRVGEAVFGTKFGSCLPQVFLLDDDFDPTDDADLLWALATRVHPDGRVVRFEDGPVLPLLTCYTPQERHAARATKVVHEALLSAPGEREPQSTFADAYPAEVRAKVRARYPN

SEQ ID NO: 53 - protein, 490 aa, Yersinia frederiksenii
  REGION 1..490: note = UbiD-like decarboxylase
MFLKSLREQINLLKRQGDLRIINRPVDSYLEAAAIIRRSTETKAPVPLMTNIKGYPGCSIVGGLAALSSNPEYPLSRVAMSLGLSTTASAQSIVQYLIDGLKKTPYLPVEIDSNSAPCKQNILRGEDASLAHFPIPQVHQHDGNRYVNTWGVFIVESPDGQWCNWSIQRVQYLNDRQMIALVFPSQHISDIWEEWVKIGKPMPYALVQGCEPIVPYIAGLPLLERNVNEAEYIGALYGKGIEVVKCETHDLRVPASSEVVIEGYFAITREGVEGPFGEFAGYIPNENSLQPILSITAISWQDDPIWPVVAEGKPVDEYHTCSGIGDAAAIQNVLLTANLPVSFVWVPLYSACHLMIVSINRHWRQTHPDLTSEQLTKKVGDVIHQTRNSIKLPKIVVLDDDVDPADTESLLWALATRIHPERKRYFYESCILPLLSCYSEEERHNRKGKRVIIDALLPENHGAISSFDYAYPDDIKQRVLMHWETDFSHD

SEQ ID NO: 54 - protein, 263 aa, Acidaminococcus fermentans
  REGION 1..263: note = Electron transfer flavoprotein alpha/beta-subunit
MNIVVCVKQVPDTAEMKIDPVTNNLVRDGVTNIMNPYDQYALETALQLKDELGAHVTVITMGPPHAESVLRDCLAVGADEAKLVSDRAFGGADTLATSAAMANTIKHFGVPDLILCGRQAIDGDTAQVGPEIAEHLGLPQVTAALKVQVKDDTVVVDRDNEQMSMTFTMKMPCVVTVMRSKDLRFASIRGKMKARKAEIPVYTAAALEIPLDIIGKAGSPTQVMKSFTPKVTQVHGEIFDDEDPAVAVDKLVNKLIEDKIITK

SEQ ID NO: 55 - protein, 524 aa, Syntrophomonas wolfei
  source 1..524: note = subsp. Wolfei
  REGION 1..524: note = SYN_02896
MAKLYPEEFYNSADWFVDRHVREGRGDNICAYTDKGNYTYRDIQKMANKMANMFKDLDIRMGDRIIMLVLDTPWFYSTFWGAVKMGAVPVPSSTMLTPADYEYYLNDSQARTLVVSSRLLPVVNQIEELRFLKNMIVVDDDGVFSTPYQQIYASASDEFQTVFTSADDVAFWLYTSGTTGGPKGAVHSQSDMQYSAEAYGKHILEITEKDICYSAARLFFTYGLGNAMFFPMSVGAAAVLNPDPPAPAHVFRLIKTYKATLFFGVPTLFGQMLITQDKIDAEKGAGADPKDHDLVSLRACPSAGEALPPDLYHKFKARYGVEILDGPGSTEMLHIYLSNKLGDVKPGSSGKPVPGYEEKIMDEEGKNELPDGEVGNLWIKGRSSLRYYWRKRDKTAATVIGEWVNSGDKYYKDAEGYYWPSGRADDMLKVGGIWVSPLEVENCLREHASVMECAVVGAMDEENLVKPKAFVVLNQGFAQSPELEKELKQWVLDRLAKFKYPRWIVFIDSLPKTATGKIQRFKLR

SEQ ID NO: 56 - protein, 524 aa, Syntrophomonas wolfei
  source 1..524: note = subsp. Wolfei
  REGION 1..524: note = SYN_02898
MAVYTEEFYNSVNYFIDRHIEEGRGDKICAYTDKGNYTYRDMQKMVNKMANMFRKLDIRIGDRVIMLVFDTPWFFSTFWGAVRIGAVPVPSNTMLTSDDYQYYLNDSQARTLVISEKLLPLIKGIKGELRYLRDVIVVDDDGEFSTPYQQMYAQASEEAETAFTTKDDVAFWQYTSGTTGAPKGAVHSHSDMQYVAEAYGKHVLGMTENDVCYSAARLFFAYGIGNGMVYPLSVGAASVLNPDPPTPERAFRLNSTYKVTLFFGIPTLFGQMLEYKVKQEKEAGITPDPKAPHELSSVRACPSAGEALPPDLYHRFKERFGVEILDGPGSTEMLHIYLSNTLGDVKAGSSGKVVPGYEAKIVGEEGETLPDGEIGTLWVKGDSSLRYYWRKKEKTASTIIGGWVNTGDKYYRDKDGYFWPSGRADDMLKVGGIWVSPLEVENCLREHPAVLETAVIGAEDEKNLVKPKAFVVLKQGFAPSPELEKELKQWVLDRLAKFKYPRWIVFMDELPKTATGKIQRFKLR

SEQ ID NO: 57 - protein, 560 aa, Pseudomonas aeruginosa
  REGION 1..560: note = PA3924
MLKTRLIPAAAGAYQYPLLIKSLMLSGRRYEKSHEIVYRDQVRYSYATFNERVARLANVLSEAGVKAGDTVAVMDWDSHRYLECMFAIPMIGAVLHTINIRLSPEQILYTMNHAEDRFVLVNSEFVPLYQAVAGQLATVERTILLTDGAEKSAELPGLVGEYESLLAAASPRYDFPDFDENSIATTFYTTGTTGNPKGVYFSHRQLVLHTLAMASTIGSLDSIRLLGTSDVYMPITPMFHVHAWGTPYVATMLGVKQVYPGRYDPELLVELWKREKVTFSHCVPTILQMVMNARAAQGVDFKGWKVIIGGSALNRSLYEAAKARGIQLTAAYGMSETCPLISCAYLNDELLAGSEDERTTYRIKAGVPVPLVDAAIMDEQGRFLPADGESQGELVLRSPWLTQGYFREPERGEELWRGGWMHTGDVATLDGMGFIEIRDRIKDVIKTGGEWLSSLELEDLISRHPAVREVAVVGVPDPQWGERPFALLVVREGQQLDARGLKEHLKPFVEQGNINKWAIPSQIAVVTDIPKTSVGKLDKKRIRIEIAQWQEAGSAFLSTV

(19) United States
(12) Patent Application Publication
(10) Pub. No.: US 2025/0327097 A1
(43) Pub. Date: Oct. 23, 2025

(54) ORGANISMS PRODUCING LESS CROTONIC ACID

(71) Applicant: Global Bioenergies, Evry Cedex (FR)

(72) Inventors: Benoît Villiers, Tours (FR); Denis Thibaut, Paris (FR)

(73) Assignee: Global Bioenergies, Evry Cedex (FR)

(21) Appl. No.: 18/689,282

(22) PCT Filed: Sep. 6, 2022
(86) PCT No.: PCT/EP2022/074752

§ 371 Date: Mar. 5, 2024

(30) Foreign Application Priority Data
Sep. 6, 2021 (EP) 21195149.6

(51) Int. Cl. C12P 5/02; C12N 9/00; C12N 9/02; C12N 9/10; C12N 9/16; C12N 9/88; C12P 7/40

(52) U.S. Cl. CPC C12P 5/026; C12N 9/001; C12N 9/13; C12N 9/16; C12N 9/88; C12N 9/93; C12P 7/40; C12Y 103/01009; C12Y 103/01044; C12Y 208/03008; C12Y 301/0202; C12Y 301/02028; C12Y 402/01017; C12Y 602/01002; C12Y 602/01025; C12Y 602/01027

(57) ABSTRACT

The present invention relates to a recombinant organism or microorganism having a decreased pool of crotonic acid compared to the organism or microorganism from which it is derived due to at least one of: (i) an increased conversion of crotonyl-CoA into butyryl-CoA; and/or an increased conversion of butyryl-CoA into butyric acid; (ii) an increased conversion of crotonyl-CoA into 3-hydroxybutyryl-CoA; and/or an increased conversion of 3-hydroxybutyryl-CoA into 3-hydroxybutyric acid; (iii) an increased conversion of crotonic acid into crotonyl-CoA; (iv) an increased conversion of crotonyl-[acyl-carrier protein] into butyryl [acyl-carrier-protein]; (v) a decreased conversion of crotonyl-CoA into crotonic acid; and/or (vi) a decreased conversion of crotonyl-[acyl-carrier protein] into crotonic acid. Moreover, the present invention relates to the use of such a recombinant organism or microorganism for the production of alkenes with the enzyme ferulic acid decarboxylase. Further, the present invention relates to a method for the production of isobutene or butadiene by culturing such a recombinant organism or microorganism in a suitable culture medium under suitable conditions.

The present invention relates to a recombinant organism or microorganism having a decreased pool of crotonic acid compared to the organism or microorganism from which it is derived due to at least one of: (i) an increased conversion of crotonyl-CoA into butyryl-CoA; and/or an increased conversion of butyryl-CoA into butyric acid; (ii) an increased conversion of crotonyl-CoA into 3-hydroxybutyryl-CoA; and/or an increased conversion of 3-hydroxybutyryl-CoA into 3-hydroxybutyric acid; (iii) an increased conversion of crotonic acid into crotonyl-CoA; (iv) an increased conversion of crotonyl-[acyl-carrier protein] into butyryl [acyl-carrier-protein]; (v) a decreased conversion of crotonyl-[acyl-carrier protein] into crotonic acid; and/or a decreased conversion of crotonyl-CoA into crotonic acid. Moreover, the present invention relates to the use of such a recombinant organism or microorganism for the production of alkenes with the enzyme ferulic acid decarboxylase. Further, the present invention relates to a method for the production of isobutene or butadiene by culturing such a recombinant organism or microorganism in a suitable culture medium under suitable conditions.

### BACKGROUND OF THE INVENTION

A large number of chemical compounds are currently derived from petrochemicals. Alkenes (such as ethylene, propylene, the different butenes, or else the pentenes, for example) are used in the plastics industry, for example for producing polypropylene or polyethylene, and in other areas of the chemical industry and that of fuels. Butylene exists in four forms, one of which, isobutene (also referred to as isobutylene), enters into the composition of methyl-tert-butyl-ether (MTBE), an anti-knock additive for automobile fuel. Isobutene can also be used to produce isooctene, which in turn can be reduced to isooctane (2,2,4-trimethylpentane); the very high octane rating of isooctane makes it the best fuel for so-called “gasoline” engines. Alkenes such as isobutene are currently produced by catalytic cracking of petroleum products (or by a derivative of the Fischer-Tropsch process in the case of hexene, from coal or gas). The production costs are therefore tightly linked to the price of oil. Moreover, catalytic cracking is sometimes associated with considerable technical difficulties which increase process complexity and production costs.

The production by a biological pathway of alkenes such as isobutene is called for in the context of a sustainable industrial operation in harmony with geochemical cycles. The first generation of biofuels consisted in the fermentative production of ethanol, as fermentation and distillation processes already existed in the food processing industry. The production of second generation biofuels is in an exploratory phase, encompassing in particular the production of long chain alcohols (butanol and pentanol), terpenes, linear alkanes and fatty acids. Two recent reviews provide a general overview of research in this field: Ladygina et al. (Process Biochemistry 41 (2006), 1001) and Wackett (Current Opinions in Chemical Biology 21 (2008), 187).

The conversion of isovalerate to isobutene by the yeast Rhodotorula minuta has been described (Fujii et al. (Appl. Environ. Microbiol. 54 (1988), 583)), but the efficiency of this reaction is far from permitting an industrial application. The reaction mechanism was elucidated by Fukuda et al. (BBRC 201 (1994), 516) and involves a cytochrome P450 enzyme which decarboxylates isovalerate by reduction of an oxoferryl group Fe^{v}═O. Large-scale biosynthesis of isobutene by this pathway seems highly unfavourable, since it would require the synthesis and degradation of one molecule of leucine to form one molecule of isobutene. Also, the enzyme catalyzing the reaction uses heme as cofactor, poorly lending itself to recombinant expression in bacteria and to improvement of enzyme parameters. For all these reasons, it appears very unlikely that this pathway can serve as a basis for industrial exploitation. Other microorganisms have been described as being marginally capable of naturally producing isobutene from isovalerate; the yields obtained are even lower than those obtained with Rhodotorula minuta (Fukuda et al. (Agric. Biol. Chem. 48 (1984), 1679)). Gogerty et al. (Appl. Environm. Microbiol. 76 (2010), 8004-8010) and van Leeuwen et al. (Appl. Microbiol. Biotechnol. 93 (2012), 1377-1387) describe the production of isobutene from acetoacetyl-CoA by enzymatic conversions wherein the last step of the proposed pathway is the conversion of 3-hydroxy-3-methylbutyric acid (also referred to as 3-hydroxyisovalerate (HIV)) by making use of a mevalonate diphosphate decarboxylase.

This reaction for the production of isobutene from 3-hydroxy-3-methylbutyric acid is also described in WO2010/001078 which, in general terms, describes methods for generating alkenes through a biological process, in particular methods for producing terminal alkenes (in particular propylene, ethylene, 1-butylene, isobutylene or isoamylene) from molecules of the 3-hydroxyalkanoate type.

WO2012/052427 also describes a method for generating alkenes through a biological process while, in particular, a method for producing alkenes (for example propylene, ethylene, 1-butylene, isobutylene or isoamylene) from molecules of the 3-hydroxyalkanoate type is described. In this context, the reaction for the production of isobutene from 3-hydroxy-3-methylbutyric acid is also described in WO2012/052427.

WO 2016/042012 describes methods for producing said 3-hydroxy-3-methylbutyric acid. In particular, WO 2016/042012 describes methods for producing 3-hydroxy-3-methylbutyric acid comprising the step of enzymatically converting 3-methylcrotonyl-CoA into 3-methylcrotonic acid and the step of enzymatically further converting the thus produced 3-methylcrotonic acid into 3-hydroxy-3-methylbutyric acid.

In Gogerty et al. (loc. cit.) and in van Leeuwen et al. (loc. cit.) the production of 3-hydroxy-3-methylbutyric acid is proposed to be achieved by the conversion of 3-methylcrotonyl-CoA via 3-hydroxy-3-methylbutyryl-CoA. In order to further improve the efficiency and variability of methods for producing isobutene from renewable resources, alternative routes for the provision of isobutene and its precursors have been developed by providing methods for the production of isobutene comprising the enzymatic conversion of 3-methylcrotonic acid (also termed 3-methyl-2-butenoic acid, 3,3-dimethylacrylic acid or senecioic acid) into isobutene.

In particular, in WO 2017/085167, methods for the production of isobutene have been described comprising the enzymatic conversion of 3-methylcrotonic acid into isobutene, wherein the enzymatic conversion of 3-methylcrotonic acid into isobutene is achieved by making use of a prenylated FMN-dependent decarboxylase associated with an FMN prenyl transferase, wherein said FMN prenyl transferase catalyzes the prenylation of a flavin cofactor (FMN or FAD) utilizing dimethylallyl phosphate (DMAP) into a flavin-derived cofactor while these enzymes have artificially been implemented in a pathway which ultimately leads to the production of isobutene. Moreover, in WO 2017/085167, methods have been described, wherein such a method further comprises (a) providing the 3-methylcrotonic acid by the enzymatic conversion of 3-methylcrotonyl-CoA into 3-methylcrotonic acid, or (b) providing the 3-methylcrotonic acid by the enzymatic conversion of 3-hydroxyisovalerate (HIV) into 3-methylcrotonic acid.

WO 2017/085167 also describes that this method which has been developed for the production of isobutene from 3-methylcrotonyl-CoA via 3-methylcrotonic acid or from 3-hydroxyisovalerate (HIV) via 3-methylcrotonic acid may be embedded in a pathway for the production of isobutene starting from acetyl-CoA which is a central component and an important key molecule in metabolism used in many biochemical reactions. The corresponding reactions are schematically shown in FIG. 1.

In WO 2018/206262 it is described that 3-methylcrotonic acid is enzymatically converted into isobutene by making use of a prenylated FMN-dependent decarboxylase associated with an FMN prenyl transferase when dimethylallyl pyrophosphate (DMAPP) instead of DMAP is used. WO 2018/206262, moreover, describes that the enzymatic conversion of 3-methylcrotonic acid into isobutene which is achieved by making use of a prenylated FMN-dependent decarboxylase associated with an FMN prenyl transferase, wherein said FMN prenyl transferase catalyzes the prenylation of a flavin cofactor (FMN or FAD) utilizing dimethylallyl phosphate (DMAP) and/or dimethylallyl pyrophosphate (DMAPP) into a flavin-derived cofactor is a key step of the above overall metabolic pathway from acetyl-CoA into isobutene. It has been found that in this key step, the availability of dimethylallyl phosphate (DMAP) and/or dimethylallyl pyrophosphate (DMAPP) as well as the availability of the flavin cofactor FMN are limiting factors while in WO 2018/206262 improved methods by increasing the pool/amount of dimethylallyl phosphate (DMAP) and/or dimethylallyl pyrophosphate (DMAPP) in order to ensure the efficient biosynthesis of the prenylated flavin cofactor (FMN or FAD) are described.

WO 2020/188033 describes an improved method for the production of isobutene from acetyl-CoA, wherein the pool of available of acetyl-CoA in the production strain is increased through an increased uptake of pantothenate and/or an increased conversion of pantothenate into CoA.

Although, as described above, various approaches have been described in the prior art for producing isobutene by enzymatic conversions in biological systems, thereby allowing to use renewable resources as raw material, there is still a need to improve efficiency and effectiveness of such methods in order to increase yield and make them commercially attractive.

### SUMMARY OF THE INVENTION

The present invention meets this demand by providing a recombinant organism or microorganism having a decreased pool of crotonic acid compared to the organism or microorganism from which it is derived due to at least:
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonic acid into crotonyl-CoA;
(iv) an increased conversion of crotonyl-[acyl-carrier protein] into butyryl [acyl-carrier-protein];
(v) a decreased conversion of crotonyl-[acyl-carrier protein] into crotonic acid; and/or
(vi) a decreased conversion of crotonyl-CoA into crotonic acid.

It has been surprisingly found by the inventors that crotonic acid, 2-pentenoic acid and 2-hexenoic acid are irreversible inhibitors of the enzyme ferulic acid decarboxylase (FDC, see FIG. 2), which is preferably used in industrial processes for the conversion of 3-methylcrotonic acid into isobutene. In recombinant organisms or microorganisms, 3-methylcrotonic acid, the substrate of FDC, can be produced from acetyl-CoA in a multistep enzymatic process. The final conversion of 3-methylcrotonic acid into isobutene can be carried out in the same recombinant organism or microorganism that has been used for the production of 3-methylcrotonic acid. In such a one-step process, it is required that the recombinant organism or microorganism encodes the enzyme FDC. Alternatively, isobutene may be produced from 3-methylcrotonic acid in a two-step process. For that, a fermentation culture medium of a recombinant organism or microorganism comprising the produced 3-methylcrotonic acid may be contacted with a recombinant organism or microorganism encoding the enzyme FDC in an in vivo or in vitro biotransformation reaction. In an alternative two-step process, a first recombinant organism or microorganism may be used to convert acetyl-CoA into 3-hydroxyisovaleric acid and the produced 3-hydroxyisovaleric acid may then be converted into isobutene by a second recombinant organism or microorganism (via 3-methylcrotonic acid and FDC).

When producing 3-methylcrotonic acid and/or isobutene in a recombinant organism or microorganism, crotonic acid, 2-pentenoic acid and/or 2-hexenoic acid could be side products and, due to their inhibitory effect on FDC, even at trace amounts, decrease the productivity of the entire process. Thus, reducing and/or depleting the intracellular and extracellular levels of crotonic acid, 2-pentenoic acid and/or 2-hexenoic acid in a recombinant organism or microorganism that is used for the production of 3-methylcrotonic acid and/or isobutene can result in significantly improved product formation. It has been successfully demonstrated by the inventors that decreasing the pool of crotonic acid in a recombinant microorganism results in increased formation of isobutene (see Examples 2 and 3).

Herein, the inventors have identified various strategies to reduce the pools of crotonic acid in a recombinant organism or microorganism. In particular, the inventors have identified strategies to decrease the pools of crotonic acid in a recombinant organism or microorganism (a) by directing metabolic flux away from crotonic acid and/or (b) by directly preventing the formation of crotonic acid.

### A Decreased Pool of Crotonic Acid

The recombinant organism or microorganism according to the present invention is characterized as having a decreased pool of crotonic acid compared to the organism or microorganism from which it is derived due to:
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonic acid into crotonyl-CoA;
(iv) an increased conversion of crotonyl-[acyl-carrier protein] into butyryl [acyl-carrier-protein];
(v) a decreased conversion of crotonyl-CoA into crotonic acid; and/or
(vi) a decreased conversion of crotonyl-[acyl-carrier protein] into crotonic acid.

The term “a decreased pool of crotonic acid” as used in the present invention means, in general terms, that the amount and/or the availability of crotonic acid in the recombinant (genetically modified) organism or microorganism is lower than in the correspondingly non-modified organism or microorganism. In preferred embodiments, in the context of the present invention, “a decreased pool of crotonic acid” means that the amount and/or the availability of crotonic acid in the genetically modified, recombinant organism or microorganism is at least 5%, 10%, 20%, 30%, 40%, 50%, 60%, 70%, 80% or 90% lower than in the corresponding non-modified organism or microorganism.

It is to be understood that crotonic acid is a hydrophobic molecule that can readily diffuse across biological membranes. Due to its diffusion behaviour, the intracellular concentration of crotonic acid is expected to correlate at least to a certain extent with the crotonic acid concentration in the culture medium. Accordingly, the “pool of crotonic acid” is not strictly limited to the “intracellular pool” of crotonic acid in an organism or microorganism, but also extends to the culture medium in which the organism or microorganism is comprised. Accordingly, a first organism or microorganism may be determined to have a decreased pool of crotonic acid compared to a second organism or microorganism if the concentration of crotonic acid in the culture medium of the first organism or microorganism is lower than the concentration of crotonic acid in the culture medium of the second organism or microorganism. When making such comparisons, it is important to compare crotonic acid concentrations in cell cultures having comparable cell densities and, preferably, comparable culture volumes.

Within the present invention, it is to be understood that a recombinant organism or microorganism having a “decreased pool of crotonic acid” may also be defined as a recombinant organism or microorganism “producing less crotonic acid”. That is, term “pool” is not to be strictly understood as the “intracellular pool”.

The term “an increased conversion of crotonyl-CoA into butyryl-CoA” as used in the present invention means, in general terms, that the expression and/or the activity of a corresponding enzyme described below in the recombinant (genetically modified) organism or microorganism is higher than in the correspondingly non-modified organism or microorganism. In preferred embodiments, in the context of the present invention, an “increased conversion of crotonyl-CoA into butyryl-CoA” means that the expression and/or the activity of a corresponding enzyme described below in the genetically modified, recombinant organism or microorganism is at least 1%, 2%, 5%, 7% or 10%, preferably at least 20%, more preferably at least 30% or 50%, even more preferably at least 70% or 80% and particularly preferred at least 90% or 100% higher than in the corresponding non-modified organism or microorganism. In even more preferred embodiments, the expression and/or the activity of an enzyme described below in the genetically modified, recombinant organism or microorganism may be at least 150%, at least 200% or at least 500% higher compared to the corresponding non-modified organism or microorganism. In particularly preferred embodiments, the expression and/or the activity of an enzyme described below in the genetically modified, recombinant organism or microorganism is at least 2-fold, 5-fold, 7-fold and more preferably at least 10-fold higher than in the corresponding non-modified organism or microorganism.

The term “an increased conversion of butyryl-CoA into butyric acid” as used in the present invention means, in general terms, that the expression and/or the activity of a corresponding enzyme described below in the recombinant (genetically modified) organism or microorganism is higher than in the correspondingly non-modified organism or microorganism. In preferred embodiments, in the context of the present invention, an “increased conversion of butyryl-CoA into butyric acid” means that the expression and/or the activity of a corresponding enzyme described below in the genetically modified, recombinant organism or microorganism is at least 1%, 2%, 5%, 7% or 10%, preferably at least 20%, more preferably at least 30% or 50%, even more preferably at least 70% or 80% and particularly preferred at least 90% or 100% higher than in the corresponding non-modified organism or microorganism. In even more preferred embodiments, the expression and/or the activity of an enzyme described below in the genetically modified, recombinant organism or microorganism may be at least 150%, at least 200% or at least 500% higher compared to the corresponding non-modified organism or microorganism. In particularly preferred embodiments, the expression and/or the activity of an enzyme described below in the genetically modified, recombinant organism or microorganism is at least 2-fold, 5-fold, 7-fold and more preferably at least 10-fold higher than in the corresponding non-modified organism or microorganism.

The term “an increased conversion of crotonyl-CoA into 3-hydroxybutyryl-CoA” as used in the present invention means, in general terms, that the expression and/or the activity of a corresponding enzyme described below in the recombinant (genetically modified) organism or microorganism is higher than in the correspondingly non-modified organism or microorganism. In preferred embodiments, in the context of the present invention, an “increased conversion of crotonyl-CoA into 3-hydroxybutyryl-CoA” means that the expression and/or the activity of a corresponding enzyme described below in the genetically modified, recombinant organism or microorganism is at least 1%, 2%, 5%, 7% or 10%, preferably at least 20%, more preferably at least 30% or 50%, even more preferably at least 70% or 80% and particularly preferred at least 90% or 100% higher than in the corresponding non-modified organism or microorganism. In even more preferred embodiments, the expression and/or the activity of an enzyme described below in the genetically modified, recombinant organism or microorganism may be at least 150%, at least 200% or at least 500% higher compared to the corresponding non-modified organism or microorganism. In particularly preferred embodiments, the expression and/or the activity of an enzyme described below in the genetically modified, recombinant organism or microorganism is at least 2-fold, 5-fold, 7-fold and more preferably at least 10-fold higher than in the corresponding non-modified organism or microorganism.

The term “an increased conversion of 3-hydroxybutyryl-CoA into 3-hydroxybutyric acid” as used in the present invention means, in general terms, that the expression and/or the activity of a corresponding enzyme described below in the recombinant (genetically modified) organism or microorganism is higher than in the correspondingly non-modified organism or microorganism. In preferred embodiments, in the context of the present invention, an “increased conversion of 3-hydroxybutyryl-CoA into 3-hydroxybutyric acid” means that the expression and/or the activity of a corresponding enzyme described below in the genetically modified, recombinant organism or microorganism is at least 1%, 2%, 5%, 7% or 10%, preferably at least 20%, more preferably at least 30% or 50%, even more preferably at least 70% or 80% and particularly preferred at least 90% or 100% higher than in the corresponding non-modified organism or microorganism. In even more preferred embodiments, the expression and/or the activity of an enzyme described below in the genetically modified, recombinant organism or microorganism may be at least 150%, at least 200% or at least 500% higher compared to the corresponding non-modified organism or microorganism. In particularly preferred embodiments, the expression and/or the activity of an enzyme described below in the genetically modified, recombinant organism or microorganism is at least 2-fold, 5-fold, 7-fold and more preferably at least 10-fold higher than in the corresponding non-modified organism or microorganism.

The term “an increased conversion of crotonic acid into crotonyl-CoA” as used in the present invention means, in general terms, that the expression and/or the activity of a corresponding enzyme described below in the recombinant (genetically modified) organism or microorganism is higher than in the correspondingly non-modified organism or microorganism. In preferred embodiments, in the context of the present invention, an “increased conversion of crotonic acid into crotonyl-CoA” means that the expression and/or the activity of a corresponding enzyme described below in the genetically modified, recombinant organism or microorganism is at least 1%, 2%, 5%, 7% or 10%, preferably at least 20%, more preferably at least 30% or 50%, even more preferably at least 70% or 80% and particularly preferred at least 90% or 100% higher than in the corresponding non-modified organism or microorganism. In even more preferred embodiments, the expression and/or the activity of an enzyme described below in the genetically modified, recombinant organism or microorganism may be at least 150%, at least 200% or at least 500% higher compared to the corresponding non-modified organism or microorganism. In particularly preferred embodiments, the expression and/or the activity of an enzyme described below in the genetically modified, recombinant organism or microorganism is at least 2-fold, 5-fold, 7-fold and more preferably at least 10-fold higher than in the corresponding non-modified organism or microorganism.

The term “an increased conversion of crotonyl-[acyl-carrier protein] into butyryl [acyl-carrier-protein]” as used in the present invention means, in general terms, that the expression and/or the activity of a corresponding enzyme described below in the recombinant (genetically modified) organism or microorganism is higher than in the correspondingly non-modified organism or microorganism. In preferred embodiments, in the context of the present invention, an “increased conversion of crotonyl-[acyl-carrier protein] into butyryl [acyl-carrier-protein]” means that the expression and/or the activity of a corresponding enzyme described below in the genetically modified, recombinant organism or microorganism is at least 1%, 2%, 5%, 7% or 10%, preferably at least 20%, more preferably at least 30% or 50%, even more preferably at least 70% or 80% and particularly preferred at least 90% or 100% higher than in the corresponding non-modified organism or microorganism. In even more preferred embodiments, the expression and/or the activity of an enzyme described below in the genetically modified, recombinant organism or microorganism may be at least 150%, at least 200% or at least 500% higher compared to the corresponding non-modified organism or microorganism. In particularly preferred embodiments, the expression and/or the activity of an enzyme described below in the genetically modified, recombinant organism or microorganism is at least 2-fold, 5-fold, 7-fold and more preferably at least 10-fold higher than in the corresponding non-modified organism or microorganism.

The term “a decreased conversion of crotonyl-[acyl-carrier protein] into crotonic acid” as used in the present invention means, in general terms, that the expression and/or the activity of a corresponding enzyme described below in the recombinant (genetically modified) organism or microorganism is lower than in the correspondingly non-modified organism or microorganism. In preferred embodiments, in the context of the present invention, a “decreased conversion of crotonyl-[acyl-carrier protein] into crotonic acid” means that the expression and/or the activity of a corresponding enzyme described below in the genetically modified, recombinant organism or microorganism is at least 5%, 10%, 20%, 30%, 40%, 50%, 60%, 70%, 80% or 90% lower than in the corresponding non-modified organism or microorganism. Alternatively, the substrate specificity of a corresponding enzyme described below may be altered such that the affinity for crotonyl-[acyl-carrier protein] is reduced by at least 5%, 10%, 20%, 30%, 40%, 50%, 60%, 70%, 80% or 90%, preferably while the affinity for other substrate remains unchanged.

The term “a decreased conversion of crotonyl-CoA into crotonic acid” as used in the present invention means, in general terms, that the expression and/or the activity of a corresponding enzyme described below in the recombinant (genetically modified) organism or microorganism is lower than in the correspondingly non-modified organism or microorganism. In preferred embodiments, in the context of the present invention, a “decreased conversion of crotonyl-CoA into crotonic acid” means that the expression and/or the activity of a corresponding enzyme described below in the genetically modified, recombinant organism or microorganism is at least 5%, 10%, 20%, 30%, 40%, 50%, 60%, 70%, 80% or 90% lower than in the corresponding non-modified organism or microorganism. Alternatively, the substrate specificity of a corresponding enzyme described below may be altered such that the affinity for crotonyl-CoA is reduced by at least 5%, 10%, 20%, 30%, 40%, 50%, 60%, 70%, 80% or 90%, preferably while the affinity for other substrate remains unchanged.

Methods and assays for measuring the pool of crotonic acid in a cell have been described in the art (Lie et al., Biosynthesis of butenoic acid through fatty acid biosynthesis pathway in Escherichia coli, Appl Microbiol Biotechnol; DOI 10.1007/s00253-014-6233-2). In brief, crotonic acid can passively diffuse across the cell membrane due to its hydrophobicity. Consequently, the pool of crotonic acid in a cell can be readily determined by culturing said cell in a liquid medium and measuring the concentration of crotonic acid in the supernatant by a suitable method, such as HPLC or GC-MS.

In certain embodiments, the pool of crotonic acid in an organism or microorganism may be determined using HPLC (Ma et al., Simultaneous determination of organic acids and saccharides in lactic acid fermentation broth from biomass using high performance liquid chromatography. Se Pu. 2012 January; 30(1):62-6. doi: 10.3724/sp.j.1123.2011.09033). For that, the supernatant of a liquid cell culture comprising an organism or microorganism according to the invention may be filtered through a 0.22-μm syringe filter for HPLC analysis. The sample may be measured by HPLC (Agilent 1260 series, Germany) equipped with an Aminex HPX-87H 300 mm×7.8 mm column (Bio-Rad) and a diode array detector at 210 nm. Analysis may be performed with a mobile phase of 6 mM H₂SO₄ at a flow rate of 0.5 mL/min at 55° C. The concentrations of crotonic acid may be quantitatively determined with a calibration curve using linear regression. The external standard method may be used to get the regression equations.

Methods and assays for measuring an (increased) conversion of crotonyl-CoA into butyryl-CoA over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of crotonyl-CoA into butyryl-CoA, the cell lysates may be contacted with crotonyl-CoA under suitable conditions and the formation of butyryl-CoA may be determined with analytic methods known in the art.

Methods and assays for measuring an (increased) conversion of butyryl-CoA into butyric acid over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of butyryl-CoA into butyric acid, the cell lysates may be contacted with butyryl-CoA under suitable conditions and the formation of butyric acid may be determined with analytic methods known in the art.

Methods and assays for measuring an (increased) conversion of crotonyl-CoA into 3-hydroxybutyryl-CoA over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of crotonyl-CoA into 3-hydroxybutyryl-CoA, the cell lysates may be contacted with crotonyl-CoA under suitable conditions and the formation of 3-hydroxybutyryl-CoA may be determined with analytic methods known in the art.

Methods and assays for measuring an (increased) conversion of 3-hydroxybutyryl-CoA into 3-hydroxybutyric acid over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of 3-hydroxybutyryl-CoA into 3-hydroxybutyric acid, the cell lysates may be contacted with 3-hydroxybutyryl-CoA under suitable conditions and the formation of 3-hydroxybutyric acid may be determined with analytic methods known in the art.

Methods and assays for measuring an (increased) conversion of crotonic acid into crotonyl-CoA over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of crotonic acid into crotonyl-CoA, the cell lysates may be contacted with crotonic acid under suitable conditions and the formation of crotonyl-CoA may be determined with analytic methods known in the art.

Methods and assays for measuring an (increased) conversion of crotonyl-[acyl-carrier protein] into butyryl [acyl-carrier-protein] over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of crotonyl-[acyl-carrier protein] into butyryl [acyl-carrier-protein], the cell lysates may be contacted with crotonyl-[acyl-carrier protein] under suitable conditions and the formation of butyryl [acyl-carrier-protein] may be determined with analytic methods known in the art.

Methods and assays for measuring a (decreased) conversion of crotonyl-[acyl-carrier protein] into crotonic acid over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of crotonyl-[acyl-carrier protein] into crotonic acid, the cell lysates may be contacted with crotonyl-[acyl-carrier protein] under suitable conditions and the formation of crotonic acid may be determined with a suitable analytic method. Suitable methods for detecting and or quantifying the levels of crotonic acid are disclosed above.

Methods and assays for measuring a (decreased) conversion of crotonyl-CoA into crotonic acid over the organism or microorganism from which it is derived are known to the person skilled in the art or could be developed without undue burden and without needing inventive skill. For example, a recombinant (genetically modified) organism or microorganism may be cultured under similar conditions as a correspondingly non-modified organism or microorganism and whole cell lysates may be prepared from both organisms or microorganisms. To determine the conversion of crotonyl-CoA into crotonic acid, the cell lysates may be contacted with crotonyl-CoA under suitable conditions and the formation of crotonic acid may be determined with a suitable analytic method. Suitable methods for detecting and or quantifying the levels of crotonic acid are disclosed above.

Generally, (i) an increased conversion of crotonyl-CoA into butyryl-CoA; and/or an increased conversion of butyryl-CoA into butyric acid; (ii) an increased conversion of crotonyl-CoA into 3-hydroxybutyryl-CoA; and/or an increased conversion of 3-hydroxybutyryl-CoA into 3-hydroxybutyric acid; (iii) an increased conversion of crotonic acid into crotonyl-CoA; and/or (iv) an increased conversion of crotonyl-[acyl-carrier protein] into butyryl [acyl-carrier-protein] can be achieved by the recombinant expression of a certain protein.

Similarly, a decreased conversion of crotonyl-[acyl-carrier protein] and/or crotonyl-CoA into crotonic acid can be achieved by reducing the metabolic flux from crotonyl-CoA and/or crotonyl-[acyl-carrier protein] to crotonic acid.

A recombinant organism or microorganism having a decreased pool of crotonic acid over the organism or microorganism from which it is derived while this decreased pool of crotonic acid is due to (i) an increased conversion of crotonyl-CoA into butyryl-CoA; and/or an increased conversion of butyryl-CoA into butyric acid; (ii) an increased conversion of crotonyl-CoA into 3-hydroxybutyryl-CoA; and/or an increased conversion of 3-hydroxybutyryl-CoA into 3-hydroxybutyric acid; (iii) an increased conversion of crotonic acid into crotonyl-CoA; (iv) an increased conversion of crotonyl-[acyl-carrier protein] into butyryl [acyl-carrier-protein]; and/or (v) a decreased conversion of crotonyl-[acyl-carrier protein] and/or crotonyl-CoA into crotonic acid can be achieved by different recombinant modifications which are described in more detail further below.

Generally, “recombinant” in this context denotes the artificial genetic modification of an organism or microorganism, either by addition, removal, or modification of a chromosomal or extra-chromosomal gene or regulatory motif such as a promoter, or by fusion of organisms, or by addition of a vector of any type, for example plasmidic.

The term “recombinant expression” denotes the production of a protein involving a genetic modification, preferably in order to produce a protein of exogenous or heterologous origin with respect to its host, that is, which does not naturally occur in the production host, or in order to produce a modified or mutated endogenous protein.

The “recombinant expression” in the context of the present invention is preferably an “overexpression”. “Overexpression” or “overexpressing” in this context denotes the recombinant expression of a protein in a host organism, preferably originating from an organism different from the one in which it is expressed, increased by at least 10% and preferably by 20%, 50%, 100%, 500% and possibly more as compared to the natural expression of said protein occurring in said host organism or microorganism. This definition also encompasses the case where there is no natural expression of said protein.

Thus, in brief, the recombinant expression according to the present invention leading to a decreased pool of crotonic acid may be due to (1) the overexpression of the respective endogenous gene, (2) the introduction of a respective heterologous gene and/or (3) the expression of a mutated protein having an increased activity, e.g., an increased activity for catalysing the corresponding reaction over the respective enzyme from which it is derived or an increased transporter activity.

In certain embodiments, increased levels of one or more of the enzymes disclosed herein can be achieved by increasing the copy number of a nucleic acid encoding said enzyme(s). In certain embodiments, the respective nucleic acids may be cloned into an expression vector. The term “expression vector”, as used herein, denotes a nucleic acid vehicle (plasmid) that is propagated autonomously within a suitable host cell (i.e. independent of chromosomal nucleic acids) and that is characterized by the presence of at least one “expression cassette”. The term “expression cassette”, as used herein, refers to a genetic construct that is capable to allow gene expression of a nucleic acid sequence of interest (i.e. a “heterologous” nucleic acid sequence). This requires that such expression cassette comprises regulatory sequence elements which contain information regarding to transcriptional and/or translational regulation, and that such regulatory sequences are “operably linked” to the nucleic acid sequence of interest. An operable linkage is a linkage in which the regulatory sequence elements and the nucleic acid sequence to be expressed are connected in a way that enables gene expression. Methods for cloning a nucleic acid molecule into an expression vector are well known in the art. Furthermore, the skilled person is aware of suitable expression vectors that may be used in the present invention.

In certain embodiments, the nucleic acid(s) may be under control of a recombinant promoter. The recombinant promoter may be any suitable inducible or constitutive promoter known in the art. That is, the skilled person is aware of a wide range of promoters that can be used for the expression of a nucleic acid in a particular organism or microorganism. Furthermore, the skilled person is capable of testing different promoters in order to identify promoters that result in the desired expression level.

In certain embodiments, two or more of the enzymes disclosed herein may be encoded in a single expression vector. Each of the two or more enzymes may be under control of a separate promoter or two more enzymes may be under control of the same promoter.

Alternatively, or in addition, increased levels of one or more enzymes may be achieved by integrating one or more copies of a nucleic acid encoding said enzyme(s) into the genome of the recombinant organism or microorganism of the invention. The nucleic acid may encode an endogenous or a heterologous enzyme. That is, genome integration may result in an increased copy number of an endogenous gene or in the introduction of a heterologous nucleic acid. The nucleic acid(s) that are integrated into the genome of the recombinant organism or microorganism of the invention may comprise their native promoter(s) or recombinant promoter(s), i.e., an inducible or a constitutive promoter. Recombination-based methods for introducing a nucleic acid molecule into the genome are well known in the art.

In certain embodiments, a nucleic acid encoding one or more of the enzymes disclosed herein may be integrated into the genome and, in addition, a nucleic acid encoding the same enzyme(s) may be additionally overexpressed from an expression vector.

Within the present invention, a decreased conversion of crotonyl-[acyl-carrier protein] and/or crotonyl-CoA to crotonic acid may be achieved by reducing the levels and/or the activity of one or more enzymes discussed below. Alternatively, a decreased conversion of crotonyl-[acyl-carrier protein] and/or crotonyl-CoA to crotonic acid may be achieved by altering the substrate specificity of one or more enzymes discussed below.

In a particular embodiment, the invention relates to the recombinant organism or microorganism according to the invention, wherein the reduced level of an enzyme is due to (i) a complete or partial deletion of a gene encoding the respective enzyme in said organism or microorganism; or (ii) a modification in a regulatory element of a gene encoding the respective enzyme in said organism or microorganism.

That is, in certain embodiments, a reduced metabolic flux from crotonyl-[acyl-carrier protein] and/or crotonyl-CoA to crotonic acid may be achieved by introducing one or more deletion mutations into the chromosome of an organism or microorganism. Preferably, such deletion mutations result in reduced expression of one or more genes encoding enzymes that are involved in the conversion of crotonyl-[acyl-carrier protein] and/or crotonyl-CoA to crotonic acid.

In certain embodiments, the deletion is a full deletion of a gene encoding an enzyme that is involved in the conversion of crotonyl-[acyl-carrier protein] and/or crotonyl-CoA to crotonic acid. A full deletion is a deletion, wherein all codons of a gene are removed from the chromosome of an organism or microorganism. A full deletion of a gene may also include the additional deletion of gene regulatory elements, such as promoters, or even neighbouring genes.

In certain embodiments, the deletion may be a partial deletion of a gene encoding an enzyme that is involved in the conversion of crotonyl-[acyl-carrier protein] and/or crotonyl-CoA to crotonic acid. In a partial deletion mutant, only a fragment of a gene is removed from the chromosome of an organism or microorganism. Preferably, the partial deletion results in a non-functional enzyme. Non-functional enzymes can be obtained by deleting parts of the gene that encode essential domains of an enzyme, such as domains comprising the active site, or by introducing a frame shift into the gene.

Apart from modifying the coding sequence of a gene that encodes an enzyme that is involved in the conversion of crotonyl-[acyl-carrier protein] and/or crotonyl-CoA to crotonic acid, the expression level of such genes may also be reduced by modifying a regulatory element of said gene. The term “regulatory element,” as used herein refers to a genetic element which controls some aspect of the expression of nucleic acid sequences. For example, a promoter is a regulatory element which facilitates the initiation of transcription of an operably linked coding region. Other regulatory elements are ribosome-binding sites, splicing signals, polyadenylation signals, termination signals, etc.

Reducing the level of an enzyme that is involved in the conversion of crotonyl-[acyl-carrier protein] and/or crotonyl-CoA to crotonic acid in an organism or microorganism may be achieved by fully or partially deleting the promoter and/or further regulatory elements of a gene encoding said enzyme. Alternatively, or in addition, the expression level of an enzyme that is involved in the conversion of crotonyl-[acyl-carrier protein] and/or crotonyl-CoA to crotonic acid may be reduced in an organism or microorganism by introducing point mutations and/or inserting nucleic acid molecules into a regulatory element of a gene encoding an enzyme that is involved in the conversion of crotonyl-[acyl-carrier protein] and/or crotonyl-CoA to crotonic acid. For example, in bacteria introducing point mutations into the ribosome binding site can drastically reduce the expression of a downstream nucleic acid.

The skilled person is well aware of methods of molecular engineering that allow deleting chromosomal DNA and/or inserting foreign DNA into the chromosome of an organism or microorganism.

To reduce the metabolic flux from crotonyl-[acyl-carrier protein] and/or crotonyl-CoA to crotonic acid, the level of at least one enzyme that is involved in the conversion of crotonyl-[acyl-carrier protein] and/or crotonyl-CoA to crotonic acid may be reduced. In particular, the expression level of an enzyme that is involved in the conversion of crotonyl-[acyl-carrier protein] and/or crotonyl-CoA to crotonic acid may be reduced by at least 10%, 20%, 30%, 40%, 50%, 60%, 70%, 80%, 90% or 100%. The skilled person is aware that the level of an enzyme can be reduced by 100%, for example by deleting the entire gene encoding said enzyme. However, in certain embodiments, only a partial reduction of enzyme levels may be desired, for example if a complete removal of an enzyme would result in reduced viability. In such embodiments, it would be preferred to reduce enzyme levels by modifying a regulatory element of a gene encoding said enzyme.

Without being bound to theory, methods and assays for measuring the (level of) expression of a protein include Western Blot, ELISA etc. In another embodiment, the measurement of the (level of) expression is done by measuring the amount of the corresponding RNA. Corresponding methods are well known to the person skilled in the art and include, e.g., Northern Blot or reverse transcription quantitative PCR (RT-qPCR).

In a particular embodiment, the invention relates to the recombinant organism or microorganism according to the invention, wherein the reduced activity of an enzyme is due to (i) an inactivating mutation in a gene encoding the respective enzyme in said organism or microorganism; and/or (ii) the addition of an inhibitor of the respective enzyme.

Instead of reducing the expression level of at least one enzyme that is involved in the conversion of crotonyl-[acyl-carrier protein] and/or crotonyl-CoA to crotonic acid, the activity of the one or more enzyme may also be modified.

That is, the metabolic flux from crotonyl-[acyl-carrier protein] and/or crotonyl-CoA to crotonic acid may also be reduced by introducing mutations into a gene encoding an enzyme that is involved in the conversion of crotonyl-[acyl-carrier protein] and/or crotonyl-CoA to crotonic acid. For example, introducing a point mutation or a foreign nucleic acid into a gene encoding an enzyme that is involved in the conversion of crotonyl-[acyl-carrier protein] and/or crotonyl-CoA to crotonic acid may result in an inactive gene product or a gene product with reduced activity. In particular, mutations in the active site of an enzyme are likely to result in enzyme variants with reduced or abolished activity.

Alternatively, the activity of an enzyme in an organism or microorganism may be reduced by adding a suitable inhibitor. The inhibitor may be an endogenous inhibitor that is produced by the organism or microorganism itself or an exogenous inhibitor that is added to the organism or microorganism. In certain embodiments, a precursor molecule of an inhibitor may be added to the organism or microorganism, which is then converted into an inhibitor by the organism or microorganism itself. An endogenous inhibitor is said to be “added” to the organism or microorganism, if the organism or microorganism comprises a genetic modification that results in increased production of said inhibitor. In contrast, addition of an exogenous inhibitor to an organism or microorganism may be achieved by adding the inhibitor, or a precursor thereof, to the culture medium.

To reduce the metabolic flux from crotonyl-[acyl-carrier protein] and/or crotonyl-CoA to crotonic acid, the activity of at least one enzyme that is involved in the conversion of crotonyl-[acyl-carrier protein] and/or crotonyl-CoA to crotonic acid may be reduced. In particular, the activity of an enzyme that is involved in the conversion of crotonyl-[acyl-carrier protein] and/or crotonyl-CoA to crotonic acid may be reduced by at least 10%, 20%, 30%, 40%, 50%, 60%, 70%, 80%, 90% or 100%. The skilled person is aware that the activity of an enzyme can be reduced by 100%, for example by replacing one or more essential amino acids in the active site of an enzyme. However, in certain embodiments, only a partial reduction of enzyme activity may be desired, for example if a complete reduction of enzyme activity would result in reduced viability.

It has to be noted that a reduced metabolic flux from crotonyl-[acyl-carrier protein] and/or crotonyl-CoA to crotonic acid may also be achieved by altering the substrate specificity of an enzyme. That is, an enzyme may be engineered such that it has only minimal affinity for crotonyl-[acyl-carrier protein] and/or crotonyl-CoA, but can still catalyse the hydrolysis of other substrates.

Methods and assays for measuring activity of an enzyme are well known to the person skilled in the art. Preferably, the term activity relates to the specific activity of an enzyme. The term “specific activity”, as used herein, is defined as the units of activity in a given amount of protein. Thus, the specific activity is not directly measured but is calculated by dividing (1) the activity in units/ml of the enzyme sample by (2) the concentration of protein in that sample, so the specific activity is expressed as units/mg.

To determine the specific activity of an enzyme, or a mutant variant thereof, the enzyme may be recombinantly expressed and purified by methods known in the art. The specific activity of said enzyme or enzyme variant may then be determined with a suitable substrate. Further, the influence of an inhibitor on the specific activity may be determined with purified enzyme.

Reducing Metabolic Flux from Crotonyl-CoA to Crotonic Acid

Within the present invention, a decreased pool of crotonic acid can be achieved by decreasing the conversion of crotonyl-CoA into crotonic acid. It is known in the art that crotonyl-CoA is converted to crotonic acid by thioester hydrolases. For example, in E. coli crotonyl-CoA may be hydrolyzed to crotonic acid by the thioester hydrolase YdiI (MenI). To prevent the formation of crotonic acid from crotonyl-CoA, the inventors have developed various strategies to reduce the metabolic flux from crotonyl-CoA to crotonic acid.

These strategies involve:
(i) increasing the conversion of crotonyl-CoA into butyryl-CoA; and/or
(ii) increasing the conversion of butyryl-CoA into butyric acid; and/or
(iii) increasing the conversion of crotonyl-CoA into 3-hydroxybutyryl-CoA; and/or
(iv) increasing the conversion of 3-hydroxybutyryl-CoA into 3-hydroxybutyric acid.
   Increasing the Conversion of Crotonyl-CoA into Butyryl-CoA

In certain embodiments, the metabolic flux from crotonyl-CoA to crotonic acid may be decreased by increasing the metabolic flux from crotonyl-CoA to butyryl-CoA. That is, by increasing the conversion of crotonyl-CoA into butyryl-CoA, smaller amounts of crotonyl-CoA will be available for the hydrolysis into crotonic acid.

To increase the conversion of crotonyl-CoA into butyryl-CoA, a gene encoding an enzyme that can catalyze the conversion of crotonyl-CoA into butyryl-CoA may be overexpressed in the recombinant organism or microorganism according to the invention. The enzyme may be an exogenous enzyme that has been shown to efficiently catalyze the conversion of crotonyl-CoA into butyryl-CoA. Alternatively, the enzyme may be an endogenous enzyme that will be available at higher concentrations due to recombinant expression. Moreover, the enzyme may be an exogenous or endogenous enzyme that has been engineered to convert crotonyl-CoA into butyryl-CoA more efficiently.

Thus, in a particular embodiment, the invention relates to the recombinant organism or microorganism according to the invention, wherein the increased conversion of crotonyl-CoA into butyryl-CoA is due to an increased level and/or activity of an enzyme capable of reducing a carbon-carbon double bond (EC 1.3) in said organism or microorganism.

That is, the recombinant organism or microorganism may overexpress any enzyme from EC class 1.3 that is capable of converting crotonyl-CoA into butyryl-CoA.

In a particular embodiment, the invention relates to the recombinant organism or microorganism according to the invention, wherein the enzyme capable of reducing a carbon-carbon double bond (EC 1.3) is NADH or NADPH-dependent (EC 1.3.1) or flavin-dependent (EC 1.3.8).

The enzyme that catalyzes the conversion of crotonyl-CoA into butyryl-CoA may depend on any cofactor or even be cofactor-independent, as long as it can catalyze the conversion of crotonyl-CoA into butyryl-CoA. However, in a preferred embodiment, the enzyme capable of reducing a carbon-carbon double bond and, in particular, reducing crotonyl-CoA to butyryl-CoA is NADH or NADPH-dependent (EC 1.3.1) or flavin-dependent (EC 1.3.8).

In certain embodiments, the enzyme that catalyzes the conversion of crotonyl-CoA into butyryl-CoA may be a crotonyl-CoA reductase (EC 1.3.1.86). A non-limiting example of a crotonyl-CoA reductase is Ccr from Streptomyces collinus. However, crotonyl-CoA reductases have been described in other organisms. The amino acid sequence of Ccr from Streptomyces collinus (Uniprot Accession No: Q53865) is set forth in SEQ ID NO:1.

Ccr from Streptomyces collinus (SEQ ID NO: 1):
MTVKDILDAIQSKDATSADFAALQLPESYRAITVHKDETEMFAGLETRDKDPRKSIHLDE
VPVPELGPGEALVAVMASSVNYNSVWTSIFEPVSTFAFLERYGKLSPLTKRHDLPYHIIG
SDLAGVVLRTGPGVNAWQPGDEVVAHCLSVELESPDGHDDTMLDPEQRIWGFETNFGGLA
EIALVKTNQLMPKPKHLTWEEAAAPGLVNSTAYRQLVSRNGAAMKQGDNVLIWGASGGLG
SYATQFALAGGANPICVVSSPQKAEICRSMGAEAIIDRNAEGYKFWKDEHTQDPKEWKRF
GKRIRELTGGEDIDIVFEHPGRETFGASVYVTRKGGTITTCASTSGYMHEYDNRYLWMSL
KRIIGSHFANYREAYEANRLIAKGKIHPTLSKTYSLEETGQAAYDVHRNLHQGKVGVLCL
APEEGLGVRDAEMRAQHIDAINRERNV

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:1. In other embodiments, the recombinant organism or microorganism according to the invention comprises a nucleic acid sequence encoding a continuous stretch of at least 50, 100, 200, 300 or 400 amino acids derived from the polypeptide set forth in SEQ ID NO:1.

In certain embodiments, the enzyme that catalyzes the conversion of crotonyl-CoA into butyryl-CoA may be a trans-2-enoyl-CoA reductase (EC 1.3.1.44). A non-limiting example of a trans-2-enoyl-CoA reductase is FabV from Treponema denticola. However, trans-2-enoyl-CoA reductases have been described in other organisms. The amino acid sequence of FabV from Treponema denticola (Uniprot Accession No: Q73Q47) is set forth in SEQ ID NO:2.

FabV from Treponema denticola (SEQ ID NO: 2):
MIVKPMVRNNICLNAHPQGCKKGVEDQIEYTKKRITAEVKAGAKAPKNVLVLGCSNGYGL
ASRITAAFGYGAATIGVSFEKAGSETKYGTPGWYNNLAFDEAAKREGLYSVTIDGDAFSD
EIKAQVIEEAKKKGIKFDLIVYSLASPVRTDPDTGIMHKSVLKPFGKTFTGKTVDPFTGE
LKEISAEPANDEEAAATVKVMGGEDWERWIKQLSKEGLLEEGCITLAYSYIGPEATQALY
RKGTIGKAKEHLEATAHRLNKENPSIRAFVSVNKGLVTRASAVIPVIPLYLASLFKVMKE
KGNHEGCIEQITRLYAERLYRKDGTIPVDEENRIRIDDWELEEDVQKAVSALMEKVTGEN
AESLTDLAGYRHDFLASNGFDVEGINYEAEVERFDRI

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:2. In other embodiments, the recombinant organism or microorganism according to the invention comprises a nucleic acid sequence encoding a continuous stretch of at least 50, 100, 200, 300 or 350 amino acids derived from the polypeptide set forth in SEQ ID NO:2.

In a certain embodiment, the enzyme that catalyzes the conversion of crotonyl-CoA into butyryl-CoA may be an enoyl-[acyl-carrier-protein] reductase (EC 1.3.1.9). A non-limiting example of an enoyl-[acyl-carrier-protein] reductase is FabI from Escherichia coli. However, enoyl-[acyl-carrier-protein] reductases have been described in other organisms. The amino acid sequence of FabI from Escherichia coli (Uniprot Accession No: P0AEK4) is set forth in SEQ ID NO:3.

FabI from Escherichia coli (SEQ ID NO: 3):
MGFLSGKRILVTGVASKLSIAYGIAQAMHREGAELAFTYQNDKLKGRVEEFAAQLGSDIV
LQCDVAEDASIDTMFAELGKVWPKFDGFVHSIGFAPGDQLDGDYVNAVTREGFKIAHDIS
SYSFVAMAKACRSMLNPGSALLTLSYLGAERAIPNYNVMGLAKASLEANVRYMANAMGPE
GVRVNAISAGPIRTLAASGIKDFRKMLAHCEAVTPIRRTVTIEDVGNSAAFLCSDLSAGI
SGEVVHVDGGFSIAAMNELELK

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:3. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:3.

In certain embodiments, the enzyme that catalyzes the conversion of crotonyl-CoA into butyryl-CoA may be a short-chain acyl-CoA dehydrogenase (EC 1.3.8.1). A non-limiting example of a short-chain acyl-CoA dehydrogenase is a short-chain acyl-CoA dehydrogenase from Megasphaera elsdenii. However, short-chain acyl-CoA dehydrogenases have been described in other organisms. The amino acid sequence of Megasphaera elsdenii short-chain acyl-CoA dehydrogenase (Uniprot Accession No: Q06319) is set forth in SEQ ID NO:4.

Megasphaera elsdenii short-chain acyl-CoA dehydrogenase (SEQ ID NO: 4):
MDFNLTDIQQDFLKLAHDFGEKKLAPTVTERDHKGIYDKELIDELLSLGITGAYFEEKYG
GSGDDGGDVLSYILAVEELAKYDAGVAITLSATVSLCANPIWQFGTEAQKEKFLVPLVEG
TKLGAFGLTEPNAGTDASGQQTIATKNDDGTYTLNGSKIFITNGGAADIYIVFAMTDKSK
GNHGITAFILEDGTPGFTYGKKEDKMGIHTSQTMELVFQDVKVPAENMLGEEGKGFKIAM
MTLDGGRIGVAAQALGIAEAALADAVEYSKQRVQFGKPLCKFQSISFKLADMKMQIEAAR
NLVYKAACKKQEGKPFTVDAAIAKRVASDVAMRVTTEAVQIFGGYGYSEEYPVARHMRDA
KITQIYEGTNEVQLMVTGGALLR

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:4. In other embodiments, the recombinant organism or microorganism according to the invention comprises a nucleic acid sequence encoding a continuous stretch of at least 50, 100, 200, 300 or 350 amino acids derived from the polypeptide set forth in SEQ ID NO:4.

In certain embodiments, the enzyme that catalyzes the conversion of crotonyl-CoA into butyryl-CoA may be a butyryl-CoA dehydrogenase (EC 1.3.8.1). A non-limiting example of a butyryl-CoA dehydrogenase is a butyryl-CoA dehydrogenase from Acidaminococcus fermentans. However, butyryl-CoA dehydrogenases have been described in other organisms. The amino acid sequence of Acidaminococcus fermentans butyryl-CoA dehydrogenase (Uniprot Accession No: D2RL84) is set forth in SEQ ID NO:5.

Acidaminococcus fermentans butyryl-CoA dehydrogenase (SEQ ID NO: 5):
MDFNLTEDQQMIKDMAAEFAEKFLAPTVEERDKAHIWDRKLIDKMGEAGFCGICFPEEYG
GMGLDVLSYILAVEELSKVDDGTGITLSANVSLCATPIYMFGTEEQKQKYLAPIAEGTHV
GAFGLTEPSAGTDASAQQTTAVLKGDKYILNGSKIFITNGKEADTYVVFAMTDKSQGVHG
ISAFILEKGMPGFRFGKIEDKMGGHTSITAELIFEDCEVPKENLLGKEGEGFKIAMETLD
GGRIGVAAQALGIAEGALAAAVKYSKEREQFGRSISKFQALQFMMADMATKIEAARYLVY
HAAMLKNEGKPYSEAAAMAKCFASDVAMEVTTDAVQIFGGYGYTVDYPAERYMRNAKITQ
IYEGTNQVMRIVTSRALLRDKKK

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:5. In other embodiments, the recombinant organism or microorganism according to the invention comprises a nucleic acid sequence encoding a continuous stretch of at least 50, 100, 200, 300 or 350 amino acids derived from the polypeptide set forth in SEQ ID NO:5.

Preferably, Acidaminococcus fermentans butyryl-CoA dehydrogenase is used in combination with an Electron Transferring Flavoprotein (Etf) from Acidaminococcus fermentans (SEQ ID NO: 54; see Chowdhury et al., Studies on the Mechanism of Electron Bifurcation Catalyzed by Electron Transferring Flavoprotein (Etf) and Butyryl-CoA Dehydrogenase (Bcd) of Acidaminococcus fermentans; (2014) J Biol Chem 289:5145-5157).

In a preferred embodiment, the enzyme that catalyzes the conversion of crotonyl-CoA into butyryl-CoA may be a trans-2-enoyl-CoA reductase (EC 1.3.1.44) or an enoyl-[acyl-carrier-protein] reductase (EC 1.3.1.9), in particular any trans-2-enoyl-CoA reductase or enoyl-[acyl-carrier-protein] reductase, or variant thereof, as defined above.

In certain embodiments, the invention relates to a recombinant organism or microorganism comprising a nucleic acid encoding an NADH or NADPH-dependent enzyme capable of reducing a carbon-carbon double bond (EC 1.3.1) and/or a flavin-dependent enzyme capable of reducing a carbon-carbon double bond (EC 1.3.8), preferably wherein the NADH or NADPH-dependent enzyme capable of reducing a carbon-carbon double bond (EC 1.3.1) is a crotonyl-CoA reductase (EC 1.3.1.86), a trans-2-enoyl-CoA reductase (EC 1.3.1.44) and/or an enoyl-[acyl-carrier-protein] reductase (EC 1.3.1.9) and wherein flavin-dependent enzyme capable of reducing a carbon-carbon double bond (EC 1.3.8) is a short-chain acyl-CoA dehydrogenase (EC 1.3.8.1), preferably wherein the NADH or NADPH-dependent enzyme capable of reducing a carbon-carbon double bond (EC 1.3.1) is a trans-2-enoyl-CoA reductase (EC 1.3.1.44) and/or an enoyl-[acyl-carrier-protein] reductase (EC 1.3.1.9), preferably wherein the NADH or NADPH-dependent enzyme capable of reducing a carbon-carbon double bond (EC 1.3.1) is a trans-2-enoyl-CoA reductase (EC 1.3.1.44). In a particularly preferred embodiment, the trans-2-enoyl-CoA reductase (EC 1.3.1.44) is FabV from Treponema denticola, or an active variant thereof. In another particularly preferred embodiment, the enoyl-[acyl-carrier-protein] reductase (EC 1.3.1.9) is FabI from Escherichia coli, or an active variant thereof. However, it is to be understood that homologs of FabV and FabI from other species, or active variants thereof, may be alternatively used in the present invention.

In certain embodiments, the level and/or activity of said enzymes are increased in the recombinant organism or microorganism in comparison to the organism or microorganism from which it is derived. Increased levels and/or activity of an enzyme may be achieved as described herein. In certain embodiments, the nucleic acid is under control of a promoter. In certain embodiments, the nucleic acid and/or the promoter are of heterologous origin. In certain embodiments, the promoter is not the natural promoter of the nucleic acid. In certain embodiments, the nucleic acid including the promoter is integrated into the genome and/or is located on an extrachromosomal element, such as a plasmid. In certain embodiments, the recombinant organism or microorganism is an organism that is capable of producing 3-methylcrotonic acid and/or isobutene, such as any one of the organisms disclosed herein. Furthermore, the recombinant organism or microorganism may comprise one or more additional modifications that result in a decreased pool of crotonic acid, as described herein.

Increasing the Conversion of Butyryl-CoA into Butyric Acid

In certain embodiments, the metabolic flux from crotonyl-CoA to crotonic acid may also be decreased by increasing the metabolic flux from butyryl-CoA to butyric acid. The conversion of butyryl-CoA into butyric acid will decrease the pool of butyryl-CoA in the recombinant organism or microorganism. This, in turn, will direct the metabolic flux from crotonyl-CoA to butyryl-CoA and away from crotonic acid, thereby decreasing the crotonic acid pool of the recombinant organism or microorganism.

To increase the conversion of butyryl-CoA into butyric acid, a gene encoding an enzyme that can catalyze the conversion of butyryl-CoA into butyric acid may be overexpressed in the recombinant organism or microorganism according to the invention. The enzyme may be an exogenous enzyme that has been shown to efficiently catalyze the conversion of butyryl-CoA into butyric acid. Alternatively, the enzyme may be an endogenous enzyme that will be available at higher concentrations due to recombinant expression. Moreover, the enzyme may be an exogenous or endogenous enzyme that has been engineered to convert butyryl-CoA into butyric acid more efficiently.

Thus, in a particular embodiment, the invention relates to the recombinant organism or microorganism according to the invention, wherein the increased conversion of butyryl-CoA into butyric acid is due to an increased level and/or activity of a thioester hydrolase (EC 3.1.2), a CoA-transferase (EC 2.8.3), an acid thiol ligase (EC 6.2.1), a phosphate acyltransferase (EC 2.3.1) and/or acid kinase (EC 2.7.2) in said organism or microorganism.

That is, the recombinant organism or microorganism may overexpress any enzyme from EC classes 3.1.2, 2.8.3, 6.2.1, 2.3.1 and/or 2.7.2 that is capable of converting butyryl-CoA into butyric acid.

In a particular embodiment, the invention relates to the recombinant organism or microorganism according to the invention, wherein the thioester hydrolase (EC 3.1.2) is a 1,4-dihydroxy-2-naphtoyl-CoA hydrolase (EC 3.1.2.28) or an acyl-CoA thioesterase 2 (EC 3.1.2.20).

The enzyme that catalyzes the conversion of butyryl-CoA into butyric acid may be any enzyme from EC class 3.1.2 that efficiently catalyzes the conversion of butyryl-CoA into butyric acid. However, in a preferred embodiment, the thioester hydrolase may be a 1,4-dihydroxy-2-naphtoyl-CoA hydrolase (EC 3.1.2.28) or an acyl-CoA thioesterase 2 (EC 3.1.2.20).

In certain embodiments, the enzyme that catalyzes the conversion of butyryl-CoA into butyric acid may be a 1,4-dihydroxy-2-naphtoyl-CoA hydrolase (EC 3.1.2.28). A non-limiting example of a 1,4-dihydroxy-2-naphtoyl-CoA hydrolase is MenI from Escherichia coli or Shigella flexneri. However, 1,4-dihydroxy-2-naphtoyl-CoA hydrolases have been described in other organisms. The amino acid sequence of MenI from Escherichia coli (Uniprot Accession No: P77781) is set forth in SEQ ID NO:6. The amino acid sequence of MenI from Shigella flexneri (Uniprot Accession No: QOT487) is set forth in SEQ ID NO:7.

MenI from Escherichia coli (SEQ ID NO: 6):
MIWKRKITLEALNAMGEGNMVGFLDIRFEHIGDDTLEATMPVDSRTKQPFGLLHGGASVV
LAESIGSVAGYLCTEGEQKVVGLEINANHVRSAREGRVRGVCKPLHLGSRHQVWQIEIFD
EKGRLCCSSRLTTAIL
MenI from Shigella flexneri (SEQ ID NO: 7):
MIWKRKITLEALNAMGEGNMVGELDIRFEHIGDDTLEATMPVDSRTKQPFGLLHGGASVV
LAESIGSVAGYLCTEGEQKVVGLEINANHVRSAREGRVRGICKPLHLGSRHQVWQIEIFD
EKGRLCCSSRLTTAIL

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:6 or 7. In other embodiments, the recombinant organism or microorganism according to the invention comprises a nucleic acid sequence encoding a continuous stretch of at least 20, 40, 60, 80 or 100 amino acids derived from the polypeptide set forth in SEQ ID NO:6 or 7.

In certain embodiments, the enzyme that catalyzes the conversion of butyryl-CoA into butyric acid may be an acyl-CoA thioesterase 2 (EC 3.1.2.20). A non-limiting example of an acyl-CoA thioesterase 2 is TesB from Escherichia coli. However, acyl-CoA thioesterase 2 have been described in other organisms. The amino acid sequence of TesB (Uniprot Accession No: P0AGG3) from Escherichia coli is set forth in SEQ ID NO:8.

TesB from Escherichia coli (SEQ ID NO: 8):
MSQALKNLLTLLNLEKIEEGLFRGQSEDLGLRQVFGGQVVGQALYAAKETVPEERLVHSF
HSYFLRPGDSKKPIIYDVETLRDGNSFSARRVAAIQNGKPIFYMTASFQAPEAGFEHQKT
MPSAPAPDGLPSETQIAQSLAHLLPPVLKDKFICDRPLEVRPVEFHNPLKGHVAEPHRQV
WIRANGSVPDDLRVHQYLLGYASDLNFLPVALQPHGIGFLEPGIQIATIDHSMWFHRPFN
LNEWLLYSVESTSASSARGFVRGEFYTQDGVLVASTVQEGVMRNHN

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:8. In other embodiments, the recombinant organism or microorganism according to the invention comprises a nucleic acid sequence encoding a continuous stretch of at least 50, 100, 200, 300 or 350 amino acids derived from the polypeptide set forth in SEQ ID NO:8.

In a particular embodiment, the invention relates to the recombinant organism or microorganism according to the invention, wherein the CoA-transferase (EC 2.8.3) is an acetate CoA-transferase or a butyryl-CoA: acetate CoA-transferase (EC 2.8.3.8).

The enzyme that catalyzes the conversion of butyryl-CoA into butyric acid may be any enzyme from EC class 2.8.3 that efficiently catalyzes the conversion of butyryl-CoA into butyric acid. However, in a preferred embodiment, the CoA-transferase may be an acetate CoA-transferase or a butyryl-CoA: acetate CoA-transferase (EC 2.8.3.8).

In certain embodiments, the enzyme that catalyzes the conversion of butyryl-CoA into butyric acid may be an acetate CoA-transferase (EC 2.8.3.8). A non-limiting example of an acetate CoA-transferase is YdiF (Pct) from Cupriavidus necator. However, acetate CoA-transferases have been described in other organisms. The amino acid sequence of YdiF (Pct) (Uniprot Accession No: Q0K874) from Cupriavidus necator is set forth in SEQ ID NO:9.

YdiF (Pct) from Cupriavidus necator (SEQ ID NO: 9):
MKVITAREAAALVQDGWTVASAGFVGAGHAEAVTEALEQRFLQSGLPRDLTLVYSAGQGD
RGARGVNHFGNAGMTASIVGGHWRSATRLATLAMAEQCEGYNLPQGVLTHLYRAIAGGKP
GVMTKIGLHTFVDPRTAQDARYHGGAVNERARQAIAEGKACWVDAVDFRGDEYLFYPSFP
IHCALIRCTAADARGNLSTHREAFHHELLAMAQAAHNSGGIVIAQVESLVDHHEILQAIH
VPGILVDYVVVCDNPANHQMTFAESYNPAYVTPWQGEAAVAEAEAAPVAAGPLDARTIVQ
RRAVMELARRAPRVVNLGVGMPAAVGMLAHQAGLDGFTLTVEAGPIGGTPADGLSFGASA
YPEAVVDQPAQFDFYEGGGIDLAILGLAELDGHGNVNVSKFGEGEGASIAGVGGFINITQ
SARAVVFMGTLTAGGLEVRAGDGGLQIVREGRVKKIVPEVSHLSFNGPYVASLGIPVLYI
TERAVFEMRAGADGEARLTLVEIAPGVDLQRDVLDQCSTPIAVAQDLREMDARLFQAGPL
HL

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:9. In other embodiments, the recombinant organism or microorganism according to the invention comprises a nucleic acid sequence encoding a continuous stretch of at least 100, 200, 300, 400 or 500 amino acids derived from the polypeptide set forth in SEQ ID NO:9.

In certain embodiments, the enzyme that catalyzes the conversion of butyryl-CoA into butyric acid may be a butyrate: acetyl-CoA-transferase (EC 2.8.3.8). Non-limiting examples of a butyrate: acetyl-CoA-transferases are SwoI_1932 and SwoI_0436 from Syntrophomonas wolfei subsp. wolfei. However, butyrate: acetyl-CoA-transferases have been described in other organisms. The amino acid sequence of SwoI_1932 (Uniprot Accession No: Q0AVM5) from Syntrophomonas wolfei subsp. wolfei is set forth in SEQ ID NO:10. The amino acid sequence of SwoI_0436 (Uniprot Accession No: Q0AZT0) from Syntrophomonas wolfei subsp. wolfei is set forth in SEQ ID NO:11.

SwoI_1932 from Syntrophomonas wolfei subsp. Wolfei (SEQ ID NO: 10):
MYQKLLEEYKSKLVTADEAAKQVKSGDWVEYGFGINCARDFDEALAKRKDELEDVKIRCD
IGAYQHFTAEVDPDNKHFTWNSWHVAGHDRKFINKNLFYIPMKFHENPMMTRKDCVPTNV
AVIQCTAMDKHGYFNFGGSSVNCCAMMETARVTILEVNEKMPRCLGGNQECLHISQVDYI
IQSKNEPIATIGSAEPSPVEIAMAQHIIERLYDGNCIQLGIGGTPNAVGSMVAASDLKDL
GVHTEMYVDAYLLMAKAGKITGARKSIDKYKQVYSFAMGSQELYDYIDDNPGLASYSVDY
TNNPWVVAQIDDFVSINACIEVDLYGQVCAESVGTRHISGTGGQLDFVEGAYKSKNGQSF
ICLPSTIEIKGEVTSRIKPILTPGAIVTDPRTATHMMVTEFGIATLKGRSTWERAEELIK
IAHPDFQDELVKEAQKMNIWRKSNKIG
SwoI_0436 from Syntrophomonas wolfei subsp. Wolfei (SEQ ID NO: 11):
MNYVNEYRKKLVSADEAVKVVNSGDWIDYGSMCGQVVMLDEALARRKEELKDVKIWTLLT
LRRPRVMDVDPEEESFVWHSWHLSAMDRKIASERAVYYSPIRYSELPRYVREHIEPLAVA
MLQVAPMDKHGYFNFGPQNSHTRAVCERARIVIVEVNNQLPRCLGGYEESIHISEVDYIV
EGNNPPLPQLPDAASSEVDKKAAELIIEEMKDECCVQLGIGGMPNAVGKMIAESDLKDLG
CHTEMLVDAYVHMYEAGRLTGRKKNIDPGKMVYDFAVGSQRLYDFIDNNPLCASYPVNYT
NRPSIAALNDNLITINNAIEVDLYGQVCAESVGTRIISGTGGQLDFVLAAYESRGGKSFV
CLPSCSEREGKLKSKIVPTLKPGAIVTDPRSVAHYIVTEYGKFAMKGMSVWQRAEGLINL
AHPQLRDELIQAAQAQGIWRRSNKIS

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:10 or 11. In other embodiments, the recombinant organism or microorganism according to the invention comprises a nucleic acid sequence encoding a continuous stretch of at least 100, 200, 300, 350 or 400 amino acids derived from the polypeptide set forth in SEQ ID NO: 10 or 11.

In a particular embodiment, the invention relates to the recombinant organism or microorganism according to the invention, wherein the acid thiol ligase (EC 6.2.1) is an acetate-CoA ligase (ADP-forming) (EC 6.2.1.13).

The enzyme that catalyzes the conversion of butyryl-CoA into butyric acid may be any enzyme from EC class 6.2.1 that efficiently catalyzes the conversion of butyryl-CoA into butyric acid. However, in a preferred embodiment, the acid thiol ligase may be an acetate-CoA ligase (ADP-forming) (EC 6.2.1.13).

In certain embodiments, the enzyme that catalyzes the conversion of butyryl-CoA into butyric acid may be an acetate-CoA ligase (ADP-forming) (EC 6.2.1.13). A non-limiting example of an acetate-CoA ligase (ADP-forming) is Q9Y1N2 from Giardia intestinalis (Giardia lamblia). Further non limiting examples of acetate-CoA ligases (ADP-forming) are encoded by the genes Caur_3920 from Chloroflexus aurantiacus or EHI_178960 from Entamoeba histolytica. The amino acid sequence of Q9Y1N2 from Giardia intestinalis (Giardia lamblia) (Uniprot Accession No: Q9Y1N2) is set forth in SEQ ID NO: 12. The amino acid sequence of an acetate-CoA ligase (ADP-forming) encoded by the gene Caur_3920 from Chloroflexus aurantiacus (Uniprot Accession No: A9WDH8) is set forth in SEQ ID NO: 13. The amino acid sequence of an acetate-CoA ligase (ADP-forming) encoded by the gene EHI_178960 from Entamoeba histolytica (Uniprot Accession No: C4LUV9) is set forth in SEQ ID NO: 14.

Q9Y1N2 from Giardia intestinalis (Giardia lamblia) (SEQ ID NO: 12):
MRQNYSTKYKKMGKLSFLINPASVAVIGASPNAGKVGNTVVTNIKESGYTGKVYPINPTA
TEILGYKTYKSVLDVPDSIDVVIVVIPSKAVLAAAKECAQKKVKSLVVITAGFKEIGGEG
VQMEQDLTKICKDAGIRLVGPNCLGIVTPNLNCTFASAKPSKGSIAFLSQSGAMLTSILD
WALTNGIGFSNFISLGNKADVDEVDLIMEVAEDPNTDIILLYLESIVDGRKFLEQIPTCV
HKKPVIILKSGTSAAGAAAASSHTGALAGNDIAFDLAFEKAGVLRAATMSDLFDLGRLFV
SHRLPKGDNFVIVTNAGGPGIVTTDAFETYHVGMAALSDKTKEALAKVLPGEASVKNPVD
IVGDAPPKRYEDALEICFKEPPETVAGAVILVTPQGQTKPCEVAELCTRMYAKYPDRLVV
SAFMGGLTMQEPSKILNNAKMPVFPFPEPAIHATGAVLKYRKIKNRKTLAEKKVEVFKVD
NERIKKIIAGARADGRTVLLSHETSEIFTLYGVNAPKTKLATNEAEAATFAKEVTFPVVM
KIVSPQIIHKSDCGGVKLNIKTEAEATAAFKEIMANAAKNGPKGAVLKGVEIQQMVDFSK
YQKTTEMIVGVNRDPTWGPMIMVGQGGIYANYIKDVAFDLAYKYDREDAEAQLKKTKIYE
ILNGVRGQPRSDIKGLLDTMVKLAQLVNDFSEITELDMNPLLVFEEQKEGKNPGIAAVDV
KITLSH
Caur_3920 from Chloroflexus aurantiacus (SEQ ID NO: 13):
MLEAIFSPQSVAVIGASPDPARLGHRVLKNILDHGYQGNIYPIHPRATEVLGLCAYPSVL
DVPNPIDLAVVVIPPQHVIAAVDECGRKGIKGLVVITAGFKEVGGAGRELERELLATVRR
YGMRMIGPNSLGIIDTISKLNASFANAMPLPGNIALMSQSGAICTAILDWSHQQGIGFSR
FVSLGNKTDVDEVALLEAWNRDEHSRVILAYLEAIDDGPGFIRVAREVIKTTPVVAIKSG
TTAAGTRAASSHTGSLAGSEAAYETAFAQSGILRARTMNELFDLALVFAYQPMIRGNRVA
IVTNAGGPGIIATDAVERSGLAMAEFTPETIRHLQETLPPNANVENPIDVIGDATPDRYA
VAIKAALADPNVDGLIILFTPQAVSEPLTTANLIIDLVKGSDKPVVTSFMGGASIEKAVQ
ALNAARIPNYLFPERAVQSLAAMYRQSLWQRRPTPTYRTFSVDRERVARLFASVREAGRV
ELGEVEAREVIEAYGMRLPKSRLAQTPDEAVAIANEFGYPVVMKISSPDILHKSDIGGVR
VGLSDASAVRDAFELIEYRARKYLPSARIWGVLVQEMVRKGREVLVGVSRDPQFGPLIAV
GMGGIYVEVLKDVAFRLAPLSVEEVQEQIRSIRAYPLLRGVRGEAPADIAAIEETVLRVS
QLVTDFPEIVEMDINPLVVHHEGEGAVVLDARIILN
EHI_178960 from Entamoeba histolytica (SEQ ID NO: 14):
MQFEPLFNPKSVAVIGASDRKESVGYAVMNNMIKGGYKGNLYPVGRKPELFGKKCYAKIG
QIEEKVDLAVIAIPAKFVPGVCIECGEAGVKGLIIITAGFAEAGEEGKKMCIEIQATCQK
YNMRMIGPNCLGIINPRDGVNASFASVMPEAGGVAFISQSGALCTAILDWAANQHVGFSY
FVSIGSSIDTDYADLFEFFAKDPKVTSILMYIESIKDAKKFVLRAREFAADKPIILLKAG
KSSEGAAAAMSHTGSLAGNDAVYDAVEDRCGCIRVDSICDLWDCAHVLATQNIPQNNRLC
IITNAGGPGVISTDRLVSVHGHLAKLSESTMNELNAFLSPFWSHSNPVDVLGDATAGVYQ
KTLDIVIKDPQIDGVVVVLTPQAMTDPVAVAKSLVEHGPYQKPVLASWMGQSEVEAGVKI
LEEGKIPNFETPERAVTAFGYIMRHPDIAAKLKEIPKYLDVQVDYEGAKKLIADVVADGR
TTFTEYEGKMMFSKYGIPIKGMAKASTEDEAVAEAMKIGTPVVMKILSPDIMHKTDVGGV
KVKLTTEEEIRKAYRDIMTSVKEKKPEARIHGVLLEKMVGFKYECIIGCKKDPLFGPVIV
FGMGGVTVELYKDTNIALPPIGLQEADRLIDGTKISKLLRGYRGMPACDVEGLKKILVQF
SKMIMDFPEISEVDINPLAVSYEEFLVLDAKIVLDKNMIGKEVPKYSHLVIQP

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:12, 13 or 14. In other embodiments, the recombinant organism or microorganism according to the invention comprises a nucleic acid sequence encoding a continuous stretch of at least 200, 300, 400, 500 or 600 amino acids derived from the polypeptide set forth in SEQ ID NO:12, 13 or 14.

In a particular embodiment, the invention relates to the recombinant organism or microorganism according to the invention, wherein the phosphate acyltransferase (EC 2.3.1) is a phosphate butyryltransferase (EC 2.3.1.19) and/or wherein the acid kinase (EC 2.7.2) is a butyrate kinase (EC 2.7.2.7).

The enzyme that catalyzes the conversion of butyryl-CoA into butyric acid may be any enzyme or combination of enzymes from EC classes 2.3.1 and 2.7.2 that efficiently catalyzes the conversion of butyryl-CoA into butyric acid. That is, the conversion of butyryl-CoA into butyric acid may be catalyzed by a single enzyme that has phosphate acyltransferase and acid kinase activity. Alternatively, the conversion of butyryl-CoA into butyric acid may be catalyzed by a combination of a phosphate acyltransferase and an acid kinase. In a preferred embodiment, the phosphate acyltransferase (EC 2.3.1) may be a phosphate butyryltransferase (EC 2.3.1.19) and the acid kinase (EC 2.7.2) may be a butyrate kinase (EC 2.7.2.7).

In certain embodiments, the enzyme that is involved in the conversion of butyryl-CoA into butyric acid may be a phosphate butyryltransferase (EC 2.3.1.19). A non-limiting example of a phosphate butyryltransferase is Ptb from Clostridium acetobutylicum. However, phosphate butyryltransferases have been described in other organisms. The amino acid sequence of Ptb from Clostridium acetobutylicum (Uniprot Accession No: P58255) is set forth in SEQ ID NO: 15.

Ptb from Clostridium acetobutylicum (SEQ ID NO: 15):
MIKSFNEIIMKVKSKEMKKVAVAVAQDEPVLEAVRDAKKNGIADAILVGDHDEIVSIALK
IGMDVNDFEIVNEPNVKKAALKAVELVSTGKADMVMKGLVNTATFLRSVLNKEVGLRTGK
TMSHVAVFETEKFDRLLFLTDVAFNTYPELKEKIDIVNNSVKVAHAIGIENPKVAPICAV
EVINPKMPSTLDAAMLSKMSDRGQIKGCVVDGPLALDIALSEEAAHHKGVTGEVAGKADI
FLMPNIETGNVMYKTLTYTTDSKNGGILVGTSAPVVLTSRADSHETKMNSIALAALVAGN
K

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:15. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:15.

In certain embodiments, the enzyme that is involved in the conversion of butyryl-CoA into butyric acid may be a butyrate kinase (EC 2.7.2.7). A non-limiting example of a butyrate kinase is Buk from Clostridium acetobutylicum. However, butyrate kinases have been described in other organisms. The amino acid sequence of Buk from Clostridium acetobutylicum (Uniprot Accession No: Q45829) is set forth in SEQ ID NO:16.

Buk from Clostridium acetobutylicum (SEQ ID NO: 16):
MYRLLIINPGSTSTKIGIYDDEKEIFEKTLRHSAEEIEKYNTIFDQFQFRKNVILDALKE
ANIEVSSLNAVVGRGGLLKPIVSGTYAVNQKMLEDLKVGVQGQHASNLGGIIANEIAKEI
NVPAYIVDPVVVDELDEVSRISGMADIPRKSIFHALNQKAVARRYAKEVGKKYEDLNLIV
VHMGGGTSVGTHKDGRVIEVNNTLDGEGPFSPERSGGVPIGDLVRLCFSNKYTYEEVMKK
INGKGGVVSYLNTIDFKAVVDKALEGDKKCALIYEAFTFQVAKEIGKCSTVLKGNVDAII
LTGGIAYNEHVCNAIEDRVKFIAPVVRYGGEDELLALAEGGLRVLRGEEKAKEYK

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:16. In other embodiments, the recombinant organism or microorganism according to the invention comprises a nucleic acid sequence encoding a continuous stretch of at least 100, 150, 200, 250 or 300 amino acids derived from the polypeptide set forth in SEQ ID NO:16.

In certain embodiments, the recombinant organism or microorganism according to the invention encodes a phosphate acyltransferase and an acid kinase. In certain embodiments, the recombinant organism or microorganism according to the invention encodes a phosphate butyryltransferase and a butyrate kinase. In certain embodiments, the recombinant organism or microorganism according to the invention encodes Ptb and Buk from Clostridium acetobutylicum or any of the sequence variants or derivatives thereof that have been defined above.

Preferably, the enzyme that catalyzes the conversion of butyryl-CoA into butyric acid has a high affinity for butyryl-CoA and a low affinity for crotonyl-CoA. That is, the enzyme that catalyzes the conversion of butyryl-CoA into butyric acid preferably has at least a 2-fold, 5-fold, 10-fold, 20-fold, 50-fold or 100-fold higher affinity (lower Kₘ) for butyryl-CoA compared to crotonyl-CoA. The skilled person is aware of methods to determine the affinity (Kₘ) of an enzyme for a substrate. In certain embodiments, an enzyme used for the conversion of butyryl-CoA to butyric acid may be genetically engineered to reduce its affinity (increasing Kₘ) for crotonyl-CoA.

In certain embodiments, the invention relates to a recombinant organism or microorganism comprising a nucleic acid encoding a thioester hydrolase (EC 3.1.2), a CoA-transferase (EC 2.8.3), an acid thiol ligase (EC 6.2.1), a phosphate acyltransferase (EC 2.3.1) and/or acid kinase (EC 2.7.2), preferably wherein the thioester hydrolase (EC 3.1.2) is a 1,4-dihydroxy-2-naphtoyl-CoA hydrolase (EC 3.1.2.28) and/or an acyl-CoA thioesterase 2 (EC 3.1.2.20), wherein the CoA-transferase (EC 2.8.3) is an acetate CoA-transferase and/or a butyryl-CoA: acetate CoA-transferase (EC 2.8.3.8), wherein the acid thiol ligase (EC 6.2.1) is an acetate-CoA ligase (ADP-forming) (EC 6.2.1.13), wherein the phosphate acyltransferase (EC 2.3.1) is a phosphate butyryltransferase (EC 2.3.1.19) and/or wherein the acid kinase (EC 2.7.2) is a butyrate kinase (EC 2.7.2.7). In certain embodiments, the level and/or activity of said enzymes is increased in the recombinant organism or microorganism of the invention in comparison to the organism or microorganism from which it is derived. Increased levels and/or activity of an enzyme may be achieved as described herein. In certain embodiments, the nucleic acid is under control of a promoter. In certain embodiments, the nucleic acid and/or the promoter are of heterologous origin. In certain embodiments, the promoter is not the natural promoter of the nucleic acid. In certain embodiments, the nucleic acid including the promoter is integrated into the genome and/or is located on an extrachromosomal element, such as a plasmid. In certain embodiments, the recombinant organism or microorganism is an organism that is capable of producing 3-methylcrotonic acid and/or isobutene, such as any one of the organisms disclosed herein. Furthermore, the recombinant organism or microorganism may comprise one or more additional modifications that result in a decreased pool of crotonic acid, as described herein.

In a preferred embodiment, the recombinant organism or microorganism according to the invention is characterized by an increased conversion of crotonyl-CoA into butyryl-CoA and an increased conversion of the produced butyryl-CoA into butyric acid in comparison to the organism or microorganism from which it has been derived. That is, the metabolic flux may be directed away from crotonic acid more efficiently by simultaneously overexpressing a first enzyme that can convert crotonyl-CoA to butyryl-CoA and a second enzyme that can convert butyryl-CoA to butyric acid.

That is, in certain embodiments, the recombinant organism or microorganism according to the invention may have a decreased pool of crotonic acid due to an increased conversion of crotonyl-CoA into butyryl-CoA and an increased conversion of butyryl-CoA into butyric acid.

For that, the increased conversion of crotonyl-CoA into butyryl-CoA may be achieved due to an increased level and/or activity of an enzyme capable of reducing a carbon-carbon double bond (EC 1.3) in said organism or microorganism and the increased conversion of butyryl-CoA into butyric acid may be achieved due to an increased level and/or activity of a thioester hydrolase (EC 3.1.2), a CoA-transferase (EC 2.8.3), an acid thiol ligase (EC 6.2.1), a phosphate acyltransferase (EC 2.3.1) and/or acid kinase (EC 2.7.2) in the same organism or microorganism.

In certain embodiments, the enzyme capable of reducing a carbon-carbon double bond (EC 1.3) may be NADH or NADPH-dependent (EC 1.3.1) or flavin-dependent (EC 1.3.8).

That is, in one embodiment, the recombinant organism or microorganism according to the invention is an organism having an increased level and/or activity of an NADH or NADPH-dependent enzyme capable of reducing a carbon-carbon double bond (EC 1.3.1) and an increased level of one or more of: a thioester hydrolase (EC 3.1.2), a CoA-transferase (EC 2.8.3), an acid thiol ligase (EC 6.2.1), a phosphate acyltransferase (EC 2.3.1) and/or an acid kinase (EC 2.7.2)

In another embodiment, the recombinant organism or microorganism according to the invention is an organism having:
a) an increased level and/or activity of one or more of: an enoyl-[acyl-carrier-protein] reductase (EC 1.3.1.9) a trans-2-enoyl-CoA reductase (EC 1.3.1.44), a crotonyl-CoA reductase (EC 1.3.1.86) and/or a short-chain acyl-CoA dehydrogenase (EC 1.3.8.1); and
b) an increased level and/or activity of one or more of: a 1,4-dihydroxy-2-naphtoyl-CoA hydrolase (EC 3.1.2.28), an acyl-CoA thioesterase 2 (EC 3.1.2.20), an acetate CoA-transferase or a butyryl-CoA: acetate CoA-transferase (EC 2.8.3.8), an acetate-CoA ligase (ADP-forming) (EC 6.2.1.13), a phosphate butyryltransferase (EC 2.3.1.19) and/or a butyrate kinase (EC 2.7.2.7).

In a preferred embodiment, the recombinant organism or microorganism according to the invention is an organism having an increased level and/or activity of an enoyl-[acyl-carrier-protein] reductase (EC 1.3.1.9) and an increased level and/or activity of one or more of: a 1,4-dihydroxy-2-naphtoyl-CoA hydrolase (EC 3.1.2.28), an acyl-CoA thioesterase 2 (EC 3.1.2.20), an acetate CoA-transferase or a butyryl-CoA: acetate CoA-transferase (EC 2.8.3.8), an acetate-CoA ligase (ADP-forming) (EC 6.2.1.13), a phosphate butyryltransferase (EC 2.3.1.19) and/or a butyrate kinase (EC 2.7.2.7).

In another preferred embodiment, the recombinant organism or microorganism according to the invention is an organism having an increased level and/or activity of a trans-2-enoyl-CoA reductase (EC 1.3.1.44) and an increased level and/or activity of one or more of: a 1,4-dihydroxy-2-naphtoyl-CoA hydrolase (EC 3.1.2.28), an acyl-CoA thioesterase 2 (EC 3.1.2.20), an acetate CoA-transferase or a butyryl-CoA: acetate CoA-transferase (EC 2.8.3.8), an acetate-CoA ligase (ADP-forming) (EC 6.2.1.13), a phosphate butyryltransferase (EC 2.3.1.19) and/or a butyrate kinase (EC 2.7.2.7).

In another preferred embodiment, the recombinant organism or microorganism according to the invention is an organism having an increased level and/or activity of a crotonyl-CoA reductase (EC 1.3.1.86) and an increased level and/or activity of one or more of: a 1,4-dihydroxy-2-naphtoyl-CoA hydrolase (EC 3.1.2.28), an acyl-CoA thioesterase 2 (EC 3.1.2.20), an acetate CoA-transferase or a butyryl-CoA: acetate CoA-transferase (EC 2.8.3.8), an acetate-CoA ligase (ADP-forming) (EC 6.2.1.13), a phosphate butyryltransferase (EC 2.3.1.19) and/or a butyrate kinase (EC 2.7.2.7).

In one embodiment, the recombinant organism or microorganism according to the invention is an organism having an increased level and/or activity of a flavin-dependent enzyme capable of reducing a carbon-carbon double bond (EC 1.3.8) and an increased level and/or activity of one or more of: a thioester hydrolase (EC 3.1.2), a CoA-transferase (EC 2.8.3), an acid thiol ligase (EC 6.2.1), a phosphate acyltransferase (EC 2.3.1) and/or an acid kinase (EC 2.7.2)

In a preferred embodiment, the recombinant organism or microorganism according to the invention is an organism having an increased level and/or activity of a short-chain acyl-CoA dehydrogenase (EC 1.3.8.1) and an increased level and/or activity of one or more of: a 1,4-dihydroxy-2-naphtoyl-CoA hydrolase (EC 3.1.2.28), an acyl-CoA thioesterase 2 (EC 3.1.2.20), an acetate CoA-transferase or a butyryl-CoA: acetate CoA-transferase (EC 2.8.3.8), an acetate-CoA ligase (ADP-forming) (EC 6.2.1.13), a phosphate butyryltransferase (EC 2.3.1.19) and/or a butyrate kinase (EC 2.7.2.7).

Increasing the Conversion of Crotonyl-CoA into 3-hydroxybutyryl-CoA

In certain embodiments, the metabolic flux from crotonyl-CoA to crotonic acid may be decreased by increasing the metabolic flux from crotonyl-CoA to 3-hydroxybutyryl-CoA. That is, by increasing the conversion of crotonyl-CoA into 3-hydroxybutyryl-CoA, smaller amounts of crotonyl-CoA will be available for the hydrolysis into crotonic acid.

To increase the conversion of crotonyl-CoA into 3-hydroxybutyryl-CoA, a gene encoding an enzyme that can catalyze the conversion of crotonyl-CoA into 3-hydroxybutyryl-CoA may be overexpressed in the recombinant organism or microorganism according to the invention. The enzyme may be an exogenous enzyme that has been shown to efficiently catalyze the conversion of crotonyl-CoA into 3-hydroxybutyryl-CoA. Alternatively, the enzyme may be an endogenous enzyme that will be available at higher concentrations due to recombinant expression. Moreover, the enzyme may be an exogenous or endogenous enzyme that has been engineered to convert crotonyl-CoA into 3-hydroxybutyryl-CoA more efficiently.

Thus, in a particular embodiment, the invention relates to the recombinant organism or microorganism according to the invention, wherein the increased conversion of crotonyl-CoA into 3-hydroxybutyryl-CoA is due to an increased level and/or activity of a hydro-lyase (EC 4.2.1) in said organism or microorganism.

That is, the recombinant organism or microorganism may overexpress any enzyme from EC class 4.2.1 that is capable of converting crotonyl-CoA into 3-hydroxybutyryl-CoA.

In a particular embodiment, the invention relates to the recombinant organism or microorganism according to the invention, wherein the hydro-lyase (EC 4.2.1) is a short-chain-enoyl-CoA hydratase (EC 4.2.1.150), a 3-hydroxybutyryl-CoA dehydratase (EC 4.2.1.55) or an enoyl-CoA hydratase (EC 4.2.1.17).

The enzyme that catalyzes the conversion of crotonyl-CoA into 3-hydroxybutyryl-CoA may be any hydro-lyase (EC 4.2.1), as long as it can catalyse the conversion of crotonyl-CoA into 3-hydroxybutyryl-CoA. However, in a preferred embodiment, the hydro-lyase is a short-chain-enoyl-CoA hydratase (EC 4.2.1.150), a 3-hydroxybutyryl-CoA dehydratase (EC 4.2.1.55) or an enoyl-CoA hydratase (EC 4.2.1.17).

In certain embodiments, the enzyme that catalyzes the conversion of crotonyl-CoA into 3-hydroxybutyryl-CoA may be a short-chain-enoyl-CoA hydratase (EC 4.2.1.150). Non-limiting examples of a short-chain-enoyl-CoA hydratases are the short-chain-enoyl-CoA hydratases from Meiothermus ruber, Metallosphaera sedula and Clostridium acetobutylicum. However, short-chain-enoyl-CoA hydratases have been described in other organisms. The amino acid sequence of the short-chain-enoyl-CoA hydratase from Meiothermus ruber (Uniprot Accession No: D3PLE5) is set forth in SEQ ID NO: 17. The amino acid sequence of the short-chain-enoyl-CoA hydratase from Metallosphaera sedula (Uniprot Accession No: A4YDS4) is set forth in SEQ ID NO:18. The amino acid sequence of the short-chain-enoyl-CoA hydratase from Clostridium acetobutylicum (Uniprot Accession No: P52046) is set forth in SEQ ID NO:19.

Short-chain-enoyl-CoA hydratase from Meiothermus ruber (SEQ ID NO: 17):
MAQTFELELPEFEYLTYRVEDGMGIVTISRPQALNALNQDLLIELASVTEVITQDPEVKV
AIFTGEGKAFVAGADIAQIASLQDVFAAREFAIMGQSVENEIAALPVPSIAAINGYALGG
GLELALACDLRVASRNAKLGLPEVGLGIIPGFGGTQRLPRLVGRGRALDLIFTGRHVPAE
EALGLGLVNRVGEDALQTAKELAAAILKNGPVALALAKEAVGRGENLDLQEALEVEADLE
GLACATQDMREGTRAFLEKRPAQFKGE
Short-chain-enoyl-CoA hydratase from Metallosphaera sedula (SEQ ID NO: 18):
MKVTVIGSGVMGHGIAELAAIAGNEVWMNDISTEILQQAMERIKWSLSKLRESGSLKEGV
EQVLARIHPETDQAQALKGSDFVIEAVKEDLELKRTIFRNAEAHASPSAVLATNTSSLPI
SEIASVLKSPQRVVGMHFFNPPVLMPLVEIVRGKDTSDEVVKTTAEMAKSMNKETIVVKD
VPGFFVNRVLLRIMEAGCYLVEKGIASIQEVDSSAIEELGFPMGVFLLADYTGLDIGYSV
WKAVTARGFKAFPCSSTEKLVSQGKLGVKSGSGYYQYPSPGKFVRPTLPSTSKKLGRYLI
SPAVNEVSYLLREGIVGKDDAEKGCVLGLGLPKGILSYADEIGIDVVVNTLEEMRQTSGM
DHYSPDPLLLSMVKEGKLGRKSGQGFHTYAHEEAKYSTIVVRVEPPLAWIVLNRPTRYNA
INGDMIREINQALDSLEEREDVRVIAITGQGRVFSAGADVTEFGSLTPVKAMIASRKFHE
VFMKIQFLTKPVIAVINGLALGGGMELALSADFRVASKTAEMGQPEINLGLIPGGGGTQR
LSRLSGRKGLELVLTGRRVKAEEAYRLGIVEFLAEPEELESEVRKLANAIAEKSPLAVAS
AKLAYKLGEETHIWTGTSLEASLFGLLFSTKDFEEGVRAFLEKRKPNERGE
Short-chain-enoyl-CoA hydratase from Clostridium acetobutylicum (SEQ ID NO: 19):
MELNNVILEKEGKVAVVTINRPKALNALNSDTLKEMDYVIGEIENDSEVLAVILTGAGEK
SFVAGADISEMKEMNTIEGRKFGILGNKVERRLELLEKPVIAAVNGFALGGGCEIAMSCD
IRIASSNARFGQPEVGLGITPGFGGTQRLSRLVGMGMAKQLIFTAQNIKADEALRIGLVN
KVVEPSELMNTAKEIANKIVSNAPVAVKLSKQAINRGMQCDIDTALAFESEAFGECESTE
DQKDAMTAFIEKRKIEGFKNR

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:17, 18 or 19. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:17, 18 or 19.

In certain embodiments, the enzyme that catalyzes the conversion of crotonyl-CoA into 3-hydroxybutyryl-CoA may be a 3-hydroxybutyryl-CoA dehydratase (EC 4.2.1.55). A non-limiting example of a 3-hydroxybutyryl-CoA dehydratase is a 3-hydroxybutyryl-CoA dehydratase from Ferroglobus placidus. However, 3-hydroxybutyryl-CoA dehydratases have been described in other organisms. The amino acid sequence of the 3-hydroxybutyryl-CoA dehydratase from Ferroglobus placidus (Uniprot Accession No: D3RX14) is set forth in SEQ ID NO:20.

3-hydroxybutyryl-CoA dehydratase from Ferroglobus placidus (SEQ ID NO: 20):
MEVKNIAVLGAGAMGHAIAELAAVSGFNVKIRDIKEEILKNAMERIKQSLEKDEKKGRLK
EDPQAVLSRITATLDLKEAVEDADMIIEAIPEIMDLKKQVFAECEEYCREDTIIATNTSS
LSITELSKALKKPERFIGLHFFNPPKVMRLVEIVWGEKTSEETVKITEEVARKMNRVIIH
VRKDVPGFVVNRIFVTMANEAAWALEKGEGSVEEIDSAVKYRMGLPMGLFELHDLLGGGC
IDVSYHVLEYFRQTLGESYRPAPPFERLFKAGHLGKKSGKGFYDWSEGKSNEVPLRAGAN
FDLLRLIAPAVNEAAWLIEKEVATAEEIDLGVIHGLNYPRGLLRMADDIGIDKIVEKLEK
LYEEYGEERYKVNPVLQKMVEENKLGRKTGEGFYKYGRGLYEFVKVEKPKENVAVIKLNR
PQRANALNETFLKELEDALEWLEEDENVRCIVITGEGRNFCAGADIAVFAEGKVERMFEF
SQLGQKVENKIEKLSKPVIAAVNGAAMGGGFELALACDLRVVSSRAILALPELNLGIFPG
WGGTQRLALAVGVSKAKQLIFMRENIDAKTAYDLGIANYIAEENEFWDKVMEVAEKIAEG
PPLAYKFTKRVMFHGIKPELEASLFMESAAGGDIVLSDDVAEGIQAFSYRRKPNEKGR

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:20. In other embodiments, the recombinant organism or microorganism according to the invention comprises a nucleic acid sequence encoding a continuous stretch of at least 100, 200, 300, 400 or 500 amino acids derived from the polypeptide set forth in SEQ ID NO:20.

In certain embodiments, the enzyme that catalyzes the conversion of crotonyl-CoA into 3-hydroxybutyryl-CoA may be an enoyl-CoA hydratase (EC 4.2.1.17). A non-limiting example of an enoyl-CoA hydratase is an enoyl-CoA hydratase from Rattus norvegicus. However, enoyl-CoA hydratases have been described in other organisms. The amino acid sequence of enoyl-CoA hydratase from Rattus norvegicus (Uniprot Accession No: P14604) is set forth in SEQ ID NO:21.

Enoyl-CoA hydratase from Rattus norvegicus (SEQ ID NO: 21):
MAALRALLPRACNSLLSPVRCPEFRRFASGANFQYIITEKKGKNSSVGLIQLNRPKALNA
LCNGLIEELNQALETFEEDPAVGAIVLIGGEKAFAAGADIKEMQNRTFQDCYSGKFLSHW
DHITRIKKPVIAAVNGYALGGGCELAMMCDIIYAGEKAQFGQPEILLGTIPGAGGTQRLT
RAVGKSLAMEMVLTGDRISAQDAKQAGLVSKIFPVETLVEEAIQCAEKIANNSKIIVAMA
KESVNAAFEMTLTEGNKLEKKLFYSTFATDDRREGMSAFVEKRKANFKDH

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:21. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:21.

In certain embodiments, the invention relates to a recombinant organism or microorganism comprising a nucleic acid encoding a hydro-lyase (EC 4.2.1), preferably wherein the hydro-lyase (EC 4.2.1) is a short-chain-enoyl-CoA hydratase (EC 4.2.1.150), a 3-hydroxybutyryl-CoA dehydratase (EC 4.2.1.55) and/or an enoyl-CoA hydratase (EC 4.2.1.17), preferably wherein the hydro-lyase (EC 4.2.1) is a short-chain-enoyl-CoA hydratase (EC 4.2.1.150). In certain embodiments, the level and/or activity of said enzymes is increased in the recombinant organism or microorganism of the invention in comparison to the organism or microorganism from which it is derived. Increased levels and/or activity of an enzyme may be achieved as described herein. In certain embodiments, the nucleic acid is under control of a promoter. In certain embodiments, the nucleic acid and/or the promoter are of heterologous origin. In certain embodiments, the promoter is not the natural promoter of the nucleic acid. In certain embodiments, the nucleic acid including the promoter is integrated into the genome and/or is located on an extrachromosomal element, such as a plasmid. In certain embodiments, the recombinant organism or microorganism is an organism that is capable of producing 3-methylcrotonic acid and/or isobutene, such as any one of the organisms disclosed herein. Furthermore, the recombinant organism or microorganism may comprise one or more additional modifications that result in a decreased pool of crotonic acid, as described herein.

Increasing the Conversion of 3-hydroxybutyryl-CoA into 3-hydroxybutyric acid

In certain embodiments, the metabolic flux from crotonyl-CoA to crotonic acid may also be decreased by increasing the metabolic flux from 3-hydroxybutyryl-CoA to 3-hydroxybutyric acid. The conversion of 3-hydroxybutyryl-CoA into 3-hydroxybutyric acid will decrease the pool of 3-hydroxybutyryl-CoA in the recombinant organism or microorganism. This, in turn, will direct the metabolic flux from crotonyl-CoA to 3-hydroxybutyryl-CoA and away from crotonic acid, thereby decreasing the crotonic acid pool of the recombinant organism or microorganism.

To increase the conversion of 3-hydroxybutyryl-CoA into 3-hydroxybutyric acid, a gene encoding an enzyme that can catalyze the conversion of 3-hydroxybutyryl-CoA into 3-hydroxybutyric acid may be overexpressed in the recombinant organism or microorganism according to the invention. The enzyme may be an exogenous enzyme that has been shown to efficiently catalyze the conversion of 3-hydroxybutyryl-CoA into 3-hydroxybutyric acid. Alternatively, the enzyme may be an endogenous enzyme that will be available at higher concentrations due to recombinant expression. Moreover, the enzyme may be an exogenous or endogenous enzyme that has been engineered to convert 3-hydroxybutyryl-CoA into 3-hydroxybutyric acid more efficiently.

Thus, in a particular embodiment, the invention relates to the recombinant organism or microorganism according to the invention, wherein the increased conversion of 3-hydroxybutyryl-CoA into 3-hydroxybutyric acid is due to an increased level and/or activity of a thioester hydrolase (EC 3.1.2) in said organism or microorganism.

That is, the recombinant organism or microorganism may overexpress any enzyme from EC classes 3.1.2 that is capable of converting 3-hydroxybutyryl-CoA into 3-hydroxybutyric acid.

In a particular embodiment, the invention relates to the recombinant organism or microorganism according to the invention, wherein the thioester hydrolase (EC 3.1.2) is a palmitoyl-CoA hydrolase (EC 3.1.2.2) or an acyl-CoA thioesterase 2 (EC 3.1.2.20).

The enzyme that catalyzes the conversion of 3-hydroxybutyryl-CoA into 3-hydroxybutyric acid may be any enzyme from EC class 3.1.2 that efficiently catalyzes the conversion of 3-hydroxybutyryl-CoA into 3-hydroxybutyric acid. However, in a preferred embodiment, the thioester hydrolase may be a palmitoyl-CoA hydrolase (EC 3.1.2.2) or an acyl-CoA thioesterase 2 (EC 3.1.2.20).

In certain embodiments, the enzyme that catalyzes the conversion of 3-hydroxybutyryl-CoA into 3-hydroxybutyric acid may be a palmitoyl-CoA hydrolase (EC 3.1.2.2). A non-limiting example of a palmitoyl-CoA hydrolase is a palmitoyl-CoA hydrolase from Photobacterium profundum. However, palmitoyl-CoA hydrolases have been described in other organisms. The amino acid sequence of the palmitoyl-CoA hydrolase from Photobacterium profundum (Uniprot Accession No: Q93CG9) is set forth in SEQ ID NO:22.

Palmitoyl-CoA hydrolase from Photobacterium
profundum (SEQ ID NO: 22):
MSKIYHHPVQIYYEDTDHSGVVYHPNFLKYFERAREHVIDSDKLATLWN
DHGLGFAVYKANMIFQDGVEFAEICDIRTSFTLDGKYKTLWRQEVWRPG
ASRAAVIGDIEMVCLDKEKRLQPVPADILASMMAD

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:22. In other embodiments, the recombinant organism or microorganism according to the invention comprises a nucleic acid sequence encoding a continuous stretch of at least 20, 40, 60, 80 or 100 amino acids derived from the polypeptide set forth in SEQ ID NO:22.

In certain embodiments, the enzyme that catalyzes the conversion of 3-hydroxybutyryl-CoA into 3-hydroxybutyric acid may be an acyl-CoA thioesterase 2 (EC 3.1.2.20). Non-limiting examples of acyl-CoA thioesterase 2 are TesB and YciA from Escherichia coli. However, acyl-CoA thioesterase 2 have been described in other organisms. The amino acid sequence of TesB from Escherichia coli (Uniprot Accession No: P0AGG2) is set forth in SEQ ID NO:23. The amino acid sequence of YciA from Escherichia coli (Uniprot Accession No: P0A8Z0) is set forth in SEQ ID NO:24.

TesB from Escherichia coli (SEQ ID NO: 23):
MSQALKNLLTLLNLEKIEEGLFRGQSEDLGLRQVFGGQVVGQALYAAKE
TVPEERLVHSFHSYFLRPGDSKKPIIYDVETLRDGNSFSARRVAAIQNG
KPIFYMTASFQAPEAGFEHQKTMPSAPAPDGLPSETQIAQSLAHLLPPV
LKDKFICDRPLEVRPVEFHNPLKGHVAEPHRQVWIRANGSVPDDLRVHQ
YLLGYASDLNFLPVALQPHGIGFLEPGIQIATIDHSMWFHRPFNLNEWL
LYSVESTSASSARGFVRGEFYTQDGVLVASTVQEGVMRNHN
YciA from Escherichia coli (SEQ ID NO: 24):
MSTTHNVPQGDLVLRTLAMPADTNANGDIFGGWLMSQMDIGGAILAKEI
AHGRVVTVRVEGMTFLRPVAVGDVVCCYARCVQKGTTSVSINIEVWVKK
VASEPIGQRYKATEALFKYVAVDPEGKPRALPVE

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:23 or 24. In other embodiments, the recombinant organism or microorganism according to the invention comprises a nucleic acid sequence encoding a continuous stretch of at least 20, 40, 60, 80 or 100 amino acids derived from the polypeptide set forth in SEQ ID NO:23 or 24.

Preferably, the enzyme that catalyzes the conversion of 3-hydroxybutyryl-CoA into 3-hydroxybutyric acid has a high affinity for 3-hydroxybutyryl-CoA and a low affinity for crotonyl-CoA. That is, the enzyme that catalyzes the conversion of 3-hydroxybutyryl-CoA into 3-hydroxybutyric acid preferably has at least a 2-fold, 5-fold, 10-fold, 20-fold, 50-fold or 100-fold higher affinity (lower Kₘ) for 3-hydroxybutyryl-CoA compared to crotonyl-CoA. The skilled person is aware of methods to determine the affinity (Kₘ) of an enzyme for a substrate. In certain embodiments, an enzyme used for the conversion of 3-hydroxybutyryl-CoA to 3-hydroxybutyric acid may be genetically engineered to reduce its affinity (increasing Kₘ) for crotonyl-CoA.

In certain embodiments, the invention relates to a recombinant organism or microorganism comprising a nucleic acid encoding a thioester hydrolase (EC 3.1.2), preferably wherein the thioester hydrolase (EC 3.1.2) is a palmitoyl-CoA hydrolase (EC 3.1.2.2) and/or an acyl-CoA thioesterase 2 (EC 3.1.2.20). In certain embodiments, the level and/or activity of said enzymes is increased in the recombinant organism or microorganism of the invention in comparison to the organism or microorganism from which it is derived. Increased levels and/or activity of an enzyme may be achieved as described herein. In certain embodiments, the nucleic acid is under control of a promoter. In certain embodiments, the nucleic acid and/or the promoter are of heterologous origin. In certain embodiments, the promoter is not the natural promoter of the nucleic acid. In certain embodiments, the nucleic acid including the promoter is integrated into the genome and/or is located on an extrachromosomal element, such as a plasmid. In certain embodiments, the recombinant organism or microorganism is an organism that is capable of producing 3-methylcrotonic acid and/or isobutene, such as any one of the organisms disclosed herein. Furthermore, the recombinant organism or microorganism may comprise one or more additional modifications that result in a decreased pool of crotonic acid, as described herein.

In a preferred embodiment, the recombinant organism or microorganism according to the invention is characterized by an increased conversion of crotonyl-CoA into 3-hydroxybutyryl-CoA and an increased conversion of the produced 3-hydroxybutyryl-CoA into 3-hydroxybutyric acid in comparison to the organism or microorganism from which it has been derived. That is, the metabolic flux may be directed away from crotonic acid more efficiently by simultaneously overexpressing a first enzyme that can convert crotonyl-CoA to 3-hydroxybutyryl-CoA and a second enzyme that can convert 3-hydroxybutyryl-CoA to 3-hydroxybutyric acid.

That is, in certain embodiments, the recombinant organism or microorganism according to the invention may have a decreased pool of crotonic acid due to an increased conversion of crotonyl-CoA into 3-hydroxybutyryl-CoA and an increased conversion of 3-hydroxybutyryl-CoA into 3-hydroxybutyric acid.

For that, the increased conversion of crotonyl-CoA into 3-hydroxybutyryl-CoA may be achieved due to an increased level and/or activity of a hydro-lyase (EC 4.2.1) in said organism or microorganism and the increased conversion of 3-hydroxybutyryl-CoA into 3-hydroxybutyric acid may be achieved due to an increased level and/or activity of a thioester hydrolase (EC 3.1.2).

In another embodiment, the recombinant organism or microorganism according to the invention is an organism having:
a) an increased level and/or activity of one or more of: a short-chain-enoyl-CoA hydratase (EC 4.2.1.150), a 3-hydroxybutyryl-CoA dehydratase (EC 4.2.1.55) and/or an enoyl-CoA hydratase (EC 4.2.1.17); and
b) an increased level and/or activity of one or more of: a palmitoyl-CoA hydrolase (EC 3.1.2.2) and/or an acyl-CoA thioesterase 2 (EC 3.1.2.20).

In a preferred embodiment, the recombinant organism or microorganism according to the invention is an organism having an increased level and/or activity of a short-chain-enoyl-CoA hydratase (EC 4.2.1.150) and an increased level and/or activity of one or more of: a palmitoyl-CoA hydrolase (EC 3.1.2.2) and/or an acyl-CoA thioesterase 2 (EC 3.1.2.20).

In another preferred embodiment, the recombinant organism or microorganism according to the invention is an organism having an increased level and/or activity of a 3-hydroxybutyryl-CoA dehydratase (EC 4.2.1.55) and an increased level and/or activity of one or more of: a palmitoyl-CoA hydrolase (EC 3.1.2.2) and/or an acyl-CoA thioesterase 2 (EC 3.1.2.20).

In another preferred embodiment, the recombinant organism or microorganism according to the invention is an organism having an increased level and/or activity of an enoyl-CoA hydratase (EC 4.2.1.17) and an increased level and/or activity of one or more of: a palmitoyl-CoA hydrolase (EC 3.1.2.2) and/or an acyl-CoA thioesterase 2 (EC 3.1.2.20).

Reducing Metabolic Flux from crotonyl-[acyl-carrier-protein] to crotonic acid

Within the present invention, a decreased pool of crotonic acid can also be achieved by decreasing the conversion of crotonyl-[acyl-carrier-protein] into crotonic acid. Crotonyl-[acyl-carrier-protein] may be converted to crotonic acid by a suitable thioester hydrolase. To prevent the formation of crotonic acid from crotonyl-[acyl-carrier-protein], the inventors have developed different strategies to reduce metabolic flux from crotonyl-[acyl-carrier-protein] to crotonic acid.

These strategies involve:
(i) increasing the conversion of crotonyl-[acyl-carrier-protein] into butyryl-[acyl-carrier-protein]; and/or
(ii) decreasing the conversion of crotonyl-[acyl-carrier-protein] into crotonic acid.
   Increasing the Conversion of Crotonyl-[Acyl-Carrier-Protein] into Butyryl-[Acyl-Carrier-Protein]

In certain embodiments, the metabolic flux from crotonyl-[acyl-carrier-protein] to crotonic acid may be decreased by increasing the metabolic flux from crotonyl-[acyl-carrier-protein] to butyryl-[acyl-carrier-protein]. That is, by increasing the conversion of crotonyl-[acyl-carrier-protein] into butyryl-[acyl-carrier-protein], smaller amounts of crotonyl-[acyl-carrier-protein] will be available for hydrolysis into crotonic acid. The produced butyryl-[acyl-carrier-protein] can enter the fatty acid elongation pathways and will thus not accumulate in the recombinant organism or microorganism according to the invention.

To increase the conversion of crotonyl-[acyl-carrier-protein] into butyryl-[acyl-carrier-protein], a gene encoding an enzyme that can catalyze the conversion of crotonyl-[acyl-carrier-protein] into butyryl-[acyl-carrier-protein] may be overexpressed in the recombinant organism or microorganism according to the invention. The enzyme may be an exogenous enzyme that has been shown to efficiently catalyze the conversion of crotonyl-[acyl-carrier-protein] into butyryl-[acyl-carrier-protein]. Alternatively, the enzyme may be an endogenous enzyme that will be available at higher concentrations due to recombinant expression. Moreover, the enzyme may be an exogenous or endogenous enzyme that has been engineered to convert crotonyl-[acyl-carrier-protein] into butyryl-[acyl-carrier-protein] more efficiently.

Thus, in a particular embodiment, the invention relates to the recombinant organism or microorganism according to the invention, wherein the increased conversion of crotonyl-[acyl-carrier protein] into butyryl-[acyl-carrier protein] is due to an increased level and/or activity of an NADH or NADPH-dependent enoyl-[acyl-carrier-protein] reductase (EC 1.3.1) in said organism or microorganism.

That is, the recombinant organism or microorganism may overexpress any enzyme from EC class 1.3.1 that is capable of converting crotonyl-[acyl-carrier-protein] into butyryl-[acyl-carrier-protein].

In certain embodiments, the enzyme that catalyzes the conversion of crotonyl-[acyl-carrier-protein] into butyryl-[acyl-carrier-protein] may be an enoyl-[acyl-carrier-protein] reductase (NADH-dependent) (EC 1.3.1.9).

In certain embodiments, the enzyme that catalyzes the conversion of crotonyl-[acyl-carrier-protein] into butyryl-[acyl-carrier-protein] may be FabI from Escherichia coli (EC 1.3.1.9 and 1.3.1.104). The amino acid sequence of FabI from Escherichia coli (Uniprot Accession No: P0AEK4) is set forth in SEQ ID NO: 3.

In certain embodiments, the enzyme that catalyzes the conversion of crotonyl-[acyl-carrier-protein] into butyryl-[acyl-carrier-protein] may be FabI from Bacillus subtilis (EC 1.3.1.9). The amino acid sequence of FabI from Bacillus subtilis (Uniprot Accession No: P54616) is set forth in SEQ ID NO: 25.

FabI from Bacillus subtilis (SEQ ID NO: 25):
MNFSLEGRNIVVMGVANKRSIAWGIARSLHEAGARLIFTYAGERLEKSV
HELAGTLDRNDSIILPCDVTNDAEIETCFASIKEQVGVIHGIAHCIAFA
NKEELVGEYLNTNRDGFLLAHNISSYSLTAVVKAARPMMTEGGSIVTLT
YLGGELVMPNYNVMGVAKASLDASVKYLAADLGKENIRVNSISAGPIRT
LSAKGISDFNSILKDIEERAPLRRTTTPEEVGDTAAFLFSDMSRGITGE
NLHVDSGFHITAR

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:25. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:25.

In certain embodiments, the enzyme that catalyzes the conversion of crotonyl-[acyl-carrier-protein] into butyryl-[acyl-carrier-protein] may be an enoyl-[acyl-carrier-protein] reductase (NADPH-dependent) (EC 1.3.1.104).

In certain embodiments, the enzyme that catalyzes the conversion of crotonyl-[acyl-carrier-protein] into butyryl-[acyl-carrier-protein] may be FabL from Bacillus subtilis (EC 1.3.1.104). The amino acid sequence of FabL from Bacillus subtilis (Uniprot Accession No: P71079) is set forth in SEQ ID NO:26.

FabL from Bacillus subtilis (SEQ ID NO: 26):
MEQNKCALVTGSSRGVGKAAAIRLAENGYNIVINYARSKKAALETAEEI
EKLGVKVLVVKANVGQPAKIKEMFQQIDETFGRLDVFVNNAASGVLRPV
MELEETHWDWTMNINAKALLFCAQEAAKLMEKNGGGHIVSISSLGSIRY
LENYTTVGVSKAALEALTRYLAVELSPKQIIVNAVSGGAIDTDALKHFP
NREDLLEDARQNTPAGRMVEIKDMVDTVEFLVSSKADMIRGQTIIVDGG
RSLLV

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:26. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:26.

In certain embodiments, the enzyme that catalyzes the conversion of crotonyl-[acyl-carrier-protein] into butyryl-[acyl-carrier-protein] may be an enoyl-[acyl-carrier-protein] reductase (NADPH-dependent, Re-specific) (EC 1.3.1.39).

In certain embodiments, the enzyme that catalyzes the conversion of crotonyl-[acyl-carrier-protein] into butyryl-[acyl-carrier-protein] may be FabI from Staphylococcus aureus (EC 1.3.1.39). The amino acid sequence of FabI from Staphylococcus aureus (Uniprot Accession No: Q2FZQ3) is set forth in SEQ ID NO: 27.

FabI from Staphylococcus aureus (SEQ ID NO: 27):
MLNLENKTYVIMGIANKRSIAFGVAKVLDQLGAKLVFTYRKERSRKELE
KLLEQLNQPEAHLYQIDVQSDEEVINGFEQIGKDVGNIDGVYHSIAFAN
MEDLRGRFSETSREGFLLAQDISSYSLIIVAHEAKKLMPEGGSIVATTY
LGGEFAVQNYNVMGVAKASLEANVKYLALDLGPDNIRVNAISASPIRTL
SAKGVGGFNTILKEIEERAPLKRNVDQVEVGKTAAYLLSDLSSGVTGEN
IHVDSGFHAIK

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:27. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:27.

In certain embodiments, the enzyme that catalyzes the conversion of crotonyl-[acyl-carrier-protein] into butyryl-[acyl-carrier-protein] may be an enoyl-[acyl-carrier-protein] reductase (NADPH-dependent, Si-specific) (EC 1.3.1.10).

In certain embodiments, the enzyme that catalyzes the conversion of crotonyl-[acyl-carrier-protein] into butyryl-[acyl-carrier-protein] may be FabK from Porphyromonas gingivalis (EC 1.3.1.10 and EC 1.3.1.39). The amino acid sequence of FabK from Porphyromonas gingivalis (Uniprot Accession No: Q7MAW0) is set forth in SEQ ID NO:28.

FabK from Porphyromonas gingivalis
(SEQ ID NO: 28):
MNRICELLGIEHPIISGGMVWCSGWKLASAVSNCGGLGLIGAGSMHPDN
LEHHIRSCKAATDKPFGVNVPLLYPEMDKIMEIIMREHVPVVVTSAGSP
KVWTAKLKAAGSKVIHVVSSATFARKSEAAGVDAIVAEGFEAGGHNGRE
ETTTLCLIPEVVDAVNIPVVAAGGIASGRAVAAALALGADAVQVGTRFA
LSEESSAHEDFKAHCRRSVEGDTMLSLKAVSPTRLLKNKFYQDVFAAEQ
RGASVEELRELLGRGRAKQGIFEGDLHEGELEIGQAVSQISHAETVAEI
MVDLVDGYKRSLAGMPTEI

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:28. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:28.

In certain embodiments, the enzyme that catalyzes the conversion of crotonyl-[acyl-carrier-protein] into butyryl-[acyl-carrier-protein] may be FabK from Streptococcus pneumoniae (EC 1.3.1.10). The amino acid sequence of FabK from Streptococcus pneumoniae (Uniprot Accession No: Q9FBC5) is set forth in SEQ ID NO:29.

FabK from Streptococcus pneumoniae
(SEQ ID NO: 29):
MKTRITELLKIDYPIFQGGMAWVADGDLAGAVSKAGGLGIIGGGNAPKE
VVKANIDKIKSLTDKPFGVNIMLLSPFVEDIVDLVIEEGVKVVTTGAGN
PSKYMERFHEAGIIVIPVVPSVALAKRMEKIGADAVIAEGMEAGGHIGK
LTTMTLVRQVATAISIPVIAAGGIADGEGAAAGFMLGAEAVQVGTRFVV
AKESNAHPNYKEKILKARDIDTTISAQHFGHAVRAIKNQLTRDFELAEK
DAFKQEDPDLEIFEQMGAGALAKAVVHGDVDGGSVMAGQIAGLVSKEET
AEEILKDLYYGAAKKIQEEASRWTGVVRND

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:29. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:29.

In certain embodiments, the enzyme that catalyzes the conversion of crotonyl-[acyl-carrier-protein] into butyryl-[acyl-carrier-protein] may be ETR1 from Saccharomyces cerevisiae (EC 1.3.1.104). The amino acid sequence of ETR1 from Saccharomyces cerevisiae (Uniprot Accession No: P38071) is set forth in SEQ ID NO:30.

ETR1 from Saccharomyces cerevisiae
(SEQ ID NO: 30):
MLPTFKRYMSSSAHQIPKHFKSLIYSTHEVEDCTKVLSVKNYTPKQDLS
QSIVLKTLAFPINPSDINQLQGVYPSRPEKTYDYSTDEPAAIAGNEGVF
EVVSLPSGSSKGDLKLGDRVIPLQANQGTWSNYRVFSSSSDLIKVNDLD
LFSAATVSVNGCTGFQLVSDYIDWNSNGNEWIIQNAGTSSVSKIVTQVA
KAKGIKTLSVIRDRDNFDEVAKVLEDKYGATKVISESQNNDKTFAKEVL
SKILGENARVRLALNSVGGKSSASIARKLENNALMLTYGGMSKQPVTLP
TSLHIFKGLTSKGYWVTEKNKKNPQSKIDTISDFIKMYNYGHIISPRDE
IETLTWNTNTTTDEQLLELVKKGITGKGKKKMVVLEW

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:30. In other embodiments, the recombinant organism or microorganism according to the invention comprises a nucleic acid sequence encoding a continuous stretch of at least 100, 150, 200, 250 or 300 amino acids derived from the polypeptide set forth in SEQ ID NO:30.

In certain embodiments, the enzyme that catalyzes the conversion of crotonyl-[acyl-carrier-protein] into butyryl-[acyl-carrier-protein] may be a trans-2-enoyl-CoA reductase (NAD+) (EC 1.3.1.44).

In certain embodiments, the enzyme that catalyzes the conversion of crotonyl-[acyl-carrier-protein] into butyryl-[acyl-carrier-protein] may be FabV from Burkholderia mallei (EC 1.3.1.9 and 1.3.1.44). The amino acid sequence of FabV from Burkholderia mallei (Uniprot Accession No: Q62L02) is set forth in SEQ ID NO:31.

FabV from Burkholderia mallei (SEQ ID NO: 31):
MIIKPRVRGFICVTTHPAGCAASVREQIAYVARRGPIERGPKKVLVIGA
STGYGLAARIAAAFGVGAATLGVFFERAPADAKPGTAGWYNSAAFHDEA
AARGLQATSVNGDAFSDEIKHKTIDAIRRDLGQVDLVVYSVAAPRRTHP
KTGVTHQSTLKPIGHAVRLRGIDTDNEAIKETLLQPATPDEIADTVAVM
GGEDWRMWIDALDAAGVLADGAKTTAFTYLGEQVTHDIYWNGSIGEAKK
DLDRTVLALRGKLAARGGDARVSVLKAVVTQASSAIPMMPLYLSLLFKV
MKARGTHEGCIEQVDGLLRDSLYSAQPHVDAEGRLRADRLELDPAVQAR
VLELWDQVTDDNLYTLTDFAGYKAEFLRLFGFGIDGVDYDAPVEPNVRI
PNLIE

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:31. In other embodiments, the recombinant organism or microorganism according to the invention comprises a nucleic acid sequence encoding a continuous stretch of at least 100, 150, 200, 250 or 300 amino acids derived from the polypeptide set forth in SEQ ID NO:31.

In certain embodiments, the enzyme that catalyzes the conversion of crotonyl-[acyl-carrier-protein] into butyryl-[acyl-carrier-protein] may be FabV from Pseudomonas aeruginosa (EC 1.3.1.9 and 1.3.1.44). The amino acid sequence of FabV from Pseudomonas aeruginosa (Uniprot Accession No: Q9HZP8) is set forth in SEQ ID NO:32.

FabV from Pseudomonas aeruginosa (SEQ ID NO: 32):
MIIKPRVRGFICVTTHPAGCEANVKQQIDYVEAKGPVVNGPKKVLVIGS
STGYGLAARITAAFGSGADTLGVFFERPGSESKPGTAGWYNSAAFEKFA
HEKGLYARSINGDAFSDEVKRLTIETIKRDLGKVDLVVYSLAAPRRTHP
KSGEVFSSTLKPIGKSVSFRGLDTDKEVIKDVVLEAASDQEVADTVAVM
GGEDWQMWIDALLEADVLADGAKTTAFTYLGEKITHDIYWNGSIGAAKK
DLDQKVLGIRDKLAPLGGDARVSVLKAVVTQASSAIPMMPLYLSLLFKV
MKEQGTHEGCIEQVDGLYRESLYGAEPRLDEEGRLRADYKELQPEVQSR
VEELWDKVTNENLYELTDFAGYKSEFLNLFGFEVAGVDYEQDVNPDVQI
ANLIQA

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:32. In other embodiments, the recombinant organism or microorganism according to the invention comprises a nucleic acid sequence encoding a continuous stretch of at least 100, 150, 200, 250 or 300 amino acids derived from the polypeptide set forth in SEQ ID NO:32.

In certain embodiments, the enzyme that catalyzes the conversion of crotonyl-[acyl-carrier-protein] into butyryl-[acyl-carrier-protein] may be FabV from Vibrio cholera (EC 1.3.1.9 and 1.3.1.44). The amino acid sequence of FabV from Vibrio cholera (Uniprot Accession No: Q9KRA3) is set forth in SEQ ID NO:33.

FabV from Vibrio cholera (SEQ ID NO: 33):
MIIKPKIRGFICTTTHPVGCEANVKEQIAYTKAQGPIKNAPKRVLVVGS
SSGYGLSSRIAAAFGGGAATIGVFFEKPGTDKKPGTAGFYNAAAFDKLA
HEAGLYAKSLNGDAFSNEAKQKAIELIKQDLGQIDLVVYSLASPVRKMP
DTGELVRSALKPIGETYTSTAVDTNKDVIIEASVEPATEQEIADTVTVM
GGQDWELWIQALEEAGVLAEGCKTVAYSYIGTELTWPIYWDGALGRAKM
DLDRAATALNEKLAAKGGTANVAVLKSVVTQASSAIPVMPLYIAMVFKK
MREQGVHEGCMEQIYRMFSQRLYKEDGSAPEVDDHNRLRLDDWELRDDI
QQHCRDLWPQITTENLRELTDYDMYKEEFIKLFGFGIEGIDYDADVNPE
VEFDVIDIE

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:33. In other embodiments, the recombinant organism or microorganism according to the invention comprises a nucleic acid sequence encoding a continuous stretch of at least 100, 150, 200, 250 or 300 amino acids derived from the polypeptide set forth in SEQ ID NO:33.

In certain embodiments, the enzyme that catalyzes the conversion of crotonyl-[acyl-carrier-protein] into butyryl-[acyl-carrier-protein] may be FabV from Treponema denticola. The amino acid sequence of FabV from Treponema denticola (Uniprot Accession No: Q73Q47) is set forth in SEQ ID NO:2.

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:2. In other embodiments, the recombinant organism or microorganism according to the invention comprises a nucleic acid sequence encoding a continuous stretch of at least 50, 100, 200, 300 or 350 amino acids derived from the polypeptide set forth in SEQ ID NO:2.

In certain embodiments, the enzyme that catalyzes the conversion of crotonyl-[acyl-carrier-protein] into butyryl-[acyl-carrier-protein] may be FabI from Pseudomonas aeruginosa (EC 1.3.1.9). The amino acid sequence of FabI from Pseudomonas aeruginosa (Uniprot Accession No: Q9ZFE4) is set forth in SEQ ID NO:34.

FabI from Pseudomonas aeruginosa (SEQ ID NO: 34):
MGFLTGKRALIVGVASKLSIASGIAAAMHREGAELAFTYQNDKLRGRVE
EFASGWGSRPELCFPCDVADDSQIEAVFAALGKHWDGLDIIVHSVGFAP
GDQLDGDFTAVTTREGFRIAHDISAYSFIALAKAGREMMKGRNGSLLTL
SYLGAERTMPNYNVMGMAKASLEAGVRYLAGSLGAEGTRVNAVSAGPIR
TLAASGIKSFRKMLAANERQTPLRRNVTIEEVGNAGAFLCSDLASGISG
EILYVDGGFNTTAMGPLDDD

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:34. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:34.

In certain embodiments, the enzyme that catalyzes the conversion of crotonyl-[acyl-carrier-protein] into butyryl-[acyl-carrier-protein] may be FabI from Burkholderia pseudomallei (EC 1.3.1.9). The amino acid sequence of FabI from Burkholderia pseudomallei (Uniprot Accession No: Q3JQY0) is set forth in SEQ ID NO:35.

Fabl from Burkholderia pseudomallei
(SEQ ID NO: 35):
MGFLDGKRILLTGLLSNRSIAYGIAKACKREGAELAFTYVGDRFKDRIT
EFAAEFGSELVFPCDVADDAQIDALFASLKTHWDSLDGLVHSIGFAPRE
AIAGDFLDGLTRENFRIAHDISAYSFPALAKAALPMLSDDASLLTLSYL
GAERAIPNYNTMGLAKAALEASVRYLAVSLGAKGVRVNAISAGPIKTLA
ASGIKSFGKILDFVESNSPLKRNVTIEQVGNAGAFLLSDLASGVTAEVM
HVDSGFNAVVGGMAGLEE

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:35. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:35.

In a preferred embodiment, the enzyme that catalyzes the conversion of crotonyl-[acyl-carrier protein] into butyryl-[acyl-carrier protein] may be a trans-2-enoyl-CoA reductase (EC 1.3.1.44), an enoyl-[acyl-carrier-protein] reductase (NADH) (EC 1.3.1.9) or an enoyl-[acyl-carrier-protein] reductase (NADPH) (EC 1.3.1.104), in particular any trans-2-enoyl-CoA reductase or enoyl-[acyl-carrier-protein] reductase, or variant thereof, as defined herein above.

In certain embodiments, the invention relates to a recombinant organism or microorganism comprising a nucleic acid encoding an NADH or NADPH-dependent enzyme capable of reducing a carbon-carbon double bond (EC 1.3.1), preferably wherein the NADH or NADPH-dependent enzyme capable of reducing a carbon-carbon double bond (EC 1.3.1) is an enoyl-[acyl-carrier-protein] reductase (NADH-dependent) (EC 1.3.1.9), an enoyl-[acyl-carrier-protein] reductase (NADPH-dependent) (EC 1.3.1.104), an enoyl-[acyl-carrier-protein] reductase (NADPH-dependent, Re-specific) (EC 1.3.1.39), an enoyl-[acyl-carrier-protein] reductase (NADPH-dependent, Si-specific) (EC 1.3.1.10), and/or a trans-2-enoyl-CoA reductase (NAD+) (EC 1.3.1.44), preferably wherein the NADH or NADPH-dependent enzyme capable of reducing a carbon-carbon double bond (EC 1.3.1) is a trans-2-enoyl-CoA reductase (EC 1.3.1.44) and/or an enoyl-[acyl-carrier-protein] reductase (NADH-dependent) (EC 1.3.1.9), preferably wherein the NADH or NADPH-dependent enzyme capable of reducing a carbon-carbon double bond (EC 1.3.1) is a trans-2-enoyl-CoA reductase (EC 1.3.1.44).

In certain embodiments, the level and/or activity of said enzymes are increased in the recombinant organism or microorganism in comparison to the organism or microorganism from which it is derived. Increased levels and/or activity of an enzyme may be achieved as described herein. In certain embodiments, the nucleic acid is under control of a promoter. In certain embodiments, the nucleic acid and/or the promoter are of heterologous origin. In certain embodiments, the promoter is not the natural promoter of the nucleic acid. In certain embodiments, the nucleic acid including the promoter is integrated into the genome and/or is located on an extrachromosomal element, such as a plasmid. In certain embodiments, the recombinant organism or microorganism is an organism that is capable of producing 3-methylcrotonic acid and/or isobutene, such as any one of the organisms disclosed herein. Furthermore, the recombinant organism or microorganism may comprise one or more additional modifications that result in a decreased pool of crotonic acid, as described herein.

Decreasing the Conversion of Crotonyl-[Acyl-Carrier-Protein] into Crotonic Acid

Instead of directing metabolic flux away from crotonic acid with any one of the strategies discussed above, the production of crotonic acid in the recombinant organism or microorganism may also be decreased by directly reducing the metabolic flux from crotonyl-[acyl-carrier-protein] to crotonic acid. For that, it is preferred that the expression level and/or the activity of one or more endogenous enzyme that can catalyze the conversion of crotonyl-[acyl-carrier-protein] into crotonic acid will be decreased.

In a particular embodiment, the invention relates to the recombinant organism or microorganism according to the invention, wherein the decreased conversion of crotonyl-[acyl-carrier-protein] into crotonic acid is due to a decreased level and/or a decreased activity of a thioester hydrolase (EC 3.1.2) in said organism or microorganism.

That is, the recombinant organism or microorganism may have decreased levels of any enzyme from EC class 3.1.2.- that is capable of converting crotonyl-[acyl-carrier-protein] into crotonic acid. Alternatively, or in addition, the activity of any enzyme from EC class 3.1.2.- that is capable of converting crotonyl-[acyl-carrier-protein] into crotonic acid may be decreased in the recombinant organism or microorganism.

In a particular embodiment, the invention relates to the recombinant organism or microorganism according to the invention, wherein the thioester hydrolase (EC 3.1.2) is a palmitoyl-CoA hydrolase (EC 3.1.2.2), an acyl-CoA thioesterase 2 (EC 3.1.2.20) or a 1,4-dihydroxy-2-naphtoyl-CoA hydrolase (EC 3.1.2.28).

In E. coli, a thioester hydrolase (EC 3.1.2) is encoded by the gene paaY. Thus, in a certain embodiment, the organism or microorganism of the present invention is E. coli having decreased levels and/or activity of the gene product of the paaY gene. The nucleic acid sequence of the paaY gene is provided in SEQ ID NO:36. In certain embodiments, the nucleic acid sequence as set forth in SEQ ID NO:36 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:36 may be deleted in the organism or microorganism according to the invention. In certain embodiments, a part of the nucleic acid sequence as set forth in SEQ ID NO:36 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 400, 500 nucleotides of the nucleic acid sequence as set forth in SEQ ID NO:36 may be deleted in the organism or microorganism of the invention.

paaY from Escherichia coli (SEQ ID NO: 36):
ATGCCAATTTATCAGATAGACGGTCTGACTCCGGTTGTGCCAGAAGAGA
GTTTTGTCCATCCGACAGCGGTATTGATCGGCGATGTTATTCTCGGCAA
GGGCGTTTACGTTGGGCCAAATGCCAGCCTGCGTGGCGATTTTGGTCGT
ATCGTGGTGAAAGATGGCGCGAACATTCAGGATAATTGCGTTATGCACG
GTTTTCCCGAGCAGGATACTGTTGTAGGAGAAGATGGACATATTGGTCA
TAGCGCTATCCTTCACGGCTGCATTATCCGCCGCAATGCATTAGTGGGA
ATGAACGCGGTAGTGATGGACGGTGCGGTGATTGGCGAGAACAGCATTG
TTGGTGCATCCGCATTTGTGAAAGCCAAAGCAGAAATGCCAGCTAATTA
CCTGATTGTCGGCAGCCCGGCGAAAGCGATTCGTGAACTCAGTGAGCAG
GAGTTGGCATGGAAAAAGCAGGGTACGCATGAGTACCAGGTGCTGGTGA
CACGCTGTAAGCAGACGTTACATCAAGTCGAGCCATTGCGGGAAATTGA
ACCTGGCAGGAAACGCCTGGTATTTGATGAGAATCTGCGACCGAAACAG
TAA

Another thioester hydrolase (EC 3.1.2) is encoded in E. coli by the gene paal. Thus, in a certain embodiment, the organism or microorganism of the present invention is E. coli having decreased levels and/or activity of the gene product of the paal gene. The nucleic acid sequence of the paal gene is provided in SEQ ID NO:37. In certain embodiments, the nucleic acid sequence as set forth in SEQ ID NO: 37 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:37 may be deleted in the organism or microorganism according to the invention. In certain embodiments, a part of the nucleic acid sequence as set forth in SEQ ID NO:37 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 400 nucleotides of the nucleic acid sequence as set forth in SEQ ID NO:37 may be deleted in the organism or microorganism of the invention.

paal from Escherichia coli (SEQ ID NO: 37):
ATGAGTCATAAGGCCTGGCAAAATGCCCATGCAATGTATGAGAACGAT
GCCTGCGCCAAAGCGCTTGGCATCGACATTATCTCAATGGATGAAGGC
TTTGCTGTAGTGACCATGACCGTCACTGCACAAATGCTTAACGGTCAT
CAAAGTTGCCACGGCGGGCAGCTATTTTCACTGGCTGATACTGCCTTT
GCCTACGCCTGCAATAGCCAGGGGCTGGCAGCCGTCGCTTCTGCCTGC
ACGATTGATTTTTTGCGTCCAGGCTTTGCCGGAGACACCTTAACTGCT
ACTGCGCAGGTACGTCATCAGGGCAAGCAAACCGGTGTTTACGACATC
GAAATTGTTAACCAACAACAAAAAACGGTTGCGCTGTTTCGCGGTAAA
TCTCACCGCATCGGCGGCACCATTACAGGAGAAGCCTGA

In E. coli, a palmitoyl-CoA hydrolase (EC 3.1.2.2) is encoded by the gene tesA. Thus, in a certain embodiment, the organism or microorganism of the present invention is E. coli having decreased levels and/or activity of the gene product of the tesA gene. The nucleic acid sequence of the tesA gene is provided in SEQ ID NO:38. In certain embodiments, the nucleic acid sequence as set forth in SEQ ID NO: 38 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:38 may be deleted in the organism or microorganism according to the invention. In certain embodiments, a part of the nucleic acid sequence as set forth in SEQ ID NO:38 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 400, 500, 600 nucleotides of the nucleic acid sequence as set forth in SEQ ID NO: 38 may be deleted in the organism or microorganism of the invention.

tesA from Escherichia coli (SEQ ID NO: 38):
ATGATGAACTTCAACAATGTTTTCCGCTGGCATTTGCCCTTCCTGTTCCTGGTCCTGTTAACCTTCCGTG
CCGCCGCAGCGGACACGTTATTGATTCTGGGTGATAGCCTGAGCGCCGGGTATCGAATGTCTGCCAGCGC
GGCCTGGCCTGCCTTGTTGAATGATAAGTGGCAGAGTAAAACGTCGGTAGTTAATGCCAGCATCAGCGGC
GACACCTCGCAACAAGGACTGGCGCGCCTTCCGGCTCTGCTGAAACAGCATCAGCCGCGTTGGGTGCTGG
TTGAACTGGGCGGCAATGACGGTTTGCGTGGTTTTCAGCCACAGCAAACCGAGCAAACGCTGCGCCAGAT
TTTGCAGGATGTCAAAGCCGCCAACGCTGAACCATTGTTAATGCAAATACGTCTGCCTGCAAACTATGGT
CGCCGTTATAATGAAGCCTTTAGCGCCATTTACCCCAAACTCGCCAAAGAGTTTGATGTTCCGCTGCTGC
CCTTTTTTATGGAAGAGGTCTACCTCAAGCCACAATGGATGCAGGATGACGGTATTCATCCCAACCGCGA
CGCCCAGCCGTTTATTGCCGACTGGATGGCGAAGCAGTTGCAGCCTTTAGTAAATCATGACTCATAA

Another palmitoyl-CoA hydrolase (EC 3.1.2.2) is encoded in E. coli by the gene yciA. Thus, in a certain embodiment, the organism or microorganism of the present invention is E. coli having decreased levels and/or activity of the gene product of the yciA gene. The nucleic acid sequence of the yciA gene is provided in SEQ ID NO:39. In certain embodiments, the nucleic acid sequence as set forth in SEQ ID NO: 39 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:39 may be deleted in the organism or microorganism according to the invention. In certain embodiments, a part of the nucleic acid sequence as set forth in SEQ ID NO:39 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 350 nucleotides of the nucleic acid sequence as set forth in SEQ ID NO:39 may be deleted in the organism or microorganism of the invention.

yciA from Escherichia coli (SEQ ID NO: 39):
ATGTCTACAACACATAACGTCCCTCAGGGCGATCTTGTTTTACGTACTTTAGCCATGCCCGCCGATACCA
ATGCCAATGGTGACATCTTTGGTGGTTGGTTAATGTCACAAATGGATATTGGCGGCGCTATTCTGGCAAA
AGAAATTGCCCACGGTCGCGTAGTGACTGTGCGGGTTGAAGGAATGACTTTCTTACGGCCGGTTGCGGTC
GGCGATGTGGTGTGCTGCTATGCACGCTGTGTCCAGAAAGGGACGACATCGGTCAGCATTAATATTGAAG
TGTGGGTGAAAAAAGTAGCGTCTGAACCAATTGGGCAACGCTATAAAGCGACAGAAGCATTATTTAAGTA
TGTCGCGGTTGATCCTGAAGGAAAACCTCGCGCCTTACCTGTTGAGTAA

Another palmitoyl-CoA hydrolase (EC 3.1.2.2) is encoded in E. coli by the gene entH. Thus, in a certain embodiment, the organism or microorganism of the present invention is E. coli having decreased levels and/or activity of the gene product of the entH gene. The nucleic acid sequence of the entH gene is provided in SEQ ID NO:40. In certain embodiments, the nucleic acid sequence as set forth in SEQ ID NO: 40 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:40 may be deleted in the organism or microorganism according to the invention. In certain embodiments, a part of the nucleic acid sequence as set forth in SEQ ID NO:40 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 350, 400 nucleotides of the nucleic acid sequence as set forth in SEQ ID NO:40 may be deleted in the organism or microorganism of the invention.

entH from Escherichia coli (SEQ ID NO: 40):
ATGATCTGGAAACGCCATTTAACGCTCGACGAACTGAACGCCACCAGCGATAACACAATGGTGGCGCATC
TGGGAATTGTGTATACCCGTCTGGGCGATGATGTGCTGGAAGCCGAAATGCCGGTTGATACCCGTACTCA
TCAGCCGTTCGGTTTACTACATGGCGGCGCGTCGGCGGCGCTGGCGGAAACGCTGGGATCGATGGCCGGA
TTTATGATGACCCGCGACGGACAGTGTGTGGTAGGCACAGAACTTAATGCAACACACCATCGCCCGGTGT
CTGAGGGAAAGGTACGCGGCGTCTGCCAGCCGCTGCATCTTGGTCGGCAAAATCAGAGCTGGGAAATCGT
CGTTTTCGATGAACAGGGGCGGCGTTGCTGCACTTGTCGGCTGGGTACGGCAGTTTTGGGATGA

In E. coli, an acyl-CoA thioesterase 2 (EC 3.1.2.20) is encoded by the gene tesB. Thus, in a certain embodiment, the organism or microorganism of the present invention is E. coli having decreased levels and/or activity of the gene product of the tesB gene. The nucleic acid sequence of the tesB gene is provided in SEQ ID NO:41. In certain embodiments, the nucleic acid sequence as set forth in SEQ ID NO: 41 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:41 may be deleted in the organism or microorganism according to the invention. In certain embodiments, a part of the nucleic acid sequence as set forth in SEQ ID NO:41 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 400, 500, 600, 700, 800 nucleotides of the nucleic acid sequence as set forth in SEQ ID NO: 41 may be deleted in the organism or microorganism of the invention.

tesB from Escherichia coli (SEQ ID NO: 41):
ATGAGTCAGGCGCTAAAAAATTTACTGACATTGTTAAATCTGGAAAAAATTGAGGAAGGACTCTTTCGCG
GCCAGAGTGAAGATTTAGGTTTACGCCAGGTGTTTGGCGGCCAGGTCGTGGGTCAGGCCTTGTATGCTGC
AAAAGAGACCGTCCCTGAAGAGCGGCTGGTACATTCGTTTCACAGCTACTTTCTTCGCCCTGGCGATAGT
AAGAAGCCGATTATTTATGATGTCGAAACGCTGCGTGACGGTAACAGCTTCAGCGCCCGCCGGGTTGCTG
CTATTCAAAACGGCAAACCGATTTTTTATATGACTGCCTCTTTCCAGGCACCAGAAGCGGGTTTCGAACA
TCAAAAAACAATGCCGTCCGCGCCAGCGCCTGATGGCCTCCCTTCGGAAACGCAAATCGCCCAATCGCTG
GCGCACCTGCTGCCGCCAGTGCTGAAAGATAAATTCATCTGCGATCGTCCGCTGGAAGTCCGTCCGGTGG
AGTTTCATAACCCACTGAAAGGTCACGTCGCAGAACCACATCGTCAGGTGTGGATCCGCGCAAATGGTAG
CGTGCCGGATGACCTGCGCGTTCATCAGTATCTGCTCGGTTACGCTTCTGATCTTAACTTCCTGCCGGTA
GCTCTACAGCCGCACGGCATCGGTTTTCTCGAACCGGGGATTCAGATTGCCACCATTGACCATTCCATGT
GGTTCCATCGCCCGTTTAATTTGAATGAATGGCTGCTGTATAGCGTGGAGAGCACCTCGGCGTCCAGCGC
ACGTGGCTTTGTGCGCGGTGAGTTTTATACCCAAGACGGCGTACTGGTTGCCTCGACCGTTCAGGAAGGG
GTGATGCGTAATCACAATTAA

Another acyl-CoA thioesterase 2 (EC 3.1.2.20) is encoded in E. coli by the gene fadM. Thus, in a certain embodiment, the organism or microorganism of the present invention is E. coli having decreased levels and/or activity of the gene product of the fadM gene. The nucleic acid sequence of the fadM gene is provided in SEQ ID NO:42. In certain embodiments, the nucleic acid sequence as set forth in SEQ ID NO: 42 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:42 may be deleted in the organism or microorganism according to the invention. In certain embodiments, a part of the nucleic acid sequence as set forth in SEQ ID NO:42 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 350 nucleotides of the nucleic acid sequence as set forth in SEQ ID NO:42 may be deleted in the organism or microorganism of the invention.

fadM from Escherichia coli (SEQ ID NO: 42):
ATGCAAACACAAATCAAAGTTCGTGGATATCATCTCGACGTTTACCAGCACGTCAACAACGCCCGCTACC
TTGAATTTCTCGAAGAAGCCCGCTGGGATGGGTTGGAAAATAGCGACAGTTTTCAGTGGATGACGGCCCA
TAACATCGCCTTCGTCGTGGTCAATATCAATATTAACTATCGTCGCCCAGCGGTATTAAGTGACCTGTTA
ACTATTACCAGTCAGTTGCAGCAATTAAACGGTAAAAGCGGCATCTTAAGCCAGGTCATTACACTGGAGC
CGGAAGGGCAGGTGGTAGCGGATGCGCTTATTACGTTTGTTTGTATTGATCTTAAAACGCAGAAAGCATT
AGCTCTGGAAGGGGAATTGCGCGAAAAGCTGGAGCAGATGGTTAAGTAA

In E. coli, a 1,4-dihydroxy-2-naphtoyl-CoA hydrolase (EC 3.1.2.28) is encoded by the gene menI (also named ydiI). Thus, in a certain embodiment, the organism or microorganism of the present invention is E. coli having decreased levels and/or activity of the gene product of the menI gene. The nucleic acid sequence of the menI gene is provided in SEQ ID NO:43. In certain embodiments, the nucleic acid sequence as set forth in SEQ ID NO:43 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:43 may be deleted in the organism or microorganism according to the invention. In certain embodiments, a part of the nucleic acid sequence as set forth in SEQ ID NO:43 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 400 nucleotides of the nucleic acid sequence as set forth in SEQ ID NO:43 may be deleted in the organism or microorganism of the invention.

menI from Escherichia coli (SEQ ID NO: 43):
ATGATATGGAAACGGAAAATCACCCTGGAAGCACTGAATGCTATGGGTGAAGGAAACATGGTGGGGTTCC
TGGATATTCGCTTTGAACATATTGGTGATGACACCCTTGAAGCGACAATGCCAGTAGACTCGCGGACAAA
GCAGCCTTTCGGGTTGCTGCATGGAGGAGCATCCGTGGTACTGGCCGAAAGTATCGGTTCCGTTGCCGGT
TATTTATGTACCGAAGGTGAGCAAAAAGTGGTTGGTCTGGAAATCAATGCTAACCACGTCCGCTCGGCAC
GAGAAGGGCGGGTGCGCGGCGTATGCAAACCGTTGCATCTCGGTTCGCGTCACCAGGTCTGGCAGATTGA
AATCTTCGATGAGAAAGGGCGTTTGTGCTGTTCGTCACGATTGACGACCGCCATTTTGTGA

As described in more detail above, reducing the level of an enzyme encoded by any of the genes described above in a recombinant organism or microorganism according to the invention may be achieved by fully or partially deleting the coding sequence of said gene or by deleting/modifying one or more regulatory elements of said genes. Alternatively, the activity of an enzyme encoded by any of the genes described above in a recombinant organism or microorganism according to the invention may be decreased by introducing inactivating mutations into said gene or through the addition of inhibitors of said enzyme.

Decreasing the Conversion of Crotonyl-CoA into Crotonic Acid

Instead of directing metabolic flux away from crotonic acid with any one of the strategies discussed above, the production of crotonic acid in the recombinant organism or microorganism may also be decreased by directly reducing the metabolic flux from crotonyl-CoA to crotonic acid. For example, it is known in E. coli that the thioester hydrolase YdiI can convert crotonyl-CoA into crotonic acid.

Thus, it is preferred that the expression level and/or the activity of one or more endogenous enzyme that can catalyze the conversion of crotonyl-CoA into crotonic acid will be decreased.

That is, in a particular embodiment, the invention relates to the recombinant organism or microorganism according to the invention, wherein the decreased conversion of crotonyl-CoA into crotonic acid is due to a decreased level and/or a decreased activity of a thioester hydrolase (EC 3.1.2) in said organism or microorganism.

That is, the recombinant organism or microorganism may have decreased levels of any enzyme from EC class 3.1.2.- that is capable of converting crotonyl-CoA into crotonic acid. Alternatively, or in addition, the activity of any enzyme from EC class 3.1.2.- that is capable of converting crotonyl-CoA into crotonic acid may be decreased in the recombinant organism or microorganism.

In a particular embodiment, the invention relates to the recombinant organism or microorganism according to the invention, wherein the thioester hydrolase (EC 3.1.2) is a palmitoyl-CoA hydrolase (EC 3.1.2.2), an acyl-CoA thioesterase 2 (EC 3.1.2.20) or a 1,4-dihydroxy-2-naphtoyl-CoA hydrolase (EC 3.1.2.28).

Preferably, the recombinant organism or microorganism has a reduced level and/or reduced activity of the enzyme YdiI (MenI). Thus, in a certain embodiment, the organism or microorganism of the present invention is E. coli having decreased levels and/or activity of the gene product of the menI gene. The nucleic acid sequence of the menI gene is provided in SEQ ID NO:42. In certain embodiments, the nucleic acid sequence as set forth in SEQ ID NO:42 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO: 42 may be deleted in the organism or microorganism according to the invention. In certain embodiments, a part of the nucleic acid sequence as set forth in SEQ ID NO:42 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 400 nucleotides of the nucleic acid sequence as set forth in SEQ ID NO:42 may be deleted in the organism or microorganism of the invention.

In another preferred embodiment, the organism or microorganism of the present invention is E. coli having decreased levels and/or activity of the gene product of the yciA gene. The nucleic acid sequence of the yciA gene is provided in SEQ ID NO:39. In certain embodiments, the nucleic acid sequence as set forth in SEQ ID NO:39 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:39 may be deleted in the organism or microorganism according to the invention. In certain embodiments, a part of the nucleic acid sequence as set forth in SEQ ID NO:39 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 350 nucleotides of the nucleic acid sequence as set forth in SEQ ID NO:39 may be deleted in the organism or microorganism of the invention.

In yet another preferred embodiment, the organism or microorganism of the present invention is E. coli having decreased levels and/or activity of the gene product of the tesB gene. The nucleic acid sequence of the tesB gene is provided in SEQ ID NO:41. In certain embodiments, the nucleic acid sequence as set forth in SEQ ID NO:41 may be deleted in the organism or microorganism according to the invention. In certain embodiments, a nucleic acid sequence having at least 30%, 40%, 50%, 60%, 70%, 80% or 90% sequence identity with the nucleic acid sequence as set forth in SEQ ID NO:41 may be deleted in the organism or microorganism according to the invention. In certain embodiments, a part of the nucleic acid sequence as set forth in SEQ ID NO:41 may be deleted in the organism or microorganism according to the invention. That is, a continuous stretch comprising at least 1, 2, 3, 4, 5, 6, 7, 8, 9, 10, 20, 30, 40, 50, 100, 150, 200, 250, 300, 400, 500, 600, 700, 800 nucleotides of the nucleic acid sequence as set forth in SEQ ID NO:41 may be deleted in the organism or microorganism of the invention.

It has to be noted that the activity of a thioester hydrolase is essential in certain production processes. For example, the thioester hydrolase YdiI (MenI) is commonly employed for the production of isobutene from acetyl-CoA. However, other processes, such as the production of 1,3-butadiene, do not require thioester hydrolase activity to convert 3-methylcrotonyl-CoA into 3-methylcrotonic acid like for isobutene production. For example, reducing the level and/or activity of YdiI (MenI) may allow decreasing the pool of crotonic acid in a recombinant organism or microorganism that is used, for example, for the production of 1,3-butadiene. Thus, it is to be understood that decreasing the pool of crotonic acid in a recombinant organism or microorganism by decreasing the conversion of crotonyl-CoA to crotonic acid is optional and may only be applied in recombinant organisms or microorganisms that do not require expression of a thioester hydrolase to convert 3-methylcrotonyl-CoA into 3-methylcrotonic acid, such as YdiI.

Alternatively, or in addition, the level and/or activity of further thioester hydrolases may be decreased. For example, the levels and/or activities of the E. coli thioester hydrolases PaaY (encoded by SEQ ID NO: 36), PaaI (encoded by SEQ ID NO:37), TesA (encoded by SEQ ID NO:38 YciA (encoded by SEQ ID NO: 39), EntH (encoded by SEQ ID NO:40), TesB (encoded by SEQ ID NO:41) or FadM (encoded by SEQ ID NO: 42) may be decreased as disclosed elsewhere herein.

In certain embodiments, the conversion of crotonyl-CoA into crotonic acid and/or the conversion of crotonyl [acyl-carrier protein] into crotonic acid is decreased by decreasing the level and/or activity of an endogenous thioester hydrolase (EC 3.1.2), in particular an endogenous palmitoyl-CoA hydrolase (EC 3.1.2.2), acyl-CoA thioesterase 2 (EC 3.1.2.20) and/or 1,4-dihydroxy-2-naphtoyl-CoA hydrolase (EC 3.1.2.28).

In certain embodiments, the invention relates to a recombinant organism or microorganism that is characterized by reduced levels and/or activity of an endogenous thioester hydrolase (EC 3.1.2), preferably wherein the endogenous thioester hydrolase (EC 3.1.2) is a palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28), preferably wherein the endogenous thioester hydrolase (EC 3.1.2) is an acyl-CoA thioesterase 2 (EC 3.1.2.20). In certain embodiments, the endogenous thioester hydrolase (EC 3.1.2) is yciA (SEQ ID NO:39) or a homolog thereof. In certain embodiments, the endogenous thioester hydrolase (EC 3.1.2) is tesB (SEQ ID NO:41) or a homolog thereof.

It is to be understood that the levels and/or activities of said enzymes are to be compared to the levels and/or activities in the organism or microorganism from which the recombinant organism or microorganism of the invention is derived. Reduced levels and/or activities of enzymes may be achieved as described herein. In certain embodiments, an endogenous nucleic acid encoding an enzyme is partially or completely deleted or replaced. In certain embodiments, the activity of an endogenous enzyme is reduced by introducing one or more mutations in the nucleic acid encoding said enzyme. In certain embodiments, the recombinant organism or microorganism is an organism that is capable of producing 3-methylcrotonic acid and/or isobutene, such as any one of the organisms disclosed herein. Furthermore, the recombinant organism or microorganism may comprise one or more additional modifications that result in a decreased pool of crotonic acid, as described herein.

In certain embodiments, the recombinant organism or microorganism of the invention has a decreased level and/or activity of one or more endogenous thioester hydrolase (EC 3.1.2), in particular an endogenous palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28); and one or more additional modifications resulting in
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonyl-[acyl-carrier protein] into butyryl [acyl-carrier-protein]; and/or
(iv) an increased conversion of crotonic acid into crotonyl-CoA;
   as described herein.

That is, in certain embodiments, the recombinant organism or microorganism of the invention comprises:
a) a decreased level and/or activity of one or more endogenous thioester hydrolase (EC 3.1.2), in particular an endogenous palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28); and
b) an increased level and/or activity of one or more NADH or NADPH-dependent enzyme capable of reducing a carbon-carbon double bond (EC 1.3.1) and/or a flavin-dependent enzyme capable of reducing a carbon-carbon double bond (EC 1.3.8), in particular wherein the NADH or NADPH-dependent enzyme capable of reducing a carbon-carbon double bond (EC 1.3.1) is a crotonyl-CoA reductase (EC 1.3.1.86), a trans-2-enoyl-CoA reductase (EC 1.3.1.44) or an enoyl-[acyl-carrier-protein] reductase (EC 1.3.1.9) and wherein flavin-dependent enzyme capable of reducing a carbon-carbon double bond (EC 1.3.8) is a short-chain acyl-CoA dehydrogenase (EC 1.3.8.1).

In a preferred embodiment, the recombinant organism or microorganism of the invention comprises:
a) a decreased level and/or activity of an acyl-CoA thioesterase 2 (EC 3.1.2.20); and
b) an increased level and/or activity of a trans-2-enoyl-CoA reductase (EC 1.3.1.44) and/or an enoyl-[acyl-carrier-protein] reductase (EC 1.3.1.9).

In a particularly preferred embodiment, the recombinant organism or microorganism of the invention is derived from E. coli and comprises:
a) a decreased level and/or activity of an acyl-CoA thioesterase 2 (EC 3.1.2.20), wherein the acyl-CoA thioesterase 2 is YciA or TesB from E. coli; and
b) an increased level and/or activity of a trans-2-enoyl-CoA reductase (EC 1.3.1.44) and/or an enoyl-[acyl-carrier-protein] reductase (EC 1.3.1.9).

Preferably the trans-2-enoyl-CoA reductase (EC 1.3.1.44) is FabV from Treponema denticola or any other suitable trans-2-enoyl-CoA reductase and the enoyl-[acyl-carrier-protein] reductase (EC 1.3.1.9) is FabI from Escherichia coli or any other suitable enoyl-[acyl-carrier-protein] reductase.

In a particularly preferred embodiment, the recombinant organism or microorganism of the invention is derived from E. coli and comprises:
a) a decreased level and/or activity of an acyl-CoA thioesterase 2 (EC 3.1.2.20), preferably wherein the acyl-CoA thioesterase 2 is YciA from E. coli; and
b) an increased level and/or activity of a trans-2-enoyl-CoA reductase (EC 1.3.1.44) and/or an enoyl-[acyl-carrier-protein] reductase (EC 1.3.1.9), preferably wherein the trans-2-enoyl-CoA reductase (EC 1.3.1.44) is FabV from Treponema denticola and wherein the enoyl-[acyl-carrier-protein] reductase (EC 1.3.1.9) is FabI from Escherichia coli.

In a particularly preferred embodiment, the recombinant organism or microorganism of the invention is derived from E. coli and comprises:
a) a decreased level and/or activity of an acyl-CoA thioesterase 2 (EC 3.1.2.20), preferably wherein the acyl-CoA thioesterase 2 is YciA from E. coli; and
b) an increased level and/or activity of a trans-2-enoyl-CoA reductase (EC 1.3.1.44), preferably wherein the trans-2-enoyl-CoA reductase (EC 1.3.1.44) is FabV from Treponema denticola.

In a particularly preferred embodiment, the recombinant organism or microorganism of the invention is derived from E. coli and comprises:
a) a decreased level and/or activity of an acyl-CoA thioesterase 2 (EC 3.1.2.20), preferably wherein the acyl-CoA thioesterase 2 is TesB from E. coli; and
b) an increased level and/or activity of a trans-2-enoyl-CoA reductase (EC 1.3.1.44) and/or an enoyl-[acyl-carrier-protein] reductase (EC 1.3.1.9), preferably wherein the trans-2-enoyl-CoA reductase (EC 1.3.1.44) is FabV from Treponema denticola and wherein the enoyl-[acyl-carrier-protein] reductase (EC 1.3.1.9) is FabI from Escherichia coli.

In a particularly preferred embodiment, the recombinant organism or microorganism of the invention is derived from E. coli and comprises:
a) a decreased level and/or activity of an acyl-CoA thioesterase 2 (EC 3.1.2.20), preferably wherein the acyl-CoA thioesterase 2 is TesB from E. coli; and
b) an increased level and/or activity of an enoyl-[acyl-carrier-protein] reductase (EC 1.3.1.9), preferably wherein the enoyl-[acyl-carrier-protein] reductase (EC 1.3.1.9) is FabI from Escherichia coli.

In certain embodiments, the recombinant organism or microorganism has
a) a decreased level and/or activity of one or more endogenous thioester hydrolase (EC 3.1.2), preferably an endogenous palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28), more preferably an acyl-CoA thioesterase 2 (EC 3.1.2.20); and
b) an increased level and/or activity of one or more hydro-lyase (EC 4.2.1), in particular wherein the hydro-lyase (EC 4.2.1) is a short-chain-enoyl-CoA hydratase (EC 4.2.1.150), a 3-hydroxybutyryl-CoA dehydratase (EC 4.2.1.55) or an enoyl-CoA hydratase (EC 4.2.1.17).

In certain embodiments, the recombinant organism or microorganism of the invention comprises:
a) a decreased level and/or activity of one or more endogenous thioester hydrolase (EC 3.1.2), preferably an endogenous palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28), more preferably an acyl-CoA thioesterase 2 (EC 3.1.2.20); and
b) an increased level and/or activity of one or more NADH or NADPH-dependent enzyme capable of reducing a carbon-carbon double bond (EC 1.3.1), in particular wherein the NADH or NADPH-dependent enzyme capable of reducing a carbon-carbon double bond (EC 1.3.1) is an enoyl-[acyl-carrier-protein] reductase (NADH-dependent) (EC 1.3.1.9), an enoyl-[acyl-carrier-protein] reductase (NADPH-dependent) (EC 1.3.1.104), an enoyl-[acyl-carrier-protein] reductase (NADPH-dependent, Re-specific) (EC 1.3.1.39), an enoyl-[acyl-carrier-protein] reductase (NADPH-dependent, Si-specific) (EC 1.3.1.10), and/or a trans-2-enoyl-CoA reductase (NAD+) (EC 1.3.1.44).

In certain embodiments, the recombinant organism or microorganism has
a) a decreased level and/or activity of one or more endogenous thioester hydrolase (EC 3.1.2), preferably an endogenous palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28), more preferably an acyl-CoA thioesterase 2 (EC 3.1.2.20); and
b) an increased level and/or activity of one or more a CoA-transferase (EC 2.8.3), preferably wherein the CoA-transferase is an acetate CoA-transferase (EC 2.8.3.8), an acid thiol ligase (EC 6.2.1), preferably wherein the acid thiol ligase (EC 6.2.1) is a medium-chain acyl-CoA ligase (EC 6.2.1.2), a benzoate-CoA ligase (EC 6.2.1.25), a 4-hydroxybenzoate-CoA ligase (EC 6.2.1.27), a 4-Hydroxybutyrate-CoA ligase (EC 6.2.1.40) or a methylmercaptopropionate (MMPA)-coenzyme A (CoA) ligase (EC 6.2.1.44), an acid kinase (EC 2.7.2), preferably wherein the acid kinase (EC 2.7.2) is a butyrate kinase (EC 2.7.2.7), and/or a phosphate acyltransferase (EC 2.3.1), preferably wherein the phosphate acyltransferase (EC 2.3.1) is a phosphate butyryltransferase (EC 2.3.1.19).
   Directly Converting Crotonic Acid into Crotonyl-CoA

Above, strategies have been discussed to reduce the metabolic flux to crotonic acid. However, the pool of crotonic acid in a recombinant organism or microorganism according to the invention may also be decreased by directly converting crotonic acid into another molecule. For example, the pool of crotonic acid in a recombinant organism or microorganism may be reduced by converting crotonic acid into crotonyl-CoA. The produced crotonyl-CoA may then further be converted into butyryl-CoA or 3-hydroxybutyryl-CoA as discussed herein.

To achieve conversion of crotonic acid into crotonyl-CoA, a gene encoding an enzyme that efficiently converts crotonic acid into crotonyl-CoA may be overexpressed. The enzyme may be a heterologous or an endogenous enzyme as discussed herein and may further be genetically engineered to improve the conversion of crotonic acid into crotonyl-CoA.

In a particular embodiment, the invention relates to the recombinant organism or microorganism according to the invention, wherein the increased conversion of crotonic acid into crotonyl-CoA is due to an increased level and/or activity of a CoA-transferase (EC 2.8.3) and/or an acid thiol ligase (EC 6.2.1) and/or an acid kinase (EC 2.7.2) and/or a phosphate acyltransferase activity (EC 2.3.1) in said organism or microorganism.

That is, the recombinant organism or microorganism may overexpress any enzyme from EC classes 2.8.3, 6.2.1, 2.3.1 and/or 2.7.2 that is capable of converting crotonic acid into crotonyl-CoA.

In a particular embodiment, the invention relates to the recombinant organism or microorganism according to the invention, wherein the CoA-transferase (EC 2.8.3) is an acetate CoA-transferase or a (EC 2.8.3.8).

The enzyme that catalyzes the conversion of crotonic acid into crotonyl-CoA may be any enzyme from EC class 2.8.3 that efficiently catalyzes the conversion of crotonic acid into crotonyl-CoA. However, in a preferred embodiment, the CoA-transferase may be an acetate CoA-transferase or a butyryl-CoA: acetate CoA-transferase (EC 2.8.3.8).

In certain embodiments, the enzyme that catalyzes the conversion of crotonic acid into crotonyl-CoA may be an acetate CoA-transferase (EC 2.8.3.8). A non-limiting example of an acetate CoA-transferase is YdiF (Pct) from Cupriavidus necator. However, acetate CoA-transferases have been described in other organisms. The amino acid sequence of YdiF (Pct) (Uniprot Accession No: Q0K874) from Cupriavidus necator is set forth in SEQ ID NO:9.

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:9. In other embodiments, the recombinant organism or microorganism according to the invention comprises a nucleic acid sequence encoding a continuous stretch of at least 100, 200, 300, 400 or 500 amino acids derived from the polypeptide set forth in SEQ ID NO:9.

In certain embodiments, the enzyme that catalyzes the conversion of crotonic acid into crotonyl-CoA may be a butyrate: acetyl-CoA-transferase (EC 2.8.3.8). Non-limiting examples of a butyrate: acetyl-CoA-transferases are SwoI_1932 and SwoI_0436 from Syntrophomonas wolfei subsp. wolfei. However, butyrate: acetyl-CoA-transferases have been described in other organisms. The amino acid sequence of SwoI_1932 (Uniprot Accession No: Q0AVM5) from Syntrophomonas wolfei subsp. wolfei is set forth in SEQ ID NO:10. The amino acid sequence of SwoI_0436 (Uniprot Accession No: Q0AZT0) from Syntrophomonas wolfei subsp. wolfei is set forth in SEQ ID NO:11.

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:10 or 11. In other embodiments, the recombinant organism or microorganism according to the invention comprises a nucleic acid sequence encoding a continuous stretch of at least 100, 200, 300, 350 or 400 amino acids derived from the polypeptide set forth in SEQ ID NO: 10 or 11.

In a particular embodiment, the invention relates to the recombinant organism or microorganism according to the invention, wherein the acid thiol ligase (EC 6.2.1) is a medium-chain acyl-CoA ligase (EC 6.2.1.2), a benzoate-CoA ligase (EC 6.2.1.25), a 4-hydroxybenzoate-CoA ligase (EC 6.2.1.27), a 4-Hydroxybutyrate-CoA ligase (EC 6.2.1.40), or a methylmercaptopropionate (MMPA)-coenzyme A (CoA) ligase (EC 6.2.1.44).

The enzyme that catalyzes the conversion of crotonic acid into crotonyl-CoA may be any enzyme from EC class 6.2.1 that efficiently catalyzes the conversion of crotonic acid into crotonyl-CoA. However, in a preferred embodiment, the acid thiol ligase may be a medium-chain acyl-CoA ligase (EC 6.2.1.2), a benzoate-CoA ligase (EC 6.2.1.25), a 4-hydroxybenzoate-CoA ligase (EC 6.2.1.27), a 4-Hydroxybutyrate-CoA ligase (EC 6.2.1.40), or a methylmercaptopropionate (MMPA)-coenzyme A (CoA) ligase (EC 6.2.1.44).

In certain embodiments, the enzyme that catalyzes the conversion of crotonic acid into crotonyl-CoA may be a 4-hydroxybenzoate-CoA ligase/benzoate-CoA ligase (EC 6.2.1.25 and 6.2.1.27). A non-limiting example of a 4-hydroxybenzoate-CoA ligase/benzoate-CoA ligase (EC 6.2.1.25 and 6.2.1.27) is encoded by the gene SYN_02896 from Syntrophus aciditrophicus (strain SB). The amino acid sequence of a 4-hydroxybenzoate-CoA ligase/benzoate-CoA ligase (EC 6.2.1.25 and 6.2.1.27) encoded by the gene SYN_02896 from Syntrophus aciditrophicus (strain SB) (Uniprot Accession No: Q2LRH0) is set forth in SEQ ID NO:55.

SYN_02896 from Syntrophus aciditrophicus (strain SB) (SEQ ID NO: 55):
MAKLYPEEFYNSADWFVDRHVREGRGDNICAYTDKGNYTYRDIQKMANKMANMFKDLDIR
MGDRIIMLVLDTPWFYSTFWGAVKMGAVPVPSSTMLTPADYEYYLNDSQARTLVVSSRLL
PVVNQIEELRFLKNMIVVDDDGVFSTPYQQIYASASDEFQTVFTSADDVAFWLYTSGTTG
GPKGAVHSQSDMQYSAEAYGKHILEITEKDICYSAARLFFTYGLGNAMFFPMSVGAAAVL
NPDPPAPAHVERLIKTYKATLFFGVPTLFGQMLITQDKIDAEKGAGADPKDHDLVSLRAC
PSAGEALPPDLYHKFKARYGVEILDGPGSTEMLHIYLSNKLGDVKPGSSGKPVPGYEEKI
MDEEGKNELPDGEVGNLWIKGRSSLRYYWRKRDKTAATVIGEWVNSGDKYYKDAEGYYWP
SGRADDMLKVGGIWVSPLEVENCLREHASVMECAVVGAMDEENLVKPKAFVVLNQGFAQS
PELEKELKQWVLDRLAKFKYPRWIVFIDSLPKTATGKIQRFKLR

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:55. In other embodiments, the recombinant organism or microorganism according to the invention comprises a nucleic acid sequence encoding a continuous stretch of at least 200, 300, 400 or 500 amino acids derived from the polypeptide set forth in SEQ ID NO:55.

Another non-limiting example of a 4-hydroxybenzoate-CoA ligase/benzoate-CoA ligase (EC 6.2.1.25 and 6.2.1.27) is encoded by the gene SYN_02898 from Syntrophus aciditrophicus (strain SB). The amino acid sequence of a 4-hydroxybenzoate-CoA ligase/benzoate-CoA ligase (EC 6.2.1.25 and 6.2.1.27) encoded by the gene SYN_02898 from Syntrophus aciditrophicus (strain SB) (Uniprot Accession No: Q2LRH7) is set forth in SEQ ID NO:56.

SYN_02898 from Syntrophus aciditrophicus (strain SB) (SEQ ID NO: 56):
MAVYTEEFYNSVNYFIDRHIEEGRGDKICAYTDKGNYTYRDMQKMVNKMANMERKLDIRI
GDRVIMLVFDTPWFFSTFWGAVRIGAVPVPSNIMLTSDDYQYYLNDSQARTLVISEKLLP
LIKGIKGELRYLRDVIVVDDDGEFSTPYQQMYAQASEEAETAFTTKDDVAFWQYTSGTTG
APKGAVHSHSDMQYVAEAYGKHVLGMTENDVCYSAARLFFAYGIGNGMVYPLSVGAASVL
NPDPPTPERAFRLNSTYKVTLFFGIPTLFGQMLEYKVKQEKEAGITPDPKAPHELSSVRA
CPSAGEALPPDLYHRFKERFGVEILDGPGSTEMLHIYLSNTLGDVKAGSSGKVVPGYEAK
IVGEEGETLPDGEIGTLWVKGDSSLRYYWRKKEKTASTIIGGWVNTGDKYYRDKDGYFWP
SGRADDMLKVGGIWVSPLEVENCLREHPAVLETAVIGAEDEKNLVKPKAFVVLKQGFAPS
PELEKELKQWVLDRLAKFKYPRWIVEMDELPKTATGKIQRFKLR

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:56.

In other embodiments, the recombinant organism or microorganism according to the invention comprises a nucleic acid sequence encoding a continuous stretch of at least 200, 300, 400 or 500 amino acids derived from the polypeptide set forth in SEQ ID NO:56.

In certain embodiments, the enzyme that catalyzes the conversion of crotonic acid into crotonyl-CoA may be a medium-chain acyl-CoA ligase (EC 6.2.1.2). A non-limiting example of a medium-chain acyl-CoA ligase (EC 6.2.1.2) is encoded by the gene PA3924 from Pseudomonas aeruginosa. The amino acid sequence of a medium-chain acyl-CoA ligase (EC 6.2.1.2) encoded by the gene PA3924 from Pseudomonas aeruginosa (Uniprot Accession No: Q9HX89) is set forth in SEQ ID NO:57.

PA3924 from Pseudomonas aeruginosa (SEQ ID NO: 57):
MLKTRLIPAAAGAYQYPLLIKSLMLSGRRYEKSHEIVYRDQVRYSYATENERVARLANVL
SEAGVKAGDTVAVMDWDSHRYLECMFAIPMIGAVLHTINIRLSPEQILYTMNHAEDRFVL
VNSEFVPLYQAVAGQLATVERTILLTDGAEKSAELPGLVGEYESLLAAASPRYDEPDFDE
NSIATTFYTTGTTGNPKGVYFSHRQLVLHTLAMASTIGSLDSIRLLGTSDVYMPITPMFH
VHAWGTPYVATMLGVKQVYPGRYDPELLVELWKREKVTFSHCVPTILQMVMNARAAQGVD
FKGWKVIIGGSALNRSLYEAAKARGIQLTAAYGMSETCPLISCAYLNDELLAGSEDERTT
YRIKAGVPVPLVDAAIMDEQGRFLPADGESQGELVLRSPWLTQGYFREPERGEELWRGGW
MHTGDVATLDGMGFIEIRDRIKDVIKTGGEWLSSLELEDLISRHPAVREVAVVGVPDPQW
GERPFALLVVREGQQLDARGLKEHLKPFVEQGNINKWAIPSQIAVVTDIPKTSVGKLDKK
RIRIEIAQWQEAGSAFLSTV

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:57. In other embodiments, the recombinant organism or microorganism according to the invention comprises a nucleic acid sequence encoding a continuous stretch of at least 200, 300, 400 or 500 amino acids derived from the polypeptide set forth in SEQ ID NO:57.

In certain embodiments, the enzyme that catalyzes the conversion of crotonic acid into crotonyl-CoA may be a 4-Hydroxybutyrate-CoA ligase (EC 6.2.1.40). A non-limiting example of a 4-Hydroxybutyrate-CoA ligase (EC 6.2.1.40) is encoded by the gene Tneu_0420 from Pyrobaculum neutrophilum (Thermoproteus neutrophilus). The amino acid sequence of a 4-Hydroxybutyrate-CoA ligase encoded by the gene Tneu_0420 from Pyrobaculum neutrophilum (Uniprot Accession No: B1YBY4) is set forth in SEQ ID NO:44.

Tneu_0420 from Pyrobaculum neutrophilum (SEQ ID NO: 44):
MSAEFVEVYRKSLEDPIGFWEKQAERLYWRERWEKTYDDSNPPFYRWFVGGKINISYNAL
DRHVKGGRANKAALIWVSADGATRVLRYWDLYREVNRFAVLLKSLGVERGDRVAIYMPMI
PEAMVAMLAVNRIGAVHTVVFSGFGPQALAERIKDAEAKVVITADGMRRRGRVIPLKPTV
DEALKIVGNDIFTVVYKHTGVEVPMKQGRDLWWQEEIAKIPPNTYIEPEWVPGEAPLFIL
YTSGTTGKPKGILHLHGQYMVWIWYAFNHLTGAERDFREDIVFFSTADIGWISGHHYGVH
GPLLNGLTVLWYEDAPDYPHPGIWWEIADTYKVTHMLFSPTAIRLLMKYGDEWPRRYKLD
SIMALYPTGEVLNEEAYNWMRREVCRGRPDCQIADIWGQTETACFVTAPGSMNLGGFRYK
YGSVGMPYPTLNLQILDDDGKPLPPGAKGHVVAKPPLPPAFLHTLWRDPERYVKSYWSRF
PGYYYTGDLGYIDQDGHLHIMGRSDDVIKVAGHRLSTREVEDILTSHPAVAEAAVVGVPD
EVRGEVLGVFVVPKQGMKITEEEVVKHLRNSLGPVAVIGKVAILDKLPKTRTGKVMRRVL
RAMATGQPVGDLSTLEDEEALEELRKKLG

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:44. In other embodiments, the recombinant organism or microorganism according to the invention comprises a nucleic acid sequence encoding a continuous stretch of at least 200, 300, 400, 500 or 600 amino acids derived from the polypeptide set forth in SEQ ID NO:44.

In certain embodiments, the enzyme that catalyzes the conversion of crotonic acid into crotonyl-CoA may be a methylmercaptopropionate (MMPA)-coenzyme A (CoA) ligase (EC 6.2.1.44). Non-limiting examples of methylmercaptopropionate (MMPA)-coenzyme A (CoA) ligases are encoded by the genes SAR11_0248 from Pelagibacter ubique, SPO0677 from Ruegeria pomeroyi, SPO2045 from Ruegeria pomeroyi, SL1157_1815 from Ruegeria lacuscaerulensis, SL1157_2728 from Ruegeria lacuscaerulensis, PA4198 from Pseudomonas aeruginosa or BTH_12141 from Burkholderia thailandensis.

The amino acid sequence of a methylmercaptopropionate (MMPA)-coenzyme A (CoA) ligase encoded by the gene SAR11_0248 from Pelagibacter ubique (Uniprot Accession No: Q4FP19) is set forth in SEQ ID NO: 45.

The amino acid sequence of a methylmercaptopropionate (MMPA)-coenzyme A (CoA) ligase encoded by the gene SPO0677 from Ruegeria pomeroyi (Uniprot Accession No: Q5LVM3) is set forth in SEQ ID NO: 46.

The amino acid sequence of a methylmercaptopropionate (MMPA)-coenzyme A (CoA) ligase encoded by the gene SPO2045 from Ruegeria pomeroyi (Uniprot Accession No: Q5LRT0) is set forth in SEQ ID NO: 47.

The amino acid sequence of a methylmercaptopropionate (MMPA)-coenzyme A (CoA) ligase encoded by the gene SL1157_1815 from Ruegeria lacuscaerulensis (Uniprot Accession No: D0CV95) is set forth in SEQ ID NO:48.

The amino acid sequence of a methylmercaptopropionate (MMPA)-coenzyme A (CoA) ligase encoded by the gene SL1157_2728 from Ruegeria lacuscaerulensis (Uniprot Accession No: D0CPY8) is set forth in SEQ ID NO:49.

The amino acid sequence of a methylmercaptopropionate (MMPA)-coenzyme A (CoA) ligase encoded by the gene PA4198 from Pseudomonas aeruginosa (Uniprot Accession No: Q9HWI3) is set forth in SEQ ID NO: 50.

The amino acid sequence of a methylmercaptopropionate (MMPA)-coenzyme A (CoA) ligase encoded by the gene BTH_12141 from Burkholderia thailandensis (Uniprot Accession No: Q2SWN7) is set forth in SEQ ID NO:51.

SAR11_0248 from Pelagibacter ubique (SEQ ID NO: 45):
MSHYDTNLDKNEANYVPLSPLIFLERTKDIYPNYEALVYESRSYTWEEVYKRCVKFASAL
DKLGVKTGDTVSIMAFNTPEIFEAHYSIPMVGAVINAINTRLDPNTISYILQHSDAKVLI
VDRQFHDVIEKALKNVKNKITIIDIDDQDIDTSSFKRIGELEYESFLNTGNENYEWKKPK
DEWEAISLGYTSGTTGNPKGVVYHHRGSYLMATGSVTAWNMPNKLNFLCVVPMFHCNGWC
YPWTLAMLHARVICLRNIDVKKMFELIDKYEVTHFGGAPIVLNMIVNAPKEDQKALKRKV
NVLTAGAPPPSIIFEKMENLGFEVMHVYGLTETYGHMLQCAWNDDWNSLEKDKKNEIKAR
QGVRYPNTEGAIVMDPETMKPVPKDGKTMGEIMIRGNIVMKGYYKDKEATDKSMAGGWFH
SGDLAVTHPDGYIKIQDRSKDIIISGGENISSIEIENAIAKHPSVSLAAVVAKPDEKWGE
TPCAFVELIKDKPATEKEIIDFCRETLAGFKLPKSVIFCDLPKTSTGKIQKFELRKKVKE
LS
SPO0677 from Ruegeria pomeroyi (SEQ ID NO: 46):
MTGSTIYDQHLDRTPANFTPLSPLSYIERTAAVYPDYPSVVYGDRRYTWAETYTRCRRLA
SALAGRGLGKGDTVSIIAANIPEMYEAHFGVPMAGAVLNAINTRLDAPIIAFILSHAEAR
VLIVDPEFSEVVRDALAQIDRPDLLVVDIEDPSFAGGAPVGTLSYDALLAEGDPDFDWSL
PGDEWDAIALNYTSGTTGNPKGVVYHHRGAALNATSNILTWGMPQHAVYLWTLPMFHCNG
WCFPWTMAANAGVSVCLRAVRDEPIYRAFREEKVTHFCGAPIVLNMLANAPDHMKDEDHQ
IKVMTAGAPPPAAVIEKMEAMGVDVTHVYGLTETYGPSVVCAWKEEWDGRPGAERAALKV
RQGVRYVALSGLMVADPETLEPVPADGETMGEIFMQGNIVMKGYLKNPDATDRAFRGGWF
ASGDLGVMHPDGYIALKDRSKDIIISGGENISSVEVEDVLYKHPAVMEAAVVARPDEKWG
ETPCAFVELKPGQSVEAADLIAHCRANMAHFKAPKTVVFGELPKTSTGKIQKELLRERAR
AL
SPO2045 from Ruegeria pomeroyi (SEQ ID NO: 47):
MLGQMMYQPLLISSLIDHAARYHGEAQIWSVSTEGGVEETNWAGIADNARRLGSVLTDAG
LAPQSRVATLAWNNRRHLEIYYGVSGAGFVLHTINPRLFPEQLVYILNHAEDRILFFDAT
FLPLVEGIRPHLTTVERLVLMGPRDEAAAARIEGLEFYDEFVATGDAGFDWPDLDERTAS
SLCYTSGTTGNPKGVLYSHRSTVLHSFGSNTRDCIGFSARDVVMPVVPMFHVNAWGTPYA
CAMSGSCMVLPGPDLHGEALVGLIDRYRVTIALGVPTIWQGLLATARAKGSTLESLTRTV
IGGAACPPSMIAEFRDRYGVDTVHAWGMSEMSPLGTTNQPLAKHGALPIEAQHKLRENQG
RPPYGVELKIVDDDGNTLPNDGQTQGDLMVRGHWVLDSYFQLQDQPILSDGWFATGDVAT
LDRDGYMTIRDRSKDIIKSGGEWISSVELENIAVAHPKLATAAVIGVPHPKWDERPLLVA
VKAEGETPDEAELLAFFDGKIAKWQVPDRVVFVEALPLNATGKVLKRTLREQFRDVLTG
SL1157_1815 from Ruegeria lacuscaerulensis (SEQ ID NO: 48):
MATHNIYQRDLDKCPANFTALSPLSYIERTAAVYPDYPAVVYGDRRYSWAQTYARCRRLA
SALAARGVGTGDTVSIIAANIPEMYEAHFGVPMAGAVLNAINTRLDAPIIAFILNHAESK
VLLVDPEFSGVVKQALEQVDHDILVIDIEDPSEDGGEKLGALTYDDLLAEGDPEFDWSLP
ADEWDAITLNYTSGTTGNPKGVVYHHRGAALNATSNILTWGMPQHSVYLWTLPMFHCNGW
CFPWTMAANAGTSVCLRAVRDAPIYRAFRDEKVTHECGAPIVLNMLANAPDHLKDFGHEI
KVMTAGAPPPAAVIAAMEGMGIEVTHVYGLTETYGPSVVCAWKDEWNDKPAEERAALKVR
QGVKYVALSGLMVADPETLEPVPADGETMGEIFMQGNIVMKGYLKNPEATRKAFRGGWFA
SGDLGVMHPDGYIALKDRSKDIIISGGENISSVEVEDILYKHPAVMEAAVVAKPDEKWGE
TPCAFVELKPGAEATEADLIAFCRDHMAHFKAPKTVVFGPLPKTSTGKIQKFKLREQARA
LG
SL1157_2728 from Ruegeria lacuscaerulensis (SEQ ID NO: 49):
MLGQMMTQPLLISSLIDHAARYHGQTEIVSVETDGTVTRTNWGEIAANARRMGSALTKLG
LQPQDRIGTLAWNNRRHLEIYYAASGAGFVCHTINPRLFPEQLVYIINHAQDRVLFFDAT
FLPLVAAIRDQLTEVKHFVLMGPRNEDALQQIPGLEFYDELIETGDTDFEWPVFDENTAS
SLCYTSGTTGHPKGVLYSHRSTVLHSFASNTRDVIGYSAMDVVMPVVPMFHVNAWGSPYG
CAMSGAQMVLPGPDLHGEALVNLIDTYGVTLAMGVPTIWQGLLAHAAKCGTKLESLERTV
IGGAACPPSMIATFREKYGVDTVHAWGMSEMSPLGTANIPLAKHRKLPIEEQHKLRENQG
RPPFGVELKIVDDDGNDLPHDGVTQGDLMVRGHWVLDSYFQLKDQELLQDGWFATGDVAT
LDPDGYMTIRDRSKDIIKSGGEWISSVELENIAVAHPKLATAAVIGVPHPKWDERPLLVA
VKAEGEDPSEAELLEFFDGKIAKWQVPDKVVFVDALPLNATGKVLKRKLRDEFKDALTG
PA4198 from Pseudomonas aeruginosa (SEQ ID NO: 50):
MSIFEQGLAPAAVNHIALTPLSFIERTASVYPHYPAVIHGNIRRDWAQTYQRCRRLASAL
AGRGIGQGDTVAVMLPNIPEMLEAHYGVPMIGAVLNTLNVRLDAEAIAFMLQHGEAKVLI
TDYEFHEVIHAAIGMLDHPPLVVDVNDPEYGEGRPVGELDYEALLAEGDPQFAWEWPRDE
WQAISLNYTSGTTGNPKGVVYHHRGAYLNALGNQMTWSMGQHPVYLWTLPMFHCNGWCYP
WTVTALAGVHVCLRRVDPQKVLTLIREHQVSHLCGAPIVLNALINMPDSAKAAIDHPVHA
MVAGAAPPAKVIGAVEEMGIRVTHVYGLTEVYGPVTVCAWHGEWDDLPLERRAAIKSRQG
VRYPTLEGVMVADPKTLEPVPRDGQSIGEIFMRGNTVMKGYLKNPSATEEAFAGGWFHTG
DLAVCHPDGYIEIRDRLKDIIISGGENISTIELEGVLYRHPAVLEAAVVARPDEKWGETP
CAFITLKSDHQGLAESEIVAFCREHLAAFKIPRTVVFSELPKTSTGKIQKYVLREWAAAL
BTH_I2141 from Burkholderia thailandensis (SEQ ID NO: 51):
MLLTGKLADIVSPRRAGRSKPPARRRAVCSRPYRTNTSGRRKTMGKPLLGQMMDMPLLVS
SLIAHAARHAGDVEIVSRRVEGDIHRYTYRDCEARSKRLAQALTRLGVGVGDRIGTLAWN
GYRHVEAYYGISGMGAVCHTINPRLFPEQIAYIVNHAEDRYVLEDLTFAPLVDQLAPQCP
NVKGWIAMTDDAHLPRGATPYLCYETLVGAQDGDYAWPLLDERQASSLCYTSGTTGHPKG
ALYSHRSTVLHAYGAALPDAMGLSSRDAALPVVPMFHVNAWGLPYTAALTGTKLVLPGKD
LDGKSLYELIESERVTFSAGVPTVWLGLLAYMREAGVRFSTLDRTVIGGSACPPSMLETE
EDVYDVRVIHAWGMTELSPLGTLSKLNWAQSQRSVDEQRRLLEKQGRVIYGIDMRIVGDD
GRELPWDGVAFGDLQVCGPWVIDRYFGIDASPLVDGWFPTGDVATIDADGFLQITDRSKD
VIKSGGEWISSIDVENVAVAHPAVAEAACIACAHPKWTERPLLVVVKRAGMDVTRGELLA
FYDGKVAKWWIPDDVVFVDALPHTATGKLQKLKLREQFRDHVLPTAVDA

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptides set forth in SEQ ID NOs: 45-51. In other embodiments, the recombinant organism or microorganism according to the invention comprises a nucleic acid sequence encoding a continuous stretch of at least 100, 200, 300, 400 or 500 amino acids derived from any one of the polypeptides set forth in SEQ ID NO:45-51.

In a particular embodiment, the invention relates to the recombinant organism or microorganism according to the invention, wherein the phosphate acyltransferase (EC 2.3.1) is a phosphate butyryltransferase (EC 2.3.1.19) and/or wherein the acid kinase (EC 2.7.2) is a butyrate kinase (EC 2.7.2.7).

The enzyme that catalyzes the conversion of crotonic acid into crotonyl-CoA may be any enzyme or combination of enzymes from EC classes 2.3.1 and 2.7.2 that efficiently catalyzes the conversion of crotonic acid into crotonyl-CoA. That is, the conversion of crotonic acid into crotonyl-CoA may be catalyzed by a single enzyme that has phosphate acyltransferase and acid kinase activity. Alternatively, the conversion of crotonic acid into crotonyl-CoA may be catalyzed by a combination of a phosphate acyltransferase and an acid kinase. In a preferred embodiment, the phosphate acyltransferase (EC 2.3.1) may be a phosphate butyryltransferase (EC 2.3.1.19) and the acid kinase (EC 2.7.2) may be a butyrate kinase (EC 2.7.2.7).

In certain embodiments, the enzyme that is involved in the conversion of crotonic acid into crotonyl-CoA may be a phosphate butyryltransferase (EC 2.3.1.19). A non-limiting example of a phosphate butyryltransferase is Ptb from Clostridium acetobutylicum. However, phosphate acyltransferases have been described in other organisms. The amino acid sequence of Ptb from Clostridium acetobutylicum (Uniprot Accession No: P58255) is set forth in SEQ ID NO:15.

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:15. In other embodiments, the recombinant organism or microorganism according to the invention comprises a nucleic acid sequence encoding a continuous stretch of at least 50, 100, 150, 200 or 250 amino acids derived from the polypeptide set forth in SEQ ID NO:15.

In certain embodiments, the enzyme that is involved in the conversion of crotonic acid into crotonyl-CoA may be a butyrate kinase (EC 2.7.2.7). A non-limiting example of a butyrate kinase is Buk from Clostridium acetobutylicum. However, butyrate kinases have been described in other organisms. The amino acid sequence of Buk from Clostridium acetobutylicum (Uniprot Accession No: Q45829) is set forth in SEQ ID NO:16.

In certain embodiments, the recombinant organism or microorganism according to the invention comprises a nucleic acid sequence encoding a polypeptide having at least 50%, 60%, 70%, 80%, 90%, 95%, 96%, 97%, 98%, 99% or 100% sequence identity with the polypeptide set forth in SEQ ID NO:16. In other embodiments, the recombinant organism or microorganism according to the invention comprises a nucleic acid sequence encoding a continuous stretch of at least 100, 150, 200, 250 or 300 amino acids derived from the polypeptide set forth in SEQ ID NO:16.

In certain embodiments, the recombinant organism or microorganism according to the invention encodes a phosphate acyltransferase and an acid kinase. In certain embodiments, the recombinant organism or microorganism according to the invention encodes a phosphate butyryltransferase and a butyrate kinase. In certain embodiments, the recombinant organism or microorganism according to the invention encodes Ptb and Buk from Clostridium acetobutylicum or any of the sequence variants or derivatives thereof that have been defined above.

In certain embodiments, the recombinant organism or microorganism according to the invention is characterized by an increased conversion of crotonic acid into crotonyl-CoA, preferably by any of the strategies disclosed herein above, and by an increased conversion of crotonyl-CoA into butyryl-CoA and/or 3-hydroxybutyryl-CoA.

That is, in certain embodiments, the recombinant organism or microorganism of the invention comprises:
a) an increased level and/or activity of one or more a CoA-transferase (EC 2.8.3), preferably wherein the CoA-transferase is an acetate CoA-transferase (EC 2.8.3.8), an acid thiol ligase (EC 6.2.1), preferably wherein the acid thiol ligase (EC 6.2.1) is a medium-chain acyl-CoA ligase (EC 6.2.1.2), a benzoate-CoA ligase (EC 6.2.1.25), a 4-hydroxybenzoate-CoA ligase (EC 6.2.1.27), a 4-Hydroxybutyrate-CoA ligase (EC 6.2.1.40) or a methylmercaptopropionate (MMPA)-coenzyme A (CoA) ligase (EC 6.2.1.44), an acid kinase (EC 2.7.2), preferably wherein the acid kinase (EC 2.7.2) is a butyrate kinase (EC 2.7.2.7), and/or a phosphate acyltransferase (EC 2.3.1), preferably wherein the phosphate acyltransferase (EC 2.3.1) is a phosphate butyryltransferase (EC 2.3.1.19); and
b) an increased level and/or activity of one or more NADH or NADPH-dependent enzyme capable of reducing a carbon-carbon double bond (EC 1.3.1) and/or a flavin-dependent enzyme capable of reducing a carbon-carbon double bond (EC 1.3.8), preferably wherein the NADH or NADPH-dependent enzyme capable of reducing a carbon-carbon double bond (EC 1.3.1) is a crotonyl-CoA reductase (EC 1.3.1.86), a trans-2-enoyl-CoA reductase (EC 1.3.1.44) or an enoyl-[acyl-carrier-protein] reductase (EC 1.3.1.9) and wherein flavin-dependent enzyme capable of reducing a carbon-carbon double bond (EC 1.3.8) is a short-chain acyl-CoA dehydrogenase (EC 1.3.8.1).

In another embodiment, the recombinant organism or microorganism of the invention comprises:
a) an increased level and/or activity of one or more a CoA-transferase (EC 2.8.3), preferably wherein the CoA-transferase is an acetate CoA-transferase (EC 2.8.3.8), an acid thiol ligase (EC 6.2.1), preferably wherein the acid thiol ligase (EC 6.2.1) is a medium-chain acyl-CoA ligase (EC 6.2.1.2), a benzoate-CoA ligase (EC 6.2.1.25), a 4-hydroxybenzoate-CoA ligase (EC 6.2.1.27), a 4-Hydroxybutyrate-CoA ligase (EC 6.2.1.40) or a methylmercaptopropionate (MMPA)-coenzyme A (CoA) ligase (EC 6.2.1.44), an acid kinase (EC 2.7.2), preferably wherein the acid kinase (EC 2.7.2) is a butyrate kinase (EC 2.7.2.7), and/or a phosphate acyltransferase (EC 2.3.1), preferably wherein the phosphate acyltransferase (EC 2.3.1) is a phosphate butyryltransferase (EC 2.3.1.19); and
b) an increased level and/or activity of one or more hydro-lyase (EC 4.2.1), preferably wherein the hydro-lyase (EC 4.2.1) is a short-chain-enoyl-CoA hydratase (EC 4.2.1.150), a 3-hydroxybutyryl-CoA dehydratase (EC 4.2.1.55) or an enoyl-CoA hydratase (EC 4.2.1.17).

In certain embodiments, the recombinant organism or microorganism according to the invention is characterized by an increased conversion of crotonic acid into crotonyl-CoA, preferably by any of the strategies disclosed herein above, and by a decreased conversion of crotonyl-CoA into crotonic acid.

That is, in certain embodiments, the recombinant organism or microorganism of the invention comprises:
a) an increased level and/or activity of one or more a CoA-transferase (EC 2.8.3), preferably wherein the CoA-transferase is an acetate CoA-transferase (EC 2.8.3.8), an acid thiol ligase (EC 6.2.1), preferably wherein the acid thiol ligase (EC 6.2.1) is a medium-chain acyl-CoA ligase (EC 6.2.1.2), a benzoate-CoA ligase (EC 6.2.1.25), a 4-hydroxybenzoate-CoA ligase (EC 6.2.1.27), a 4-Hydroxybutyrate-CoA ligase (EC 6.2.1.40) or a methylmercaptopropionate (MMPA)-coenzyme A (CoA) ligase (EC 6.2.1.44), an acid kinase (EC 2.7.2), preferably wherein the acid kinase (EC 2.7.2) is a butyrate kinase (EC 2.7.2.7), and/or a phosphate acyltransferase (EC 2.3.1), preferably wherein the phosphate acyltransferase (EC 2.3.1) is a phosphate butyryltransferase (EC 2.3.1.19); and
b) a decreased level and/or activity of one or more endogenous thioester hydrolase (EC 3.1.2), preferably wherein the endogenous thioester hydrolase (EC 3.1.2) is an endogenous palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28), more preferably wherein the endogenous thioester hydrolase (EC 3.1.2) is an acyl-CoA thioesterase 2 (EC 3.1.2.20).
   The Enzymatic Conversion of Acetyl-CoA into Isobutene

Within the present invention, it is preferred that the organism or microorganism is capable of producing isobutene, preferably from acetyl-CoA. As described above, crotonic acid has been surprisingly identified to inhibit the conversion of 3-methylcrotonic acid to isobutene by the enzyme ferulic acid decarboxylase, despite an extensive evolution and improvement of its 3-methylcrotonic acid decarboxylase activity and the close proximity between the structures of the substrates crotonic acid and 3-methylcrotonic acid.

Methods for the production of isobutene from 3-methylcrotonyl-CoA via 3-methylcrotonic acid or from 3-hydroxyisovalerate (HIV) via 3-methylcrotonic acid have been described (see FIG. 1 for an overview). Methods as well as recombinant organisms and microorganisms utilizing these pathways and enzymatic conversions have, in particular, been described in WO 2017/085167, WO 2018/206262 and WO 2020/188033.

In the following, the major reactions of the individual enzymatic conversions as described in the prior art WO 2017/085167, WO 2018/206262, WO2010/001078, WO2012/052427 and WO 2016/042012, respectively, and as schematically illustrated in FIG. 1 are described in more detail.

However, the present invention is not limited to these major reactions but also relates to all other routes for the individual steps of the conversion of acetyl-CoA into isobutene as described in the prior art documents WO 2017/085167, WO 2018/206262, WO2010/001078, WO2012/052427 and WO 2016/042012. The disclosure of these documents, in particular with respect to preferred embodiments of the enzymes for the individual conversions of the pathways described therein, is herewith incorporated by reference in its entirety. Accordingly, in preferred embodiments, it is preferable to use the enzymes selected from the preferred embodiments described in these prior art documents in connection with the respective enzymatic conversion. Thus, the same applies to the enzymatic conversions of the present invention described in the following as has been set forth in WO 2017/085167, WO 2018/206262, WO2010/001078, WO2012/052427 and WO 2016/042012, respectively.

The Enzymatic Conversion of Acetyl-CoA into Acetoacetyl-CoA

According to the present invention, the conversion of acetyl-CoA into acetoacetyl-CoA can be achieved by different routes. One possibility is to first convert acetyl-CoA into malonyl-CoA (step XIV as shown in FIG. 1) and then to further condense said malonyl-CoA and acetyl-CoA into acetoacetyl-CoA (step XV as shown in FIG. 1). Another possibility is to directly condense in a single enzymatic reaction two molecules of acetyl-CoA into acetoacetyl-CoA (step XIII as shown in FIG. 1).

The enzymatic conversion of acetyl-CoA into malonyl-CoA preferably makes use of an acetyl-CoA carboxylase (EC 6.4.1.2) (step XIV as shown in FIG. 1). This naturally occurring reaction fixes CO₂ on acetyl-CoA utilizing ATP resulting in malonyl-CoA.

Moreover, the enzymatic condensation of malonyl-CoA and acetyl-CoA into said acetoacetyl-CoA preferably makes use of an acetoacetyl-CoA synthase (EC 2.3.1.194) (step XV as shown in FIG. 1). This is a natural occurring reaction and condenses malonyl-CoA and acetyl-CoA in a decarboxylation reaction.

Alternatively, the enzymatic conversion of acetyl-CoA into said acetoacetyl-CoA consists of a single enzymatic reaction in which acetyl-CoA is directly converted into acetoacetyl-CoA by the enzymatic condensation of two molecules of acetyl-CoA into acetoacetyl-CoA. Preferably, this enzymatic conversion is achieved by making use of an acetyl-CoA acetyltransferase (EC 2.3.1.9). This reaction is a naturally occurring reaction (step XIII as shown in FIG. 1).

The Enzymatic Conversion of Acetoacetyl-CoA into 3-hydroxy-3-methylglutaryl-CoA

The enzymatic conversion of acetoacetyl-CoA into 3-hydroxy-3-methylglutaryl-CoA is an enzymatic condensation of acetoacetyl-CoA and acetyl-CoA into said 3-hydroxy-3-methylglutaryl-CoA (see step IX of FIG. 1).

This condensation preferably makes use of a 3-hydroxy-3-methylglutaryl-CoA synthase (also referred to as HMG-COA synthase). HMG-COA synthases are classified in EC 2.3.3.10 (formerly, HMG-COA synthase has been classified as EC 4.1.3.5 but has been transferred to EC 2.3.3.10). The term “HMG-CoA synthase” refers to any enzyme which is able to catalyze the reaction where acetyl-CoA condenses with acetoacetyl-CoA to form 3-hydroxy-3-methylglutaryl-CoA (HMG-COA). HMG-CoA synthase is part of the mevalonate pathway. Several pathways have been identified for the synthesis of isopentenyl pyrophosphate (IPP), i.e. the mevalonate pathway and the 2-C-methyl-D-erythritol 4-phosphate/1-deoxy-D-xylulose 5-phosphate (MEP/DOXP) pathway. HMG-COA synthase catalyzes the biological Claisen condensation of acetyl-CoA with acetoacetyl-CoA and is a member of a superfamily of acyl-condensing enzymes that includes beta-ketothiolases, fatty acid synthases (beta-ketoacyl carrier protein synthase) and polyketide synthases.

The Enzymatic Conversion of 3-hydroxy-3-methylglutaryl-CoA into 3-methylglutaconyl-CoA

The enzymatic conversion of 3-hydroxy-3-methylglutaryl-CoA into 3-methylglutaconyl-CoA is an enzymatic dehydration reaction which occurs naturally, and which is catalyzed, e.g., by enzymes classified as 3-methylglutaconyl-coenzyme A hydratase (EC 4.2.1.18). Accordingly, the enzymatic conversion of 3-hydroxy-3-methylglutaryl-CoA into 3-methylglutaconyl-CoA preferably makes use of a 3-methylglutaconyl-coenzyme A hydratase (EC 4.2.1.18) (as shown in step VIII of FIG. 1).

The conversion of 3-hydroxy-3-methylglutaryl-CoA into 3-methylglutaconyl-CoA can also be achieved by making use of a 3-hydroxy-3-methylglutaryl-coenzyme A dehydratase activity which has been identified, e.g., in Myxococcus xanthus and which is encoded by the liuC gene (Li et al., Angew. Chem. Int. Ed. 52 (2013), 1304-1308). The 3-hydroxy-3-methylglutaryl-coenzyme A dehydratase derived from Myxococcus xanthus has the Uniprot accession number Q1D5Y4.

The enzymatic conversion of 3-hydroxy-3-methylglutaryl-CoA into 3-methylglutaconyl-CoA can also be achieved by making use of a 3-hydroxyacyl-CoA dehydratase or an enoyl-CoA hydratase. 3-hydroxyacyl-CoA dehydratases and enoyl-CoA hydratases catalyze the same reaction while the name of one of these enzymes denotes one direction of the corresponding reaction while the other name denotes the reverse reaction. As the reaction is reversible, both enzyme names can be used. 3-hydroxyacyl-CoA dehydratases and enoyl-CoA hydratases belong to enzymes classified as EC 4.2.1.

The Enzymatic Conversion of 3-methylglutaconyl-CoA into 3-methylcrotonyl-CoA

The conversion of 3-methylglutaconyl-CoA into 3-methylcrotonyl-CoA may be catalyzed by different enzymes, e.g., by making use of (i) a methylcrotonyl-CoA carboxylase (EC 6.4.1.4); or (ii) a geranoyl-CoA carboxylase (EC 6.4.1.5) (as shown in step VII of FIG. 1).

In another preferred embodiment the conversion of 3-methylglutaconyl-CoA via decarboxylation into 3-methylcrotonyl-CoA is catalyzed by a 3-methylglutaconyl-CoA decarboxylase, e.g. a 3-methylglutaconyl-CoA decarboxylase of Myxococcus xanthus encoded by the liuB gene. This gene codes for an enzyme having the two subunits AibA and AibB (Li et al., Angew. Chem. Int. Ed. 52 (2013), 1304-1308).

The Enzymatic Conversion of 3-methylcrotonyl-CoA into Isobutene Via 3-methylcrotonic acid

The conversion of 3-methylcrotonyl-CoA into 3-methylcrotonic acid can, e.g., be achieved in different ways, e.g., by three alternative enzymatic routes described in the following and as shown in FIG. 1 (step VIa, step VIb or step VIc as shown in FIG. 1).

Thus, the enzymatic conversion of 3-methylcrotonyl-CoA into 3-methylcrotonic acid may be achieved by
(a) a single enzymatic reaction in which 3-methylcrotonyl-CoA is directly converted into 3-methylcrotonic acid, preferably by making use of a CoA transferase (EC 2.8.3.-), preferably a propionate: acetate-CoA transferase (EC 2.8.3.1), an acetate CoA-transferase (EC 2.8.3.8) or a succinyl-CoA: acetate CoA-transferase (EC 2.8.3.18) (step VIa as shown in FIG. 1);
(b) a single enzymatic reaction in which 3-methylcrotonyl-CoA is directly converted into 3-methylcrotonic acid, preferably by making use of a thioester hydrolase (EC 3.1.2), preferably an acetyl-CoA hydrolase (EC 3.1.2.1), an ADP-dependent short-chain-acyl-CoA hydrolase (EC 3.1.2.18) or an acyl-CoA hydrolase (EC 3.1.2.20) (step VIb as shown in FIG. 1); or
(c) two enzymatic steps comprising
   (i) first enzymatically converting 3-methylcrotonyl-CoA into 3-methylcrotonyl phosphate; and
   (ii) then enzymatically converting the thus obtained 3-methylcrotonyl phosphate into said 3-methylcrotonic acid (step VIc as shown in FIG. 1).

As regards (c), the enzymatic conversion of 3-methylcrotonyl-CoA into 3-methylcrotonic acid is achieved by two enzymatic steps comprising (i) first enzymatically converting 3-methylcrotonyl-CoA into 3-methylcrotonyl phosphate; and (ii) then enzymatically converting the thus obtained 3-methylcrotonyl phosphate into said 3-methylcrotonic acid.

The conversion of 3-methylcrotonyl-CoA into 3-methylcrotonyl phosphate can, e.g., be achieved by the use of a phosphate butyryltransferase (EC 2.3.1.19) or a phosphate acetyltransferase (EC 2.3.1.8).

The conversion of 3-methylcrotonyl phosphate into 3-methylcrotonic acid can, e.g., be achieved by making use of an enzyme which is classified as EC 2.7.2.-, i.e., a phosphotransferase. Such enzymes use a carboxy group as acceptor. Thus, the conversion of 3-methylcrotonyl phosphate into 3-methylcrotonic acid can, e.g., be achieved by making use of an enzyme with a carboxy group as acceptor (EC 2.7.2.-). In a preferred embodiment, the conversion of 3-methylcrotonyl phosphate into 3-methylcrotonic acid is achieved by the use of a propionate kinase (EC 2.7.2.15), an acetate kinase (EC 2.7.2.1), a butyrate kinase (EC 2.7.2.7) or a branched-chain-fatty-acid kinase (EC 2.7.2.14).

As mentioned above, the conversion of 3-methylcrotonyl-CoA into 3-methylcrotonic acid can also be achieved by two alternative conversions wherein 3-methylcrotonyl-CoA is directly converted into 3-methylcrotonic acid.

Preferably, in one embodiment, 3-methylcrotonyl-CoA is directly converted into 3-methylcrotonic acid by hydrolyzing the thioester bond of 3-methylcrotonyl-CoA into 3-methylcrotonic acid by making use of an enzyme which belongs to the family of thioester hydrolases (in the following referred to as thioesterases (EC 3.1.2.-)); step VIb as shown in FIG. 1.

Thioesterases (TEs; also referred to as thioester hydrolases) are enzymes which are classified as EC 3.1.2. Presently thioesterases are classified as EC 3.1.2.1 through EC 3.1.2.30 while TEs which are not yet classified/unclassified are grouped as enzymes belonging to EC 3.1.2.-. Cantu et al. (Protein Science 19 (2010), 1281-1295) describe that there are 23 families of thioesterases which are unrelated to each other as regards the primary structure. However, it is assumed that all members of the same family have essentially the same tertiary structure. Thioesterases hydrolyze the thioester bond between a carbonyl group and a sulfur atom.

In a preferred embodiment, a thioesterase employed according to the present invention for converting 3-methylcrotonyl-CoA into 3-methylcrotonic acid is selected from the group consisting of:
acetyl-CoA hydrolase (EC 3.1.2.1);
palmitoyl-CoA hydrolase (EC 3.1.2.2);
3-hydroxyisobutyryl-CoA hydrolase (EC 3.1.2.4);
oleoyl-[acyl-carrier-protein] hydrolase (EC 3.1.2.14);
ADP-dependent short-chain-acyl-CoA hydrolase (EC 3.1.2.18);
ADP-dependent medium-chain-acyl-CoA hydrolase (EC 3.1.2.19);
1,4-dihydroxy-2-naphthoyl-CoA hydrolase (EC 3.1.2.28); and acyl-CoA hydrolase (EC 3.1.2.20).

In more preferred embodiments, a thioesterase/thioester hydrolase (EC 3.1.2.-) employed according to the present invention is an acetyl-CoA hydrolase (EC 3.1.2.1), an ADP-dependent short-chain-acyl-CoA hydrolase (EC 3.1.2.18), a 1,4-dihydroxy-2-naphthoyl-CoA hydrolase (EC 3.1.2.28), and an acyl-CoA hydrolase (EC 3.1.2.20).

In an alternative embodiment, 3-methylcrotonyl-CoA is directly converted into 3-methylcrotonic acid, preferably by making use of an enzyme which belongs to the family of CoA-transferases (EC 2.8.3.-) capable of transferring the CoA group of 3-methylcrotonyl-CoA to a carboxylic acid (step VIa as shown in FIG. 1).

CoA-transferases are found in organisms from all lines of descent. Most of the CoA-transferases belong to two well-known enzyme families (referred to in the following as families I and II) and there exists a third family which had been identified in anaerobic metabolic pathways of bacteria. A review describing the different families can be found in Heider (FEBS Letters 509 (2001), 345-349).

Preferably, the CoA-transferase employed according to the present invention for the direct conversion of 3-methylcrotonyl-CoA into 3-methylcrotonic acid is selected from the group consisting of:
propionate: acetate-CoA transferase (EC 2.8.3.1);
acetate CoA-transferase (EC 2.8.3.8); and
butyrate-acetoacetate CoA-transferase (EC 2.8.3.9).

In more preferred embodiments, CoA transferases (EC 2.8.3.-) are a propionate: acetate-CoA transferase (EC 2.8.3.1), an acetate CoA-transferase (EC 2.8.3.8) and a succinyl-CoA: acetate CoA-transferase (EC 2.8.3.18).

Exemplary combinations of enzymes to achieve the conversion of acetyl-CoA to isobutene are listed in Tables 1 and 2.

TABLE 1

| | | | NCBI | Uniprot Accession |
|---|---|---|---|---|
| Step | Enzyme | Gene | reference | number |
| I | Acetyl-CoA transferase from | thlA | WP_010966157.1 | P45359 |
| | Clostridium acetobulyticum | | | |
| II | Hydroxymethylglutaryl-CoA synthase | mvaS | WP_002357756.1 | Q9FD71 |
| | from Enterococcus faecalis | | | |
| III | Enoyl-CoA hydratase/isomerase | PputUW4_01474 | WP_015094072.1 | K9NHK2 |
| | from Pseudomonas sp. UW4 (ECH) | | | |
| IV | Glutaconate CoA-transferase from | MXAN_4264 | WP_011554268.1 | Q1D4I3 |
| | Myxococcus xanthus (AibA/B) | MXAN_4265 | WP_011554267.1 | Q1D4I4 |
| V | 1,4-Dihydroxy-2-naphtoyl-CoA | Ydil, menl | NP_416201.1 | P77781 |
| | hydrolase from Escherichia coli | | | |
| VI | Variant of UbiD-like decarboxylase | GZL_07100 | | A0A0A8EV26 |
| | decarboxylase from Streptomyces | | | |
| | sp.769 (UbiD) | | | |
| | (A241D-G402A-S403C-C404L- | | | |
| | P406A-L448W) | | | |
| | Flavin prenyl transferase from | ubiX | WP_000825700.1 | P0AG03 |
| | Escherichia coli (UbiX) | | | |

TABLE 2

| | | | NCBI | Uniprot Accession |
|---|---|---|---|---|
| Step | Enzyme | Gene | reference | number |
| I | Acetyl-CoA transferase from | thlA3 | EDK35683.1 | A5N3I7 |
| | Clostridium kluyverii | | | |
| II | Hydroxymethylglutaryl-CoA synthase | mvaS | WP_002357756.1 | Q9FD71 |
| | from Enterococcus faecalis | | | |
| III | Enoyl-CoA hydratase/isomerase from | PputUW4_01474 | WP_015094072.1 | K9NHK2 |
| | Pseudomonas sp. UW4 (ECH) | | | |
| IV | Glutaconate CoA-transferase from | MXAN_4264 | WP_011554268.1 | Q1D4I3 |
| | Myxococcus xanthus (AibA/B) | MXAN_4265 | WP_011554267.1 | Q1D4I4 |
| V | Acyl-CoA thioesterase | tesB | AAC73555.1 | P0AGG2 |
| VI | Variant of UbiD-like decarboxylase | FDC1 | XP_013946967.1 | G9NLP8 |
| | from Hypocrea atroviridis | | | |
| | (Trichoderma atroviride) | | | |
| | (UbiD) with N-terminal MBP fusion | | | |
| | Flavin prenyl transferase from | ubiX | WP_000825700.1 | P0AG03 |
| | Escherichia coli (UbiX) | | | |

An Alternative Route for the Provision of 3-methylcrotonic acid

As outlined above, 3-methylcrotonic acid (which is then further enzymatically converted into isobutene as described in detail further below) can be enzymatically provided from acetyl-CoA by the enzymatic conversion of acetyl-CoA into acetoacetyl-CoA (step XIV, step XV, step XIII as shown in FIG. 1), the enzymatic conversion of acetoacetyl-CoA into 3-hydroxy-3-methylglutaryl-CoA (step IX of FIG. 1), the enzymatic conversion of 3-hydroxy-3-methylglutaryl-CoA into 3-methylglutaconyl-CoA (step VIII of FIG. 1), the enzymatic conversion of 3-methylglutaconyl-CoA into 3-methylcrotonyl-CoA (step VII of FIG. 1) and the enzymatic conversion of 3-methylcrotonyl-CoA into 3-methylcrotonic acid.

In an alternative route, according to the present invention, 3-methylcrotonic acid can be provided by another possible pathway from acetyl-CoA. In this pathway, acetyl-CoA is enzymatically converted into acetoacetyl-CoA as described above.

According to this alternative route, acetoacetyl-CoA is then enzymatically converted into acetoacetate (step Va or Vb of FIG. 1), acetoacetate is further enzymatically converted into acetone (step IV of FIG. 1), acetone is further enzymatically converted into 3-hydroxyisovalerate (HIV) (step III of FIG. 1), which is then further enzymatically converted into said 3-methylcrotonic acid.

The individual enzymatic steps of this alternative pathway are described in more detail in the following.

The Enzymatic Conversion of Acetoacetyl-CoA into Acetoacetate

The conversion of acetoacetyl-CoA into acetoacetate can be achieved by two different routes. One possibility is the conversion of acetoacetyl-CoA into acetoacetate by hydrolysing the CoA thioester of acetoacetyl-CoA into acetoacetate (step Va as shown in FIG. 1). In another, more preferred, aspect the CoA group of acetoacetyl-CoA is transferred on acetate, resulting in the formation of acetoacetate and acetyl-CoA (step Vb as shown in FIG. 1).

As mentioned, in one aspect, the CoA thioester of acetoacetyl-CoA is hydrolyzed to result in acetoacetate. According to this aspect of the present invention, the enzymatic conversion of acetoacetyl-CoA into acetoacetate is achieved by preferably making use of an acetoacetyl-CoA hydrolase (EC 3.1.2.11) which naturally catalyzes this reaction.

As mentioned, in another, more preferred, possibility, the CoA group of acetoacetyl-CoA is transferred on acetate, resulting in the formation of acetoacetate and acetyl-CoA. According to this possibility of the present invention, the enzymatic conversion of acetoacetyl-CoA into acetoacetate is achieved by preferably making use of an enzyme which is capable of transferring the CoA group of acetoacetyl-CoA on acetate.

Preferably, such an enzyme capable of transferring the CoA group of acetoacetyl-CoA on acetate belongs to the family of CoA transferases (EC 2.8.3.-).

Thus, the present invention relates to a method for the enzymatic conversion of acetoacetyl-CoA into acetoacetate by making use of an enzyme capable of transferring the CoA group of acetoacetyl-CoA on acetate, preferably a CoA transferase (EC 2.8.3.-). A preferred example of an enzyme catalysing the conversion of acetoacetyl-CoA into acetoacetate which can be employed in the method of the present invention is an enzyme classified as an acetate CoA transferase (EC 2.8.3.8).

The Enzymatic Conversion of Acetoacetate into Acetone

The conversion of acetoacetate into acetone is schematically illustrated in step IV of FIG. 1. This reaction is a decarboxylation reaction and is a natural occurring reaction in organisms capable of producing acetone, i.e., organisms of the genus Clostridia. According to the present invention, the conversion of acetoacetate into said acetone preferably makes use of an acetoacetate decarboxylase (EC 4.1.1.4).

The Enzymatic Condensation of Acetone and Acetyl-CoA into 3-hydroxyisovalerate (HIV)

The condensation of acetone and acetyl-CoA into said 3-hydroxyisovalerate (HIV) is schematically illustrated in step Ill of FIG. 1. This condensation preferably makes use of an enzyme which is capable of catalyzing the formation of a covalent bond between the carbon atom of the oxo (i.e., the C═O) group of acetone and acetyl-CoA, in particular the methyl group of acetyl-CoA. According to this reaction scheme, the oxo group of acetone reacts as an electrophile and the methyl group of acetyl-CoA reacts as a nucleophile.

Enzymes which are capable of enzymatically condensing acetone and acetyl-CoA into 3-hydroxyisovalerate (HIV) are known in the art and have, e.g., been described in WO 2011/032934.

Preferably, the enzyme employed in the enzymatic condensation of acetone and acetyl-CoA into 3-hydroxyisovalerate (HIV) is an enzyme with the activity of a HMG CoA synthase (EC 2.3.3.10) and/or a PksG protein and/or an enzyme with the activity of a C—C bond cleavage/condensation lyase (preferably enzymes classified as isopropylmalate synthase (EC 2.3.3.13), as homocitrate synthase (EC 2.3.3.14) or as 4-hydroxy-2-ketovalerate aldolase (EC 4.1.3.39)), such as a HMG CoA lyase (EC 4.1.3.4).

The Enzymatic Conversion of 3-hydroxyisovalerate (HIV) into 3-methylcrotonic acid

The enzymatic conversion of 3-hydroxyisovalerate (HIV) into 3-methylcrotonic acid is schematically illustrated in step II of FIG. 1. This conversion preferably makes use of an enzyme catalyzing the dehydration of a β-hydroxy acid (i.e., e.g., 3-hydroxyisovalerate (HIV)) into an α,β-unsaturated acid (i.e., e.g., 3-methylcrotonic acid). The term “dehydration” generally refers to a reaction involving the removal of H₂O. Preferably, such an enzyme belongs to the family of hydro-lyases (EC 4.2.-.-).

Preferred examples of such enzymes which are classified as EC 4.2 . . . (i.e., hydro-lyases) are:
aconitase (EC 4.2.1.3);
fumarase (EC 4.2.1.2); and
enoyl-CoA hydratase/dehydratase (EC 4.2.1.17).
   The Enzymatic Conversion of 3-methylcrotonic acid into Isobutene

The enzymatic conversion of 3-methylcrotonic acid into isobutene is schematically shown in step I of FIG. 1). This conversion can be achieved by a decarboxylation by making use of a prenylated FMN-dependent decarboxylase associated with an FMN prenyl transferase. “Decarboxylation” is generally a chemical reaction that removes a carboxyl group and releases carbon dioxide (CO₂).

The enzymatic conversion of 3-methylcrotonic acid into isobutene utilizing a prenylated FMN-dependent decarboxylase associated with an FMN prenyl transferase relies on a reaction of two consecutive steps catalyzed by the two enzymes, i.e., the prenylated FMN-dependent decarboxylase (catalyzing the actual decarboxylation of 3-methylcrotonic acid into isobutene) with an associated FMN prenyl transferase which provides the modified flavin cofactor.

The flavin cofactor may preferably be FMN or FAD. FMN (flavin mononucleotide; also termed riboflavin-5′-phosphate) is a biomolecule produced from riboflavin (vitamin B2) by the enzyme riboflavin kinase and functions as prosthetic group of various reactions. FAD (flavin adenine dinucleotide) is a redox cofactor, more specifically a prosthetic group, involved in several important reactions in metabolism.

Thus, in the conversion of 3-methylcrotonic acid into isobutene, in a first step, a flavin cofactor (FMN or FAD) is modified into a (modified) flavin-derived cofactor. This modification is catalyzed by said FMN prenyl transferase. FMN prenyl transferase prenylates the flavin ring of the flavin cofactor (FMN or FAD) into a (modified) prenylated flavin cofactor. More specifically, FMN prenyl transferase catalyzes the prenylation of a flavin cofactor (FMN or FAD) utilizing dimethylallyl phosphate (DMAP) or dimethylallyl pyrophosphate (DMAPP) into a flavin-derived cofactor.

In a second step, the actual conversion of 3-methylcrotonic acid into isobutene is catalyzed by said prenylated FMN-dependent decarboxylase via a 1,3-dipolar cycloaddition based mechanism wherein said prenylated FMN-dependent decarboxylase uses the prenylated flavin cofactor (FMN or FAD) provided by the associated FMN prenyl transferase.

In a preferred embodiment, said FMN prenyl transferase which modifies the flavin cofactor (FMN or FAD) into a (modified) flavin-derived cofactor (utilizing dimethylallyl phosphate (DMAP) or dimethylallyl pyrophosphate (DMAPP)) is a phenylacrylic acid decarboxylase (PAD)-type protein, or the closely related prokaryotic enzyme UbiX, an enzyme which is involved in ubiquinone biosynthesis in prokaryotes.

In Escherichia coli, the protein UbiX (also termed 3-octaprenyl-4-hydroxybenzoate carboxy-lyase) has been shown to be involved in the third step of ubiquinone biosynthesis.

In a preferred embodiment, the modification of a flavin cofactor (FMN or FAD) into the corresponding (modified) flavin-derived cofactor is catalyzed by the FMN-containing protein phenylacrylic acid decarboxylase (PAD). The enzymes involved in the modification of the flavin cofactor (FMN or FAD) into the corresponding modified flavin-derived cofactor were initially annotated as decarboxylases (EC 4.1.1.-). Some phenylacrylic acid decarboxylases (PAD) are now annotated as flavin prenyl transferases as EC 2.5.1.-. Enzymes capable of catalyzing the enzymatic reaction described herein for flavin prenyl transferases have recently also been annotated as flavin prenyl transferases as EC 2.5.1.129.

In a more preferred embodiment, the conversion of 3-methylcrotonic acid into isobutene makes use of a phenylacrylic acid decarboxylase (PAD)-type protein as the FMN prenyl transferase which modifies a flavin cofactor (FMN or FAD) into the corresponding (modified) flavin-derived cofactor wherein said phenylacrylic acid decarboxylase (PAD)-type protein is derived from Candida albicans (Uniprot accession number Q5A8L8), Aspergillus niger (Uniprot accession number A3F715), Saccharomyces cerevisiae (Uniprot accession number P33751) or Cryptococcus gattii (Uniprot accession number E6R9Z0).

In another preferred embodiment, the modification of a flavin cofactor (FMN or FAD) into the corresponding (modified) flavin-derived cofactor is catalyzed by the FMN-containing protein 3-octaprenyl-4-hydroxybenzoate carboxy-lyase also termed UbiX (initially annotated EC 4.1.1.-). As mentioned above, the enzymes involved in the modification of the flavin cofactor (FMN or FAD) into the corresponding modified flavin-derived cofactor were initially annotated as decarboxylases. Some phenylacrylic acid decarboxylases (PAD) are now annotated as flavin prenyl transferases as EC 2.5.1.-.

As mentioned above, enzymes capable of catalyzing the enzymatic reaction described herein for flavin prenyl transferases have recently also been annotated as flavin prenyl transferases as EC 2.5.1.129.

In a more preferred embodiment, the conversion of 3-methylcrotonic acid into isobutene makes use of a 3-octaprenyl-4-hydroxybenzoate carboxy-lyase (also termed UbiX) as the FMN prenyl transferase which modifies the flavin cofactor (FMN or FAD) into the corresponding (modified) flavin-derived cofactor wherein said 3-octaprenyl-4-hydroxybenzoate carboxy-lyase (also termed UbiX) is derived from Escherichia coli (Uniprot accession number P0AG03), Bacillus subtilis (Uniprot accession, number A0A086WXG4), Pseudomonas aeruginosa (Uniprot accession number A0A072ZCW8) or Enterobacter sp. DC4 (Uniprot accession number W7P6B1).

In another preferred embodiment, the modification of a flavin cofactor (FMN or FAD) into the corresponding (modified) flavin-derived cofactor is catalyzed by an Ubx-like flavin prenyl transferase derived from E. coli encoded by kpdB and ecdB, respectively (UniProt accession number A0A023LDW3 and UniProt accession number P69772, respectively), and an Ubx-like flavin prenyl transferase derived from Klebsiella pneumoniae encoded by kpdB (UniProt accession number Q462H4).

In another preferred embodiment, the modification of a flavin cofactor (FMN or FAD) into the corresponding (modified) flavin-derived cofactor is catalyzed by a flavin prenyl transferase.

As mentioned above, the actual decarboxylation, i.e., the conversion of 3-methylcrotonic acid into isobutene is catalyzed by a prenylated FMN-dependent decarboxylase via a 1,3-dipolar cycloaddition based mechanism wherein said prenylated FMN-dependent decarboxylase uses the prenylated flavin cofactor (FMN or FAD) provided by any of the above described associated FMN prenyl transferases.

In a preferred embodiment, said prenylated FMN-dependent decarboxylase catalyzing the decarboxylation of 3-methylcrotonic acid into isobutene is catalyzed by a ferulic acid decarboxylase (FDC). Ferulic acid decarboxylases (FDC) belong to the enzyme class EC 4.1.1.-.

In an even more preferred embodiment, the conversion of 3-methylcrotonic acid into isobutene makes use of a ferulic acid decarboxylases (FDC) which is derived from Saccharomyces cerevisiae (Uniprot accession number Q03034), Enterobacter sp. (Uniprot accession number V3P7UO), Bacillus pumilus (Uniprot accession number Q45361), Aspergillus niger (Uniprot accession number A2R0P7) or Candida dubliniensis (Uniprot accession number B9WJ66).

In another more preferred embodiment, the conversion of 3-methylcrotonic acid into isobutene makes use of a protocatechuate decarboxylase (EC 4.1.1.63).

In a preferred embodiment of the present invention, the PCA decarboxylase employed in the method of the present invention is a PCA decarboxylase which is derived from Klebsiella pneumoniae (Uniprot accession number B9AM6), Leptolyngbya sp. (Uniprot accession number A0A0S3U6D8), or Phascolarctobacterium sp. (Uniprot accession number R6IIV6).

In another preferred embodiment, said prenylated FMN-dependent decarboxylase catalyzing the decarboxylation of 3-methylcrotonic acid into isobutene is an enzyme which is closely related to the above ferulic acid decarboxylase (FDC), namely a 3-polyprenyl-4-hydroxybenzoate decarboxylase (also termed UbiD). 3-polyprenyl-4-hydroxybenzoate decarboxylase belongs to the UbiD decarboxylase family classified as EC 4.1.1.-.

In a more preferred embodiment, the conversion of 3-methylcrotonic acid into isobutene makes use of a 3-polyprenyl-4-hydroxybenzoate decarboxylase (UbiD) which is derived from Hypocrea atroviridis (UniProt Accession number G9NLP8), Sphaerulina musiva (UniProt Accession number M3DF95), Penecillinum requeforti (UniProt Accession number W6QKP7), Fusarium oxysporum f. sp. lycopersici (UniProt Accession number W9LTH3), Saccharomyces kudriavzevii (UniProt Accession number J8TRN5), Saccaromyces cerevisiae, Aspergillus parasiticus, Candida albicans, Grosmannia clavigera, Escherichia coli (Uniprot accession number P0AAB4), Bacillus megaterium (Uniprot accession number D5DTL4), Methanothermobacter sp. CaT2 (Uniprot accession number T2GKK5), Mycobacterium chelonae 1518 (Uniprot accession number X8EX86) or Enterobacter cloacae (Uniprot accession number V3DX94).

In another more preferred embodiment, the conversion of 3-methylcrotonic acid into isobutene makes use of an UbiD-like decarboxylase which is derived from Streptomyces sp (UniProt Accession number A0A0A8EV26).

In an even more preferred embodiment, the UbiD-like decarboxylase which is derived from Streptomyces sp. is an enzyme comprising the amino acid sequence of SEQ ID NO:52 or a sequence which is at least n % identical to SEQ ID NO:52 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene.

Streptomyces sp. UbiD (SEQ ID NO: 52)
MYVQMVFVKEVPVQHPSDLREHIEALEKLGDLDRVHDEVDWNLEAAAQTRYSTEHHLPAP
LFENVAGVAEGFRLLGAPAALSSDPSRPYARVALSVGLRPEATGREVVEHLVAARHRPGV
PPVAVAAEAAPVKANVLLGDEADLNRFPVPFVHEGDGNRYANTYGVIIAQTPDGSWINWS
IARIMMIDGKHMTGLVMHPQHIAQVWQQWADLGKPMPYALVQGGDPAIPYVGGIPIGDGV
AESAYIGALIGRPLEVVKAELSDLMVPAGAEIVIEGHLSVQRDGVEGPFGEFAGYIPRET
SLQPVYTVEAITHRDAPIWPLVAEGKPTDDFHTVTGIGEAAGALDAIREAGLPAASAWAP
LSAASHWLVVTAPGNWRELLPGVSEEQYARRVGEAVFGTKFGSCLPQVFLLDDDFDPTDD
ADLLWALATRVHPDGRVVRFEDGPVLPLLTCYTPQERHAARATKVVHEALLSAPGEREPQ
STFADAYPAEVRAKVRARYPN

In another more preferred embodiment, the conversion of 3-methylcrotonic acid into isobutene makes use of an UbiD-like decarboxylase which is derived from Yersinia frederiksenii (Uniprot Accession Number: A0A0T9UUQ9).

In an even more preferred embodiment, the UbiD-like decarboxylase which is derived from Yersinia frederiksenii is an enzyme comprising the amino acid sequence of SEQ ID NO:53 or a sequence which is at least n % identical to SEQ ID NO:53 with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99 and wherein the enzyme has the enzymatic activity of converting 3-methylcrotonic acid into isobutene.

Yersinia frederiksenii UbiD (SEQ ID NO: 53)
MFLKSLREQINLLKRQGDLRIINRPVDSYLEAAAIIRRSTETKAPVPLMTNIKGYPGCSI
VGGLAALSSNPEYPLSRVAMSLGLSTTASAQSIVQYLIDGLKKTPYLPVEIDSNSAPCKQ
NILRGEDASLAHFPIPQVHQHDGNRYVNTWGVFIVESPDGQWCNWSIQRVQYLNDRQMIA
LVFPSQHISDIWEEWVKIGKPMPYALVQGCEPIVPYIAGLPLLERNVNEAEYIGALYGKG
IEVVKCETHDLRVPASSEVVIEGYFAITREGVEGPFGEFAGYIPNENSLQPILSITAISW
QDDPIWPVVAEGKPVDEYHTCSGIGDAAAIQNVLLTANLPVSFVWVPLYSACHLMIVSIN
RHWRQTHPDLTSEQLTKKVGDVIHQTRNSIKLPKIVVLDDDVDPADTESLLWALATRIHP
ERKRYFYESCILPLLSCYSEEERHNRKGKRVIIDALLPENHGAISSFDYAYPDDIKQRVL
MHWETDFSHD

Preferably, the UbiD-like decarboxylase which is derived from Yersinia frederiksenii has been genetically engineered to increase the conversion of 3-methylcrotonic acid into isobutene.

That is, in a particular embodiment, the UbiD-like decarboxylase which is derived from Yersinia frederiksenii may be an engineered enzyme having an improved activity in converting 3-methylcrotonic acid into isobutene over the corresponding enzyme from which it is derived and having an amino acid sequence as shown in SEQ ID NO:53 or an amino acid sequence having at least 55% sequence identity to SEQ ID NO:53.

In particular, one or more amino acid residues at a position selected from the group consisting of positions 7, 17, 27, 33, 35, 45, 46, 48, 51, 140, 144, 183, 184, 185, 222, 227, 284, 285, 286, 287, 288, 289, 290, 292, 321, 322, 327, 329, 330, 331, 337, 338, 355, 365, 378, 380, 384, 387, 388, 389, 391, 392, 393, 394, 395, 419, 424, 425, 428, 429, 431, 432, 434, 436, 437, 438, 439, 443, 444, 446, 447, 448, 449, 451, 453, 457, 458, 459, 460, 462, 464, 465, 467, 468, 470 and 471 in the amino acid sequence shown in SEQ ID NO:53 or at a position corresponding to any of these positions, may be substituted with another amino acid residue or deleted or wherein an insertion has been effected at one or more of these positions.

Specific mutant variants of an UbiD-like decarboxylase which is derived from Yersinia frederiksenii with improved activity with 3-methylcrotonic acid are provided in EP 21 16 6368.7, which is fully incorporated herein by reference.

Preferably, the engineered UbiD-like decarboxylase which is derived from Yersinia frederiksenii comprises any of the mutations listed below in Table 3. The list of improved variants is presented in the following Table 3. The increase in activity is described relative to the wild-type enzyme (with “+” representing a low increase in activity and “++++” representing a high increase in activity). The mutations are described as followed: R387A means the wild-type amino-acid arginine (R) at position 387 is replaced by an alanine (A); dG462 means the wild-type amino-acid glycine (G) at position 462 is deleted; 1443aR means an arginine (R) has been inserted after the amino-acid at position 443 (if a second amino-acid is inserted, it will be annotated with i443b). All amino acid positions in Table 3 are given with respect to SEQ ID NO:53.

TABLE 3

| | Activity |
|---|---|
| Mutants | relative to WT |
| R387A | + |
| L432A | + |
| L434V | + |
| S436A | + |
| G380V-K391L-L432W-L434M | + |
| L432F-L434W | + |
| L432F-L434F | + |
| L432W-L434W | + |
| K391I-L432W-L434M | + |
| L432A-S436A | + |
| L432F-L434W-S436A | + |
| R387A-L432F-L434W-S436A | + |
| N388M-L432F-L434W | + |
| K394R-L432F-L434W | + |
| C430S-L432F-L434W | + |
| L432F-L434W-S436N | + |
| N388M-L432F-L434W-S436A | + |
| C430P-L432F-L434W-S436A | + |
| C430S-L432F-L434W-S436A | + |
| K394R-L432F-L434W-S436A | + |
| N388M-L432F-L434W-S436N | + |
| C430P-L432F-L434W-S436N | + |
| C430S-L432F-L434W-S436N | + |
| K394R-L432F-L434W-S436N | + |
| N388M-C430P-L432F-L434W-S436A | + |
| N388M-C430S-L432F-L434W-S436A | + |
| N388M-K394R-L432F-L434W-S436A | + |
| K394R-C430P-L432F-L434W-S436A | + |
| K394R-C430S-L432F-L434W-S436A | + |
| N388M-C430P-L432F-L434W-S436N | + |
| N388M-C430S-L432F-L434W-S436N | + |
| N388M-K394R-L432F-L434W-S436N | + |
| K394R-C430S-L432F-L434W-S436N | + |
| K394R-C430P-L432F-L434W-S436N | + |
| N388M-K394R-C430S-L432F-L434W-S436A | + |
| N388M-K394R-C430P-L432F-L434W-S436A | + |
| N388M-K394R-C430P-L432F-L434W-S436N | + |
| N388M-K394R-C430S-L432F-L434W-S436N | + |
| H141Y-N388M-K394R-C430P-L432F-L434W-S436N | + |
| N388M-K394R-C430K-L432F-L434W-S436N | + |
| N388M-K394R-C430N-L432F-L434W-S436N | + |
| N388M-K394R-I395V-C430P-L432F-L434W-S436N | + |
| N388M-C430A-L432F-L434W-S436N | + |
| N388M-K394A-C430P-L432F-L434W-S436N | + |
| N388M-K394C-C430P-L432F-L434W-S436N | + |
| N388M-K394H-C430P-L432F-L434W-S436N | + |
| N388Q-C430P-L432F-L434W-S436N | + |
| S185T-N388M-C430P-L432F-L434W-S436N | + |
| N388M-K394H-C430S-L432F-L434W-S436N | + |
| S185T-N388M-C430S-L432F-L434W-S436N | + |
| N388M-K394R-C430A-L432F-L434W-S436N | + |
| N388M-K394R-C430N-L432F-L434W-S436N-H444L | + |
| N388M-K394R-C430N-L432F-L434W-S436N-H444N | + |
| N388M-K394R-C430N-L432F-L434W-S436N-H444R | + |
| N388M-K394R-C430N-L432F-L434W-S436N-H444T | + |
| N388M-K394R-C430N-L432F-L434W-S436N-H444V | + |
| N285M-N388M-K394R-C430N-L432F-L434W-S436N | + |
| N287I-N388M-K394R-C430N-L432F-L434W-S436N | + |
| N287K-N388M-K394R-C430N-L432F-L434W-S436N | + |
| N287Q-N388M-K394R-C430N-L432F-L434W-S436N | + |
| N287R-N388M-K394R-C430N-L432F-L434W-S436N | + |
| N287T-N388M-K394R-C430N-L432F-L434W-S436N | + |
| N287V-N388M-K394R-C430N-L432F-L434W-S436N | + |
| N332H-N388M-K394R-C430N-L432F-L434W-S436N | + |
| P284E-N388M-K394R-C430N-L432F-L434W-S436N | + |
| S322T-N388M-K394R-C430N-L432F-L434W-S436N | + |
| N388M-K394R-S429D-C430N-L432F-L434W-S436N | + |
| V382A-N388M-K394R-C430N-L432F-L434W-S436N | + |
| V382L-N388M-K394R-C430N-L432F-L434W-S436N | + |
| P284E-N285M-N388M-K394R-C430N-L432F-L434W-S436N | ++ |
| P284E-N287Q-N388M-K394R-C430N-L432F-L434W-S436N | ++ |
| P284E-N287V-N388M-K394R-C430N-L432F-L434W-S436N | ++ |
| P284E-N287R-N388M-K394R-C430N-L432F-L434W-S436N | ++ |
| P284E-N287T-N388M-K394R-C430N-L432F-L434W-S436N | ++ |
| P284E-N287K-N388M-K394R-C430N-L432F-L434W-S436N | ++ |
| P284E-S322T-N388M-K394R-C430N-L432F-L434W-S436N | ++ |
| N285M-N287Q-N388M-K394R-C430N-L432F-L434W-S436N | ++ |
| N285M-N287V-N388M-K394R-C430N-L432F-L434W-S436N | ++ |
| N285M-N287R-N388M-K394R-C430N-L432F-L434W-S436N | ++ |
| N285M-N287T-N388M-K394R-C430N-L432F-L434W-S436N | ++ |
| N285M-N287K-N388M-K394R-C430N-L432F-L434W-S436N | ++ |
| N285M-S322T-N388M-K394R-C430N-L432F-L434W-S436N | ++ |
| N287Q-S322T-N388M-K394R-C430N-L432F-L434W-S436N | ++ |
| N287V-S322T-N388M-K394R-C430N-L432F-L434W-S436N | ++ |
| N287R-S322T-N388M-K394R-C430N-L432F-L434W-S436N | ++ |
| N287T-S322T-N388M-K394R-C430N-L432F-L434W-S436N | ++ |
| N287K-S322T-N388M-K394R-C430N-L432F-L434W-S436N | ++ |
| P284E-N285M-N287Q-N388M-K394R-C430N-L432F-L434W-S436N | ++ |
| P284E-N285M-N287V-N388M-K394R-C430N-L432F-L434W-S436N | ++ |
| P284E-N285M-N287Q-S322T-N388M-K394R-C430N-L432F-L434W-S436N | ++ |
| P284E-N285M-N287T-N388M-K394R-C430N-L432F-L434W-S436N | ++ |
| P284E-N285M-N287K-N388M-K394R-C430N-L432F-L434W-S436N | ++ |
| P284E-N285M-S322T-N388M-K394R-C430N-L432F-L434W-S436N | ++ |
| P284E-N287Q-S322T-N388M-K394R-C430N-L432F-L434W-S436N | ++ |
| P284E-N287V-S322T-N388M-K394R-C430N-L432F-L434W-S436N | ++ |
| P284E-N287R-S322T-N388M-K394R-C430N-L432F-L434W-S436N | ++ |
| P284E-N287T-S322T-N388M-K394R-C430N-L432F-L434W-S436N | ++ |
| P284E-N287K-S322T-N388M-K394R-C430N-L432F-L434W-S436N | ++ |
| N285M-N287Q-S322T-N388M-K394R-C430N-L432F-L434W-S436N | ++ |
| N285M-N287V-S322T-N388M-K394R-C430N-L432F-L434W-S436N | ++ |
| N285M-N287R-S322T-N388M-K394R-C430N-L432F-L434W-S436N | ++ |
| N285M-N287T-S322T-N388M-K394R-C430N-L432F-L434W-S436N | ++ |
| N285M-N287K-S322T-N388M-K394R-C430N-L432F-L434W-S436N | ++ |
| P284E-N285M-N287R-N388M-K394R-C430N-L432F-L434W-S436N | ++ |
| P284E-N285M-N287V-S322T-N388M-K394R-C430N-L432F-L434W-S436N | ++ |
| P284E-N285M-N287R-S322T-N388M-K394R-C430N-L432F-L434W-S436N | ++ |
| P284E-N285M-N287T-S322T-N388M-K394R-C430N-L432F-L434W-S436N | ++ |
| P284E-N285M-N287K-S322T-N388M-K394R-C430N-L432F-L434W-S436N | ++ |
| I283T-N285P-N287T-Q290E-S322T-N388M-K394R-C430A-L432F-L434W-S436N-dG462-dA463 | ++ |
| S189C-P284E-N287R-S322T-N388M-K394R-C430N-L432F-L434W-S436N | +++ |
| I283S-P284E-N287R-S322T-N388M-K394R-C430N-L432F-L434W-S436N | +++ |
| I283F-P284E-N287R-S322T-N388M-K394R-C430N-L432F-L434W-S436N | +++ |
| I283H-P284E-N287R-S322T-N388M-K394R-C430N-L432F-L434W-S436N | +++ |
| I283V-P284E-N287R-S322T-N388M-K394R-C430N-L432F-L434W-S436N | +++ |
| P284D-N287R-S322T-N388M-K394R-C430N-L432F-L434W-S436N | +++ |
| I283T-P284E-N287R-S322T-N388M-K394R-C430N-L432F-L434W-S436N | +++ |
| I283L-P284E-N287R-S322T-N388M-K394R-C430N-L432F-L434W-S436N | +++ |
| P284E-N285T-N287R-S322T-N388M-K394R-C430N-L432F-L434W-S436N | +++ |
| P284E-E286W-N287R-S322T-N388M-K394R-C430N-L432F-L434W-S436N | +++ |
| P284E-N287S-S322T-N388M-K394R-C430N-L432F-L434W-S436N | +++ |
| P284E-N285H-N287R-S322T-N388M-K394R-C430N-L432F-L434W-S436N | +++ |
| P284E-N287P-S322T-N388M-K394R-C430N-L432F-L434W-S436N | +++ |
| P284E-N287R-S288N-S322T-N388M-K394R-C430N-L432F-L434W-S436N | +++ |
| P284E-N287R-C321V-S322T-N388M-K394R-C430N-L432F-L434W-S436N | +++ |
| P284E-N287R-S322T-A329S-N388M-K394R-C430N-L432F-L434W-S436N | +++ |
| L6M-P284E-N287R-S322T-N388M-K394R-C430N-L432F-L434W-S436N | +++ |
| P284E-N287R-S322T-N338G-N388M-K394R-C430N-L432F-L434W-S436N | +++ |
| P284E-N287R-S322T-V379I-N388M-K394R-C430N-L432F-L434W-S436N | +++ |
| P284E-N287R-S322T-V382L-N388M-K394R-C430N-L432F-L434W-S436N | +++ |
| P284E-N287R-S322T-N388Q-K394R-C430N-L432F-L434W-S436N | +++ |
| P284E-N287R-S322T-N388M-K394R-F426V-C430N-L432F-L434W-S436N | +++ |
| P284E-N287R-S322T-N388M-K394R-F426Y-C430N-L432F-L434W-S436N | +++ |
| P284E-N287R-S322T-N388M-K394R-F426W-C430N-L432F-L434W-S436N | +++ |
| V46I-P284E-N287R-S322T-N388M-K394R-C430N-L432F-L434W-S436N | +++ |
| P284E-N287R-S322T-N388M-K394R-C430N-L432F-L434W-S436W | +++ |
| P284E-N287R-S322T-N388M-K394R-C430N-L432F-L434W-S436N-C437M | +++ |
| P284E-N287R-S322T-N388M-K394R-C430N-L432F-L434W-S436N-E441D | +++ |
| P284E-N287R-S322T-N388M-K394R-C430N-L432F-L434W-S436N-E441T | +++ |
| P284E-N287R-S322T-N388M-K394R-C430N-L432F-L434W-S436N-E441H | +++ |
| P284E-N287R-S322T-N388M-K394R-C430N-L432F-L434W-S436N-H444L | +++ |
| P284E-N287R-S322T-N388M-K394R-C430N-L432F-L434W-S436N-K447W | +++ |
| P284E-N287R-S322T-N388M-K394R-C430N-L432F-L434W-S436N-K447F | +++ |
| F183Y-N285M-N287T-S322T-N388M-K394R-C430N-L432F-L434W-S436N | +++ |
| N285M-N287T-S322T-V333M-N388M-K394R-C430N-L432F-L434W-S436N | +++ |
| I283T-N285M-N287T-S322T-N388M-K394R-C430N-L432F-L434W-S436N | +++ |
| I283V-N285M-N287T-S322T-N388M-K394R-C430N-L432F-L434W-S436N | +++ |
| P284D-N285M-N287T-S322T-N388M-K394R-C430N-L432F-L434W-S436N | +++ |
| N285P-N287T-S322T-N388M-K394R-C430N-L432F-L434W-S436N | +++ |
| N285M-N287T-Q290E-S322T-N388M-K394R-C430N-L432F-L434W-S436N | +++ |
| N285M-N287T-I292L-S322T-N388M-K394R-C430N-L432F-L434W-S436N | +++ |
| N285M-N287T-I292V-S322T-N388M-K394R-C430N-L432F-L434W-S436N | +++ |
| N51D-N285M-N287T-S322T-N388M-K394R-C430N-L432F-L434W-S436N | +++ |
| N285M-N287T-C321T-S322T-N388M-K394R-C430N-L432F-L434W-S436N | +++ |
| N285M-N287T-C321V-S322T-N388M-K394R-C430N-L432F-L434W-S436N | +++ |
| N285M-N287T-S322T-N332R-N388M-K394R-C430N-L432F-L434W-S436N | +++ |
| N285M-N287T-S322T-N332S-N388M-K394R-C430N-L432F-L434W-S436N | +++ |
| N285M-N287T-S322T-T366A-Q385W-N388M-K394R-C430N-L432F-L434W-S436N | +++ |
| N285M-N287T-S322T-Q385L-N388M-K394R-C430N-L432F-L434W-S436N | +++ |
| N285M-N287T-S322T-Q385P-N388M-K394R-C430N-L432F-L434W-S436N | +++ |
| N285M-N287T-S322T-Q385S-N388M-K394R-C430N-L432F-L434W-S436N | +++ |
| N285M-N287T-S322T-N388M-S389A-K394R-C430N-L432F-L434W-S436N | +++ |
| N285M-N287T-S322T-N388M-S389Q-K394R-C430N-L432F-L434W-S436N | +++ |
| N285M-N287T-S322T-N388M-L392Y-K394R-C430N-L432F-L434W-S436N | +++ |
| R7C-N285M-N287T-S322T-N388M-K394Q-C430N-L432F-L434W-S436N | +++ |
| N285M-N287T-S322T-N388M-K394H-C430N-L432F-L434W-S436N | +++ |
| N285M-N287T-S322T-N388M-K394L-C430N-L432F-L434W-S436N | +++ |
| N285M-N287T-S322T-N388M-K394R-F426H-C430N-L432F-L434W-S436N | +++ |
| N285M-N287T-S322T-N388M-K394R-F426I-C430N-L432F-L434W-S436N | +++ |
| N285M-N287T-S322T-N388M-K394R-F426L-C430N-L432F-L434W-S436N | +++ |
| N285M-N287T-S322T-N388M-K394R-F426T-C430N-L432F-L434W-S436N | +++ |
| N285M-N287T-S322T-N388M-K394R-F426V-C430N-L432F-L434W-S436N | +++ |
| N285M-N287T-S322T-N388M-K394R-F426W-C430N-L432F-L434W-S436N | +++ |
| N285M-N287T-S322T-N388M-K394R-C430A-L432F-L434W-S436N | +++ |
| N285M-N287T-S322T-N388M-K394R-C430N-I431P-L432F-L434W-S436N | +++ |
| N285M-N287T-S322T-N388M-K394R-C430N-L432F-L434W-S436N-C437L | +++ |
| N285M-N287T-S322T-N388M-K394R-C430N-L432F-L434W-S436N-C437T | +++ |
| N285M-N287T-S322T-N388M-K394R-C430N-L432F-L434W-S436N-C437V | +++ |
| N285M-N287T-S322T-N388M-K394R-C430N-L432F-L434W-S436N-E441G | +++ |
| N285M-N287T-S322T-N388M-K394R-C430N-L432F-L434W-S436N-E441I | +++ |
| N285M-N287T-S322T-N388M-K394R-C430N-L432F-L434W-S436N-E441L | +++ |
| N285M-N287T-S322T-N388M-K394R-C430N-L432F-L434W-S436N-E441P | +++ |
| N285M-N287T-S322T-N388M-K394R-C430N-L432F-L434W-S436N-R443H | +++ |
| N285M-N287T-S322T-N388M-K394R-C430N-L432F-L434W-S436N-H444F | +++ |
| N285M-N287T-S322T-N388M-K394R-C430N-L432F-L434W-S436N-H444I | +++ |
| N285M-N287T-S322T-N388M-K394R-C430N-L432F-L434W-S436N-H444L | +++ |
| N285M-N287T-S322T-N388M-K394R-C430N-L432F-L434W-S436N-H444M | +++ |
| N285M-N287T-S322T-N388M-K394R-C430N-L432F-L434W-S436N-H444V | +++ |
| N285M-N287T-S322T-N388M-K394R-C430N-L432F-L434W-S436N-N445A | +++ |
| N285M-N287T-S322T-N388M-K394R-C430N-L432F-L434W-S436N-N445Q | +++ |
| N285M-N287T-S322T-N388M-K394R-C430N-L432F-L434W-S436N-N445T | +++ |
| N285M-N287T-S322T-N388M-K394R-C430N-L432F-L434W-S436N-R446D | +++ |
| N285M-N287T-S322T-N388M-K394R-C430N-L432F-L434W-S436N-R446G | +++ |
| N285M-N287T-S322T-N388M-K394R-C430N-L432F-L434W-S436N-R446P | +++ |
| N285M-N287T-S322T-N388M-K394R-C430N-L432F-L434W-S436N-K449W | +++ |
| N285M-N287T-S322T-N388M-K394R-C430N-L432F-L434W-S436N-K449N | +++ |
| E72K-P284E-N287Q-S322T-N388M-K394R-C430N-L432F-L434W-S436N | +++ |
| P284E-N287Q-S322T-N388M-K394R-C430N-L432F-L434W-S436N-K447H | +++ |
| P284E-N287Q-S322T-N332S-N388M-K394R-C430N-L432F-L434W-S436N | +++ |
| P284E-N287Q-S322T-N388M-K394R-C430N-L432F-L434W-S436M | +++ |
| P284E-N287Q-S322T-N388M-K394R-C430N-L432F-L434W-S436W | +++ |
| P284E-N287Q-S322T-T336H-N388M-K394R-C430N-L432F-L434W-S436N | +++ |
| P284E-N287Q-S322T-T336N-N388M-K394R-C430N-L432F-L434W-S436N | +++ |
| P284E-N287Q-S322T-T336Q-N388M-K394R-C430N-L432F-L434W-S436N | +++ |
| S89N-P284E-N287Q-S322T-A327S-N388M-K394R-C430N-L432F-L434W-S436N | +++ |
| I283C-P284E-N287Q-S322T-N388M-K394R-C430N-L432F-L434W-S436N | +++ |
| I283H-P284E-N287Q-S322T-N388M-K394R-C430N-L432F-L434W-S436N | +++ |
| I283S-P284E-N287Q-S322T-N388M-K394R-C430N-L432F-L434W-S436N | +++ |
| I283T-P284E-N287Q-S322T-N388M-K394R-C430N-L432F-L434W-S436N | +++ |
| I283V-P284E-N287Q-S322T-N388M-K394R-C430N-L432F-L434W-S436N | +++ |
| I283V-P284D-N287Q-S322T-N388M-K394R-C430N-L432F-L434W-S436N | +++ |
| P284E-N287Q-S322T-Q331G-N388M-K394R-C430N-L432F-L434W-S436N | +++ |
| P284E-N287Q-S322T-Q385F-N388M-K394R-C430N-L432F-L434W-S436N | +++ |
| P284E-N287Q-S322T-Q385P-N388M-K394R-C430N-L432F-L434W-S436N | +++ |
| P284E-N287Q-S322T-Q385R-N388M-K394R-C430N-L432F-L434W-S436N | +++ |
| P284E-N287Q-S322T-H384S-N388M-K394R-C430N-L432F-L434W-S436N | +++ |
| P284E-N287Q-S322T-N388M-K394G-C430N-L432F-L434W-S436N | +++ |
| P284E-N287Q-S322T-N388M-C430N-L432F-L434W-S436N | +++ |
| P284E-N287Q-S322T-N388M-K394L-C430N-L432F-L434W-S436N | +++ |
| R7C-P284E-N287Q-S322T-N388M-K394R-C430N-L432F-L434W-S436N | +++ |
| L48M-P284E-N287Q-S322T-N388M-K394R-C430N-L432F-L434W-S436N | +++ |
| P284E-N287Q-S322T-N388M-K394R-C430N-L432F-L434W-S436N-E440D | +++ |
| P284E-N287Q-S322T-N388M-K394R-C430N-L432F-L434W-S436N-E440I | +++ |
| P284E-N287Q-S322T-N388M-K394R-C430N-L432F-L434W-S436N-E441L | +++ |
| P284E-N287Q-S322T-N388M-K394R-C430N-L432F-L434W-S436N-E441T | +++ |
| P284E-N287Q-S322T-N388M-K394R-E428M-C430N-L432F-L434W-S436N | +++ |
| P284E-N287Q-S322T-N388M-K394R-E428T-C430N-L432F-L434W-S436N | +++ |
| P284E-N287Q-S322T-A327S-N388M-K394R-C430N-L432F-L434W-S436N | +++ |
| S189C-P284E-N287Q-S322T-N388M-K394R-C430N-L432F-L434W-S436N | +++ |
| P284E-N287Q-C321V-S322T-N388M-K394R-C430N-L432F-L434W-S436N | +++ |
| P284E-N287Q-S322T-N388M-K394R-F426L-C430N-L432F-L434W-S436N | +++ |
| P284E-N287Q-S322T-N388M-K394R-F426Y-C430N-L432F-L434W-S436N | +++ |
| N285M-N287T-Q290E-S322T-N388M-K394R-C430A-L432F-L434W-S436N | +++ |
| I283T-N285M-N287T-Q290E-S322T-N388M-K394R-C430A-L432F-L434W-S436N | +++ |
| N285P-N287T-Q290E-S322T-N388M-K394R-C430A-L432F-L434W-S436N | +++ |
| I283T-N285P-N287T-Q290E-S322T-N388M-K394R-C430A-L432F-L434W-S436N | +++ |
| P284D-N285P-N287T-Q290E-S322T-N388M-K394R-C430A-L432F-L434W-S436N | +++ |
| I283T-N285P-N287T-S322T-N388M-K394R-C430N-L432F-L434W-S436N | +++ |
| I283T-N285M-N287T-Q290E-S322T-N388M-K394R-C430N-L432F-L434W-S436N | +++ |
| I283T-N285M-N287T-S322T-N388M-K394R-C430A-L432F-L434W-S436N | +++ |
| P284D-N285P-N287T-S322T-N388M-K394R-C430N-L432F-L434W-S436N | +++ |
| I283T-N285P-N287T-S322T-N388M-K394R-C430A-L432F-L434W-S436N | +++ |
| P284D-N285P-N287T-Q290E-S322T-N388M-K394R-C430N-L432F-L434W-S436N | +++ |
| P284D-N285P-N287T-S322T-N388M-K394R-C430A-L432F-L434W-S436N | +++ |
| I283T-P284D-N285M-N287T-S322T-N388M-K394R-C430N-L432F-L434W-S436N | +++ |
| I283T-P284D-N285P-N287T-S322T-N388M-K394R-C430N-L432F-L434W-S436N | +++ |
| I283T-P284D-N285M-N287T-Q290E-S322T-N388M-K394R-C430N-L432F-L434W-S436N | +++ |
| I283T-P284D-N285M-N287T-S322T-N388M-K394R-C430A-L432F-L434W-S436N | +++ |
| I283T-P284D-N285P-N287T-Q290E-S322T-N388M-K394R-C430N-L432F-L434W-S436N | +++ |
| I283T-P284D-N285P-N287T-S322T-N388M-K394R-C430A-L432F-L434W-S436N | +++ |
| I283T-P284D-N285M-N287T-Q290E-S322T-N388M-K394R-C430A-L432F-L434W-S436N | +++ |
| I283T-P284D-N285P-N287T-Q290E-S322T-N388M-K394R-C430A-L432F-L434W-S436N | +++ |
| P284D-N285M-N287T-Q290E-S322T-N388M-K394R-C430N-L432F-L434W-S436N | +++ |
| P284D-N285M-N287T-S322T-N388M-K394R-C430A-L432F-L434W-S436N | +++ |
| N285P-N287T-Q290E-S322T-N388M-K394R-C430N-L432F-L434W-S436N | +++ |
| N285P-N287T-S322T-N388M-K394R-C430A-L432F-L434W-S436N | +++ |
| I283T-N285P-N287T-Q290E-S322T-N388M-K394R-C430N-L432F-L434W-S436N | +++ |
| I283T-P284D-N287R-S322T-N388M-K394R-C430N-L432F-L434W-S436N | +++ |
| I283T-P284D-N287P-S322T-N388M-K394R-C430N-L432F-L434W-S436N | +++ |
| I283T-P284D-N287R-S322T-N388M-C430N-L432F-L434W-S436N | +++ |
| I283T-P284D-E286W-N287P-S322T-N388M-K394R-C430N-L432F-L434W-S436N | +++ |
| I283T-P284D-N287P-S322T-N388M-C430N-L432F-L434W-S436N | +++ |
| I283C-P284E-E286W-N287P-S322T-N388M-K394R-C430N-L432F-L434W-S436N | +++ |
| I283T-P284E-E286W-N287P-S322T-N388M-K394R-C430N-L432F-L434W-S436N | +++ |
| I283C-P284E-E286W-N287P-S322T-N388M-C430N-L432F-L434W-S436N | +++ |
| I283T-P284E-E286W-N287P-S322T-N388M-C430N-L432F-L434W-S436N | +++ |
| I283T-P284D-E286W-N287P-S322T-N388M-C430N-L432F-L434W-S436N | +++ |
| I283C-P284E-N287R-S322T-N388M-K394R-C430N-L432F-L434W-S436N | +++ |
| I283C-P284D-N287P-S322T-N388M-K394R-C430N-L432F-L434W-S436N | +++ |
| I283C-P284D-N287R-S322T-N388M-C430N-L432F-L434W-S436N | +++ |
| I283C-P284D-N287P-S322T-N388M-C430N-L432F-L434W-S436N | +++ |
| I283C-P284E-N287R-S322T-N388M-C430N-L432F-L434W-S436N | +++ |
| I283T-P284E-N287R-S322T-N388M-C430N-L432F-L434W-S436N | +++ |
| P284D-N287R-S322T-N388M-C430N-L432F-L434W-S436N | +++ |
| I283C-P284E-E286W-N287R-S322T-N388M-C430N-L432F-L434W-S436N | +++ |
| I283C-P284E-N287P-S322T-N388M-C430N-L432F-L434W-S436N | +++ |
| I283T-P284E-N287P-S322T-N388M-C430N-L432F-L434W-S436N | +++ |
| I283T-P284E-E286W-N287R-S322T-N388M-K394R-C430N-L432F-L434W-S436N | +++ |
| I283T-P284E-N287P-S322T-N388M-K394R-C430N-L432F-L434W-S436N | +++ |
| P284E-E286W-N287R-S322T-N388M-C430N-L432F-L434W-S436N | +++ |
| P284E-N287P-S322T-N388M-C430N-L432F-L434W-S436N | +++ |
| I283T-P284E-E286W-N287R-S322T-N388M-C430N-L432F-L434W-S436N | +++ |
| P284D-N287P-S322T-N388M-C430N-L432F-L434W-S436N | +++ |
| I283C-P284E-E286W-N287R-S322T-N388M-K394R-C430N-L432F-L434W-S436N | +++ |
| I283C-P284E-N287P-S322T-N388M-K394R-C430N-L432F-L434W-S436N | +++ |
| P284D-N287P-S322T-N388M-K394R-C430N-L432F-L434W-S436N | +++ |
| I283C-P284D-N287Q-S322T-N388M-K394R-C430N-L432F-L434W-S436N | +++ |
| I283C-P284E-N287Q-S322T-N388M-C430N-L432F-L434W-S436N | +++ |
| I283C-P284D-N287Q-S322T-N388M-C430N-L432F-L434W-S436N | +++ |
| I283C-P284E-E286W-N287Q-S322T-N388M-C430N-L432F-L434W-S436N | +++ |
| I283T-P284D-N287Q-S322T-N388M-K394R-C430N-L432F-L434W-S436N | +++ |
| I283T-P284E-E286W-N287Q-S322T-N388M-K394R-C430N-L432F-L434W-S436N | +++ |
| I283T-P284E-N287Q-S322T-N388M-C430N-L432F-L434W-S436N | +++ |
| I283T-P284D-N287Q-S322T-N388M-C430N-L432F-L434W-S436N | +++ |
| I283T-P284E-E286W-N287Q-S322T-N388M-C430N-L432F-L434W-S436N | +++ |
| P284D-N287Q-S322T-N388M-C430N-L432F-L434W-S436N | +++ |
| P284E-E286W-N287Q-S322T-N388M-C430N-L432F-L434W-S436N | +++ |
| N285M-N287T-S322T-N388M-K394R-C430N-L432F-L434W-S436N-P458E | +++ |
| N285M-N287T-S322T-N388M-K394R-C430N-L432F-L434W-S436N-G462V | +++ |
| N285M-N287T-S322T-N388M-K394R-C430N-L432F-L434W-S436N-E459P | +++ |
| N285M-N287T-S322T-N388M-K394R-C430N-L432F-L434W-S436N-E459R | +++ |
| N285M-N287T-S322T-N388M-K394R-C430N-L432F-L434W-S436N-H461A | +++ |
| N285M-N287T-S322T-N388M-K394R-C430N-L432F-L434W-S436N-H461D | +++ |
| N285M-N287T-S322T-N388M-K394R-C430N-L432F-L434W-S436N-H461E | +++ |
| N285M-N287T-S322T-N388M-K394R-C430N-L432F-L434W-S436N-H461G | +++ |
| N285M-N287T-S322T-N388M-K394R-C430N-L432F-L434W-S436N-H461N | +++ |
| P45Q-N285M-N287T-S322T-N388M-K394R-C430N-L432F-L434W-S436N-H461S | +++ |
| N285M-N287T-S322T-N388M-K394R-C430N-L432F-L434W-S436N-G462A | +++ |
| N285M-N287T-S322T-N388M-K394R-C430N-L432F-L434W-S436N-G462C | +++ |
| N285M-N287T-S322T-N388M-K394R-C430N-L432F-L434W-S436N-G462P | +++ |
| N285M-N287T-S322T-N388M-K394R-C430N-L432F-L434W-S436N-G462I | +++ |
| N285M-N287T-S322T-N388M-K394R-C430N-L432F-L434W-S436N-A463L | +++ |
| N285M-N287T-S322T-N388M-K394R-C430N-L432F-L434W-S436N-A463P | +++ |
| N285M-N287T-S322T-N388M-K394R-C430N-L432F-L434W-S436N-Y469D | +++ |
| N285M-N287T-S322T-N388M-K394R-C430N-L432F-L434W-S436N-Y469E | +++ |
| N285M-N287T-S322T-N388M-K394R-C430N-L432F-L434W-S436N-Y469S | +++ |
| N285M-N287T-S322T-N388M-K394R-C430N-L432F-L434W-S436N-Y469T | +++ |
| N285M-N287T-S322T-N388M-K394R-C430N-L432F-L434W-S436N-Y471F | +++ |
| H141L-N285M-N287T-S322T-N388M-K394R-C430N-L432F-L434W-S436N | +++ |
| N144S-N285M-N287T-S322T-N388M-K394R-C430N-L432F-L434W-S436N | +++ |
| R15H-N285M-N287T-S322T-N388M-K394R-C430N-L432F-L434W-S436N | +++ |
| P284E-N287Q-S322T-N388M-K394R-C430N-L432F-L434W-S436N-E459L | +++ |
| V46I-P284E-N287Q-S322T-N388M-K394R-C430N-L432F-L434W-S436N-N460V | +++ |
| P284E-N287Q-S322T-N388M-K394R-C430N-L432F-L434W-S436N-N460G | +++ |
| P284E-N287Q-S322T-N388M-K394R-C430N-L432F-L434W-S436N-N460I | +++ |
| P284E-N287Q-S322T-N388M-K394R-C430N-L432F-L434W-S436N-H461A | +++ |
| P284E-N287Q-S322T-N388M-K394R-C430N-L432F-L434W-S436N-H461G | +++ |
| P284E-N287Q-S322T-N388M-K394R-C430N-L432F-L434W-S436N-H461N | +++ |
| P284E-N287Q-S322T-N388M-K394R-C430N-L432F-L434W-S436N-H461T | +++ |
| P284E-N287Q-S322T-N388M-K394R-C430N-L432F-L434W-S436N-H461D | +++ |
| P284E-N287Q-S322T-N388M-K394R-C430N-L432F-L434W-S436N-A463E | +++ |
| P284E-N287Q-S322T-N388M-K394R-C430N-L432F-L434W-S436N-A463G | +++ |
| P284E-N287Q-S322T-N388M-K394R-C430N-L432F-L434W-S436N-F467W | +++ |
| P284E-N287Q-S322T-N388M-K394R-C430N-L432F-L434W-S436N-Y469D | +++ |
| P284E-N287Q-S322T-N388M-K394R-C430N-L432F-L434W-S436N-Y469N | +++ |
| P284E-N287Q-S322T-N388M-K394R-C430N-L432F-L434W-S436N-Y469S | +++ |
| P284E-N287Q-S322T-N388M-K394R-C430N-L432F-L434W-S436N-Y469T | +++ |
| P284E-N287Q-S322T-N388M-K394R-C430N-L432F-L434W-S436N-A470G | +++ |
| P284E-N287Q-S322T-N388M-K394R-C430N-L432F-L434W-S436N-Y471F | +++ |
| H141Q-P284E-N287Q-S322T-N388M-K394R-C430N-L432F-L434W-S436N | +++ |
| H141S-P284E-N287Q-S322T-N388M-K394R-C430N-L432F-L434W-S436N | +++ |
| H141Y-P284E-N287Q-S322T-N388M-K394R-C430N-L432F-L434W-S436N | +++ |
| N285M-N287T-S322T-N388M-K394R-C430N-L432F-L434W-S436N-dP458 | +++ |
| N285M-N287T-S322T-N388M-K394R-C430N-L432F-L434W-S436N-dE459 | +++ |
| N285M-N287T-S322T-N388M-K394R-C430N-L432F-L434W-S436N-dN460 | +++ |
| N285M-N287T-S322T-N388M-K394R-C430N-L432F-L434W-S436N-dH461 | +++ |
| N285M-N287T-S322T-N388M-K394R-C430N-L432F-L434W-S436N-dG462 | +++ |
| N285M-N287T-S322T-N388M-K394R-C430N-L432F-L434W-S436N-dA463 | +++ |
| P284E-N287Q-S322T-N388M-K394R-C430N-L432F-L434W-S436N-dP458 | +++ |
| P284E-N287Q-S322T-N388M-K394R-C430N-L432F-L434W-S436N-dE459 | +++ |
| P284E-N287Q-S322T-N388M-K394R-C430N-L432F-L434W-S436N-dN460 | +++ |
| P284E-N287Q-S322T-N388M-K394R-C430N-L432F-L434W-S436N-dH461 | +++ |
| P284E-N287Q-S322T-N388M-K394R-C430N-L432F-L434W-S436N-dG462 | +++ |
| P284E-N287Q-S322T-N388M-K394R-C430N-L432F-L434W-S436N-dA463 | +++ |
| P284E-N287Q-S322T-N388M-K394R-C430N-L432F-L434W-S436N-H461G-Y469D | +++ |
| P284E-N287Q-S322T-N388M-K394R-C430N-L432F-L434W-S436N-A463G-Y469D | +++ |
| P284E-N287Q-S322T-N388M-K394R-C430N-L432F-L434W-S436N-H461G-A463G-Y469D | +++ |
| P284E-N287Q-S322T-N388M-K394R-C430N-L432F-L434W-S436N-N460G-H461G-A463G-Y469D | +++ |
| P284D-N287P-S322T-N388M-C430N-L432F-L434W-S436N-N460G | +++ |
| P284D-N287P-S322T-N388M-C430N-L432F-L434W-S436N-N460I | +++ |
| P284D-N287P-S322T-N388M-C430N-L432F-L434W-S436N-H461G | +++ |
| P284D-N287P-S322T-N388M-C430N-L432F-L434W-S436N-A463G | +++ |
| P284D-N287P-S322T-N388M-C430N-L432F-L434W-S436N-Y469D | +++ |
| P284D-N287P-S322T-N388M-C430N-L432F-L434W-S436N-N460G-H461G | +++ |
| P284D-N287P-S322T-N388M-C430N-L432F-L434W-S436N-N460G-A463G | +++ |
| P284D-N287P-S322T-N388M-C430N-L432F-L434W-S436N-N460G-Y469D | +++ |
| P284D-N287P-S322T-N388M-C430N-L432F-L434W-S436N-N460I-A463G | +++ |
| P284D-N287P-S322T-N388M-C430N-L432F-L434W-S436N-N460I-Y469D | +++ |
| P284D-N287P-S322T-N388M-C430N-L432F-L434W-S436N-H461G-A463G | +++ |
| P284D-N287P-S322T-N388M-C430N-L432F-L434W-S436N-H461G-Y469D | +++ |
| P284D-N287P-S322T-N388M-C430N-L432F-L434W-S436N-A463G-Y469D | +++ |
| P284D-N287P-S322T-N388M-C430N-L432F-L434W-S436N-N460G-H461G-A463G | +++ |
| P284D-N287P-S322T-N388M-C430N-L432F-L434W-S436N-N460G-H461G-Y469D | +++ |
| P284D-N287P-S322T-N388M-C430N-L432F-L434W-S436N-N460G-A463G-Y469D | +++ |
| P284D-N287P-S322T-N388M-C430N-L432F-L434W-S436N-N460I-H461G-Y469D | +++ |
| P284D-N287P-S322T-N388M-C430N-L432F-L434W-S436N-N460I-A463G-Y469D | +++ |
| P284D-N287P-S322T-N388M-C430N-L432F-L434W-S436N-H461G-A463G-Y469D | +++ |
| P284D-N287P-S322T-N388M-C430N-L432F-L434W-S436N-N460G-H461G-A463G-Y469D | +++ |
| P284D-N287P-S322T-N388M-C430N-L432F-L434W-S436N-N460I-H461G-A463G-Y469D | +++ |
| I283T-P284D-N287P-S322T-N388M-C430N-L432F-L434W-S436N-N460G | +++ |
| I283T-P284D-N287P-S322T-N388M-C430N-L432F-L434W-S436N-A463G | +++ |
| I283T-P284D-N287P-S322T-N388M-C430N-L432F-L434W-S436N-Y469D | +++ |
| I283T-P284D-N287P-S322T-N388M-C430N-L432F-L434W-S436N-N460G-A463G | +++ |
| I283T-P284D-N287P-S322T-N388M-C430N-L432F-L434W-S436N-N460G-Y469D | +++ |
| I283T-P284D-N287P-S322T-N388M-C430N-L432F-L434W-S436N-N460G-A463G-Y469D | +++ |
| I283T-P284D-N287P-S322T-N388M-C430N-L432F-L434W-S436N-N460I-H461G-Y469D | +++ |
| I283T-P284D-N287P-S322T-N388M-C430N-L432F-L434W-S436N-N460I-A463G-Y469D | +++ |
| I283T-P284D-N287P-S322T-N388M-C430N-L432F-L434W-S436N-H461G-A463G-Y469D | +++ |
| I283T-P284D-N287P-S322T-N388M-C430N-L432F-L434W-S436N-N460G-H461G-A463G-Y469D | +++ |
| I283T-P284D-N287P-S322T-N388M-C430N-L432F-L434W-S436N-N460I-H461G | +++ |
| I283T-P284D-N287P-S322T-N388M-C430N-L432F-L434W-S436N-N460I-A463G | +++ |
| I283T-P284D-N287P-S322T-N388M-C430N-L432F-L434W-S436N-N460I-Y469D | +++ |
| I283T-P284D-N287P-S322T-N388M-C430N-L432F-L434W-S436N-H461G-A463G | +++ |
| I283T-P284D-N287P-S322T-N388M-C430N-L432F-L434W-S436N-H461G-Y469D | +++ |
| I283T-P284D-N287P-S322T-N388M-C430N-L432F-L434W-S436N-A463G-Y469D | +++ |
| I283T-P284D-N287P-S322T-N388M-C430N-L432F-L434W-S436N-N460G-H461G-A463G | +++ |
| I283T-P284D-N287P-S322T-N388M-C430N-L432F-L434W-S436N-N460G-H461G-Y469D | +++ |
| I283T-P284D-N287Q-S322T-N388M-C430N-L432F-L434W-S436N-H461G | +++ |
| I283T-P284D-N287Q-S322T-N388M-C430N-L432F-L434W-S436N-A463G | +++ |
| I283T-P284D-N287Q-S322T-N388M-C430N-L432F-L434W-S436N-Y469D | +++ |
| I283T-P284D-N287Q-S322T-N388M-C430N-L432F-L434W-S436N-N460G-H461G | +++ |
| I283T-P284D-N287Q-S322T-N388M-C430N-L432F-L434W-S436N-N460G-A463G | +++ |
| I283T-P284D-N287Q-S322T-N388M-C430N-L432F-L434W-S436N-N460G-Y469D | +++ |
| I283T-P284D-N287Q-S322T-N388M-C430N-L432F-L434W-S436N-N460I-Y469D | +++ |
| I283T-P284D-N287Q-S322T-N388M-C430N-L432F-L434W-S436N-H461G-Y469D | +++ |
| I283T-P284D-N287Q-S322T-N388M-C430N-L432F-L434W-S436N-A463G-Y469D | +++ |
| I283T-P284D-N287Q-S322T-N388M-C430N-L432F-L434W-S436N-N460G-H461G-A463G | +++ |
| I283T-P284D-N287Q-S322T-N388M-C430N-L432F-L434W-S436N-N460G-H461G-Y469D | +++ |
| I283T-P284D-N287Q-S322T-N388M-C430N-L432F-L434W-S436N-N460I-H461G-Y469D | +++ |
| I283T-P284D-N287Q-S322T-N388M-C430N-L432F-L434W-S436N-N460G-H461G-A463G-Y469D | +++ |
| P284D-N287P-S322T-N388M-C430N-L432F-L434W-S436N-dG462 | +++ |
| I283T-P284D-N287P-S322T-N388M-C430N-L432F-L434W-S436N-dG462 | +++ |
| P284D-N287P-S322T-N388M-C430N-L432F-L434W-S436N-dA463 | +++ |
| I283T-P284D-N287P-S322T-N388M-C430N-L432F-L434W-S436N-dA463 | +++ |
| I283T-P284D-N287Q-S322T-N388M-C430N-L432F-L434W-S436N-dG462 | +++ |
| I283T-P284D-N287Q-S322T-N388M-C430N-L432F-L434W-S436N-dA463 | +++ |
| I283T-P284D-N285P-N287T-S322T-N388M-K394R-C430N-L432F-L434W-S436N-G462A | +++ |
| I283T-P284D-N285P-N287T-S322T-N388M-K394R-C430N-L432F-L434W-S436N-G462P | +++ |
| I283T-P284D-N285P-N287T-S322T-N388M-K394R-C430N-L432F-L434W-S436N-G462V | +++ |
| I283T-P284D-N285P-N287T-S322T-N388M-K394R-C430N-L432F-L434W-S436N-Y469D | +++ |
| I283T-P284D-N285P-N287T-S322T-N388M-K394R-C430N-L432F-L434W-S436N-G462A-A463P | +++ |
| I283T-P284D-N285P-N287T-S322T-N388M-K394R-C430N-L432F-L434W-S436N-G462A-Y469D | +++ |
| I283T-P284D-N285P-N287T-S322T-N388M-K394R-C430N-L432F-L434W-S436N-G462P-A463P | +++ |
| I283T-P284D-N285P-N287T-S322T-N388M-K394R-C430N-L432F-L434W-S436N-G462P-Y469D | +++ |
| N285M-N287T-S322T-N388M-K394R-C430N-L432F-L434W-S436N-G462V-A463P | +++ |
| N285M-N287T-S322T-N388M-K394R-C430N-L432F-L434W-S436N-G462V-Y469D | +++ |
| N285M-N287T-S322T-N388M-K394R-C430N-L432F-L434W-S436N-A463P-Y469D | +++ |
| N285M-N287T-S322T-N388M-K394R-C430N-L432F-L434W-S436N-G462A-A463P-Y469D | +++ |
| N285M-N287T-S322T-N388M-K394R-C430N-L432F-L434W-S436N-G462P-A463P-Y469D | +++ |
| I283T-P284D-N285P-N287T-S322T-N388M-K394R-C430N-L432F-L434W-S436N-G462V-A463P | +++ |
| I283T-P284D-N285P-N287T-S322T-N388M-K394R-C430N-L432F-L434W-S436N-G462V-Y469D | +++ |
| I283T-P284D-N285P-N287T-S322T-N388M-K394R-C430N-L432F-L434W-S436N-A463P-Y469D | +++ |
| I283T-P284D-N285P-N287T-S322T-N388M-K394R-C430N-L432F-L434W-S436N-G462A-A463P-Y469D | +++ |
| I283T-P284D-N285P-N287T-S322T-N388M-K394R-C430N-L432F-L434W-S436N-G462P-A463P-Y469D | +++ |
| I283T-P284D-N285P-N287T-S322T-N388M-K394R-C430N-L432F-L434W-S436N-G462V-A463P-Y469D | +++ |
| I283T-P284D-N285M-N287T-S322T-N388M-K394R-C430A-L432F-L434W-S436N-G462A | +++ |
| I283T-P284D-N285M-N287T-S322T-N388M-K394R-C430A-L432F-L434W-S436N-G462P | +++ |
| I283T-P284D-N285M-N287T-S322T-N388M-K394R-C430A-L432F-L434W-S436N-G462V | +++ |
| I283T-P284D-N285M-N287T-S322T-N388M-K394R-C430A-L432F-L434W-S436N-Y469D | +++ |
| I283T-P284D-N285M-N287T-S322T-N388M-K394R-C430A-L432F-L434W-S436N-G462A-A463P | +++ |
| I283T-P284D-N285M-N287T-S322T-N388M-K394R-C430A-L432F-L434W-S436N-G462A-Y469D | +++ |
| I283T-P284D-N285M-N287T-S322T-N388M-K394R-C430A-L432F-L434W-S436N-G462P-A463P | +++ |
| I283T-P284D-N285M-N287T-S322T-N388M-K394R-C430A-L432F-L434W-S436N-G462P-Y469D | +++ |
| I283T-P284D-N285M-N287T-S322T-N388M-K394R-C430A-L432F-L434W-S436N-G462V-A463P | +++ |
| I283T-P284D-N285M-N287T-S322T-N388M-K394R-C430A-L432F-L434W-S436N-G462V-Y469D | +++ |
| I283T-P284D-N285M-N287T-S322T-N388M-K394R-C430A-L432F-L434W-S436N-A463P-Y469D | +++ |
| I283T-P284D-N285M-N287T-S322T-N388M-K394R-C430A-L432F-L434W-S436N-G462A-A463P-Y469D | +++ |
| I283T-P284D-N285M-N287T-S322T-N388M-K394R-C430A-L432F-L434W-S436N-G462P-A463P-Y469D | +++ |
| I283T-P284D-N285M-N287T-S322T-N388M-K394R-C430A-L432F-L434W-S436N-G462V-A463P-Y469D | +++ |
| I283T-N285M-N287T-Q290E-S322T-N388M-K394R-C430A-L432F-L434W-S436N-G462A | +++ |
| I283T-N285M-N287T-Q290E-S322T-N388M-K394R-C430A-L432F-L434W-S436N-G462P | +++ |
| I283T-N285M-N287T-Q290E-S322T-N388M-K394R-C430A-L432F-L434W-S436N-G462V | +++ |
| I283T-N285M-N287T-Q290E-S322T-N388M-K394R-C430A-L432F-L434W-S436N-A463P | +++ |
| I283T-N285M-N287T-Q290E-S322T-N388M-K394R-C430A-L432F-L434W-S436N-Y469D | +++ |
| I283T-N285M-N287T-Q290E-S322T-N388M-K394R-C430A-L432F-L434W-S436N-G462A-A463P | +++ |
| I283T-N285M-N287T-Q290E-S322T-N388M-K394R-C430A-L432F-L434W-S436N-G462A-Y469D | +++ |
| I283T-N285M-N287T-Q290E-S322T-N388M-K394R-C430A-L432F-L434W-S436N-G462P-A463P | +++ |
| I283T-N285M-N287T-Q290E-S322T-N388M-K394R-C430A-L432F-L434W-S436N-G462P-Y469D | +++ |
| I283T-N285M-N287T-Q290E-S322T-N388M-K394R-C430A-L432F-L434W-S436N-G462V-A463P | +++ |
| I283T-N285M-N287T-Q290E-S322T-N388M-K394R-C430A-L432F-L434W-S436N-G462V-Y469D | +++ |
| I283T-N285M-N287T-Q290E-S322T-N388M-K394R-C430A-L432F-L434W-S436N-A463P-Y469D | +++ |
| I283T-N285M-N287T-Q290E-S322T-N388M-K394R-C430A-L432F-L434W-S436N-G462A-A463P-Y469D | +++ |
| I283T-N285M-N287T-Q290E-S322T-N388M-K394R-C430A-L432F-L434W-S436N-G462P-A463P-Y469D | +++ |
| I283T-N285M-N287T-Q290E-S322T-N388M-K394R-C430A-L432F-L434W-S436N-G462V-A463P-Y469D | +++ |
| I283T-N285P-N287T-Q290E-S322T-N388M-K394R-C430A-L432F-L434W-S436N-G462A | +++ |
| I283T-N285P-N287T-Q290E-S322T-N388M-K394R-C430A-L432F-L434W-S436N-G462P | +++ |
| I283T-N285P-N287T-Q290E-S322T-N388M-K394R-C430A-L432F-L434W-S436N-G462V | +++ |
| I283T-N285P-N287T-Q290E-S322T-N388M-K394R-C430A-L432F-L434W-S436N-A463P | +++ |
| I283T-N285P-N287T-Q290E-S322T-N388M-K394R-C430A-L432F-L434W-S436N-Y469D | +++ |
| I283T-N285P-N287T-Q290E-S322T-N388M-K394R-C430A-L432F-L434W-S436N-G462A-A463P | +++ |
| I283T-N285P-N287T-Q290E-S322T-N388M-K394R-C430A-L432F-L434W-S436N-G462A-Y469D | +++ |
| I283T-N285P-N287T-Q290E-S322T-N388M-K394R-C430A-L432F-L434W-S436N-G462P-A463P | +++ |
| I283T-N285P-N287T-Q290E-S322T-N388M-K394R-C430A-L432F-L434W-S436N-G462P-Y469D | +++ |
| I283T-N285P-N287T-Q290E-S322T-N388M-K394R-C430A-L432F-L434W-S436N-G462V-A463P | +++ |
| I283T-N285P-N287T-Q290E-S322T-N388M-K394R-C430A-L432F-L434W-S436N-G462V-Y469D | +++ |
| I283T-N285P-N287T-Q290E-S322T-N388M-K394R-C430A-L432F-L434W-S436N-A463P-Y469D | +++ |
| I283T-N285P-N287T-Q290E-S322T-N388M-K394R-C430A-L432F-L434W-S436N-G462A-A463P-Y469D | +++ |
| I283T-N285P-N287T-Q290E-S322T-N388M-K394R-C430A-L432F-L434W-S436N-G462P-A463P-Y469D | +++ |
| I283T-N285P-N287T-Q290E-S322T-N388M-K394R-C430A-L432F-L434W-S436N-G462V-A463P-Y469D | +++ |
| N285M-N287T-S322T-N388M-K394R-C430N-L432F-L434W-S436N-G462A-A463P | +++ |
| N285M-N287T-S322T-N388M-K394R-C430N-L432F-L434W-S436N-G462A-Y469D | +++ |
| N285M-N287T-S322T-N388M-K394R-C430N-L432F-L434W-S436N-G462P-A463P | +++ |
| N285M-N287T-S322T-N388M-K394R-C430N-L432F-L434W-S436N-G462P-Y469D | +++ |
| R7C-I283T-N285P-N287T-Q290E-S322T-Q385D-N388M-K394R-C430A-L432F-L434W-S436N-dG462-dA463 | +++ |
| I283T-N285P-N287T-Q290E-S322T-N388M-K394R-i421aP-C430A-L432F-L434W-S436N-dG462-dA463 | +++ |
| P184A-I283T-N285P-N287T-Q290E-S322T-N388M-K394R-C430A-L432F-L434W-S436N-dG462-dA463 | +++ |
| I283T-N285P-N287T-Q290E-S322T-N388M-K394C-C430A-L432F-L434W-S436N-dG462-dA463 | +++ |
| I283T-N285P-N287T-Q290E-S322T-N388M-K394R-R422S-C430A-L432F-L434W-S436N-dG462-dA463 | +++ |
| I283T-N285P-N287T-Q290E-S322T-N388M-K394R-H419P-C430A-L432F-L434W-S436N-dG462-dA463 | +++ |
| I283T-N285P-N287T-Q290E-S322T-N388M-K394R-C430A-L432F-L434W-S436N-N460L-dG462-dA463 | +++ |
| I283T-N285P-N287T-Q290E-S322T-N388M-K394R-C430A-L432F-L434W-S436N-S439L-dG462-dA463 | +++ |
| I283T-N285P-N287T-Q290E-S322T-N388M-K394R-C430A-L432F-L434W-S436N-P458L-dG462-dA463 | +++ |
| I283T-N285P-N287T-Q290E-S322T-N388M-K394R-C430A-L432F-L434W-S436N-L457A-dG462-dA463 | +++ |
| I283T-N285P-N287T-Q290E-S322T-N388M-K394R-C430A-L432F-L434W-S436N-P458C-dG462-dA463 | +++ |
| I283T-N285P-N287T-Q290E-S322T-N388M-K394R-C430A-L432F-L434W-S436N-E459T-dG462-dA463 | +++ |
| I283T-N285P-N287T-Q290E-S322T-N388M-K394R-C430A-L432F-L434W-S436N-P458M-dG462-dA463 | +++ |
| I283T-N285P-N287T-Q290E-S322T-N388M-K394R-C430A-L432F-L434W-S436N-P458I-dG462-dA463 | +++ |
| I283T-N285P-N287T-Q290E-S322T-N388M-K394R-C430A-L432F-L434W-S436N-E459G-dG462-dA463 | +++ |
| I283T-N285P-N287T-Q290E-S322T-N388M-K394R-C430A-L432F-L434W-S436N-P458R-dG462-dA463 | +++ |
| I283T-N285P-N287T-Q290E-S322T-N388M-K394R-C430A-L432F-L434W-S436N-P458Q-dG462-dA463 | +++ |
| I283T-N285P-N287T-Q290E-S322T-N388M-K394R-C430A-L432F-L434W-S436N-N460H-dG462-dA463 | +++ |
| I283T-N285P-N287T-Q290E-S322T-N388M-K394R-C430A-L432F-L434W-S436N-N460Y-dG462-dA463 | +++ |
| I283T-N285P-N287T-Q290E-S322T-N388M-K394R-C430A-L432F-L434W-S436N-N460I-dG462-dA463 | +++ |
| I283T-N285P-N287T-Q290E-S322T-N388M-K394R-C430A-L432F-L434W-S436N-N460M-dG462-dA463 | +++ |
| I283T-P284D-N287P-S322T-N388M-K423C-C430N-L432F-L434W-S436N-A463G-Y469D | ++++ |
| I283T-P284D-N285A-N287P-S322T-N388M-C430N-L432F-L434W-S436N-A463G-Y469D | ++++ |
| I283T-P284D-N285I-N287P-S322T-N388M-C430N-L432F-L434W-S436N-A463G-Y469D | ++++ |
| I283T-P284D-N285L-N287P-S322T-N388M-C430N-L432F-L434W-S436N-A463G-Y469D | ++++ |
| I283T-P284D-N287P-S322T-N388M-C430N-L432F-L434W-S436T-A463G-Y469D | ++++ |
| I283F-P284D-N287P-S322T-N388M-C430N-L432F-L434W-S436N-A463G-Y469D | ++++ |
| I283S-P284D-N287P-S322T-N388M-C430N-L432F-L434W-S436N-A463G-Y469D | ++++ |
| I283T-P284D-N287P-S288T-S322T-N388M-C430N-L432F-L434W-S436N-A463G-Y469D | ++++ |
| I283T-P284D-N287P-S322T-N388M-S429A-C430N-L432F-L434W-S436N-A463G-Y469D | ++++ |
| I283T-P284D-N287P-S322T-H384A-N388M-C430N-L432F-L434W-S436N-A463G-Y469D | ++++ |
| I283T-P284D-N287P-S322T-H384T-N388M-C430N-L432F-L434W-S436N-A463G-Y469D | ++++ |
| I283T-P284D-N287P-S322T-N388M-C430N-L432F-L434W-S436N-H444R-A463G-Y469D | ++++ |
| I283T-P284D-N287P-S322T-N388M-P393A-C430N-L432F-L434W-S436N-A463G-Y469D | ++++ |
| I283T-P284D-N287P-S322T-N388M-R422F-C430N-L432F-L434W-S436N-A463G-Y469D | ++++ |
| I283T-P284D-N287P-S322T-N388M-R422Q-C430N-L432F-L434W-S436N-A463G-Y469D | ++++ |
| I283T-P284D-N287P-S322T-N388M-R422V-C430N-L432F-L434W-S436N-A463G-Y469D | ++++ |
| I283T-P284D-N287P-S322T-N388M-E421T-C430N-L432F-L434W-S436N-A463G-Y469D | ++++ |
| I283T-P284D-N287P-S322T-N388M-E421V-C430N-L432F-L434W-S436N-A463G-Y469D | ++++ |
| I283T-P284D-N287P-S322T-N388M-C430N-L432F-L434W-S436N-E459P-A463G-Y469D | ++++ |
| I283T-P284D-N287P-S322T-N388M-C430N-L432F-L434W-S436N-A463G-Y469T | ++++ |
| I283T-P284D-E286C-N287P-S322T-N388M-C430N-L432F-L434W-S436N-A463G-Y469D | ++++ |
| I283T-P284D-N287P-S322T-N388M-C430N-L432F-L434W-S436N-A463F-Y469D | ++++ |
| I283T-P284D-N287P-S322T-N388M-C430N-L432F-L434W-S436N-G462P-A463G-Y469D | ++++ |
| I283T-P284D-N287P-S322T-N388M-i421aG-C430N-L432F-L434W-S436N-A463G-Y469D | ++++ |
| I283F-P284E-N287Q-S322T-N388M-K394R-C430N-L432F-L434W-S436N-dG462 | ++++ |
| P284E-N287Q-S322T-N388M-K394R-C430N-L432F-L434W-S436N-dG462-Y469S | ++++ |
| P284E-N287Q-S322T-N388M-K394R-C430N-L432F-L434W-S436N-dG462-Y469D | ++++ |
| I283L-N285M-N287T-S322T-A337T-N388M-K394R-C430N-L432F-L434W-S436N-dG462 | ++++ |
| N285M-N287T-S322T-N388M-K394R-C430N-L432F-L434W-S436N-N460G-dG462 | ++++ |
| N285M-N287T-S322T-N388M-K394R-C430N-L432F-L434W-S436N-dG462-A463M | ++++ |
| N285M-N287T-S322T-N388M-K394R-C430N-L432F-L434W-S436N-dG462-A463F | ++++ |
| N285M-N287T-S322T-N388M-K394R-C430N-L432F-L434W-S436N-dG462-A463V | ++++ |
| N285M-N287T-S322T-N388M-K394R-C430N-L432F-L434W-S436N-dG462-Y469S | ++++ |
| N285M-N287T-S322T-N388M-K394R-C430N-L432F-L434W-S436N-dG462-Y469T | ++++ |
| N285M-N287T-S322T-N388M-K394R-C430N-L432F-L434W-S436N-dG462-Y469N | ++++ |
| N285M-N287T-S322T-N388M-K394R-C430N-L432F-L434W-S436N-dG462-A470S | ++++ |
| N285M-N287T-S322T-N388M-K394R-C430N-L432F-L434W-S436N-dA463-A470S | ++++ |
| N285M-N287T-S322T-N388M-K394R-C430N-L432F-L434W-S436N-dA463-S465I | ++++ |
| N285M-N287T-S322T-N388M-K394R-C430N-L432F-L434W-S436N-dA463-S465V | ++++ |
| I283Q-P284E-N287Q-S322T-N388M-K394R-C430N-L432F-L434W-S436N-dA463 | ++++ |
| I283C-P284E-N287Q-S322T-N388M-K394R-C430N-L432F-L434W-S436N-dA463 | ++++ |
| I283L-P284E-N287Q-S322T-N388M-K394R-C430N-L432F-L434W-S436N-dA463 | ++++ |
| P284D-N287Q-S322T-N388M-K394R-C430N-L432F-L434W-S436N-dA463 | ++++ |
| P284E-N287Q-L289H-S322T-N388M-K394R-C430N-L432F-L434W-S436N-dA463 | ++++ |
| P284E-N287Q-L289R-S322T-N388M-K394R-C430N-L432F-L434W-S436N-dA463 | ++++ |
| P284E-N287Q-S322T-N388M-K394R-R422H-C430N-L432F-L434W-S436N-dA463 | ++++ |
| P284E-N287Q-S322T-N388M-K394R-C430N-L432F-L434W-S436N-dA463-A470T | ++++ |
| P284E-N287Q-S322T-N388M-K394R-C430N-L432F-L434W-S436N-G462R-dA463 | ++++ |
| P284E-N287Q-S322T-N388M-K394R-C430N-L432F-L434W-S436N-dA463-S465A | ++++ |
| P284E-N287Q-S322T-N388M-K394R-C430N-L432F-L434W-S436N-dA463-Y469T | ++++ |
| P284E-N287Q-S322T-N388M-K394R-C430N-L432F-L434W-S436N-dA463-Y469E | ++++ |
| P284E-N287Q-S322T-N388M-K394R-C430N-L432F-L434W-S436N-dA463-A470S | ++++ |
| I283T-N285P-E286D-N287T-Q290E-S322T-N388M-K394R-C430A-L432F-L434W-S436N-G462P-A463P | ++++ |
| I283T-N285P-N287T-L289Y-Q290E-S322T-N388M-K394R-C430A-L432F-L434W-S436N-G462P-A463P | ++++ |
| I283T-N285P-N287T-L289A-Q290E-S322T-N388M-K394R-C430A-L432F-L434W-S436N-G462P-A463P | ++++ |
| I283T-N285P-N287T-Q290E-S322T-Q365M-N388M-K394R-C430A-L432F-L434W-S436N-G462P-A463P | ++++ |
| I283T-N285P-N287T-Q290E-S322T-Q365S-N388M-K394R-C430A-L432F-L434W-S436N-G462P-A463P | ++++ |
| I283L-N285P-N287T-Q290E-S322T-N388M-K394R-C430A-L432F-L434W-S436N | ++++ |
| I283H-P284D-N285M-N287T-S322T-N388M-K394R-C430A-L432F-L434W-S436N-G462P-A463P | ++++ |
| I283Q-P284D-N285M-N287T-S322T-N388M-K394R-C430A-L432F-L434W-S436N-G462P-A463P | ++++ |
| I283T-P284D-N285H-N287T-S322T-N388M-K394R-C430A-L432F-L434W-S436N-G462P-A463P | ++++ |
| I283T-P284D-N285V-N287T-S322T-N388M-K394R-C430A-L432F-L434W-S436N-G462P-A463P | ++++ |
| I283T-P284D-N285L-N287T-S322T-N388M-K394R-C430A-L432F-L434W-S436N-G462P-A463P | ++++ |
| I283T-P284D-N285T-N287T-S322T-N388M-K394R-C430A-L432F-L434W-S436N-G462P-A463P | ++++ |
| I283T-P284D-N285S-N287T-S322T-N388M-K394R-C430A-L432F-L434W-S436N-G462P-A463P | ++++ |
| I283T-P284D-N285M-E286N-N287T-S322T-N388M-K394R-C430A-L432F-L434W-S436N-G462P-A463P | ++++ |
| I283T-P284D-N285M-E286A-N287T-S322T-N388M-K394R-C430A-L432F-L434W-S436N-G462P-A463P | ++++ |
| I283T-P284D-N285M-E286G-N287T-S322T-N388M-K394R-C430A-L432F-L434W-S436N-G462P-A463P | ++++ |
| I283T-P284D-N285M-E286H-N287T-S322T-N388M-K394R-C430A-L432F-L434W-S436N-G462P-A463P | ++++ |
| I283T-P284D-N285M-E286D-N287T-S322T-N388M-K394R-C430A-L432F-L434W-S436N-G462P-A463P | ++++ |
| I283T-P284D-N285M-N287T-S288T-S322T-N388M-K394R-C430A-L432F-L434W-S436N-G462P-A463P | ++++ |
| I283T-P284D-N285M-N287T-L289Q-S322T-N388M-K394R-C430A-L432F-L434W-S436N-G462P-A463P | ++++ |
| I283T-P284D-N285M-N287T-L289R-S322T-N388M-K394R-C430A-L432F-L434W-S436N-G462P-A463P | ++++ |
| I283T-P284D-N285M-N287T-L289M-S322T-N388M-K394R-C430A-L432F-L434W-S436N-G462P-A463P | ++++ |
| I283T-P284D-N285M-N287T-S322T-N388M-K394R-C430T-L432F-L434W-S436N-G462P-A463P | ++++ |
| I283T-P284D-N287P-S322T-H384A-N388M-C430N-L432F-L434W-S436N-dG462 | ++++ |
| P25L-I283T-P284D-N287P-S322T-N388M-C430N-L432F-L434W-S436N-dG462 | ++++ |
| I283T-P284D-N287P-S322T-N388M-R422G-C430N-L432F-L434W-S436N-dG462 | ++++ |
| I283T-P284D-N287P-S322T-P368S-N388M-E421V-C430N-L432F-L434W-S436N-dG462 | ++++ |
| I283T-P284D-N287P-S322T-N388M-R422L-C430N-L432F-L434W-S436N-dG462 | ++++ |
| I283T-P284D-N287P-S322T-N388M-C430N-L432F-L434W-S436N-dG462-A463I | ++++ |
| I283T-P284D-N287P-S322T-N388M-C430N-L432F-L434W-S436N-dG462-A463T | ++++ |
| I283T-P284D-N287P-S322T-N388M-C430N-L432F-L434W-S436N-dG462-A463F | ++++ |
| I283T-P284D-N287P-S322T-N388M-C430N-L432F-L434W-S436N-H461V-dG462 | ++++ |
| I283T-P284D-N287P-S322T-N388M-C430N-L432F-L434W-S436N-dG462-S465V | ++++ |
| I283T-P284D-N287P-S322T-N388M-C430N-L432F-L434W-S436N-H461A-dG462 | ++++ |
| I283T-P284D-N287P-S322T-N388M-C430N-L432F-L434W-S436N-H461T-dG462 | ++++ |
| I283T-P284D-N287P-S322T-N388M-C430N-L432F-L434W-S436N-H461Y-dG462 | ++++ |
| I283T-P284D-N287P-S322T-N388M-C430N-L432F-L434W-S436N-dG462-A463S | ++++ |
| I283T-P284D-N287P-S322T-N388M-C430N-L432F-L434W-S436N-H461A-dG462-A463T | ++++ |
| I283T-P284D-N287P-S322T-N388M-C430N-L432F-L434W-S436N-H461A-dG462-S465V | ++++ |
| I283T-P284D-N287P-S322T-N388M-C430N-L432F-L434W-S436N-H461A-dG462-S465I | ++++ |
| I283T-P284D-N287P-S322T-N388M-C430N-L432F-L434W-S436N-H461Y-dG462-A463T | ++++ |
| I283T-P284D-N287P-S322T-N388M-C430N-L432F-L434W-S436N-H461Y-dG462-S465V | ++++ |
| I283T-P284D-N287P-S322T-N388M-C430N-L432F-L434W-S436N-H461V-dG462-A463T | ++++ |
| I283T-P284D-N287P-S322T-N388M-C430N-L432F-L434W-S436N-H461V-dG462-S465V | ++++ |
| I283T-P284D-N287P-S322T-N388M-C430N-L432F-L434W-S436N-dG462-A463T-S465V | ++++ |
| I283T-P284D-N287P-S322T-N388M-C430N-L432F-L434W-S436N-H461A-dG462-A463T-S465V | ++++ |
| I283T-P284D-N287P-S322T-N388M-C430N-L432F-L434W-S436N-H461Y-dG462-A463T-S465V | ++++ |
| I283T-P284D-N287P-S322T-N388M-C430N-L432F-L434W-S436N-H461V-dG462-A463T-S465V | ++++ |
| I283T-P284D-N287P-S322T-N388M-E421V-R422F-C430N-L432F-L434W-S436N-dG462 | ++++ |
| I283T-P284D-N287P-S322T-N388M-E421V-C430N-L432F-L434W-S436N-H461T-dG462 | ++++ |
| I283T-P284D-N287P-S322T-N388M-E421V-C430N-L432F-L434W-S436N-H461V-dG462 | ++++ |
| I283T-P284D-N287P-S322T-N388M-E421V-C430N-L432F-L434W-S436N-dG462-A463T | ++++ |
| I283T-P284D-N287P-S322T-N388M-R422F-C430N-L432F-L434W-S436N-H461T-dG462 | ++++ |
| I283T-P284D-N287P-S322T-N388M-R422F-C430N-L432F-L434W-S436N-H461V-dG462 | ++++ |
| I283T-P284D-N287P-S322T-N388M-R422F-C430N-L432F-L434W-S436N-dG462-A463T | ++++ |
| I283T-P284D-N287P-S322T-N388M-C430N-L432F-L434W-S436N-H461T-dG462-A463T | ++++ |
| I283T-P284D-N287P-S322T-N388M-E421V-R422F-C430N-L432F-L434W-S436N-H461T-dG462 | ++++ |
| I283T-P284D-N287P-S322T-N388M-E421V-R422F-C430N-L432F-L434W-S436N-H461V-dG462 | ++++ |
| I283T-P284D-N287P-S322T-N388M-E421V-R422F-C430N-L432F-L434W-S436N-dG462-A463T | ++++ |
| I283T-P284D-N287P-S322T-N388M-E421V-C430N-L432F-L434W-S436N-H461T-dG462-A463T | ++++ |
| I283T-P284D-N287P-S322T-N388M-E421V-C430N-L432F-L434W-S436N-H461V-dG462-A463T | ++++ |
| I283T-P284D-N287P-S322T-N388M-R422F-C430N-L432F-L434W-S436N-H461T-dG462-A463T | ++++ |
| I283T-P284D-N287P-S322T-N388M-R422F-C430N-L432F-L434W-S436N-H461V-dG462-A463T | ++++ |
| I283T-P284D-N287P-S322T-N388M-E421V-R422F-C430N-L432F-L434W-S436N-H461T-dG462-A463T | ++++ |
| I283T-P284D-N287P-S322T-N388M-E421V-R422F-C430N-L432F-L434W-S436N-H461V-dG462-A463T | ++++ |
| I283T-P284D-N287P-S322T-N388M-E421V-C430N-L432F-L434W-S436N-dG462 | ++++ |
| I283T-P284D-N287P-S322T-N388M-R422F-C430N-L432F-L434W-S436N-dG462 | ++++ |
| I283T-N285P-N287T-Q290E-S322T-N388M-K394R-C430A-L432F-L434W-S436N-dH461 | ++++ |
| I283T-N285P-N287T-Q290E-S322T-N388M-K394R-C430A-L432F-L434W-S436N-dG462 | ++++ |
| I283T-N285P-N287T-Q290E-S322T-N388M-K394R-C430A-L432F-L434W-S436N-dA463 | ++++ |
| I283T-P284D-i284aP-N287P-S322T-N388M-C430N-L432F-L434W-S436N-dG462 | ++++ |
| I283T-P284D-N285M-N287T-S322T-N388M-K394R-C430A-L432F-L434W-S436N-C437M-G462P-A463P | ++++ |
| I283T-P284D-N285M-N287T-S322T-N388M-K394R-C430A-L432F-L434W-S436N-G462P-A463P-I464P | ++++ |
| I283T-P284D-N285M-N287T-L289H-S322T-N388M-K394R-C430A-L432F-L434W-S436N-G462P-A463P | ++++ |
| I283T-P284D-N285I-N287T-i287aS-S322T-N388M-K394R-C430A-L432F-L434W-S436N-G462P-A463P | ++++ |
| I283T-P284D-N285M-N287T-S322T-N388M-K394R-C430A-L432F-L434W-S436N-G462T-A463P | ++++ |
| I283T-N285P-N287T-Q290E-S322T-N388M-K394R-dR422-C430A-L432F-L434W-S436N-dG462 | ++++ |
| L223Y-I283T-N285P-N287T-Q290E-S322T-N388M-K394R-K423G-C430A-L432F-L434W-S436N-dG462 | ++++ |
| I283T-P284D-i284aP-N287P-S322T-N388M-E421V-C430N-L432F-L434W-S436N-dG462-A463T | ++++ |
| I283T-P284D-i284aP-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-H461V-dG462-A463T | ++++ |
| I283T-P284D-i284aP-N287P-S322T-N388M-E421V-R422F-C430N-L432F-L434W-S436N-dG462-A463T | ++++ |
| I283T-P284D-i284aP-N287P-S322T-N388M-E421V-R422F-C430N-L432F-L434W-S436N-H461T-dG462-A463T | ++++ |
| I283T-P284D-i284aP-N287P-S322T-N388M-E421V-R422F-C430N-L432F-L434W-S436N-H461T-dG462 | ++++ |
| I283T-P284D-i284aP-N287P-S322T-N388M-C430N-L432F-L434W-S436N-H461A-dG462-A463T | ++++ |
| I283T-P284D-i284aP-N287P-S322T-N388M-C430N-L432F-L434W-S436N-H461A-dG462 | ++++ |
| E126K-H141Q-I283T-N285P-N287T-Q290E-S322T-N388M-K394R-C430A-L432F-L434W-S436N-dG462 | ++++ |
| P184S-I283T-N285P-N287T-Q290E-S322T-N388M-K394R-C430A-L432F-L434W-S436N-dG462 | ++++ |
| L223Y-I283T-N285P-N287T-Q290E-S322T-N388M-K394R-C430A-L432F-L434W-S436N-dG462 | ++++ |
| V227Y-I283T-N285P-N287T-Q290E-S322T-N388M-K394R-C430A-L432F-L434W-S436N-dG462 | ++++ |
| I283L-N285P-N287T-Q290E-S322T-N388M-K394R-C430A-L432F-L434W-S436N-dG462 | ++++ |
| I283T-N285P-N287T-L289W-Q290E-S322T-N388M-K394R-C430A-L432F-L434W-S436N-dG462 | ++++ |
| I283T-N285P-N287T-L289R-Q290E-S322T-N388M-K394R-C430A-L432F-L434W-S436N-dG462 | ++++ |
| I283T-N285P-N287T-L289C-Q290E-S322T-N388M-K394R-C430A-L432F-L434W-S436N-dG462 | ++++ |
| I283T-N285P-N287T-Q290E-S322T-Q365T-N388M-K394R-C430A-L432F-L434W-S436N-dG462 | ++++ |
| I283T-N285P-N287T-Q290E-S322T-N388M-K394R-K423G-C430A-L432F-L434W-S436N-dG462 | ++++ |
| I283T-N285P-N287T-Q290E-S322T-N388M-K394R-C430V-L432F-L434W-S436N-dG462 | ++++ |
| I283T-N285P-N287T-Q290E-S322T-N388M-K394R-C430A-L432F-L434W-S436N-H444T-dG462 | ++++ |
| I283T-N285P-N287T-Q290E-S322T-N388M-K394R-C430A-L432F-L434W-S436N-H444R-dG462 | ++++ |
| I283T-N285P-N287T-Q290E-S322T-N388M-K394R-C430A-L432F-L434W-S436N-H444F-dG462 | ++++ |
| I283T-N285P-N287T-Q290E-S322T-N388M-K394R-C430A-L432F-L434W-S436N-N460G-dG462 | ++++ |
| I283T-N285P-N287T-Q290E-S322T-N388M-K394R-C430A-L432F-L434W-S436N-dG462-A463F | ++++ |
| I283T-N285P-N287T-Q290E-S322T-N388M-K394R-C430A-L432F-L434W-S436N-dG462-A470S | ++++ |
| I283T-N285P-N287T-Q290E-S322T-N388M-K394R-R422D-C430A-L432F-L434W-S436N-dG462 | ++++ |
| I283T-N285P-N287T-Q290E-S322T-N388M-K394R-C430A-L432F-L434W-S436N-dG462-A463V | ++++ |
| I283T-N285P-N287T-Q290E-S322T-N388M-K394R-C430A-L432F-L434W-S436N-dG462-Y471F | ++++ |
| I35V-Q140L-I283T-N285P-N287T-Q290E-S322T-N388M-K394R-C430A-L432F-L434W-S436N-dG462 | ++++ |
| E224P-I283T-N285P-N287T-Q290E-S322T-N388M-K394R-C430A-L432F-L434W-S436N-dG462 | ++++ |
| E224Q-I283T-N285P-N287T-Q290E-S322T-N388M-K394R-C430A-L432F-L434W-S436N-dG462 | ++++ |
| L222V-I283T-N285P-N287T-Q290E-S322T-N388M-K394R-C430A-L432F-L434W-S436N-dG462 | ++++ |
| V227L-I283T-N285P-N287T-Q290E-S322T-N388M-K394R-C430A-L432F-L434W-S436N-dG462 | ++++ |
| I283T-N285P-E286A-N287T-Q290E-S322T-N388M-K394R-C430A-L432F-L434W-S436N-dG462 | ++++ |
| I283T-N285P-N287T-Q290E-S322T-R361K-N388M-K394R-C430A-L432F-L434W-S436N-dG462 | ++++ |
| I283T-N285P-N287T-Q290E-S322T-M355V-N388M-K394R-C430A-L432F-L434W-S436N-dG462 | ++++ |
| I283T-N285P-N287T-Q290E-S322T-M355T-N388M-K394R-C430A-L432F-L434W-S436N-dG462 | ++++ |
| I283T-N285P-N287T-Q290E-S322T-M355C-N388M-K394R-C430A-L432F-L434W-S436N-dG462 | ++++ |
| I283T-N285P-N287T-Q290E-S322T-N388M-K394R-E421W-C430A-L432F-L434W-S436N-dG462 | ++++ |
| I283T-N285P-N287T-Q290E-S322T-N388S-K394R-C430A-L432F-L434W-S436N-dG462 | ++++ |
| I283T-N285P-N287T-Q290E-S322T-N388M-K394H-C430A-L432F-L434W-S436N-dG462 | ++++ |
| I283T-N285P-N287T-Q290E-S322T-N388M-K394R-H419P-C430A-L432F-L434W-S436N-dG462 | ++++ |
| I283T-N285P-N287T-Q290E-S322T-N388M-K394R-H419A-C430A-L432F-L434W-S436N-dG462 | ++++ |
| I283T-N285P-N287T-Q290E-S322T-N388M-K394R-E421L-C430A-L432F-L434W-S436N-dG462 | ++++ |
| I283T-N285P-N287T-Q290E-S322T-N388M-K394R-S429V-C430A-L432F-L434W-S436N-dG462 | ++++ |
| I283T-N285P-N287T-Q290E-S322T-N388M-K394R-C430A-I431K-L432F-L434W-S436N-dG462 | ++++ |
| I283T-N285P-N287T-Q290E-S322T-N388M-K394R-C430A-I431N-L432F-L434W-S436N-dG462 | ++++ |
| I283T-N285P-N287T-Q290E-S322T-N388M-K394R-C430A-I431P-L432F-L434W-S436N-dG462 | ++++ |
| I283T-N285P-N287T-Q290E-S322T-N388M-K394R-C430A-I431C-L432F-L434W-S436N-dG462 | ++++ |
| I283T-N285P-N287T-Q290E-S322T-N388M-K394R-C430A-I431T-L432F-L434W-S436N-dG462 | ++++ |
| I283T-N285P-N287T-Q290E-S322T-N388M-K394R-C430A-I431A-L432F-L434W-S436N-dG462 | ++++ |
| I283T-N285P-N287T-Q290E-S322T-N388M-K394R-C430S-L432F-L434W-S436N-dG462 | ++++ |
| I283T-N285P-N287T-Q290E-S322T-N388M-K394R-C430A-L432F-L434W-S436N-C437L-dG462 | ++++ |
| I283T-N285P-N287T-Q290E-S322T-N388M-K394R-C430A-L432F-L434W-S436N-Y438W-dG462 | ++++ |
| I283T-N285P-N287T-Q290E-S322T-N388M-K394R-C430A-L432F-L434W-S436N-C437T-dG462 | ++++ |
| I283T-N285P-N287T-Q290E-S322T-N388M-K394R-C430A-L432F-L434W-S436N-C437M-dG462 | ++++ |
| I283T-N285P-N287T-Q290E-S322T-N388M-K394R-C430A-L432F-L434W-S436N-C437I-dG462 | ++++ |
| I283T-N285P-N287T-Q290E-S322T-N388M-K394R-C430A-L432F-L434W-S436N-Y438M-dG462 | ++++ |
| I283T-N285P-N287T-Q290E-S322T-N388M-K394R-C430A-L432F-L434W-S436N-S439P-dG462 | ++++ |
| I283T-N285P-N287T-Q290E-S322T-N388M-K394R-C430A-L432F-L434W-S436N-K447T-dG462 | ++++ |
| D27N-I283T-N285P-N287T-Q290E-S322T-N388M-K394R-C430A-L432F-L434W-S436N-G448T-dG462 | ++++ |
| I283T-N285P-N287T-Q290E-S322T-N388M-K394R-C430A-L432F-L434W-S436N-G448C-dG462 | ++++ |
| I283T-N285P-N287T-Q290E-S322T-N388M-K394R-E421F-C430A-L432F-L434W-S436N-dG462 | ++++ |
| I283T-N285P-N287T-Q290E-S322T-N388M-K394R-i421aD-C430A-L432F-L434W-S436N-dG462 | ++++ |
| I283T-N285P-N287T-Q290E-S322T-N388M-K394R-i421aT-C430A-L432F-L434W-S436N-dG462 | ++++ |
| I283T-N285P-N287T-Q290E-S322T-N388M-K394R-i421aS-C430A-L432F-L434W-S436N-dG462 | ++++ |
| I283T-N285P-N287T-Q290E-S322T-N388M-K394R-i421aI-C430A-L432F-L434W-S436N-dG462 | ++++ |
| I283T-N285P-N287T-Q290E-S322T-N388M-K394R-i421aE-C430A-L432F-L434W-S436N-dG462 | ++++ |
| I283T-N285P-N287T-Q290E-S322T-N388M-K394R-i421aR-C430A-L432F-L434W-S436N-dG462 | ++++ |
| I283T-N285P-N287T-Q290E-S322T-N388M-K394R-i421aP-C430A-L432F-L434W-S436N-dG462 | ++++ |
| I283T-N285P-N287T-Q290E-S322T-N388M-K394R-i424aA-C430A-L432F-L434W-S436N-dG462 | ++++ |
| I283T-N285P-N287T-Q290E-S322T-N388M-K394R-i424aW-C430A-L432F-L434W-S436N-dG462 | ++++ |
| I283T-N285P-N287T-Q290E-S322T-N388M-K394R-i424aL-C430A-L432F-L434W-S436N-dG462 | ++++ |
| I283T-N285P-N287T-Q290E-S322T-N388M-K394R-i424aS-C430A-L432F-L434W-S436N-dG462 | ++++ |
| I283T-N285P-N287T-Q290E-S322T-N388M-K394R-i424aD-C430A-L432F-L434W-S436N-dG462 | ++++ |
| I283T-N285P-N287T-Q290E-S322T-N388M-K394R-i426aL-C430A-L432F-L434W-S436N-dG462 | ++++ |
| I283T-N285P-N287T-Q290E-S322T-N388M-K394R-i429aN-C430A-L432F-L434W-S436N-dG462 | ++++ |
| I283T-N285P-N287T-Q290E-S322T-N388M-K394R-C430A-i430aC-L432F-L434W-S436N-dG462 | ++++ |
| I283T-N285P-N287T-Q290E-S322T-N388M-K394R-C430A-i430aG-L432F-L434W-S436N-dG462 | ++++ |
| I283T-N285P-N287T-Q290E-S322T-N388M-K394R-C430A-i430aN-L432F-L434W-S436N-dG462 | ++++ |
| I283T-N285P-N287T-Q290E-S322T-N388M-K394R-C430A-i430aS-L432F-L434W-S436N-dG462 | ++++ |
| I283T-N285P-N287T-Q290E-S322T-N388M-K394R-C430A-i430aR-L432F-L434W-S436N-dG462 | ++++ |
| I283T-N285P-N287T-Q290E-S322T-N388M-K394R-C430A-i430aL-L432F-L434W-S436N-dG462 | ++++ |
| I283T-N285P-N287T-Q290E-S322T-N388M-K394R-C430A-i430aI-L432F-L434W-S436N-dG462 | ++++ |
| I283T-N285P-N287T-Q290E-S322T-N388M-K394R-C430A-i430aQ-L432F-L434W-S436N-dG462 | ++++ |
| I283T-N285P-N287T-Q290E-S322T-N388M-K394R-C430A-i430aH-L432F-L434W-S436N-dG462 | ++++ |
| I283T-N285P-N287T-Q290E-S322T-N388M-K394R-C430A-L432F-L434W-S436N-i438aD-dG462 | ++++ |
| I283T-N285P-N287T-Q290E-S322T-N388M-K394R-C430A-L432F-L434W-S436N-i443aR-dG462 | ++++ |
| I283T-N285P-N287T-Q290E-S322T-N388M-K394R-C430A-L432F-L434W-S436N-i443aV-dG462 | ++++ |
| I283T-N285P-N287T-Q290E-S322T-N388M-K394R-C430A-L432F-L434W-S436N-i443aL-dG462 | ++++ |
| I283T-N285P-N287T-Q290E-S322T-N388M-K394R-C430A-L432F-L434W-S436N-i443aT-dG462 | ++++ |
| I283T-N285P-N287T-Q290E-S322T-N388M-K394R-C430A-L432F-L434W-S436N-i444aH-dG462 | ++++ |
| I283T-N285P-N287T-Q290E-S322T-N388M-K394R-C430A-L432F-L434W-S436N-i444aP-dG462 | ++++ |
| I283T-N285P-N287T-Q290E-S322T-N388M-K394R-C430A-L432F-L434W-S436N-i444aV-dG462 | ++++ |
| I283T-N285P-N287T-Q290E-S322T-N388M-K394R-C430A-L432F-L434W-S436N-i444aR-dG462 | ++++ |
| I283T-N285P-N287T-Q290E-S322T-N388M-K394R-C430A-L432F-L434W-S436N-i444aN-dG462 | ++++ |
| I283T-N285P-N287T-Q290E-S322T-N388M-K394R-C430A-L432F-L434W-S436N-i444aG-dG462 | ++++ |
| I283T-N285P-N287T-Q290E-S322T-N388M-K394R-C430A-L432F-L434W-S436N-i444aT-dG462 | ++++ |
| I283T-N285P-N287T-Q290E-S322T-N388M-K394R-C430A-L432F-L434W-S436N-i444aL-dG462 | ++++ |
| I283T-N285P-N287T-Q290E-S322T-N388M-K394R-C430A-L432F-L434W-S436N-i444aE-dG462 | ++++ |
| I283T-N285P-N287T-Q290E-S322T-N388M-K394R-C430A-L432F-L434W-S436N-i444aK-dG462 | ++++ |
| I283T-N285P-N287T-Q290E-S322T-N388M-K394R-C430A-L432F-L434W-S436N-i444aS-dG462 | ++++ |
| I283T-N285P-N287T-Q290E-S322T-N388M-K394R-C430A-L432F-L434W-S436N-i445aQ-dG462 | ++++ |
| I283T-N285P-N287T-Q290E-S322T-N388M-K394R-C430A-L432F-L434W-S436N-i447aY-dG462 | ++++ |
| I283T-N285P-N287T-Q290E-S322T-N388M-K394R-C430A-L432F-L434W-S436N-i447aL-dG462 | ++++ |
| I283T-N285P-N287T-Q290E-S322T-N388M-K394R-C430A-L432F-L434W-S436N-i447aK-dG462 | ++++ |
| I283T-N285P-N287T-Q290E-S322T-N388M-K394R-C430A-L432F-L434W-S436N-i447aP-dG462 | ++++ |
| I283T-N285P-N287T-Q290E-S322T-N388M-K394R-C430A-L432F-L434W-S436N-i448aS-dG462 | ++++ |
| I283T-N285P-N287T-Q290E-S322T-N388M-K394R-C430A-i431aG-L432F-L434W-S436N-dG462 | ++++ |
| I283T-N285P-N287T-Q290E-S322T-N388M-K394R-C430A-i431aL-L432F-L434W-S436N-dG462 | ++++ |
| I283T-N285P-N287T-Q290E-S322T-N388M-K394R-C430A-i431aN-L432F-L434W-S436N-dG462 | ++++ |
| I283T-N285P-N287T-Q290E-S322T-N388M-K394R-C430A-L432F-L434W-S436N-i449aS-dG462 | ++++ |
| L223Y-I283T-N285P-N287T-Q290E-S322T-N388M-K394R-E421F-C430A-L432F-L434W-S436N-dG462 | ++++ |
| I283T-N285P-N287T-Q290E-S322T-N388M-K394R-E421F-C430A-i430aG-L432F-L434W-S436N-dG462 | ++++ |
| I283T-N285P-N287T-Q290E-S322T-N388M-K394R-E421F-C430A-i430aL-L432F-L434W-S436N-dG462 | ++++ |
| I283T-N285P-N287T-Q290E-S322T-N388M-K394R-E421F-C430A-i430aN-L432F-L434W-S436N-dG462 | ++++ |
| I283T-N285P-N287T-Q290E-S322T-N388M-K394R-E421F-C430A-L432F-L434W-S436N-i448aS-dG462 | ++++ |
| L223Y-I283T-N285P-N287T-Q290E-S322T-N388M-K394R-E421F-K423G-C430A-L432F-L434W-S436N-dG462 | ++++ |
| L223Y-I283T-N285P-N287T-Q290E-S322T-N388M-K394R-K423G-C430A-i430aG-L432F-L434W-S436N-dG462 | ++++ |
| L223Y-I283T-N285P-N287T-Q290E-S322T-N388M-K394R-K423G-C430A-i430aL-L432F-L434W-S436N-dG462 | ++++ |
| L223Y-I283T-N285P-N287T-Q290E-S322T-N388M-K394R-K423G-C430A-i430aN-L432F-L434W-S436N-dG462 | ++++ |
| L223Y-I283T-N285P-N287T-Q290E-S322T-N388M-K394R-K423G-C430A-L432F-L434W-S436N-i448aS-dG462 | ++++ |
| L223Y-I283T-N285P-N287T-Q290E-S322T-N388M-K394R-i429aN-C430A-L432F-L434W-S436N-dG462 | ++++ |
| L223Y-I283T-N285P-N287T-Q290E-S322T-N388M-K394R-C430A-i430aG-L432F-L434W-S436N-dG462 | ++++ |
| L223Y-I283T-N285P-N287T-Q290E-S322T-N388M-K394R-i429aN-i429bG-C430A-L432F-L434W-S436N-dG462 | ++++ |
| I283T-N285P-N287T-Q290E-S322T-N388M-K394R-C430A-i430aG-L432F-L434W-S436N-C437L-dG462 | ++++ |
| L223Y-I283T-N285P-N287T-Q290E-S322T-N388M-K394R-C430A-i430aG-L432F-L434W-S436N-C437L-dG462 | ++++ |
| L223Y-I283T-N285P-N287T-Q290E-S322T-N388M-K394R-C430A-i430aN-L432F-L434W-S436N-dG462 | ++++ |
| L223Y-I283T-N285P-N287T-Q290E-S322T-N388M-K394R-C430A-L432F-L434W-S436N-C437L-dG462 | ++++ |
| L223Y-I283T-N285P-N287T-Q290E-S322T-N388M-K394R-C430A-i430aL-L432F-L434W-S436N-dG462 | ++++ |
| I283T-N285P-N287T-Q290E-S322T-N388M-K394R-C430A-i430aG-L432F-L434W-S436N-V451S-dG462 | ++++ |
| I283T-N285P-N287T-Q290E-S322T-N388M-K394R-i425aW-C430A-i430aG-L432F-L434W-S436N-dG462 | ++++ |
| H141C-I283T-N285P-N287T-Q290E-S322T-N388M-K394R-C430A-i430aG-L432F-L434W-S436N-dG462 | ++++ |
| L222V-I283T-N285P-N287T-Q290E-S322T-N388M-K394R-C430A-i430aG-L432F-L434W-S436N-dG462 | ++++ |
| I283T-N285P-N287T-Q290E-S322T-I330V-N388M-K394R-C430A-i430aG-L432F-L434W-S436N-dG462 | ++++ |
| I283T-N285P-N287T-Q290E-S322T-N388M-S389H-K394R-C430A-i430aG-L432F-L434W-S436N-dG462 | ++++ |
| H141C-I283T-N285P-N287T-Q290E-S322T-N388M-S389H-K394R-C430A-i430aG-L432F-L434W-S436N-dG462 | ++++ |
| I283T-N285P-N287T-Q290E-S322T-I330V-N388M-S389H-K394R-C430A-i430aG-L432F-L434W-S436N-dG462 | ++++ |
| H141C-I283T-N285P-N287T-Q290E-S322T-I330V-N388M-S389H-K394R-C430A-i430aG-L432F-L434W-S436N-dG462 | ++++ |
| I283T-N285P-N287T-Q290E-S322T-I330V-N388M-S389H-K394R-C430A-i430aG-L432F-L434W-S436N-V451S-dG462 | ++++ |
| E224P-I283T-N285P-N287T-Q290E-S322T-N388M-K394R-C430A-i430aG-L432F-L434W-S436N-dG462 | ++++ |
| L222V-E224P-I283T-N285P-N287T-Q290E-S322T-N388M-K394R-C430A-i430aG-L432F-L434W-S436N-dG462 | ++++ |
| I283T-N285P-N287T-Q290E-S322T-N388M-S389H-K394R-C430A-i430aG-L432F-L434W-S436N-I453L-dG462 | ++++ |
| E224A-I283T-N285P-N287T-Q290E-S322T-N388M-S389H-K394R-C430A-i430aG-L432F-L434W-S436N-dG462 | ++++ |
| E224P-I283T-N285P-N287T-Q290E-S322T-N388M-S389H-K394R-C430A-i430aG-L432F-L434W-S436N-dG462 | ++++ |
| I283T-P284D-i284aP-N287P-S322T-N388M-E421V-i421aR-C430N-L432F-L434W-S436N-H461T-dG462-A463T | ++++ |
| I283T-P284D-i284aP-N287P-S322T-N388M-E421V-i422aK-C430N-L432F-L434W-S436N-H461T-dG462-A463T | ++++ |
| I283T-P284D-i284aP-N287P-S322T-N388M-E421V-C430N-1431V-L432F-L434W-S436N-H461T-dG462-A463T | ++++ |
| I283T-P284D-i284aP-N287P-S322T-Q365I-N388M-E421V-C430N-L432F-L434W-S436N-H461T-dG462-A463T | ++++ |
| I283T-P284D-i284aP-N287P-S322T-Q385G-N388M-E421V-C430N-L432F-L434W-S436N-H461T-dG462-A463T | ++++ |
| I283T-P284D-i284aP-N287P-S322T-Q385L-N388M-E421V-C430N-L432F-L434W-S436N-H461T-dG462-A463T | ++++ |
| I283T-P284D-i284aP-N287P-S322T-Q385S-N388M-E421V-C430N-L432F-L434W-S436N-H461T-dG462-A463T | ++++ |
| I283T-P284D-i284aP-N287P-S322T-Q385T-N388M-E421V-C430N-L432F-L434W-S436N-H461T-dG462-A463T | ++++ |
| I283T-P284D-i284aP-N287P-S322T-H384G-N388M-E421V-C430N-L432F-L434W-S436N-H461T-dG462-A463T | ++++ |
| I283T-P284D-i284aP-N287P-S322T-H384L-N388M-E421V-C430N-L432F-L434W-S436N-H461T-dG462-A463T | ++++ |
| I283T-P284D-i284aP-N287P-L289R-S322T-N388M-E421V-C430N-L432F-L434W-S436N-H461T-dG462-A463T | ++++ |
| I283T-P284D-i284aP-E286A-N287P-S322T-N388M-E421V-C430N-L432F-L434W-S436N-H461T-dG462-A463T | ++++ |
| I283T-P284D-i284aP-E286S-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-H461T-dG462-A463T-D468L | ++++ |
| I283T-P284D-i284aP-N287P-S322T-N388M-E421V-C430N-L432F-L434W-S436N-G448C-H461T-dG462-A463T | ++++ |
| I283T-P284D-i284aP-N287P-S322T-N388M-E421V-C430N-L432F-L434W-S436N-G448E-H461T-dG462-A463T | ++++ |
| I283T-P284D-i284aP-N287P-S322T-N388M-E421V-C430N-L432F-L434W-S436N-G448V-H461T-dG462-A463T | ++++ |
| I283T-P284D-i284aP-N287P-S322T-N388M-E421V-C430N-L432F-L434W-S436N-H461T-dG462-A463T-Y469A | ++++ |
| I283T-P284D-i284aP-N287P-S322T-N388M-E421V-C430N-L432F-L434W-S436N-H461T-dG462-A463T-Y469H | ++++ |
| I283T-P284D-i284aP-N287P-S322T-N388M-E421V-C430N-L432F-L434W-S436N-C437F-H461T-dG462-A463T | ++++ |
| I283T-P284D-i284aP-N287P-S322T-N388M-E421V-C430N-L432F-L434W-S436N-C437G-H461T-dG462-A463T | ++++ |
| I283T-P284D-i284aP-N287P-S322T-N388M-E421V-C430N-i430aR-L432F-L434W-S436N-H461T-dG462-A463T | ++++ |
| I283T-P284D-i284aP-N287P-S322T-N388M-E421V-C430N-I431M-L432F-L434W-S436N-H461T-dG462-A463T | ++++ |
| P45A-I283T-P284D-i284aP-N287P-S322T-N388M-E421V-C430N-L432F-L434W-S436N-i443aG-i443bA-H461T-dG462-A463T | ++++ |
| I283T-P284D-i284aP-N287P-S322T-N388M-E421V-C430N-L432F-L434W-S436N-H461T-G462R-i462aA-A463T | ++++ |
| R15L-I283T-P284D-i284aP-N287P-S322T-K378M-N388M-E421V-C430N-L432F-L434W-S436N-H461T-dG462-A463T | ++++ |
| V272L-I283T-P284D-i284aP-N287P-S322T-N388M-E421V-C430N-L432F-L434W-S436N-H461T-dG462-A463T | ++++ |
| V272R-I283T-P284D-i284aP-N287P-S322T-N388M-E421V-C430N-L432F-L434W-S436N-H461T-dG462-A463T | ++++ |
| V272I-I283T-P284D-i284aP-N287P-S322T-N388M-E421V-C430N-L432F-L434W-S436N-H461T-dG462-A463T | ++++ |
| I283T-P284D-i284aP-N287P-S322T-N388M-E421V-C430N-L432F-L434W-S436N-C437F-G448E-H461T-dG462-A463T | ++++ |
| I283T-P284D-i284aP-N287P-S322T-N388M-E421V-C430N-L432F-L434W-S436N-C437F-G448E-H461T-G462R-i462aA-A463T | ++++ |
| I283T-P284D-i284aP-N287P-S322T-N388M-E421V-C430N-L432F-L434W-S436N-C437F-H461T-G462R-i462aA-A463T | ++++ |
| I283T-P284D-i284aP-N287P-S322T-N388M-E421V-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-C437F-H461T-dG462-A463T | ++++ |
| V272R-I283T-P284D-i284aP-N287P-S322T-N388M-E421V-C430N-L432F-L434W-S436N-C437F-G448E-H461T-dG462-A463T | ++++ |
| V272R-I283T-P284D-i284aP-N287P-S322T-N388M-E421V-C430N-L432F-L434W-S436N-C437F-G448E-H461T- | ++++ |
| G462R-i462aA-A463T | |
| V272R-I283T-P284D-i284aP-N287P-S322T-N388M-E421V-C430N-L432F-L434W-S436N-C437F-H461T-G462R-i462aA-A463T | ++++ |
| V272R-I283T-P284D-i284aP-N287P-S322T-N388M-E421V-C430N-L432F-L434W-S436N-G448E-H461T-dG462-A463T | ++++ |
| V272R-I283T-P284D-i284aP-N287P-S322T-N388M-E421V-C430N-L432F-L434W-S436N-G448E-H461T-G462R-i462aA-A463T | ++++ |
| V272R-I283T-P284D-i284aP-N287P-S322T-N388M-E421V-C430N-L432F-L434W-S436N-H461T-G462R-i462aA-A463T | ++++ |
| V272I-I283T-P284D-i284aP-N287P-S322T-N388M-E421V-C430N-L432F-L434W-S436N-C437F-H461T-G462R-i462aA-A463T | ++++ |
| V272K-I283T-P284D-i284aP-N287P-S322T-N388M-E421V-C430N-L432F-L434W-S436N-C437F-H461T-G462R-i462aA-A463T | ++++ |
| A33P-I283T-P284D-i284aP-N287P-S322T-N388M-E421V-C430N-L432F-L434W-S436N-C437F-H461T-G462R-i462aA-A463T | ++++ |
| I283T-P284D-i284aP-N287P-S322T-H384S-N388M-E421V-C430N-L432F-L434W-S436N-C437F-H461T-G462R-i462aA-A463T | ++++ |
| I283T-P284D-i284aP-N287P-S322T-N388M-I418V-E421V-C430N-L432F-L434W-S436N-C437F-H461T-G462R-i462aA-A463T | ++++ |
| K43E-I283T-P284D-i284aP-N287P-S322T-N388M-E421V-C430P-L432F-L434W-S436N-C437F-H461T-G462R-i462aA-A463T | ++++ |
| I283T-P284D-i284aP-N287P-S322T-N388M-E421V-C430R-L432F-L434W-S436N-C437F-H461T-G462R-i462aA-A463T | ++++ |
| I283T-P284D-i284aP-N287P-S322T-N388M-E421V-C430N-L432F-L434W-S436N-C437F-Y438L-H461T-G462R-i462aA-A463T | ++++ |
| I283T-P284D-i284aP-N287P-S322T-N388M-E421V-C430N-L432F-L434W-S436Q-C437F-H461T-G462R-i462aA-A463T | ++++ |
| I283T-P284D-i284aP-N287P-S322T-N388M-E421V-C430N-L432F-L434W-S436N-C437F-E441V-H461T-G462R-i462aA-A463T | ++++ |
| I283T-P284D-i284aP-N287P-S322T-N388M-E421V-C430N-L432F-L434W-S436N-C437F-i444aT-H461T-G462R-i462aA-A463T | ++++ |
| I283T-P284D-i284aP-N287P-S322T-N388M-E421V-C430R-L432F-L434W-S436N-C437F-Y438L-H461T-G462R-i462aA-A463T | ++++ |
| V272I-I283T-P284D-i284aP-N287P-S322T-N388M-E421V-C430R-L432F-L434W-S436N-C437F-H461T-G462R-i462aA-A463T | ++++ |
| V272I-I283T-P284D-i284aP-N287P-S322T-N388M-E421V-C430R-L432F-L434W-S436N-C437F-Y438L-H461T- | ++++ |
| G462R-i462aA-A463T | |
| V272I-I283T-P284D-i284aP-N287P-S322T-N388M-E421V-C430N-L432F-L434W-S436N-C437F-Y438L-H461T- | ++++ |
| G462R-i462aA-A463T | |
| V272R-I283T-P284D-i284aP-N287P-S322T-N388M-I418V-E421V-C430N-L432F-L434W-S436N-G448E-H461T- | ++++ |
| G462R-i462aA-A463T | |
| G17S-V272R-I283T-P284D-i284aP-N287P-S322T-N388M-E421K-C430N-L432F-L434W-S436N-G448E-H461T- | ++++ |
| G462R-i462aA-A463T | |
| V272R-I283T-P284D-i284aP-N287P-S322T-N388M-E421V-E428T-C430N-L432F-L434W-S436N-G448E-H461T- | ++++ |
| G462R-i462aA-A463T | |
| V272R-I283T-P284D-i284aP-N287P-S322T-N388M-E421V-C430N-L432F-L434W-S436N-E441H-G448E-H461T- | ++++ |
| G462R-i462aA-A463T | |
| V46A-V272R-I283T-P284D-i284aP-N287P-S322T-N388M-E421V-C430N-L432F-L434W-S436N-G448E-i458aG- | ++++ |
| H461T-G462R-i462aA-A463T | |
| S39G-V272R-I283T-P284D-i284aP-N287P-S322T-N388M-E421V-F426L-E428T-C430N-L432F-L434W-S436N- | ++++ |
| G448E-H461T-G462R-i462aA-A463T | |
| V272R-I283T-P284D-i284aP-N287P-S322T-N388M-E421V-E428T-C430N-L432F-L434W-S436N-K447I-G448E- | ++++ |
| H461T-G462R-i462aA-A463T | |
| E224P-I283T-N285P-N287T-Q290E-S322T-N388M-S389H-K394R-C430A-i430aG-L432F-L434W-S436N-I453L-dG462 | ++++ |

As regards the determination of sequence identity, the following should apply: When the sequences which are compared do not have the same length, the degree of identity either refers to the percentage of amino acid residues in the shorter sequence which are identical to amino acid residues in the longer sequence or to the percentage of amino acid residues in the longer sequence which are identical to amino acid residues in the shorter sequence. Preferably, it refers to the percentage of amino acid residues in the shorter sequence which are identical to amino acid residues in the longer sequence. The degree of sequence identity can be determined according to methods well known in the art using preferably suitable computer algorithms such as CLUSTAL.

When using the Clustal analysis method to determine whether a particular sequence is, for instance, at least 60% identical to a reference sequence default settings may be used or the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

Preferably, the degree of identity is calculated over the complete length of the sequence.

### Increasing the Pool of Coenzyme a in the Organism or Microorganism

It has been disclosed in WO 2020/188033 that increasing the pool of coenzyme A (CoA) can result in an increased production of isobutene. In WO 2020/188033, increased pools of CoA are achieved due to:
(i) an increased uptake of pantothenate; and/or
(ii) an increased conversion of pantothenate into CoA.

Accordingly, the recombinant organism or microorganism according to the invention may further be improved by increasing the uptake of pantothenate and/or by increasing the conversion of pantothenate into CoA. The transporters and enzymes to achieve an increased uptake of pantothenate and/or an increased conversion of pantothenate into CoA are disclosed in WO 2020/188033, which is fully incorporated herein by reference.

### The Organism or Microorganism

Within the present invention, the organism may be any organism, preferably any organism that is suitable for the use in biotechnological processes at an industrial scale. Preferably, the organism is an organism that can be used for the biotechnological production of isobutene or its precursor molecule 3-methylcrotonic acid at an industrial scale. More preferably, the organism according to the invention is a microorganism.

The term “microorganism” in the context of the present invention refers to bacteria, as well as to fungi, such as yeasts, and also to algae and archaea. In one preferred embodiment, the microorganism is a bacterium. In principle any bacterium can be used. Preferred bacteria to be employed in the present invention are bacteria of the genus Bacillus, Clostridium, Corynebacterium, Pseudomonas, Zymomonas or Escherichia. In a particularly preferred embodiment, the bacterium belongs to the genus Escherichia and even more preferred to the species Escherichia coli. In another preferred embodiment, the bacterium belongs to the species Pseudomonas putida or to the species Zymomonas mobilis or to the species Corynebacterium glutamicum or to the species Bacillus subtilis. It is also possible to employ an extremophilic bacterium such as Thermus thermophilus, or anaerobic bacteria from the family Clostridiae.

In another particularly preferred embodiment, the microorganism is a microorganism which is capable using carbon monoxide (CO) and gaseous substrates comprising CO like, e.g., syngas, as the source of carbon and energy. Syngas or synthesis gas is a mixture of CO and CO₂ as well as H₂. Thus, in a particularly preferred embodiment, the microorganism is a C1-fixing microorganism. As mentioned above, corresponding naturally occurring (or genetically modified) microorganisms that are capable of utilizing CO and converting it into acetyl-CoA are known in the art. These organisms are often referred to as acetogenic microorganisms (sometimes also termed carboxydotrophic, acetogenic microorganisms) and are referred to as “C1-fixing microorganisms” herein. These microorganisms use the Wood-Ljungdahl pathway to fix CO and convert it into acetyl-CoA. Examples of such microorganisms belong to the family Clostridiae and are, e.g., described in WO 2009/094485; WO 2012/05905; WO 2013/180584; US 2011/0236941; PNAS 107(29):13087-13092 (2010); Current Opinion in Biotechnology 23:364-381 (2012); Applied and Environmental Microbiology 77(15):5467-5475 (2011). The use of C1-fixing microorganisms is extensively discussed in WO 2020/188033, which is incorporated herein in its entirety.

In another preferred embodiment the microorganism is a fungus, more preferably a fungus of the genus Saccharomyces, Schizosaccharomyces, Aspergillus, Trichoderma, Kluyveromyces, Clostridium or Pichia and even more preferably of the species Saccharomyces cerevisiae, Schizosaccharomyces pombe, Aspergillus niger, Trichoderma reesei, Kluyveromyces marxianus, Kluyveromyces lactis, Pichia pastoris, Pichia torula or Pichia utilis.

In another embodiment, the present invention makes use of a photosynthetic microorganism expressing at least one enzyme for the conversion according to the invention as described above. Preferably, the microorganism is a photosynthetic bacterium, or a microalgae. In a further embodiment the microorganism is an algae, more preferably an algae belonging to the diatomeae.

It is also conceivable to use in accordance with the present invention a combination of microorganisms, wherein different microorganisms express different enzymes as described above.

In a preferred embodiment, the organism, preferably the microorganism, of the present invention is an organism which is capable of consuming CO and/or syngas. In another preferred embodiment, the organism, preferably the microorganism, of the present invention is an organism which is capable of consuming a mixture of CO and/or CO₂ as well as H₂.

In another embodiment, said organism and/or microorganism is genetically modified in order to be capable of consuming glucose, fructose, xylose, mannose and/or CO (or syngas) and/or genetically modified in order to increase the organism's and/or microorganism's capability of consuming glucose, fructose, xylose, mannose and/or CO (or syngas). Corresponding genetic modifications are known in the art.

In another preferred embodiment, the organism, preferably the microorganism, of the present invention is an organism which is capable of consuming sugar through a Phosphotransferase Transport System (PTS).

In a preferred embodiment, the organism, preferably the microorganism, of the present invention is an organism which is capable of consuming sugar through a non-Phosphotransferase Transport System (non-PTS).

Organisms and/or microorganisms which are capable of consuming sugar through a Phosphotransferase Transport System (PTS) and/or through a non-Phosphotransferase Transport System (non-PTS) are known in the art.

In another embodiment, said organism and/or microorganism is genetically modified in order to be capable of consuming sugar through a Phosphotransferase Transport System (PTS) or through a non-Phosphotransferase Transport System (non-PTS). In another preferred embodiment, said organism and/or microorganism is genetically modified in order to increase the organism's and/or microorganism's capability of consuming sugar through a Phosphotransferase Transport System (PTS) or through a non-Phosphotransferase Transport System (non-PTS). Corresponding genetic modifications are known in the art.

In another preferred embodiment, the organism, preferably the microorganism, of the present invention is an organism having a diminished or inactivated Phosphotransferase Transport System (PTS). Such an organism, preferably a microorganism, may preferably be genetically modified by deletion or inactivation (a) gene(s) of said Phosphotransferase Transport System (PTS). Corresponding genetic modifications are known in the art.

In another preferred embodiment, the organism, preferably the microorganism, of the present invention is an organism having an enhanced non-Phosphotransferase Transport System (non-PTS) for sugar uptake. Such an organism, preferably a microorganism, may preferably be genetically modified by overexpression (a) gene(s) of said non-Phosphotransferase Transport System (non-PTS) for sugar uptake. Corresponding genetic modifications are known in the art.

In another preferred embodiment, the organism, preferably the microorganism, of the present invention is an organism having a diminished or inactivated Phosphotransferase Transport System (PTS) and an enhanced non-Phosphotransferase Transport System (non-PTS) for sugar uptake.

In another preferred embodiment, the organism, preferably the microorganism, of the present invention is an organism which is capable of consuming sucrose through a non-Phosphotransferase Transport System (non-PTS).

In another preferred embodiment, the organism, preferably the microorganism, of the present invention is an organism consuming sucrose, wherein said organism, preferably said microorganism, has genetically been modified by the introduction of at least one gene of a non-Phosphotransferase Transport System (non-PTS). Without being bound to theory, such an organism and/or microorganism has genetically been modified by introducing a gene selected from the group consisting of cscA, cscB, and cscK from Escherichia coli W (M. Bruschi et al., Biotechnology Advances 30 (2012) 1001-1010).

In another preferred embodiment, the organism, preferably the microorganism, of the present invention is an organism which has genetically been modified to have a diminished or inactivated Phosphotransferase Transport System (PTS) and an overexpression of at least one gene selected from the group consisting of galP, glk and glf.

In a preferred embodiment, the organism, preferably the microorganism, of the present invention is an organism which is genetically modified in order to avoid the leakage of acetyl-CoA, thereby increasing the intracellular concentration of acetyl-CoA. Genetic modifications leading to an increase in the intracellular concentration of acetyl-CoA are known in the art. Such an organism, preferably a microorganism, may preferably be genetically modified by deleting or inactivating the following genes: ΔackA (acetate kinase), Δldh (lactate dehydrogenase), ΔadhE (alcohol dehydrogenase), ΔfrdB and/or ΔfrdC (fumarate reductase and fumarate dehydrogenase).

In another embodiment, the method of the invention comprises the step of providing the organism, preferably the microorganism, of the present invention in the form of a (cell) culture, preferably in the form of a liquid cell culture, a subsequent step of cultivating the organism, preferably the microorganism, in a fermenter (often also referred to a bioreactor) under suitable conditions and further comprising the step of effecting an enzymatic conversion of a method of the invention as described herein. Suitable fermenter or bioreactor devices and fermentation conditions are known to the person skilled in the art. A bioreactor or a fermenter refers to any manufactured or engineered device or system known in the art that supports a biologically active environment. Thus, a bioreactor or a fermenter may be a vessel in which a chemical/biochemical reaction like the method of the present invention is carried out which involves organisms, preferably microorganisms and/or biochemically active substances. In a bioreactor or a fermenter, this process can either be aerobic or anaerobic. These bioreactors are commonly cylindrical, and may range in size from litres to cubic metres, and are often made of stainless steel. In this respect, without being bound by theory, the fermenter or bioreactor may be designed in a way that it is suitable to cultivate the organisms, preferably microorganisms, in, e.g., a batch-culture, fed-batch-culture, perfusion culture or chemostate-culture, all of which are generally known in the art.

The culture medium can be any culture medium suitable for cultivating the respective organism or microorganism. When carried out by making use of an organism or microorganism, the method according to the present invention may, e.g. be designed as a continuous fermentation culturing method or as a batch culture or any suitable culture method known to the person skilled in the art.

The Production of 3-methylcrotonic acid and/or Isobutene

In a particular embodiment, the invention relates to a method for the production of 3-methylcrotonic acid and/or isobutene, the method comprising a step of culturing a recombinant organism or microorganism according to the invention in a suitable culture medium under suitable conditions.

That is, the organism or microorganism according to the invention may be used in an industrial process for the production of isobutene. In certain embodiments, all enzymatic steps for the synthesis of isobutene will be carried out in the organism or microorganism of the invention. In such embodiments, the organism or microorganism preferably comprises the enzyme ferulic acid decarboxylase, which catalyzes the conversion of 3-methylcrotonic acid into isobutene.

Alternatively, isobutene may be produced in a two-step process. That is, the organism or microorganism according to the invention may be used for the production of the precursor molecule 3-methylcrotonic acid. The produced 3-methylcrotonic acid may then be converted into isobutene in vivo or in vitro in a biotransformation reaction.

That is, in a particular embodiment, the invention relates to a method for the production of isobutene, the method comprising the steps of: a) producing 3-methylcrotonic acid by culturing a recombinant organism or microorganism of the invention in a suitable culture medium under suitable conditions; and b) enzymatically converting said produced 3-methylcrotonic acid into isobutene in vivo or in vitro.

In a preferred embodiment, the method according to the present invention also comprises the step of recovering the isobutene produced by the method. For example, when the method according to the present invention is carried out in vivo by fermenting a corresponding organism or microorganism expressing the necessary enzymes/transporters, the isobutene can be recovered from the fermentation off-gas by methods known to the person skilled in the art.

As mentioned above, the recombinant organism and microorganism as well as the method according to the present invention is in particular useful for large scale production of isobutene in vivo, in particular for a commercial production. The present invention describes novel means and ways to commercially and cost-effectively produce large quantities of isobutene which has not been obtainable to date. The generated large quantities of isobutene can then be further converted, in a commercial setting, to produce large quantities of, e.g., drop- in gasoline (e.g. isooctane, ETBE, MTBE), jet-fuel, cosmetics, chemicals, such as methacrylic acid, polyisobutene, or butyl rubber. As used herein, “large scale production”, “commercial production” and “bioprocessing” of isobutene in a fermentation reactor or in vitro is carried out at a capacity greater than at least 100 liters, and preferably greater than at least 400 liters, or more preferably production of 1,000 liters of scale or more, even more preferably production of 5,000 liters of scale or more. As used herein, “large quantities” specifically excludes trace amounts that may be produced inherently in an organism or microorganism.

For example, in preferred embodiments, the yields for continuous cultures according to methods described herein are at least about 0.2 grams of isobutene per liter per day, at least about 0.3 grams of isobutene per liter per day, at least about 0.4 grams of isobutene per liter per day, at least about 0.5 grams of isobutene per liter per day, at least about 0.6 grams of isobutene per liter per day, at least about 0.7 grams of isobutene per liter per day, at least about 0.8 grams of isobutene per liter per day, or at least about 1.0 grams of isobutene per liter per day. In further embodiments, the yields for continuous cultures according to methods described herein are between about 0.3 grams and about 1.0 grams of isobutene per liter per day, between about 0.4 grams to about 1.0 grams of isobutene per liter per day, and between about 0.5 grams and about 1.0 grams of isobutene per liter per day. In other specific embodiments, the yields for continuous cultures according to methods described herein are between about 0.5 grams to about 0.75 grams of isobutene per liter per day. In other specific embodiments, the yields for continuous cultures according to methods described herein are between about 0.5 grams and about 1.5 grams of isobutene per liter per day.

In further embodiments, the yields for batch cultures according to methods described herein are at least about 2 grams per liter in batch culture, at least about 5 grams per liter in batch culture, at least about 10 grams per liter in batch culture, and at least about 15 grams per liter in batch culture. In some embodiments, the yields for batch cultures according to methods described herein are between about 2 grams and about 5 grams per liter in batch culture, between about 5 grams and about 10 grams per liter in batch culture, and still more preferably between about 10 grams and about 20 grams per liter in batch culture. In other specific embodiments, the yields for batch cultures according to methods described herein are between about 2.4 grams and about 4.8 grams per liter, and preferably between about 4.8 grams and about 9.4 grams per liter in batch culture, and still more preferably between about 9.4 grams and about 18.6 grams per liter in batch culture.

### The Production of Other Alkenes

Within the present invention, the recombinant organism or microorganism is preferably used for the production of isobutene, wherein 3-methylcrotonic acid is converted to isobutene in a final reaction step by the enzyme ferulic acid decarboxylase (FDC). However, it is known in the art that FDCs can be used for the production of other alkenes.

For example, Mori et al. (Direct 1,3-butadiene biosynthesis in Escherichia coli via a tailored ferulic acid decarboxylase mutant, Nature Communications volume 12, Article number: 2195 (2021)) disclosed the production of 1,3-butadiene biosynthesis from cis, cis-muconic acid and pentadienoic acid using FDC. As it is shown herein that crotonic acid and 2-hexenoic acid can inhibit both wild type and highly engineered FDC variants, it is likely that also other biotechnological processes that involve activity of an FDC variant, such as the production of 1,3-butadiene, will be significantly inhibited in the presence of even trace amounts of crotonic acid and/or 2-hexenoic acid. Thus, the recombinant organism or microorganism according to this invention may be used as a universal platform for FDC-based alkene production.

Accordingly, in a particular embodiment, the invention relates to a recombinant organism or microorganism having a decreased pool of crotonic acid compared to the organism or microorganism from which it is derived due to at least one of:
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonic acid into crotonyl-CoA;
(iv) an increased conversion of crotonyl-[acyl-carrier protein] into butyryl [acyl-carrier-protein];
(v) a decreased conversion of crotonyl-[acyl-carrier protein] into crotonic acid; and/or
(vi) a decreased conversion of crotonyl-CoA into crotonic acid;
   wherein the recombinant organism of microorganism encodes a ferulic acid decarboxylase.

The FDC may be any one of the FDC variants that have been disclosed herein for the production of isobutene. Further, the FDC may be an FDC from Aspergillus niger (AnFDC) or Saccharomyces cerevisiae (scFDC) or any mutant variant thereof. In particular, the FDC may be any variant of AnFDC or ScFDC that has been disclosed by Mori et al. (see citation above) for the production of 1-3-butadiene.

In a particular embodiment, the invention relates to a recombinant organism or microorganism having a decreased pool of crotonic acid compared to the organism or microorganism from which it is derived due to at least one of:
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonic acid into crotonyl-CoA;
(iv) an increased conversion of crotonyl-[acyl-carrier protein] into butyryl [acyl-carrier-protein];
(v) a decreased conversion of crotonyl-[acyl-carrier protein] into crotonic acid; and/or
(vi) a decreased conversion of crotonyl-CoA into crotonic acid;
   wherein the recombinant organism or microorganism is used in the production of an alkene.

In a particular embodiment, the invention relates to a recombinant organism or microorganism having a decreased pool of crotonic acid compared to the organism or microorganism from which it is derived due to at least one of:
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonic acid into crotonyl-CoA;
(iv) an increased conversion of crotonyl-[acyl-carrier protein] into butyryl [acyl-carrier-protein];
(v) a decreased conversion of crotonyl-[acyl-carrier protein] into crotonic acid; and/or
(vi) a decreased conversion of crotonyl-CoA into crotonic acid;
   wherein the organism or microorganism is capable of producing a substrate of a ferulic acid decarboxylase.

The alkene may be any alkene that can be produced with a wild type or engineered FDC variant. In a particular embodiment, the alkene is isobutene. In another embodiment, the alkene is 1,3-butadiene. It is to be noted that the recombinant organism or microorganism does not necessarily need to comprise a gene encoding an FDC. That is, the recombinant organism or microorganism may also be used for the production of a precursor of an alkene, which is then converted into the alkene by an FDC in a subsequent step. Thus, the recombinant organism or microorganism may be used only for a certain part of an alkene production process.

For example, a recombinant organism or microorganism according to the invention may be used in the production of the precursor molecules 3-methylcrotonic acid or cis, cis-muconic acid or pentadienoic acid. Producing these precursors in the recombinant organism or microorganism according to the invention will minimize the formation of crotonic acid and/or 2-hexenoic acid as side products. As a result, the fermentation broth can be used directly for the production of the corresponding alkenes by an FDC.

In certain embodiments, 1,3-butadiene is obtained by:
(i) enzymatically converting phosphoenolpyruvate and erythrose-4-phosphate into 3-deoxy-D-heptulosonate-7-phosphate into,
(ii) enzymatically converting 3-deoxy-D-heptulosonate-7-phosphate into 3-dehydroshikimate,
(iii) enzymatically converting 3-dehydroshikimate into protocatechuic acid,
(iv) enzymatically converting protocatechuic acid into catechol,
(v) enzymatically converting catechol into cis,cis-muconate;
(vi) enzymatically converting cis,cis-muconate into (Z)-pentadienoate; and
(vii) enzymatically converting (Z)-pentadienoate into 1,3-butadiene.

Preferably, the conversion of cis, cis-muconate into (Z)-pentadienoate and (Z)-pentadienoate into 1,3-butadiene are catalyzed by a ferulic acid decarboxylase. In certain embodiments, the ferulic acid decarboxylase is an engineered AnFDC or ScFDC as disclosed by Mori et al.

In a particular embodiment, the invention relates to a recombinant organism or microorganism having a decreased pool of crotonic acid compared to the organism or microorganism from which it is derived due to at least:
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonic acid into crotonyl-CoA;
(iv) a decreased conversion of crotonyl-CoA into crotonic acid;
(v) an increased conversion of crotonyl-[acyl-carrier protein] into butyryl [acyl-carrier-protein]; and/or
(vi) a decreased conversion of crotonyl-[acyl-carrier protein] into crotonic acid;
   wherein said recombinant organism or microorganism is capable of converting phosphoenolpyruvate and erythrose-4-phosphate into 1,3-butadiene, wherein in said microorganism:
(i) phosphoenolpyruvate and erythrose-4-phosphate are enzymatically converted into 3-deoxy-D-heptulosonate-7-phosphate,
(ii) 3-deoxy-D-heptulosonate-7-phosphate is enzymatically converted into 3-dehydroshikimate,
(iii) 3-dehydroshikimate is enzymatically converted into protocatechuic acid,
(iv) protocatechuic acid is enzymatically converted into catechol,
(v) catechol is enzymatically converted into cis,cis-muconate;
(vi) cis,cis-muconate is enzymatically converted into (Z)-pentadienoate; and
(vii) (Z)-pentadienoate is enzymatically converted into 1,3-butadiene.

Preferably, phosphoenolpyruvate is obtained from glucose-6-phosphate during glycolysis and erythrose-4-phosphate is obtained from glucose-6-phosphate during the pentose-6-phosphate pathway.

### List of Embodiments

1. A recombinant organism or microorganism having a decreased pool of crotonic acid compared to the organism or microorganism from which it is derived due to at least one of:
   (i) an increased conversion of crotonyl-CoA into butyryl-CoA; and/or an increased conversion of butyryl-CoA into butyric acid;
   (ii) an increased conversion of crotonyl-CoA into 3-hydroxybutyryl-CoA; and/or an increased conversion of 3-hydroxybutyryl-CoA into 3-hydroxybutyric acid;
   (iii) an increased conversion of crotonic acid into crotonyl-CoA;
   (iv) an increased conversion of crotonyl-[acyl-carrier protein] into butyryl [acyl-carrier-protein];
   (v) a decreased conversion of crotonyl-[acyl-carrier protein] into crotonic acid; and/or
   (vi) a decreased conversion of crotonyl-CoA into crotonic acid.
2. The recombinant organism or microorganism according to embodiment 1, wherein said recombinant organism or microorganism is a recombinant microorganism.
3. The recombinant organism or microorganism according to embodiment 1 or 2, wherein said recombinant microorganism is a fungus or a bacterium.
4. The recombinant organism or microorganism according to embodiment 3, wherein said bacterium is Escherichia coli.
5. The recombinant organism or microorganism according to any one of embodiments 1 to 4, wherein the increased conversion of crotonyl-CoA into butyryl-CoA is due to an increased level and/or activity of at least one enzyme capable of reducing a carbon-carbon double bond (EC 1.3) in said organism or microorganism.
6. The recombinant organism or microorganism according to embodiment 5, wherein the enzyme capable of reducing a carbon-carbon double bond (EC 1.3) is NADH or NADPH-dependent (EC 1.3.1) or flavin-dependent (EC 1.3.8).
7. The recombinant organism or microorganism according to embodiment 5 or 6, wherein the NADH or NADPH-dependent enzyme capable of reducing a carbon-carbon double bond (EC 1.3.1) is a crotonyl-CoA reductase (EC 1.3.1.86), a trans-2-enoyl-CoA reductase (EC 1.3.1.44) and/or an enoyl-[acyl-carrier-protein] reductase (EC 1.3.1.9).
8. The recombinant organism or microorganism according to embodiment 7, wherein
   (i) the trans-2-enoyl-CoA reductase (EC 1.3.1.44) is FabV from Treponema denticola;
   (ii) the crotonyl-CoA reductase (EC 1.3.1.86) is Ccr from Streptomyces collinus; and/or
   (iii) the enoyl-[acyl-carrier-protein] reductase (EC 1.3.1.9) is FabI from Escherichia coli.
9. The recombinant organism or microorganism according to embodiment 5 or 6, wherein the flavin-dependent enzyme capable of reducing a carbon-carbon double bond (EC 1.3.8) is a short-chain acyl-CoA dehydrogenase (EC 1.3.8.1).
10. The recombinant organism or microorganism according to embodiment 9, wherein the short-chain acyl-CoA dehydrogenase (EC 1.3.8.1) is a short-chain acyl-CoA dehydrogenase from Megasphaera elsdenii; and/or a butyryl-CoA dehydrogenase (Bcd) with the electron transferring flavoprotein (Etf) from Acidaminococcus fermentans.
11. The recombinant organism or microorganism according to any one of embodiments 1 to 10, wherein the increased conversion of butyryl-CoA into butyric acid is due to an increased level and/or activity of a thioester hydrolase (EC 3.1.2), a CoA-transferase (EC 2.8.3), an acid thiol ligase (EC 6.2.1), a phosphate acyltransferase (EC 2.3.1) and/or acid kinase (EC 2.7.2) in said organism or microorganism.
12. The recombinant organism or microorganism according to embodiment 11, wherein the thioester hydrolase (EC 3.1.2) is a 1,4-dihydroxy-2-naphtoyl-CoA hydrolase (EC 3.1.2.28) and/or an acyl-CoA thioesterase 2 (EC 3.1.2.20).
13. The recombinant organism or microorganism according to embodiment 12, wherein
   (i) the 1,4-dihydroxy-2-naphtoyl-CoA hydrolase (EC 3.1.2.28) is MenI from Escherichia coli; and/or
   (ii) the acyl-CoA thioesterase 2 (EC 3.1.2.20) is TesB from Escherichia coli.
14. The recombinant organism or microorganism according to embodiment 11, wherein the CoA-transferase (EC 2.8.3) is an acetate CoA-transferase and/or a butyryl-CoA: acetate CoA-transferase (EC 2.8.3.8).
15. The recombinant organism or microorganism according to embodiment 14, wherein
   (i) the acetate CoA-transferase (EC 2.8.3.8) is YdiF (Pct) from Cupriavidus necator; and/or
   (ii) the butyrate: acetyl-CoA-transferase (EC 2.8.3.8) is encoded by SwoI_1932 or SwoI_0436 from Syntrophomonas wolfei subsp. wolfei.
16. The recombinant organism or microorganism according to embodiment 11, wherein the acid thiol ligase (EC 6.2.1) is an acetate-CoA ligase (ADP-forming) (EC 6.2.1.13).
17. The recombinant organism or microorganism according to embodiment 16, wherein the acetate-CoA ligase (ADP-forming) (EC 6.2.1.13) is encoded by the gene Caur_3920 from Chloroflexus aurantiacus and/or the gene EHI_178960 from Entamoeba histolytica and/or wherein the acetate-CoA ligase (ADP-forming) (EC 6.2.1.13) is the protein Q9Y1N2 from Giardia intestinalis (Giardia lamblia).
18. The recombinant organism or microorganism according to embodiment 11, wherein the phosphate acyltransferase (EC 2.3.1) is a phosphate butyryltransferase (EC 2.3.1.19) and/or wherein the acid kinase (EC 2.7.2) is a butyrate kinase (EC 2.7.2.7).
19. The recombinant organism or microorganism according to embodiment 18, wherein
   (i) the phosphate butyryltransferase (EC 2.3.1.19) is Ptb from Clostridium acetobutylicum; and/or
   (ii) the butyrate kinase (EC 2.7.2.7) is Buk from Clostridium acetobutylicum.
20. The recombinant organism or microorganism according to any one of embodiments 1 to 19, wherein the increased conversion of crotonyl-CoA into 3-hydroxybutyryl-CoA is due to an increased level and/or activity of a hydro-lyase (EC 4.2.1) in said organism or microorganism.
21. The recombinant organism or microorganism according to embodiment 20, wherein the hydro-lyase (EC 4.2.1) is a short-chain-enoyl-CoA hydratase (EC 4.2.1.150), a 3-hydroxybutyryl-CoA dehydratase (EC 4.2.1.55) and/or an enoyl-CoA hydratase (EC 4.2.1.17).
22. The recombinant organism or microorganism according to embodiment 21, wherein
   (i) the short-chain-enoyl-CoA hydratases (EC 4.2.1.150) is a short-chain-enoyl-CoA hydratase from Meiothermus ruber, Metallosphaera sedula or Clostridium acetobutylicum;
   (ii) the 3-hydroxybutyryl-CoA dehydratase (EC 4.2.1.55) is a 3-hydroxybutyryl-CoA dehydratase from Ferroglobus placidus; and/or
   (iii) the enoyl-CoA hydratase (EC 4.2.1.17) is an enoyl-CoA hydratase from Rattus norvegicus.
23. The recombinant organism or microorganism according to any one of embodiments 1 to 22, wherein the increased conversion of 3-hydroxybutyryl-CoA into 3-hydroxybutyric acid is due to an increased level and/or activity of a thioester hydrolase (EC 3.1.2) in said organism or microorganism.
24. The recombinant organism or microorganism according to embodiment 23, wherein the thioester hydrolase (EC 3.1.2) is a palmitoyl-CoA hydrolase (EC 3.1.2.2) and/or an acyl-CoA thioesterase 2 (EC 3.1.2.20).
25. The recombinant organism or microorganism according to embodiment 24, wherein
   (i) the palmitoyl-CoA hydrolase (EC 3.1.2.2) is a palmitoyl-CoA hydrolase from Photobacterium profundum; and/or
   (ii) the acyl-CoA thioesterase 2 (EC 3.1.2.20) is TesB or YciA from Escherichia coli.
26. The recombinant organism or microorganism according to any one of embodiments 1 to 25, wherein the increased conversion of crotonic acid into crotonyl-CoA is due to an increased level and/or activity of a CoA-transferase (EC 2.8.3) and/or an acid thiol ligase (EC 6.2.1) and/or an acid kinase (EC 2.7.2) and/or a phosphate acyltransferase (EC 2.3.1) in said organism or microorganism.
27. The recombinant organism or microorganism according to embodiment 26, wherein the CoA-transferase (EC 2.8.3) is an acetate CoA-transferase (EC 2.8.3.8).
28. The recombinant organism or microorganism according to embodiment 27, wherein
   (i) the acetate CoA-transferase (EC 2.8.3.8) is YdiF (Pct) from Cupriavidus necator; and
   (ii) the butyrate: acetyl-CoA-transferase (EC 2.8.3.8) is encoded by SwoI_1932 or SwoI_0436 from Syntrophomonas wolfei subsp. wolfei.
29. The recombinant organism or microorganism according to embodiment 26, wherein the acid thiol ligase (EC 6.2.1) is a medium-chain acyl-CoA ligase (EC 6.2.1.2), a 4-hydroxybenzoate-CoA ligase/benzoate-CoA ligase (EC 6.2.1.25 and 6.2.1.27), a 4-Hydroxybutyrate-CoA ligase (EC 6.2.1.40), or a methylmercaptopropionate (MMPA)-coenzyme A (CoA) ligase (EC 6.2.1.44).
30. The recombinant organism or microorganism according to embodiment 29, wherein
   (i) the medium-chain acyl-CoA ligase (EC 6.2.1.2) is encoded by the gene PA3924 from Pseudomonas aeruginosa,
   (ii) the 4-hydroxybenzoate-CoA ligase/benzoate-CoA ligase (EC 6.2.1.25 and 6.2.1.27) is encoded by the gene SYN_02896 from Syntrophus aciditrophicus (strain SB) or by the gene SYN_02898 from Syntrophus aciditrophicus (strain SB),
   (iii) the 4-Hydroxybutyrate-CoA ligase (EC 6.2.1.40) is encoded by the gene Tneu_0420 from Pyrobaculum neutrophilum (Thermoproteus neutrophilus); and/or
   (iv) the methylmercaptopropionate (MMPA)-coenzyme A (CoA) ligase (EC 6.2.1.44) is encoded by the gene SAR11_0248 from Pelagibacter ubique; or by the gene SPO0677 from Ruegeria pomeroyi; or by the gene SPO2045 from Ruegeria pomeroyi; or by the gene SL1157_1815 from Ruegeria lacuscaerulensis; or by the gene SL1157_2728 from Ruegeria lacuscaerulensis or by the gene PA4198 from Pseudomonas aeruginosa; or by the gene BTH_12141 from Burkholderia thailandensis.
31. The recombinant organism or microorganism according to embodiment 26, wherein the phosphate acyltransferase (EC 2.3.1) is a phosphate butyryltransferase (EC 2.3.1.19) and/or wherein the acid kinase (EC 2.7.2) is a butyrate kinase (EC 2.7.2.7).
32. The recombinant organism or microorganism according to embodiment 31, wherein
   (i) the phosphate butyryltransferase (EC 2.3.1.19) is Ptb from Clostridium acetobutylicum; and/or
   (ii) the butyrate kinase (EC 2.7.2.7) is Buk from Clostridium acetobutylicum.
33. The recombinant organism or microorganism according to any one of embodiments 1 to 32, wherein the increased conversion of crotonyl-[acyl-carrier protein] into butyryl-[acyl-carrier protein] is due to an increased level and/or activity of an NADH or NADPH-dependent enoyl-[acyl-carrier-protein] reductase (EC 1.3.1) in said organism or microorganism, in particular wherein the NADH or NADPH-dependent enoyl-[acyl-carrier-protein] reductase (EC 1.3.1) is an enoyl-[acyl-carrier-protein] reductase (NADH-dependent) (EC 1.3.1.9), an enoyl-[acyl-carrier-protein] reductase (NADPH-dependent) (EC 1.3.1.104), an enoyl-[acyl-carrier-protein] reductase (NADPH-dependent, Re-specific) (EC 1.3.1.39), an enoyl-[acyl-carrier-protein] reductase (NADPH-dependent, Si-specific) (EC 1.3.1.10), and/or a trans-2-enoyl-CoA reductase (NAD+) (EC 1.3.1.44).
34. The recombinant organism or microorganism according to embodiment 33, wherein the NADH or NADPH-dependent enoyl-[acyl-carrier-protein] reductase is FabI from Escherichia coli (EC 1.3.1.9 and 1.3.1.104), FabI from Bacillus subtilis (EC 1.3.1.9), FabL from Bacillus subtilis (EC 1.3.1.104), FabI from Staphylococcus aureus (EC 1.3.1.39), FabK from Porphyromonas gingivalis (EC 1.3.1.10 and EC 1.3.1.39), FabK from Streptococcus pneumoniae (EC 1.3.1.10), ETR1 from Saccharomyces cerevisiae (EC 1.3.1.104), FabV from Burkholderia mallei (EC 1.3.1.9 and 1.3.1.44), FabV from Pseudomonas aeruginosa (EC 1.3.1.9 and 1.3.1.44), FabV from Vibrio cholera (EC 1.3.1.9 and 1.3.1.44), FabV from Treponema denticola (EC 1.3.1.44), FabI from Pseudomonas aeruginosa (EC 1.3.1.9), and/or FabI from Burkholderia pseudomallei (EC 1.3.1.9).
35. The recombinant organism or microorganism according to any one of embodiments 5 to 34, wherein the increased level of an enzyme is achieved by expressing a gene encoding the respective enzyme from a recombinant promoter and/or from an improved ribosome binding site.
36. The recombinant organism or microorganism according to any one of embodiments 5 to 35, wherein the increased activity of an enzyme is due to one or more activating mutations in the gene encoding the respective enzyme.
37. The recombinant organism or microorganism according to any one of embodiments 1 to 36, wherein the decreased conversion of crotonyl-[acyl-carrier-protein] and/or crotonyl-CoA into crotonic acid is due to a decreased level and/or a decreased activity of a thioester hydrolase (EC 3.1.2) in said organism or microorganism.
38. The recombinant organism or microorganism according to embodiment 37, wherein the thioester hydrolase (EC 3.1.2) is a palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28).
39. The recombinant organism or microorganism according to embodiment 38, wherein
   (i) the thioester hydrolase (EC 3.1.2) is PaaY or PaaI from Escherichia coli;
   (ii) the palmitoyl-CoA hydrolase (EC 3.1.2.2) is TesA, YciA or EntH from Escherichia coli;
   (iii) the acyl-CoA thioesterase 2 (EC 3.1.2.20) is TesB or FadM from Escherichia coli; and/or
   (iv) the 1,4-dihydroxy-2-naphtoyl-CoA hydrolase (EC 3.1.2.28) is MenI from Escherichia coli.
40. The recombinant organism or microorganism according to any one of embodiments 37 to 39, wherein the decreased level of an enzyme is due to
   (i) a complete or partial deletion of a gene encoding the respective enzyme in said organism or microorganism; and/or
   (ii) a deletion or an inactivating mutation in a regulatory element of a gene encoding the respective enzyme in said organism or microorganism.
41. The recombinant organism or microorganism according to any one of embodiments 37 to 40, wherein the decreased activity of an enzyme is due to
   (i) an inactivating mutation in a gene encoding the respective enzyme in said organism or microorganism; and/or
   (ii) the addition of an inhibitor of the respective enzyme.
42. The recombinant organism or microorganism according to any one of claims 1 to 41,
   wherein the increased conversion of crotonyl-CoA into butyryl-CoA is due to an increased level and/or activity of a trans-2-enoyl-CoA reductase (EC 1.3.1.44) and/or an enoyl-[acyl-carrier-protein] reductase (EC 1.3.1.9); and/or
   wherein the decreased conversion of crotonyl-[acyl-carrier-protein] and/or crotonyl-CoA into crotonic acid is due to a decreased level and/or a decreased activity of a thioester hydrolase (EC 3.1.2) in said organism or microorganism.
43. The recombinant organism or microorganism according to claim 42, wherein the thioester hydrolase (EC 3.1.2) is a palmitoyl-CoA hydrolase (EC 3.1.2.2) or an acyl-CoA thioesterase 2 (EC 3.1.2.20).
44. The recombinant organism or microorganism according to any one of embodiments 1 to 43 further encoding a ferulic acid decarboxylase.
45. The recombinant organism or microorganism according to embodiment 44, wherein the ferulic acid decarboxylase catalyzes the formation of an alkene from a corresponding carboxylic acid.
46. The recombinant organism or microorganism according to any one of claims 1 to 45, wherein the organism or microorganism is capable of producing a substrate of a ferulic acid decarboxylase.
47. The recombinant organism or microorganism according to any one of embodiments 1 to 46, wherein the organism or microorganism is capable of
   (i) enzymatically converting acetyl-CoA into 3-methylcrotonic acid and/or isobutene; and/or
   (ii) enzymatically converting 3-methylcrotonic acid into isobutene; and/or
   (iii) enzymatically converting cis, cis-muconic acid into 1-3-butadiene; and/or
   (iv) enzymatically converting pentadienoic acid into 1-3-butadiene.
48. The recombinant organism or microorganism according to embodiment 47, wherein the conversion of acetyl-CoA into 3-methylcrotonic acid comprises the steps of:
   (i) enzymatically converting acetyl-CoA into acetoacetyl-CoA,
   (ii) enzymatically converting said produced acetoacetyl-CoA into 3-hydroxy-3-methylglutaryl-CoA,
   (iii) enzymatically converting said produced 3-hydroxy-3-methylglutaryl-CoA into 3-methylglutaconyl-CoA,
   (iv) enzymatically converting said produced 3-methylglutaconyl-CoA into 3-methylcrotonyl-CoA, and
   (v) enzymatically converting said produced 3-methylcrotonyl-CoA into 3-methylcrotonic acid.
49. The recombinant organism or microorganism according to embodiment 48, wherein the recombinant organism or microorganism is capable of enzymatically converting the produced 3-methylcrotonic acid into isobutene.
50. The recombinant organism or microorganism according to embodiment 49, wherein the conversion of 3-methylcrotonic acid into isobutene is catalyzed by a ferulic acid decarboxylase.
51. Use of the recombinant organism or microorganism according to any one of embodiments 1 to 50 for the production of an alkene.
52. The use according to embodiment 51, wherein the alkene is produced by a ferulic acid decarboxylase.
53. The use according to embodiment 51 or 52, wherein the alkene is isobutene or 1,3-butadiene.
54. A method for the production of 3-methylcrotonic acid and/or isobutene, the method comprising a step of culturing a recombinant organism or microorganism as defined in any one of embodiments 48 to 50 in a suitable culture medium under suitable conditions.
55. A method for the production of isobutene, the method comprising the steps of:
   a) producing 3-methylcrotonic acid by culturing a recombinant organism or microorganism as defined in any one of embodiments 48 to 50 in a suitable culture medium under suitable conditions; and
   b) enzymatically converting said produced 3-methylcrotonic acid into isobutene.
56. The method according to embodiment 55, wherein the conversion of 3-methylcrotonic acid into isobutene is catalyzed by a ferulic acid decarboxylase.

### BRIEF DESCRIPTION OF THE DRAWINGS

FIG. 1 shows artificial pathways for isobutene production from acetyl-CoA via 3-methylcrotonic acid. Moreover, enzymatic recycling of metabolites which may occur during the pathway are shown in steps Xa, Xb, XI and XII.

FIG. 2 shows that an FDC variant that has been evolved to specifically decarboxylate 3-methylcrotonic acid cannot undergo the cycloelimination step when bound to crotonic acid, resulting in an irreversible inhibition of the enzyme.

FIG. 3(A) Routes to isobutene via modified mevalonate pathways. Previously published mevalonate diphosphate decarboxylase (MVD) and mevalonate-3-kinase (M3K) produce isobutene via 3-hydroxyisovaleric acid. Fdc1, co-expressed with UbiX, catalyses the decarboxylation of 3-methylcrotonic acid to give isobutene. (B) Fdc decarboxylation reaction mechanism with 3-methylcrotonic acid and common Fdc substrates with a conjugated R-group. First, the 1,3-dipolar cycloaddition of the substrate to prFMNiminium leads to the first pyrrolidine cycloadduct, Int1. Decarboxylation and ring-opening forms the noncyclic alkene adduct Int2. Protonation by a conserved glutamic acid residue yields the second pyrrolidine cycloadduct Int3 followed by cycloelimination to give the product.

FIG. 4: The directed evolution of TaFdc wild-type to TaFdcV with superior isobutene production.

FIG. 5: (A) Overlay of TaFdc wild-type and TaFdcV active sites in two orientations (separated by dotted line). Comparison of TaFdc (B) and TaFdcV (C) binding pockets. The mobile L449 and E292 gate the entrance to the active site and the Q448W mutation narrows the entrance to the active site.

FIG. 6: Crotonic (A) and 3-methylcrotonic (B) acid binding at the active site of TaFdcV (blue), overlayed with TaFdc wild-type (green) modelled with Vina docking. (C) TaFdcV (blue) and TaFdc wild-type (green) overlayed with AnFdc wild-type in complex with alpha-fluorocinnamic acid (PDB: 4ZAB) demonstrating the clash between the M405 residue and the phenyl ring.

FIG. 7: (A) UV-Vis spectra of TaFdcV before and after incubating with 2-butynoic acid. (B) Crystal structure of TaFdcV with prFMN-butynoic adduct (7NF1 [10.2210/pdb7NF1/pdb]). (C) UV-Vis spectra of TaFdcV as is and incubated with crotonic acid. (D) Crystal structure of TaFdcV prFMN-crotonic adduct (7NF2 [10.2210/pdb7NF2/pdb]).

FIG. 8: TaFdcV kinetics with crotonic acid A-gradual split of the 380 nm prFMN peak upon addition of crotonic acid. B—the observed rate of cycloadduct formation based on decrease in the 380 nm peak measured with 1 mM, 10 mM, 20 mM, 40 mM and 50 mM crotonic acid showing a linear relationship.

FIG. 9: Overlay of TaFdcV with (A) AnFdc wild-type; (B) AnFdcI and (C) AnFdcII crystal structures (AnFdc variant residue numbering according to AnFdc).

FIG. 10: 3-methylcrotonate decarboxylation assay with purified enzyme comparing isobutene production as detected by GC by TaFdc variants (wild-type, I, II, V) and AnFdc variants (wild-type, I, II), both with N-terminal His-tags, over 2 and 4 hours. 10 mM 3-methylcrotonate with 0.3 mg/ml enzyme.

FIG. 11: 3-methylcrotonate decarboxylation assay with purified enzyme comparing isobutene production as detected by GC by TaFdc and AnFdc variants, both with N-terminal His-tags, over 2 and 4 h with 10 mM 3-methylcrotonate and 0.3 mg/ml enzyme. Fold increase comparison is shown in FIG. 10.

FIG. 12: A comparison of isobutene in vitro production levels, using E. coli cell lysate. An equal amount of lysate obtained from cells expressing respectively Picrophilus torridus mevalonate 3-kinase (PtM3K), Saccharomyces cerevisiae mevalonate diphosphate decarboxylase (ScMDD) or T. atroviride FdcI/V (in combination with UbiX) was incubated with 50 mM of respectively HIV (3-hydroxyisovalerate)/ATP, PIV (3-phosphonooxy-isovalerate)/ADP or 3-methylcrotonate. Following 4 h incubation at 37 (dark grey) and 50° C. (light grey), the isobutene content of the gas phase was analysed using GC. Under these conditions, TaFdcV mediated isobutene levels exceeded the highest PtM3K/ScMDD levels by ˜50-fold at 37° C. The highest TaFdcV isobutene conversion was approximately 11.5% of the substrate provided.

FIG. 13: (A) Model of the active site of TaFdcV used for DFT calculations based on the crystal structure of TaFdcV with crotonic acid in Int3. Asterisks denote fixed atoms. (B) Overlay of TaFdcV crystal structure with crotonic acid adduct and DFT optimized models of 3-methylcrotonic acid transition state and cycloelimination product isobutene. (C) Overlay of TaFdcV crystal structure with crotonic acid adduct and DFT optimized models of crotonic acid transition state and cycloelimination product propene.

FIG. 14: Contour map of the potential energy (kJ mol-1) landscape for 3-methylcrotonic acid (A) and crotonic acid (B) conversion to isobutene and propene, respectively, from Int3 by TaFdcV, projected transition state is marked by X (C) Zero-point energy corrected potential energy (kJ mol-1) scheme for 3-methylcrotonic and crotonic acid with the Int3 set as 0 and the projected approximate transition state denoted with double daggers (D) overlay of the DFT optimized transition states between Int3 and product for 3-methylcrotonic (C_{α}-C_{1′} and C_{β}-C₄ₐ bond lengths of 1.96 and 2.97 Å, respectively) and crotonic acid (C_{α}-C_{1′} and C_{β}-C₄ₐ bond lengths of 1.95 and 2.77 Å, respectively).

FIG. 15: Strategies for directing metabolic flux away from crotonic acid.

FIG. 16: Strategies for depleting crotonic acid pools.

FIG. 17: MCA and CA production in the culture medium of a strain with fabV overexpression (chromosome and pSC vector) in a 15 L bioreactor (F2250).

FIG. 18: MCA and CA production in the culture medium of a strain with yciA deletion in a 1 L bioreactor (F2253).

FIG. 19: MCA and CA production in the culture medium of a strain with yciA deletion and fabV overexpression in a 1 L bioreactor (F2255).

FIG. 20: MCA and CA production in the culture medium of a strain with fabI overexpression alone in a 1 L bioreactor (F2226).

FIG. 21: MCA and CA production in the culture medium of a strain with tesB deletion and fabI overexpression in a 1 L bioreactor (F2221).

FIG. 22: IBN volumetric productivity from MCA produced in a strain with fabV overexpression (in the chromosome and in the pSC vector, see Example 2A) (F2262).

FIG. 23: Total IBN production from MCA produced in a strain with fabV overexpression (in the chromosome and in the pSC vector, see Example 2A) (F2262).

FIG. 24: Crotonic acid (CA) production in the culture medium of an IBN-producing strain (from sucrose) with fabV overexpression (in the chromosome) (F2238).

FIG. 25: IBN volumetric productivity from sucrose in a strain with fabV overexpression (in the chromosome) (F2238).

FIG. 26: Total IBN production from sucrose in a strain with fabV overexpression (in the chromosome) (F2238).

FIG. 27: Use of an acid-CoA ligase and FabV to convert crotonic acid to butyryl-CoA.

FIG. 28: Use of a CoA-transferase and FabV to convert crotonic acid to butyryl-CoA.

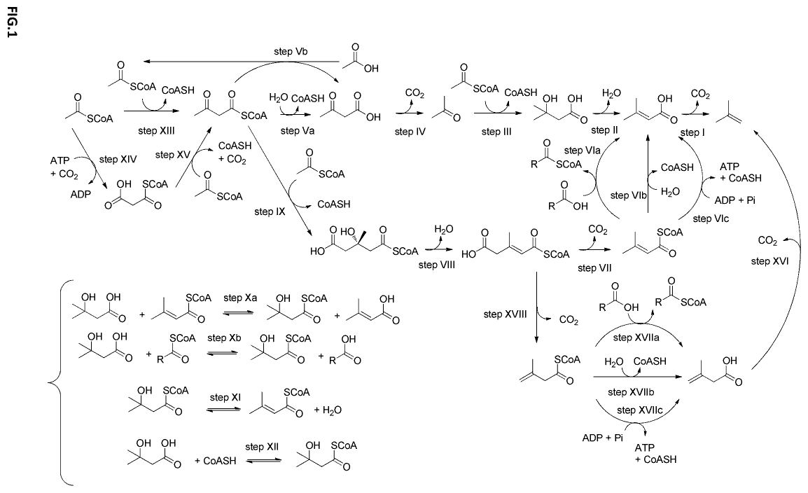

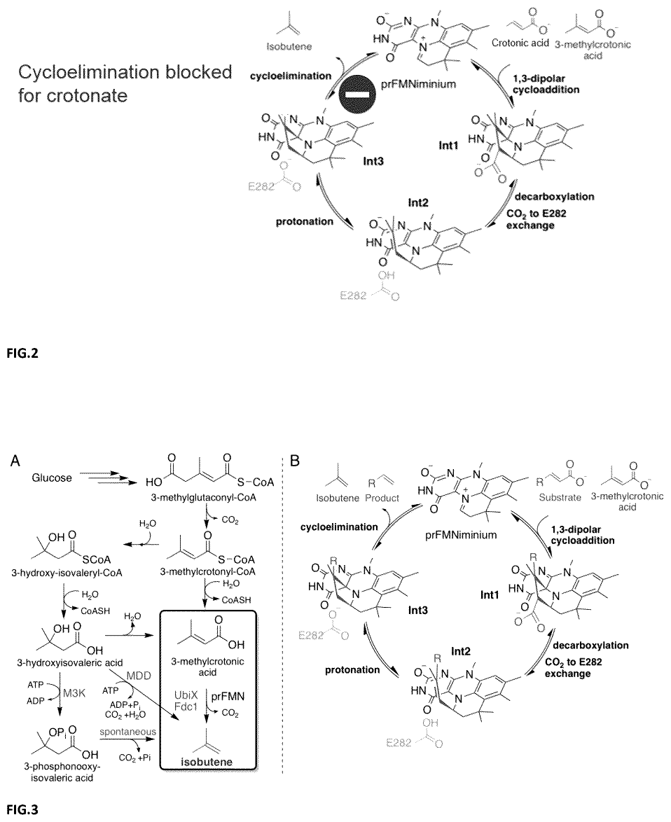

### EXPERIMENTAL EXAMPLES

### Example 1: Identification of Crotonic Acid as Inhibitor of Ferulic Acid Decarboxylases

The irrefutable harmful environmental effects and depleting reserves of fossil fuels have powered an extensive amount of research to seek sustainable alternatives for the production of petrochemicals, including the gaseous alkene isobutene. Due to the favourable reactivity, isobutene is widely used as a building block for fuel additives, rubber, plastic and a broad range of fine chemicals. Over 10 million tons of isobutene are produced every year, primarily by steam cracking crude oil. Low levels of microbial production of isobutene were first detected in the 1980s. More recently, isobutene production via a modified mevalonate (MVA) pathway using mevalonate diphosphate decarboxylase (MVD) to decarboxylate 3-hydroxyisovaleric acid was reported (FIG. 3A). Further studies highlighted a more efficient route using mevalonate-3-kinase (M3K, Picrophilus torridus) that catalyses isobutene formation through an unstable phosphorylated intermediate. The highest reported whole-cell isobutene production rate of 507 pmol min⁻¹ g cells⁻¹ was reached using E. coli engineered with M3K, however, this remains about 10-fold lower than is economically viable. The slow conversion could be surpassed by an alternative route, such as the more direct conversion of methylcrotonyl-CoA to isobutene through a combination of a thioesterase with a non-oxidative decarboxylase. The prenylated flavin (prFMN)-dependent ferulic acid decarboxylases (Fdc) catalyse reversible non-oxidative (de) carboxylation of a range of acrylic acids with extended conjugation. Recently, a reversible 1,3-dipolar cycloaddition mechanism was conclusively shown to underpin catalysis in Aspergillus niger Fdc (AnFdc). First, the cycloaddition of the substrate results in cycloadduct Int1 (FIG. 3B). Decarboxylation occurs concomitantly with ring opening to form Int2. Following the exchange of CO₂ with E282, protonation by E282 results in cycloadduct Int3 that releases the product through cycloelimination. Cycloadduct strain, mediated by a clash between the substrate R group and enzyme residues is key in ensuring reversible 1,3-dipolar cycloaddition]. Recent studies have shown rational engineering of AnFdc can expand substrate scope to include aromatic substrates such as naphthoic acid. However, acrylic acid substrates lacking extended conjugation have rarely been reported in the wider UbiD enzyme family. Arguably, the natural UbiD substrate closest to 3-methylcrotonic acid is trans-anhydromevalonate 5-phosphate (tAHMP), which is decarboxylated by a UbiD decarboxylase from a hyperthermophilic archaeon Aeropyrum pernix in an alternative mevalonate pathway. Both 3-methylcrotonic acid and tAHMP contain a secondary beta carbon and lack extended conjugation, however, the phosphate group in tAHMP may facilitate strain manipulation in cycloadduct intermediates.

Herein, the inventors report on discovery and optimization through directed evolution of Fdc decarboxylation activity with 3-methylcrotonic acid to produce isobutene. The inventors seek to understand how a substrate lacking extended conjugation and bulk can be decarboxylated by Fdc. The inventors discuss the structural basis for an increase in activity and selectivity in Trichoderma atroviride Fdc (TaFdc) evolved by directed evolution. Surprisingly, the optimized variants remain unable to decarboxylate crotonic acid, suggesting that in the case of the substrate 3-methylcrotonic acid the single additional methyl group plays a key role in the cycloelimination process. Computational studies were used to rationalize the effect of the 3-methyl substitution on product formation.

### Initial Screening of Fdc Homologues

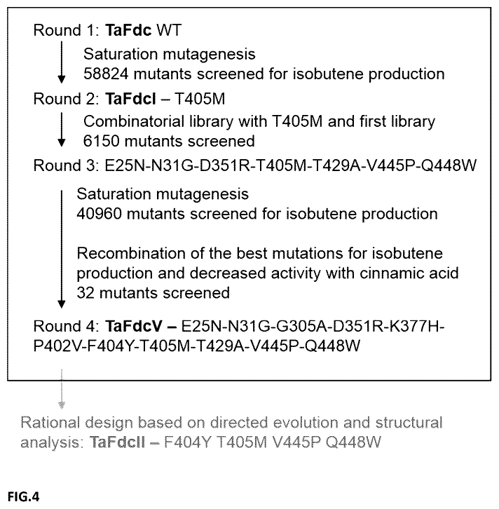

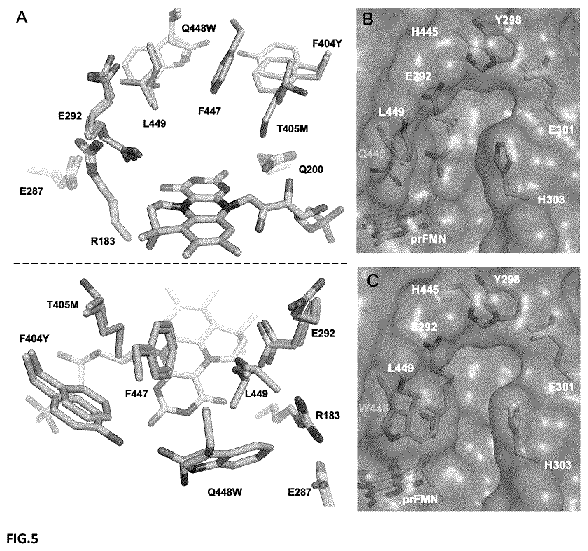

Initial in vivo screening tested 15 UbiD homologues co-expressed with UbiX (E. coli K-12) in E. coli for conversion of 3-methylcrotonic acid into isobutene as detected by gas chromatography. TaFdc exhibited over twice the isobutene production compared to other homologues (Table 4) and a directed evolution approach was taken to generate a variant of TaFdc with superior isobutene production activity and selectivity for 3-methylcrotonic acid over cinnamic acid (FIG. 4). TaFdcI, with a T405M mutation, was the first variant with a considerable increase in isobutene. TaFdcV generated by 4 rounds of evolution has 11 mutations: E25N, N31G, G305A, D351R, K377H, AQ6 P402V, F404Y, T405M, T429A, V445P and Q448W.

TABLE 4
Initial in vivo screening of Fdc variants for wild-type
3-methylcrotonic acid decarboxylation activity. The Fdc
was co-expressed with UbiX in a pETDuet plasmid with Fdc
in MCS1 (with N-terminal 6His-tag) and UbiX in MCS2.

| | |
|---|---|
| FdcUniprot | ppm |

| pETDuet | code | Average | StdDev |
|---|---|---|---|
| CiFdc-UbiX | A0A0D2AQI6 | 16.4 | 0.8 |
| SmFdc-UbiX | M3DF95 | 5.9 | 0.7 |
| PrFdc-UbiX | W6QKP7 | 9.2 | 1.5 |
| ApFdc-UbiX | A0A0F0IHE5 | 108.9 | 8.4 |
| NfFdc-UbiX | A1DCG7 | 108.0 | 7.4 |
| CcFdc-UbiX | W9YNA8 | 125.2 | 9.2 |
| PcFdc-UbiX | A0A0G4P429 | 19.8 | 2.9 |
| AnFdc-UbiX | A2QHE5 | 13.2 | 3.1 |
| BfFdc-UbiX | M7THT1 | 7.8 | 1.1 |
| PpFdc-UbiX | A0A094IED9 | 8.8 | 2.4 |
| CsFdc-UbiX | A0A0D2DPQ1 | 13.4 | 1.1 |
| CpFdc-UbiX | W9WWR1 | 18.0 | 2.2 |
| CdFdc-UbiX | B9WJ66 | 4.8 | 1.3 |
| ScFdc-UbiX | Q03034 | 17.6 | 1.3 |
| TaFdc-UbiX | G9NLP8 | 330.0 | 29.1 |
| UbiX alone | NA | 3.3 | 1.4 |
| empty plasmid | NA | 3.2 | 1.2 |

### Characterization of TaFdc and TaFdcV

TaFdc wild-type and TaFdcV with an N-terminal hexa-histidine tag were co-expressed with E. coli K-12 UbiX in E. coli and purified with Ni-NTA resin. UV-Vis spectra of both purified proteins exhibit a distinct peak at 380 nm, thought to correspond to the cofactor active form prFMNiminium. ESI-MS confirmed the presence of prFMNiminium in both enzyme variants. The shape of the 380 nm peak and cofactor content (assessed by the ratio of absorbances at 280 and 380 nm) varied from batch to batch.

TaFdc showed decarboxylation activity with cinnamic and sorbic acid, with rates k_{obs}=7.2±0.3 and 3.2±0.3 s, respectively (reported for a batch with a 380:280 nm ratio of 0.067). These values are comparable to those reported for AnFdc. In contrast, the TaFdcV variant showed compromised activity with sorbic acid (k_{obs}=0.33±0.03 s) and no activity was detected with cinnamic acid. When exposed to light, TaFdc sorbic acid decarboxylation activity steadily deteriorates with a half-life of 1 h compared to enzyme stored in dark. This is consistent with Fdc light-sensitivity as described previously. Upon irradiation with a 405 nm LED lamp, the characteristic 380 nm peak in the UV-visible absorbance spectra of TaFdc and TaFdcV irreversibly splits to peaks at 365 and 425 nm.

Incubation of both TaFdc and TaFdcV with 3-methylcrotonic acid triggered a change in the protein UV-Vis spectrum to reveal peaks at 340 and 425 nm, suggestive of a covalent substrate: prFMN adduct accumulating under turnover conditions. Following a desalting step, the spectrum returns to the as-isolated 380 nm single feature, confirming that a long-lived, inhibitory covalent complex with 3-methylcrotonic acid is not formed. Incubation of 80 μmol TaFdcV with 10 mM 3-methylcrotonic acid led to a complete shift in the corresponding UV-Vis spectrum. In contrast, the wild-type TaFdc required prolonged incubation with 50 mM 3-methylcrotonic acid to achieve full spectral conversion, suggesting a substantially higher K_{D}
and/or adduct formation rate for the wild-type enzyme. An ESI-MS spectrum of the desalted sample showed peaks corresponding to both prFMNiminium and a putative Int3 prFMN cycloadduct with 3-methylcrotonic acid. This may be due to a small proportion of 3-methylcrotonic acid remains bound to prFMN as Int3, suggesting Int3 elimination is the rate limiting step, or that a proportion of the Int3 species has irreversibly isomerized to a more stable conformation.

In order to assess the scope for activity with acrylic acids lacking extended conjugation, TaFdc and TaFdcV were incubated with trans-2-pentenoic and trans-2-hexenoic acid, compounds that have previously been reported to undergo some AnFdc-mediated decarboxylation. UV-Vis absorbance spectra indicated that TaFdc bound both acids, whereas the TaFdcV variant preferred the smaller pentenoic acid and required higher concentrations to fully bind hexenoic acid. In contrast to samples incubated with 3-methylcrotonic acid, the UV-Vis spectra of samples incubated with pentenoic or hexenoic acid were unaffected by a desalting step, indicating that pentenoic and hexenoic acid irreversibly binds to TaFdc/TaFdcV. Quantitative GC assay indicates that pentene production from hexenoic acid by AnFdc is limited to a single turnover.

### Crystal Structures of TaFdc and TaFdcV Reveal Mutation Impact on the Substrate-Binding Pocket

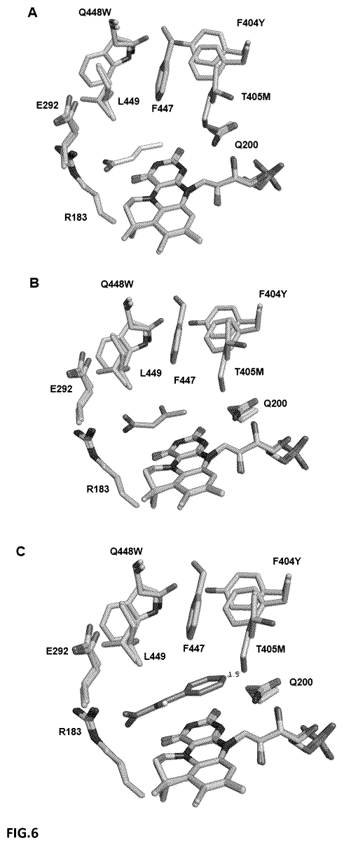

In order to understand how TaFdcV mutations aid in isobutene production, crystal structures of TaFdc and TaFdcV were solved at a resolution of 1.74 and 1.89 Å, respectively. An overlay of the wild-type and the variant crystal structures shows that the key residues F447, Q200 and the catalytic network of E287-R183-E292 are unaffected by the mutations (FIG. 5A). The T405M mutation is located at the active site, extending towards the space above the prFMN uracil ring while the Q448W and F404Y mutations are situated in the second shell from the active site. The E292 residue side chain occupies ‘up’ and ‘down’ conformations, while weak electron density suggests a high degree of mobility for the L449 side chain. The mobile E292 and L449 gate access to the active site (FIG. 5B) while the Q448W mutation in TaFdcV narrows the binding pocket (FIG. 5C). The T405M and Q448W mutations are likely to be responsible for the increased selectivity for 3-methylcrotonic acid in TaFdcV by enhancing the substrate/active site shape complementarity, blocking access to larger substrates (FIG. 6). While comparison of TaFdc and TaFdcV crystal structures reveals the basis for increased selectivity in the evolved enzyme, it is not immediately clear why 3-methylcrotonic acid can yield isobutene from Int3.

Formation of Stable Cycloadducts with Inhibitors

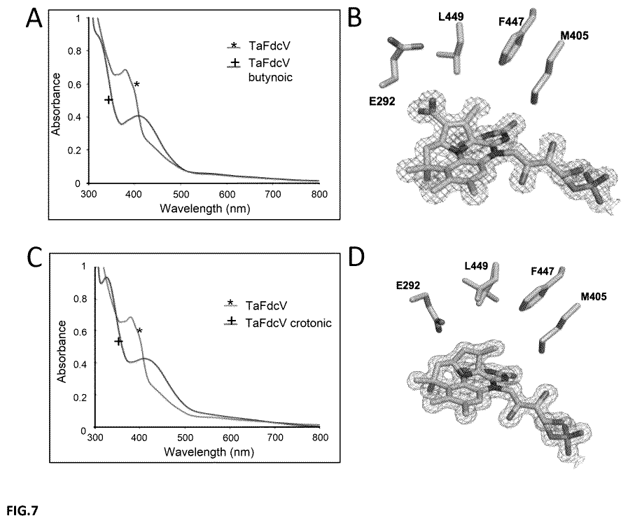

The effects of crotonic and 2-butynoic acid on TaFdcV were studied to determine whether the mutations that increase in 3-methylcrotonic acid turnover also affected activity with related compounds. Incubation of TaFdcV with 2-butynoic and crotonic acid led to the familiar split of the 380 nm prFMN peak in the UV-Vis spectrum (FIGS. 7A and D), similar to 3-methylcrotonic acid. However, as previously observed with pentenoic and hexenoic acid, the spectrum did not recover the following desalting, suggesting that a covalent inhibitory adduct is formed. Similar trends were observed with TaFdc, however, incubation at higher inhibitor concentration was required to drive changes in the UV-Vis spectrum.

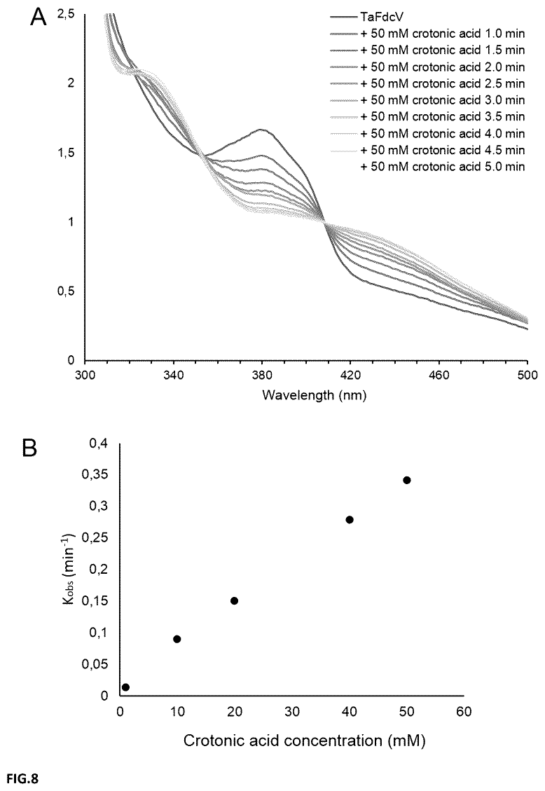

Upon addition of crotonic acid, a gradual shift in UV-Vis spectrum occurs over minutes, allowing estimation of adduct formation rate (FIG. 8A). The observed rate remains first order with respect to crotonic acid, with k_{obs}=0.34±0.03 min at the highest concentration tested (50 mM) (FIG. 8B). In contrast, a similar shift in UV-Vis spectrum upon addition of the substrate 3-methylcrotonic acid occurs rapidly within seconds, and at substantially lower 3-methylcrotonic acid concentrations. This suggests that crotonic acid adduct formation is hindered by a higher K_{D} and/or slower rate of cycloaddition.

ESI-MS and co-crystallization studies confirmed that the TaFdcV 2-butynoic acid adduct stalls as Int1, while the TaFdcV crotonic acid adduct undergoes decarboxylation to stall at the Int3 species (FIG. 7). No decarboxylation of the Int1 with 2-butynoic acid was detected, even in 1-month-old crystals.

AnFdcII with Three Point-Mutations has an Identical Active Site Conformation to TaFdcV

To further understand how the architecture of the active site affects the decarboxylation of 3-methylcrotonic acid, corresponding key mutations from TaFdcV were introduced in AnFdc. AnFdc has been established as a model system due to the fact that it readily yields atomic resolution crystal structures. Two variants were studied: AnFdc T395M (AnFdcI) and the triple mutant AnFdc T395M R435P P438W (AnFdcII). Overlay of the AnFdc wild-type and TaFdcV crystal structures reveals a downward shift of the Y404 residue in TaFdcV in the secondary shell compared to the corresponding Y394 in AnFdc (FIG. 9A). The Y394 residue is unaffected in the AnFdcI variant compared to wild-type (FIG. 9B). In contrast, the active site of the AnFdcII variant matches that of TaFdcV in the conformation of Y394 and M395 (FIG. 9C).

As expected, neither AnFdcI nor AnFdcII were active with cinnamic acid, likely due to a clash between the substrate phenyl ring and M395. While binding of crotonic acid in AnFdc wild-type cannot be detected by the UV-Vis spectra over 2-h incubation, both mutants AnFdcI and AnFdcII readily bind the inhibitor, evident from UV-Vis spectra, demonstrating increased selectivity towards smaller substrates.

### TaFdcII has Comparable Isobutene Production Activity to TaFdcV

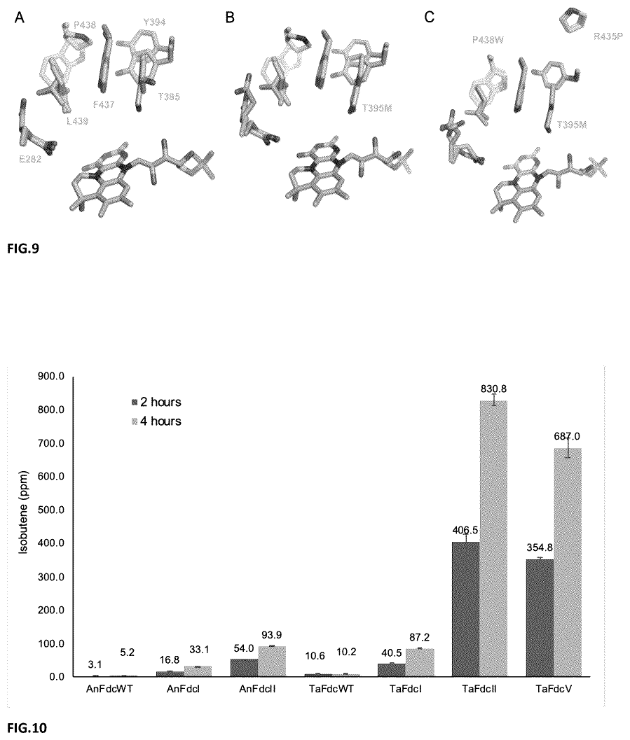

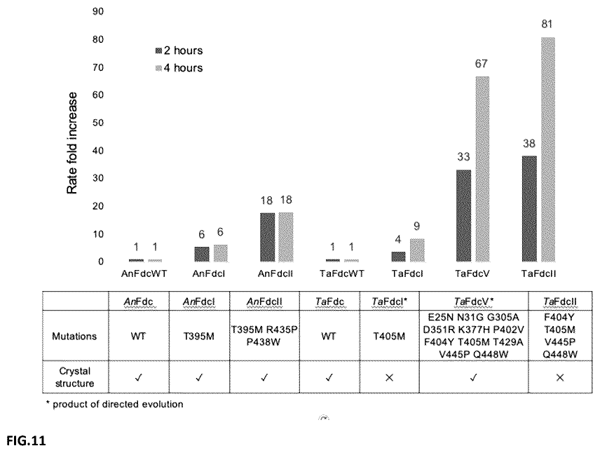

Selected TaFdc variants (wild-type, TaFdcI i.e. T405M, TaFdcV, TaFdcII), and AnFdc variants (wildtype, AnFdcI, AnFdcII) were purified and assayed for isobutene production. TaFdcII (F404Y, T405M, V445P, Q448W) was created by rational design based on the structural analysis of TaFdc wild-type, TaFdcV and AnFdcII. A comparison of the isobutene titre obtained following 2 and 4 h incubation revealed TaFdcI and AnFdcI produced 4-9 times the amount of isobutene compared to the wild-type enzymes. Additional mutations in AnFdcII and TaFdcII led to a substantial further increase of 18 and 81 fold, respectively, in isobutene production (FIG. 10). Surprisingly, the in vitro titer obtained with TaFdcII was slightly higher than the corresponding TaFdcV levels obtained (FIG. 11). Thus, the 4 point mutations in TaFdcII (F404Y, T405M, V445P, Q448W) and 3 point mutations in AnFdcII (T395M, R435P, P438W), that create an active site architecture identical to TaFdcV (FIG. 9), appear largely responsible for the increased isobutene activity compared to wild-type TaFdc and AnFdc.

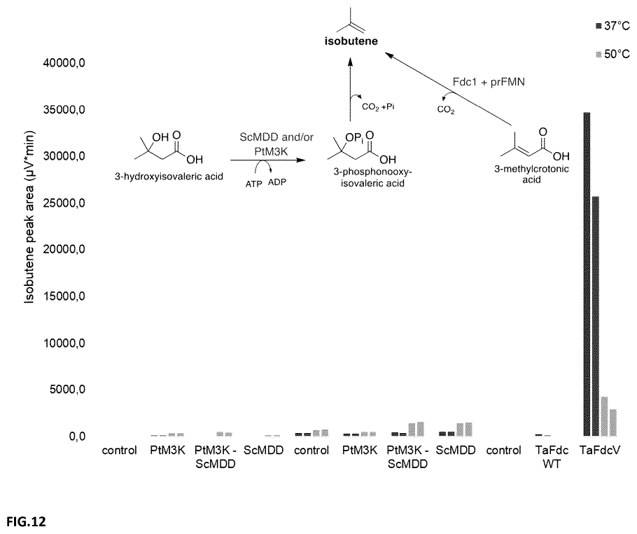

While AnFdc wild-type was included in the initial UbiD screen, the AnFdc wild-type was 90 times lower in activity in vivo compared to TaFdc. Hence, AnFdc was not selected for further directed evolution, despite having comparable in vitro activity to TaFdc. The disparate and lower activity in vivo might be attributed to AnFdc-specific inhibition by metabolites such as phenylacetaldehyde. An initial comparison of in vitro isobutene production levels using crude cell lysate from cells expressing TaFdc variants with those expressing MVD and/or M3K reveals a ^{˜}50-fold increase is observed for TaFdcV compared to MVD/M3K levels (FIG. 12). This demonstrates that the evolved TaFdcV is vastly superior in catalysing the decarboxylative step compared to the previously described enzyme systems.

### Computational Studies Reveal a Mechanistic Basis for Isobutene Production

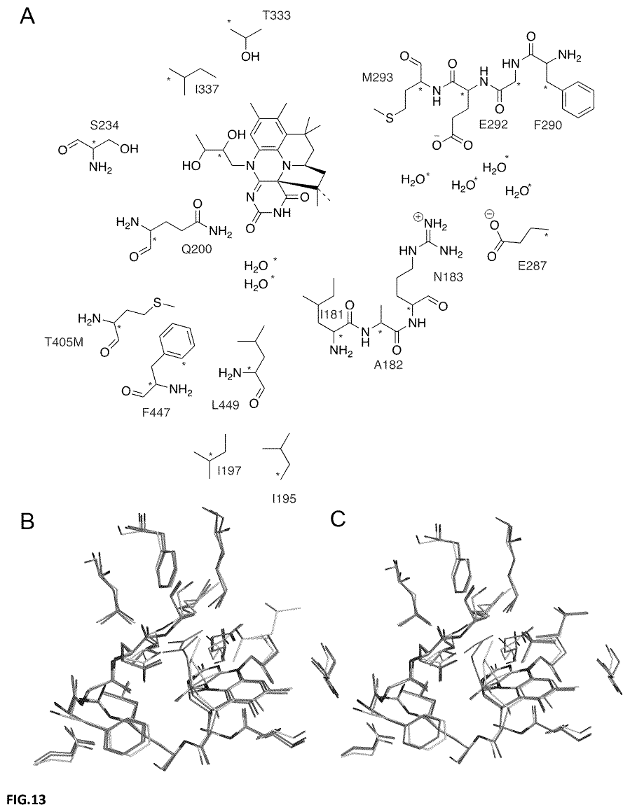

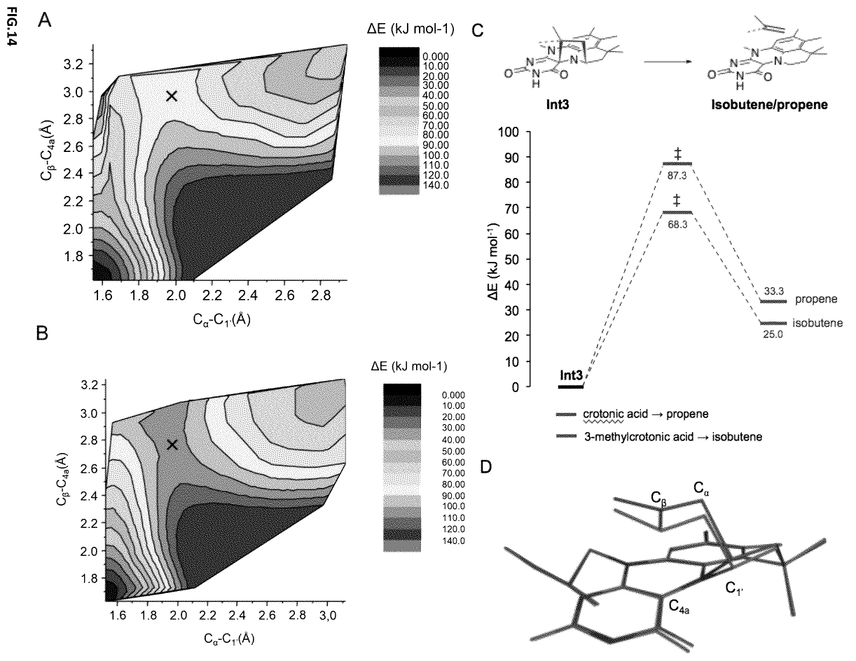

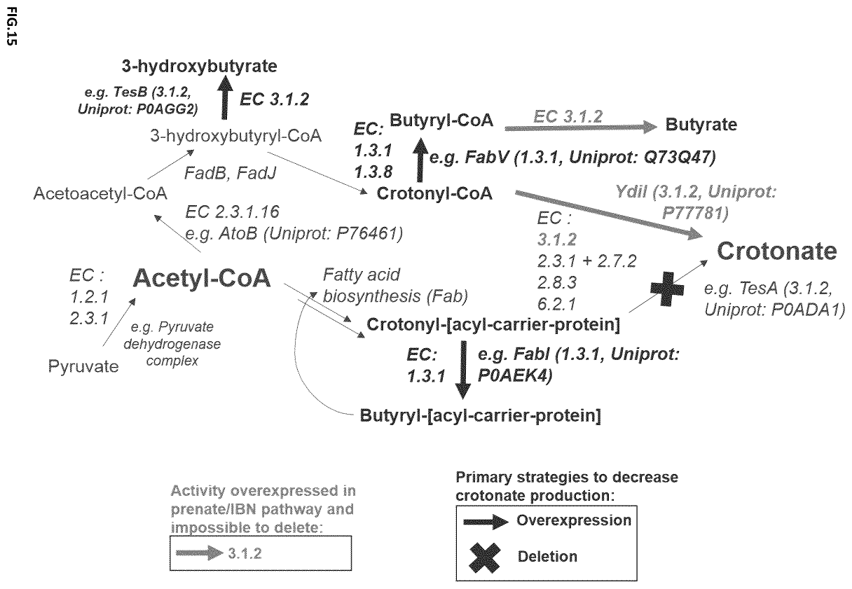

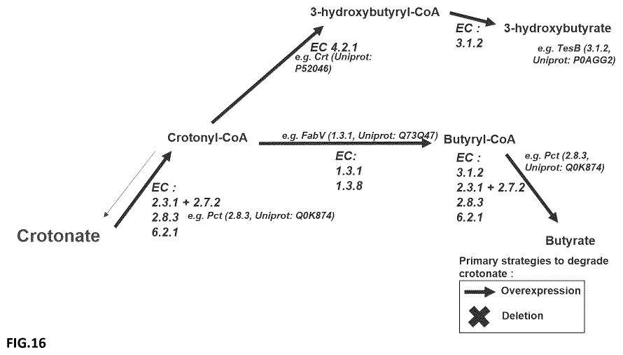

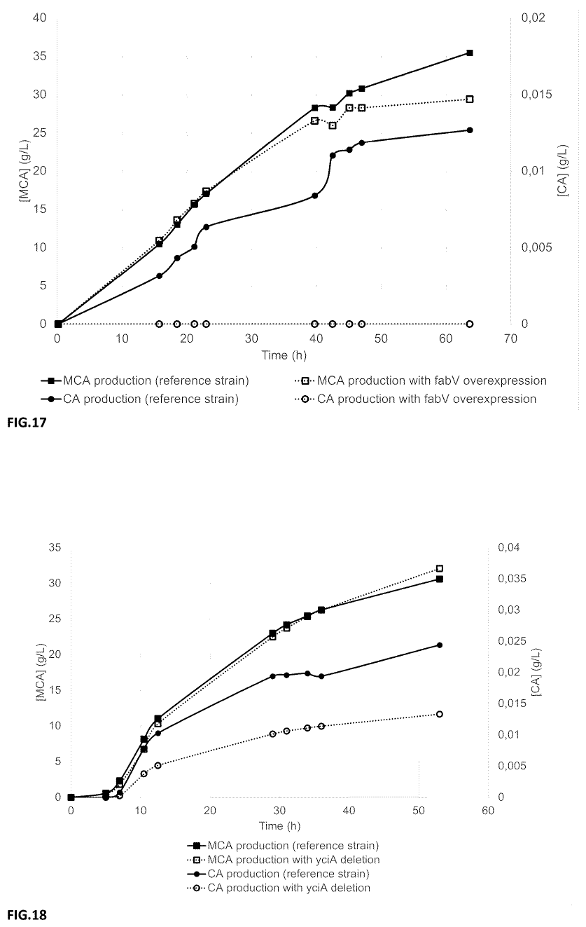

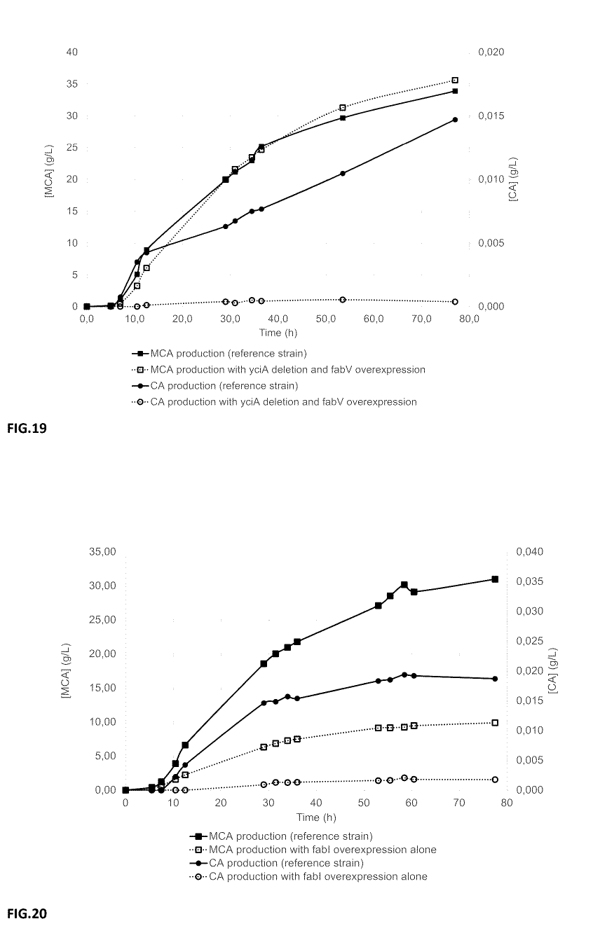

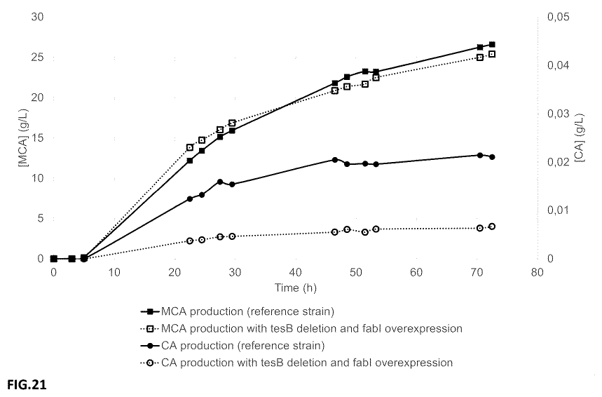

It is curious that a single methyl group difference, as occurs between crotonic acid and 3-methylcrotonic acid, determines whether the compound is a substrate or inhibitor for the evolved Fdc variants. The marked influence of the additional methyl group on Int3 cycloelimination suggests this step may proceed via a cationic or radical beta carbon stabilized through additional hyperconjugation effects. A density functional theory (DFT) active site ‘cluster’ model (FIG. 13) was used to investigate why 3-methylcrotonic acid is decarboxylated and eliminated by TaFdcV in contrast to crotonic acid.

The potential energy surface for the cycloelimination of Int3 to the non-covalent product complex was computed for both crotonic acid and 3-methylcrotonic acid by varying the C_{α}-C₁ and C_{β}-C₄ₐ bond lengths (FIG. 14). These suggest that 3-methylcrotonic acid undergoes a more asynchronous elimination, with the transition state C_{α}-C₁ and C_{β}-C₄ₐ bond lengths of 1.96 and 2.97 Å, respectively, compared to 1.95 and 2.77 Å for crotonic acid, respectively. This is linked to an increased charge separation occurring between the C and prFMN for the 3-methylcrotonic acid compared to crotonic acid (Tables 5 and 6), possibly affected by additional hyperconjugation in the case of 3-methylcrotonic acid. The release of propene from crotonic acid Int3 cycloadduct is more endothermic by ^{˜}8 KJ mol-1 and has a higher energy barrier by 19 kJ mol⁻¹ compared to the release of isobutene from Int3 with 3-methylcrotonic acid. If the activation entropy is similar for the two reactions then the transition state energy difference translates to a ^{˜}2200 slower rate for the release of propene from Int3 at 293 K, explaining the lack of crotonic acid turnover under conditions tested.

TABLE 5
Natural charge analysis for DFT models with 3-methylcrotonic acid

summed natural charges

| | | | | Prod − | TS − |
|---|---|---|---|---|---|
| moiety | Int3 | TS | Product | Int3 | Int3 |
| substrate (3- | 0.11 | 0.26 | −0.02 | −0.13 | 0.15 |
| methylcrotonic | | | | | |
| acid) | | | | | |
| Cα | −0.47 | −0.53 | −0.49 | −0.02 | −0.06 |
| Cβ | −0.03 | 0.15 | −0.05 | −0.02 | 0.18 |
| C4a | 0.01 | −0.07 | −0.05 | −0.06 | −0.09 |
| N5 | −0.46 | −0.36 | −0.30 | 0.16 | 0.11 |
| C1′ | −0.04 | 0.08 | 0.15 | 0.19 | 0.12 |
| prFMN | 0.01 | 0.05 | 0.05 | 0.05 | 0.05 |
| isoalloxazine | −0.62 | −0.89 | −0.69 | −0.07 | −0.27 |
| additional | 0.25 | 0.43 | 0.51 | 0.26 | 0.18 |
| prenyl carbons | | | | | |
| ‘tail’ | 0.26 | 0.25 | 0.26 | −0.01 | −0.01 |
| Cγ | −0.69 | −0.72 | −0.69 | 0.00 | −0.03 |
| Cγ′ | −0.70 | −0.73 | −0.70 | 0.00 | −0.03 |

TABLE 6
Natural charge analysis for DFT models with crotonic acid

| | summed natural charges | | | | |
|---|---|---|---|---|---|
| | | | | Prod − | TS − |
| moiety | Int3 | TS | Product | Int3 | Int3 |
| substrate | 0.12 | 0.21 | −0.02 | −0.14 | 0.09 |
| (crotonic | | | | | |
| acid) | | | | | |
| Cα | −0.47 | −0.52 | −0.49 | −0.02 | −0.05 |
| Cβ | −0.23 | −0.10 | −0.24 | −0.01 | 0.14 |
| C4a | 0.01 | −0.06 | −0.06 | −0.06 | −0.06 |
| N5 | −0.47 | −0.35 | −0.29 | 0.18 | 0.12 |
| C1′ | −0.04 | 0.06 | 0.17 | 0.21 | 0.11 |
| prFMN | 0.03 | 0.05 | 0.06 | 0.03 | 0.02 |
| isoalloxazine | −0.62 | −0.83 | −0.70 | −0.08 | −0.21 |
| additional | 0.26 | 0.42 | 0.53 | 0.27 | 0.16 |
| prenyl | | | | | |
| carbons | | | | | |
| ‘tail’ | 0.27 | 0.25 | 0.25 | −0.01 | −0.01 |
| Cγ | −0.69 | −0.72 | −0.71 | −0.02 | −0.03 |
| H | 0.26 | 0.24 | 0.23 | −0.03 | −0.02 |

The Limit of prFMN-Dependent (De)Carboxylation by UbiD Enzymes

Directed evolution of TaFdc to TaFdcV resulted in a marked increase in activity with 3-methylcrotonic acid. Surprisingly, the evolved mutant remained unable to convert crotonic acid to the corresponding propene. This contrasts with previous evolved studies aimed at expanding the AnFdc substrate repertoire to include (hetero) aromatic compounds. In this case, the evolution of activity against heteroaromatic bicyclic compounds yielded a broad specificity variant able to convert even naphthoic acid. It is thus possible that 3-methylcrotonic acid represents a limit for bona fide UbiD-substrates, indicating that prFMN-dependent catalysis requires more than an α,β-unsaturated acrylic acid (i.e. a secondary C carbon) to yield reversible cycloelimination.

Indeed, crotonic acid readily forms irreversible adducts with (evolved) Fdc that proceed to the last step prior to product formation. In the case of smaller substrates such as (3-methyl) crotonic acid, the scope for enzyme-induced strain as a tool to optimize the energy landscape is minimal. In this case, cycloelimination of isobutene appears feasible at ambient conditions whereas propene production is not. Computational studies provide a rationale behind these observations, suggesting a ^{˜}2200 fold slower rate for the release of propene from Int3. Thus, further optimization of isobutene production and future evolution of propene producing Fdc variants will need to focus on the energetics of the hydrocarbon elimination step.

### Methods

### In Vivo Isobutene Assay

In vivo screenings were carried out on a 96-well plate (DW96, 2.2 mL wells, sealed with a foil sheet). TaFdc and other UbiD homologues were co-expressed with Ubix (E. coli, K-12) in a petDuet vector (UbiD in MCS1 and UbiX in MCS2) in E. coli (BL21, DE3). Isobutene production from 0.4 mL reaction mix with 10 mM 3-methylcrotonic acid was detected from the headspace by gas chromatography. The GC method consisted of 100 μL of headspace with a split ratio of 10 injected to RTX-1 column (15 m, 0.32 mm internal diameter, 5 μm film thickness, from RESTEK 10178-111) using nitrogen as a carrier gas (1 mL/min flow rate). The oven temperature was held at 100° C. and the injector and detector were maintained at 250° C. Isobutene was calibrated at 1000, 5000 and 10,000 ppm with standards from Messer.

### Mutagenesis

Point mutations (TaFdcI, TaFdcII, AnFdcI, AnFdcII) were generated with a Q5 mutagenesis kit from New England Biolabs. Primers were designed with NEBaseChanger (New England Biolabs). The presence of the point mutation was confirmed by sequencing (Eurofins).

### Protein Expression

A pETDuet-1 vector containing genes for T. atroviride Fdc (with an N-terminal 6-histidine affinity tag) and UbiX (E. coli, K-12) was transformed into BL21 (DE3) competent cells following the manufacturer's protocol (Novagen). A colony was inoculated into Lysogeny Broth (supplemented with 100 μg/mL ampicillin) and incubated by shaking overnight at 37° C. 5 mL of LB culture was inoculated into 1 L of Terrific Broth (TB, Formedium), supplemented with 100 μg/mL ampicillin. The culture was incubated by shaking at 37° C. until the optical density of 0.6-0.8. The cells were induced with 0.4 mM isopropyl β-D-1-thiogalactopyranoside (IPTG) and supplemented with 0.5 mM MnCl₂. The cultures were incubated by shaking at 18° C. for 24 h. The cells were harvested by centrifugation (10 min, 8939×g) and frozen.

### Protein Purification

Frozen cells were supplemented with EDTA-free protease inhibitor mixture (Roche Applied Science), lysozyme, DNAse, and RNAse (Sigma) and resuspended (50 mM HEPES, 300 mM KCl, pH 6.8). The cells were lysed by sonication on ice (Bandelin Sonoplus sonicator, TT13/F2 tip, 30% power with 20 s on/40 s off for 15 min) and centrifuged (1 h, 174,000-185,500×g,

Beckman Optima LE-80k ultracentrifuge, TiS0.2 rotor). The cell-free extract was loaded on Ni-NTA resin, washed with 4 column volumes of 40 mM imidazole buffer (40 mM imidazole, 50 mM HEPES, 300 mM KCl, pH 6.8) and eluted in 1 mL fractions with 250 mM imidazole buffer (250 mM imidazole, 50 mM HEPES, 300 mM KCl, pH 6.8). Fractions containing protein were combined and desalted into 25 mM HEPES, 150 mM KCl, pH 6.8. Exposure to light was minimized by covering with foil and using black Eppendorf tubes.

### Cycloadduct Formation

TaFdcV (500 μL, 0.45 mM protein, 25 mM HEPES, 150 mM KCl, pH 6.8) was incubated with crotonic or 2-butynoic acid. The formation of the prFMN-crotonic acid cycloadduct was followed by UV-Vis spectroscopy and additional acid were added until full conversion (complete loss of the 380 nm peak). The protein was desalted to 25 mM HEPES, 150 mM KCl, pH 6.8 and plated for crystal trials.

### Crystallization and X-Ray Structure Determination

Crystallization was performed by sitting-drop vapour diffusion. Screening of 0.3 μL of 1 mg/ml TaFdcV in 25 mM HEPES, 150 mM KCl, pH 6.8, and 0.3 μL of reservoir solution at 4° C. resulted in a number of hits in the BCS plate from molecular dimensions. Seed stocks were used to reproduce TaFdc wild-type crystals and co-crystals with 2-butynoic and crotonic acids in the BCS plate. Crystals were cryoprotected with PEG200 and flash-frozen in liquid nitrogen. AnFdc wild-type and variants were crystallized in 0.2 M potassium thiocyanate, Bis-Tris propane 6.5, 20% w/v PEG 3350 at 4° C. Diffraction data were collected at Diamond beamlines and processed using the CCP4 suite version 7.1. Phaser MR version 2.8.3 was used to perform molecular replacement using 4ZA4 [https://doi.org/10.2210/pdb4ZA4/pdb] as a model. Refinement was carried out with REFMAC5 and manual rebuilding in COOT version 0.9.5. Ligand definitions and coordinates were generated with AceDRG.

### In Vitro Isobutene Assay Comparing TaFdc and AnFdc Variants

All variants were grown in BL21 (DE3) cells with a pETDuet plasmid with the Fdc variant (N-terminal 6-His-tag) in the first multiple cloning site and UbiX (untagged, wild-type from E. coli K12) in second multiple cloning site. The cells were grown in a ZYM-5052 autoinducing medium (30° C. for 6 h, followed by 18° C. for 24 h). The Fdc variants were purified with Protino Ni-IDA column and stored at −80° C. (in 50 mM Tris-HCl pH 7.5, 1 mM MnCl₂, 20 mM NaCl, 200 mM KCl, 10% glycerol). Decarboxylation of 3-methylcrotonate was set up in triplicates in 50 mM Tris-HCl PH 7.5, 1 mM MnCl₂, 20 mM NaCl, 200 mM KCl with 10 mM 3-methylcrotonate and 0.3 mg/ml enzyme in DW384 plates (40 μL per well, sealed with foil sheet). Isobutene production was measured from headspace by gas chromatography after 2 and 4 h.

### In Vitro Isobutene Assay Comparing TaFdc, ScMVD and PtM3K

An equal amount of E. coli cells containing either empty pETDuet (as control) or one of the following plasmids: pETDuet TaFdc_UbiX, pETDuet TaFdcV_UbiX, pETDuet PtM3K (P. torridus mevalonate 3-kinase, Uniprot: Q6KZB1), pETDuet ScMVD (S. cerevisiae MVD, Uniprot: P32377) or pETDuet PtM3K-ScMVD, were lysed in 50 mM Tris-HCl pH 7.5, 20 mM KCl, 2 mM MgCl₂, 1 g/L lysozyme, 0.03 g/L DNAse for 1 h at 37° C. A total of 150 μL of lysate was transferred to a 2 mL GC-vial and MgCl₂ (10 mM final concentration) was added. Substrates were added to 50 mM final concentration and 200 μL total volume, and consisted of either 3-hydroxyisovalerate/ATP, 3-phosphonooxy-isovalerate/ADP or 3-methylcrotonate. Following 4 h of incubation at either 37 or 50° C., the reaction mixture was inactivated by incubation at 90° C. for 5 min. GC analysis of the gas phase was carried out as described above to determine isobutene levels produced. All reactions were carried out in duplicates.

### DFT Calculations

TaFdcV active site cluster model with crotonic AQ7 (365 atoms) and 3-methylcrotonic acid (368 atoms) was built based on the TaFdcV crystal structure with crotonic acid bound as Int3 adduct and modelled at the B3LYP/6-31 G (d,p) level of theory with the D3 version of Grimme's dispersion with Becke-Johnson damping and a generic polarizable continuum with E=5.7 using the polarizable continuum model [25]. C_{α}-C₁ and C_{β}-C₄ₐ bonds were both fixed for any single DFT optimization and substrate release was modelled using Gaussian 09 revision D.01. by lengthening one bond by 0.05 Å at a time, resulting in a 3D energy landscape consisting of ˜900 DFT optimized models (for 3-methylcrotonic acid).

### Example 2: In Vivo 3-Methylcrotonic Acid (MCA) Production from Acetyl-CoA with a Decreased Pool of Crotonic Acid (CA)

This Example shows the production of 3-methylcrotonic acid by a recombinant E. coli strain which expresses exogenous genes, thereby constituting the 3-methylcrotonic acid pathway.

Like most microorganisms, E. coli converts glucose into acetyl-CoA. The enzymes used in this study to convert acetyl-CoA into 3-methylcrotonic acid are summarized in the following.

Expression of a 3-methylcrotonic Acid Biosynthetic Pathway in E. coli

The following genes were codon-optimized for the expression in E. coli and synthesized by GeneArt (Life Technologies):
thl (thiolase) from Clostridium acetobutylicum (Uniprot Accession number P45359)
mvaS (Hydroxymethylglutaryl-CoA synthase) from Schizosaccharomyces pombe (Uniprot Accession number P54874)
ech (enoyl-CoA hydratase) from Pseudomonas sp. (Uniprot Accession number K9NHK2)
aibA and aibB that code for the two subunits of 3-methylglutaconyl-CoA decarboxylase from Myxococcus hansupus (Uniprot Accession number A0A0H4WQB1 and A0A0H4WWJ4)
menI (ydiI) (1,4-dihydroxy-2-naphthoyl-CoA hydrolase) from Escherichia coli (strain K12) (Uniprot Accession number P77781)

An expression vector containing the origin of replication of pSC101 (reference: http://www.ncbi.nlm.nih.gov/pmc/articles/PMC320470/) was used for the expression of the genes: mvaS, ech, aibA, aibB, ydiI according to the procedure described in WO2017/085167, Example 12, except for the integration of the FDC1 (UbiD-like) gene, and for thl (thiolase) expression the strain MG1655 was modified by integration of the thl gene from Clostridium acetobutylicum into the ssrs locus.

### Genetic Modifications to Decrease the Crotonic Acid Pool

To decrease the crotonic acid pool in this 3-methylcrotonic acid-producing strain additional genetic modifications were carried out in the chromosome (genome editing: gene deletions, gene integrations) or in expression vectors (gene cloning). The genome editing method was described by Datsenko and Wanner (Datsenko K A, Wanner B L. One-step inactivation of chromosomal genes in Escherichia coli K-12 using PCR products. Proc Natl Acad Sci USA. 2000 Jun. 6; 97(12):6640-5. doi: 10.1073/pnas.120163297. PMID: 10829079; PMCID: PMC18686.). DNA cloning was carried out with type II restriction enzymes.

### A/FabV Overexpression Alone:

For this Example 2A, fabV from Treponema denticola (EC 1.3.1.44) (Uniprot Accession No: Q73Q47) was integrated into the mgsA locus of E. coli MG1655 following the transformation with an integration vector linearized by restriction digestion. As described by Datsenko and Wanner the integration vector contained a conditional origin of replication (oriRγ) requiring the trans-acting π protein (the pir gene product) for replication (e.g. EC100D pir+ cells, Lucigen). The integration vector contained the following parts assembled by successive cloning steps and stated here in the 5′ to 3′ order:
homology 1 DNA sequence (for recombination): the 987 bp which are upstream of the mgsA gene in the chromosome (containing the mgsA promoter)
PN25 constitutive promoter
mgsA truncated gene (the last 459 bp of the mgsA gene, including the stop codon)
RBST7 (ribosome-binding site)
fabV from Treponema denticola (EC 1.3.1.44) (Uniprot Accession No: Q73Q47), codon-optimized for the expression in E. coli and synthesized by GeneArt (Life Technologies)
PN25 terminator
FRT-SpecR-FRT (selection cassette, excisable with the pCP20 plasmid containing the recombinase flippase FLP)
homology 2 DNA sequence (for recombination): the 891 bp which are downstream of the mgsA gene in the chromosome (containing the 3′ end of the helD gene, which is in opposite direction to the mgsA gene)

To further increase fabV expression, an additional copy of the gene was cloned in the expression vector pSC101 with a P7 constitutive promoter, a RBST7 ribosome-binding site and a PN25 terminator. The resulting 3-methylcrotonic acid (MCA)-producing strain with a decreased pool of crotonic acid (CA) was tested in a 15L bioreactor in fermentation no F2250.

### B/YciA Deletion Alone:

For this Example 2B, yciA from Escherichia coli (EC 3.1.2.20) (Uniprot Accession No: P0A8Z0) was deleted in the chromosome following transformation with a PCR product containing 50 bp homology sequences (for recombination) flanking an excisable selection cassette (FRT-antibiotic resistance gene-FRT), as described by Datsenko and Wanner. The resulting 3-methylcrotonic acid (MCA)-producing strain with a decreased pool of crotonic acid (CA) was tested in a 1 L bioreactor in fermentation no F2253.

C/FabV Overexpression and yciA Deletion

For this Example 2C, fabV from Treponema denticola (EC 1.3.1.44) (Uniprot Accession No: Q73Q47) was cloned in the expression vector pSC101 with a P7 constitutive promoter, a RBST7 ribosome-binding site and a PN25 terminator, and yciA from Escherichia coli (EC 3.1.2.20) (Uniprot Accession No: P0A8Z0) was deleted in the chromosome (see example 1B). The resulting 3-methylcrotonic acid (MCA)-producing strain with a decreased pool of crotonic acid (CA) was tested in a 1 L bioreactor in fermentation no F2255.

### D/FabI Overexpression Alone

For this Example 2D, fabI from Escherichia coli (EC 1.3.1.9 and 1.3.1.104) (Uniprot Accession No: P0AEK4) was cloned in a modified version of pUC18 (New England Biolabs), containing a modified Multiple Cloning Site (pUC18 MCS) (WO 2013/007786), and expressed from the lac promoter. The 3-methylcrotonic acid (MCA)-producing strain with the pSC101 expression vector was transformed with this additional expression vector for fabI overexpression, which conferred ampicillin resistance to the recombinant strain. This was tested in a 1 L bioreactor in fermentation n° F.2226. The resulting 3-methylcrotonic acid (MCA)-producing strain showed a 10-fold decreased pool of crotonic acid (CA), but also a 3-fold decreased production of 3-methylcrotonic acid (MCA).

E/FabI Overexpression and tesB Deletion

For this example 2E, fabI from Escherichia coli (EC 1.3.1.9 and 1.3.1.104) (Uniprot Accession No: P0AEK4) was cloned in a modified version of pUC18 (New England Biolabs), containing a modified Multiple Cloning Site (pUC18 MCS) (WO 2013/007786), and expressed from the lac promoter.

tesB from Escherichia coli (EC 3.1.2.20) (Uniprot Accession No: P0AGG2) was deleted in the chromosome following transformation with a PCR product containing 50 bp homology sequences (for recombination) flanking an excisable selection cassette (FRT-antibiotic resistance gene-FRT), as described by Datsenko and Wanner.

A 3-methylcrotonic acid (MCA)-producing strain with tesB deletion was transformed with the additional expression vector for fabI overexpression. This was tested in a 1 L bioreactor in fermentation no F2221. The resulting strain showed a similar production of 3-methylcrotonic acid (MCA) compared to the reference strain, with a decreased pool of crotonic acid (CA). So a beneficial effect was observed when both genetic modifications (fabI overexpression and tesB deletion) were used together.

For each example, the resulting strains were made electro-competent and were transformed with the corresponding plasmids.

The transformed cells were then plated on LB plates (with appropriate antibiotics) and the plates were incubated overnight at 30° C. An isolated colony was used to prepare a pre-culture as described in the following.

Production of 3-methylcrotonic acid in 15 L Bioreactor (Example 2A, F2250) and its Purification

A 15 L vessel was filled with 7.5 L of a culture medium containing 15 g/L yeast extract, 50 mM sodium glutamate, 6.25 mM potassium phosphate monobasic and 6.25 mM sodium phosphate dibasic and sterilized at 121° C. for 20 minutes. After cooling, filter sterilized trace metals were added at a final concentration of 10 μM iron III chloride, 4 μM calcium chloride, 2 μM manganese chloride, 2 μM zinc sulfate, and 0.4 μM copper chloride. Then filter sterilized glucose was added at a final concentration of 5 g/L.

In addition to the batch culture medium, a fed batch solution was prepared consisting in a 600 g/L glucose solution.

The culture medium was inoculated with 500 ml of a pre-culture of strain previously grown at 30° C. in LB medium containing 50 mM sodium glutamate, 34 g/l magnesium sulfate heptahydrate and 10 mg/l tetracycline. Temperature was kept at 32° C. for 10 hours and then increased up to 34° C. In the same manner pH was first regulated at 7.2 for 10 hours and then at 7.6 with 5M phosphoric acid and 30% ammonia. Aeration was set at 4 liters per min and agitation was regulated to maintain dissolved oxygen at 20% of saturation. 5 g/l glucose were added three times after 7, 8 and 9 hours of culture.

A glucose fed batch was started 10h after the start of the culture. The initial feed rate was adjusted to deliver 4.5 g/l/h glucose and then it was decreased linearly to deliver 3.5 g/l/h after 10 h. The feed rate was again decreased linearly to deliver 2 g/l/h glucose after a new period of 24 hours. After that the feed rate of glucose was adjusted to maintain glucose at low levels around 1 to 3 g/l.

Fermentation was stopped after 64h of culture. The culture medium was then clarified by centrifugation and used for purification of 3-methylcrotonate as described in WO2022/136207. Briefly, the resulting supernatant was acidified by the addition of 98% sulfuric acid until the pH was adjusted to pH 3.5. Then evaporation was run using a rotavapor R300 (Buchi) at heating temperature of 80° C., cooling temperature of 10° C. and a pressure of 150 mbar. Crystals of 3-methylcrotonic acid were recovered on the condenser. They were removed by washing with water and mixed with the distillate. Evaporation was run until the residue became viscous. Distillate containing 3-methylcrotonic acid was recovered. Then 3M sodium hydroxide was added to the distillate, in order to adjust the pH at a value of 9.1. The obtained solution of sodium 3-methylcrotonate was evaporated at a heating temperature of 80° C., cooling temperature of 10° C., and at a pressure of 150 mbar until reaching a concentration of product close to 3 M.

The concentrated solutions were analyzed by HPLC (Hiplex column at 40° C. with a mobile phase of 5 mM sulfuric acid at 0.8 mL/min) and their composition is reported in the following table:

| | Reference | Strain with fabV |
|---|---|---|
| | strain | overexpression |
| 3-methylcrotonate (M) | 2.9 | 3.3 |
| Acetate (M) | 0.89 | 0.83 |
| Crotonate (μM) | 1 289 | 41 |

Production of 3-methylcrotonic acid in 1 L Bioreactor (Example 2B, F2253; Example 2C, F2255; Example D, F2226; Example 2E, F2221)

A 1 L vessel was filled with 0.5 L of a culture medium containing 15 g/L yeast extract, 71.6 mM sodium glutamate, 6.2 mM potassium phosphate monobasic, 7.8 mM sodium phosphate dibasic, 80 μL/L antifoam (Struktol J673, Schill und Seilacher) and trace metals (added at a final concentration of 10 μM iron III chloride, 4 μM calcium chloride, 2 μM manganese chloride, 2 μM zinc sulfate, 0.4 μM copper chloride) and sterilized at 121° C. for 20 minutes. After cooling, filter sterilized glucose was added at a final concentration of 5 g/L (in addition to the batch culture medium, a fed batch solution of 600 g/L filter sterilized glucose was prepared).

The culture medium was inoculated with 30 ml of a pre-culture at OD600=0.5 (previously grown at 30° C. in LB medium containing 50 mM sodium glutamate, 300 mM magnesium sulfate, 10 mg/L tetracycline and 100 mg/L ampicillin when appropriate).

All along the culture, the pH of the culture medium was regulated with an acid solution of 5 M phosphoric acid and a base solution of 10 M sodium hydroxide.

For the first 9 hours of culture, temperature was kept at 32° C., pH regulated at pH7.2, aeration was set at 0.25 L/min, agitation was regulated to maintain dissolved oxygen at 15% of saturation, and 5 g/L of glucose was added at t=6 h, t=7 h and t=8 h (3×5 g/L). At t=9 h, temperature was increased up to 34° C., pH regulated at pH7.6 and 3 g/l of glucose was added. The feed rate of glucose was set at 5.5 g/l/h for 6h, and then decreased to reach 2 g/l/h in 24 h. The feed rate of glucose was also adjusted to avoid the accumulation of glucose (1-3 g/L) and acetic acid in the culture medium.

The concentrations of 3-methylcrotonic acid and crotonic acid in the culture medium were monitored by HPLC (Hiplex column at 40° C. with a mobile phase of 5 mM sulfuric acid at 0.8 mL/min). The fermentation was stopped when acetic acid started to accumulate instead of the desired product.

### Example 3: Bioconversion of 3-Methylcrotonic Acid (MCA) to Isobutene (IBN) from 3-Methylcrotonic Acid with a Decreased Pool of Crotonic Acid (CA)

This Example shows the production of isobutene by a recombinant E. coli strain which expresses exogenous genes allowing the production of isobutene from 3-methylcrotonic acid: The following genes were codon-optimized for the expression in E. coli and synthesized by GeneArt (Life Technologies):
FDC1 (ferulic acid decarboxylase) from Yersinia frederiksenii (Uniprot Accession number A0A0T9UUQ9)
kpdB (UbiX-like flavin prenyltransferase) from Klebsiella pneumoniae (Uniprot Accession number Q462H4)

An expression vector containing the origin of replication of pSC101 (reference: http://www.ncbi.nlm.nih.gov/pmc/articles/PMC320470/) was used for the expression of the genes.

E. coli MG1655 cells were transformed with the constructed plasmid and grown to a cell density of about 35 g/L on a rich medium containing yeast extract and mineral salts with glucose as a carbon source. Cells were collected by centrifugation and resuspended in the supernatant at a concentration of 100 g/L and kept at 4° C. for up to 3 weeks before use.

One liter bioreactors were filled with 37.5 ml of the 100 g/L cell concentrate and 262.5 ml of water. Temperature was set at 37° C. and agitation at 400 rpm. The vessels were ventilated with nitrogen through a sparger to flush the air from the headspace of the bioreactor. Outlet gas was analyzed and when oxygen was no longer detected the gas supply nitrogen was set at 0.3 liter per minute. The bioreactors were fed with the sodium 3-methylcrotonate concentrate solutions obtained in Example 2A (F2250) (see above). The feed rate was adjusted to deliver 7.1 mmoles/h sodium 3-methylcrotonate as long as 3-methylcrotonate did not accumulate in the broth, then feed rate was decreased gradually in order to maintain concentration of 3-methylcrotonate in the broth below 50 mM. pH was regulated at 6.5 with phosphoric acid 30%. The composition of exhaust gas was measured at least 4 times per hour to calculate the production of isobutene. The bioconversion of 3-methylcrotonate (MCA) into isobutene was run for 172 h.

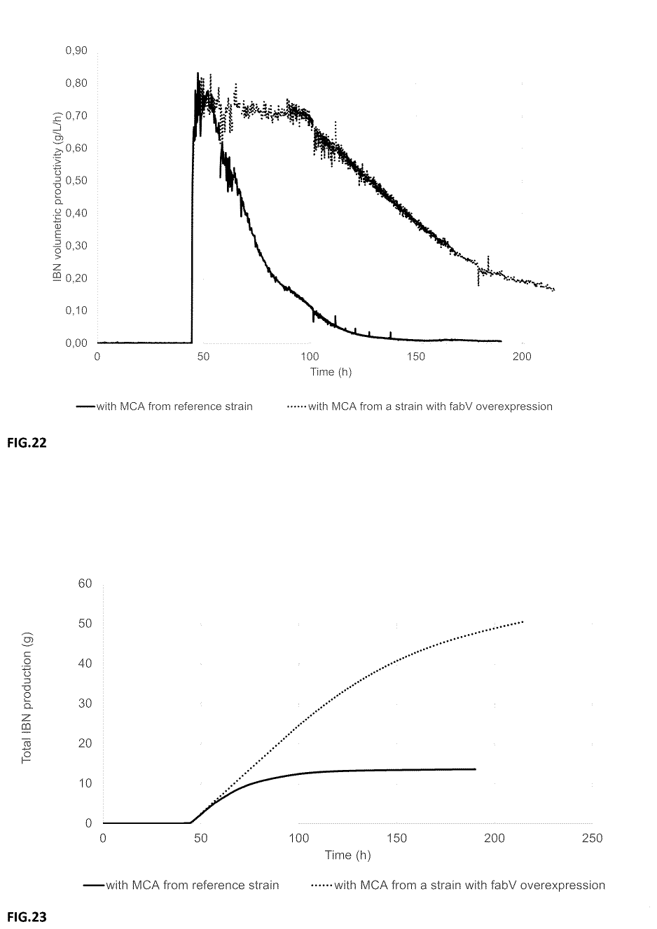

The isobutene volumetric productivities (g/L/h) of the resulting bioconversions are shown in Example 3A (FIG. 22) and the isobutene total productions (g/L) are shown in Example 3B (FIG. 23; F2262).

### Example 4: In Vivo Isobutene (IBN) Production from Acetyl-Coa from a Strain with a Decreased Pool of Crotonic Acid (CA)

This Example shows the direct production of isobutene by a recombinant E. coli strain which expresses exogenous genes, thereby constituting the isobutene pathway.

Like most microorganisms, E. coli converts glucose into acetyl-CoA. The enzymes used in this study to convert acetyl-CoA into isobutene are summarized in the following.

Expression of an Isobutene Biosynthetic Pathway in E. coli

The following genes were codon-optimized for the expression in E. coli and synthesized by GeneArt (Life Technologies):
thl (thiolase) from Clostridium acetobutylicum (Uniprot Accession number P45359)
mvaS (Hydroxymethylglutaryl-CoA synthase) from Enterococcus faecalis (Uniprot Accession number Q835L4)
ech (enoyl-CoA hydratase) from Pseudomonas sp. (Uniprot Accession number K9NHK2)
aibA and aibB that code for the two subunits of 3-methylglutaconyl-CoA decarboxylase from Myxococcus hansupus (Uniprot Accession number A0A0H4WQB1 and A0A0H4WWJ4)
menI (ydiI) (1,4-dihydroxy-2-naphthoyl-CoA hydrolase) from Escherichia coli (strain K12) (Uniprot Accession number P77781)
FDC1 (ferulic acid decarboxylase) from Streptomyces sp. 769 (Uniprot Accession number A0A0A8EV26)

An expression vector containing the origin of replication of pSC101 (reference: http://www.ncbi.nlm.nih.gov/pmc/articles/PMC320470/) was used for the expression of the genes: mvaS, ech, aibA, aibB, ydiI, FDC1 according to the procedure described in WO2017/085167, Example 12.

An additional copy of the mvaS gene from Enterococcus faecalis and UbiX from Escherichia coli (strain K12) (Uniprot Accession number POAG03) were cloned in a modified version of pUC18 (New England Biolabs), containing a modified Multiple Cloning Site (pUC18 MCS) (WO 2013/007786), and expressed from the lac promoter.

The strain MG1655 was modified by integration of the thl gene from Clostridium acetobutylicum into the ssrS locus and the sucrose operon cscAKB from Escherichia coli W was integrated in the zwf locus for sucrose utilization as carbon source.

### Genetic Modification to Decrease the Crotonic Acid Production and Increase Isobutene Production

To decrease the crotonic acid production in the isobutene-producing strain an additional genetic modification was carried out in the chromosome. FabV from Treponema denticola (EC 1.3.1.44) (Uniprot Accession No: Q73Q47) was integrated into the mgsA locus of E. coli, as described in Example 2A.

The resulting strain was made electro-competent and transformed with both expression vectors. The transformed cells were then plated on LB plates with 10 mg/L tetracycline and 100 mg/L ampicillin, and the plates were incubated overnight at 30° C. An isolated colony was used to prepare a pre-culture as described in the following.

Production of Isobutene from Sucrose in 1 L Bioreactor (F2238)

A 1 L vessel was filled with 0.5 L of a culture medium containing 5 g/L yeast extract, 10 g/L tryptone, 50 mM L-glutamic acid monosodium monohydrate, 5 mM sodium sulfate, 10 mM ammonium sulfate, 25 mM potassium phosphate monobasic, 31.3 mM sodium phosphate dibasic, 80 μL/L antifoam (Struktol J673, Schill und Seilacher) and sterilized at 121° C. for 20 minutes. After cooling, filter sterilized solutions were added to obtain the following final concentrations: 4 mM magnesium sulfate, 1× trace metals solution (50 μM iron III chloride, 20 μM calcium chloride, 10 μM manganese chloride, 10 UM zinc sulfate, 2 μM cobalt II chloride, 2 μM copper chloride, 2 μM nickel II chloride, 2 μM sodium molybdate, 2 μM sodium selenite, 2 μM boric acid), 10 mg/L tetracycline, 100 mg/L ampicillin, 0.2 g/L thiamine hydrochloride, 1.2 g/L calcium pantothenate, 5 g/L glycerol, 1 g/L sucrose. A filter sterilized fed batch solution was also prepared (700 g/L sucrose, 107 mM L-glutamic acid monosodium monohydrate, 4.15 mM magnesium sulfate, 10× trace metals solution).

The culture medium was inoculated with 50 ml of a pre-culture at OD600=0.5 (previously grown at 30° C. in LB medium containing 50 mM L-glutamic acid monosodium monohydrate, 10 mg/L tetracycline, 100 mg/L ampicillin).

All along the culture, the pH of the culture medium was regulated with an acid solution of 5 M phosphoric acid and a base solution of 30% ammonium hydroxide.

For the first 40 hours of culture, temperature was kept at 32° C., pH regulated at pH6.5, aeration was set at 1 L/min and agitation was regulated to maintain dissolved oxygen at 15% of saturation. During growth phase, yeast extract was also added at 5 g/L at t=12 h, t=14 h, t=16 h, t=18 h, t=20 h, t=22 h, t=24 h, and t=26h. At t=12 h, an exponential feed of the fed batch solution was started at 0.1 g sucrose/g DCW/h to reach 0.35 g sucrose/g DCW/h at t=40 h. At t=40 h, the temperature was increased to 34° C. and agitation was regulated to maintain dissolved oxygen at 5% of saturation. The feed of the fed batch solution was kept at 0.35 g sucrose/g DCW/h for 8 hours and then decreased to reach 0.25 g sucrose/g DCW/h after 16 hours. The feed rate was also adjusted to avoid the accumulation of sucrose (<2 g/L) in the culture medium.

The production of isobutene in the gas phase was measured by mass spectrometry and the concentration of crotonic acid in the culture medium was monitored by HPLC (Hiplex column at 40° C. with a mobile phase of 5 mM sulfuric acid at 0.8 mL/min).

### Results (F2238)

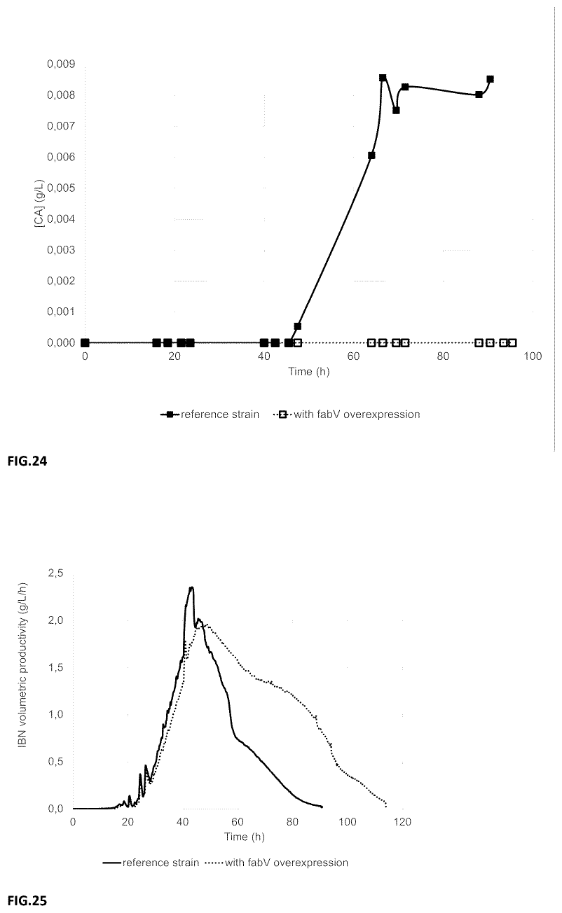

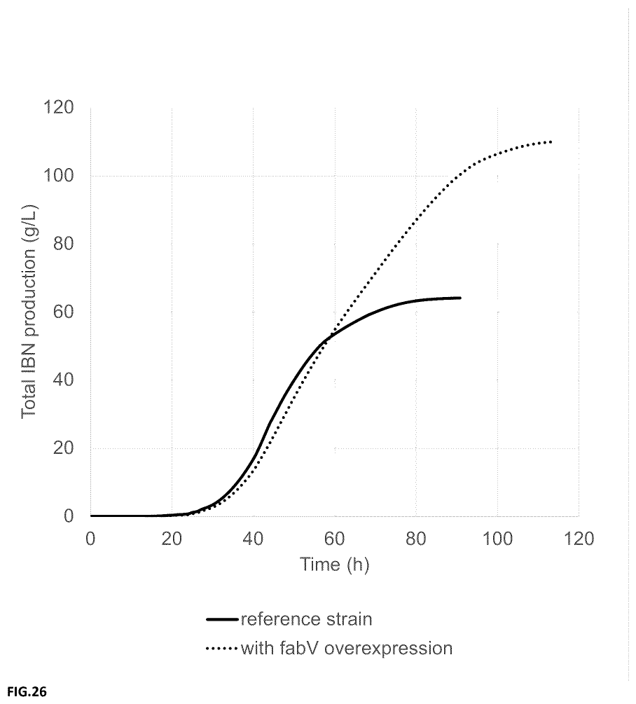

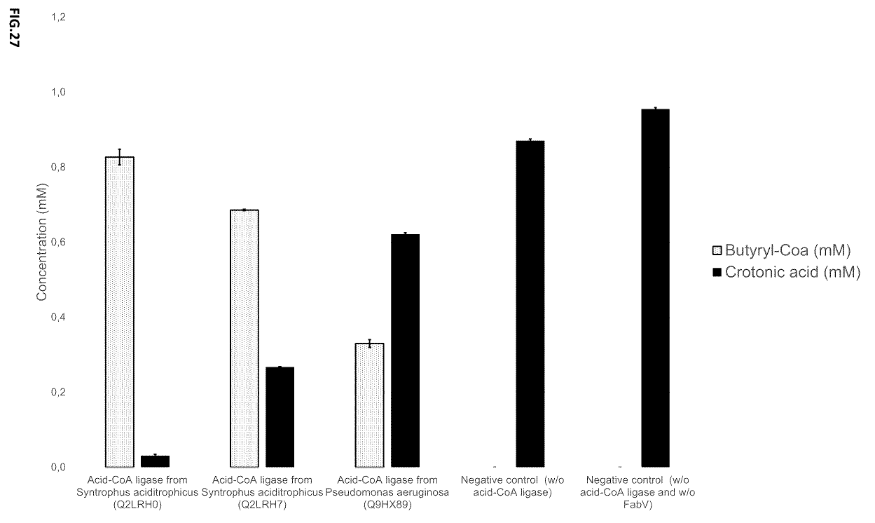

With the integration of fabV from Treponema denticola (EC 1.3.1.44) (Uniprot Accession No: Q73Q47) into the mgsA locus of an isobutene-producing strain of Escherichia coli, crotonic acid was no longer detectable in the culture medium (Example 4A; FIG. 24) and isobutene production could be maintained for a longer period (Example 4B; FIG. 25), which increased isobutene total production (Example 4C; FIG. 26).

### Example 5: Identification of Enzymes Converting Crotonic Acid (CA) to Crotonyl-CoA

Two enzyme classes were used to convert crotonic acid (CA) to crotonyl-CoA:

### 1/EC 6.2.1: Acid-Thiol Ligases

This enzyme class catalyses the formation of a thioester bond from a carboxylate and a thiol donor (e.g. coenzyme A or an [acyl-carrier protein]), at the expense of one nucleoside triphosphate molecule (e.g. ATP):
Carboxylate+NTP+CoA→acyl-CoA+NMP+diphosphate
Carboxylate+NTP+CoA→acyl-CoA+NDP+phosphate (e.g. ADP-forming)
Carboxylate+NTP+[acyl-carrier protein]→acyl-[acyl-carrier protein]+NMP+diphosphate
Carboxylate+NTP+[acyl-carrier protein]→acyl-[acyl-carrier protein]+NDP+phosphate (e.g. ADP-forming)

Three acid-CoA ligases were used to convert crotonic acid (CA) to crotonyl-CoA: two acid-CoA ligases from Syntrophus aciditrophicus (Uniprot: Q2LRH0 (SEQ ID NO:55) and Q2LRH7 (SEQ ID NO:56)) (Kung J W, Seifert J, von Bergen M, Boll M. Cyclohexanecarboxyl-coenzyme A (CoA) and cyclohex-1-ene-1-carboxyl-CoA dehydrogenases, two enzymes involved in the fermentation of benzoate and crotonate in Syntrophus aciditrophicus. J Bacteriol. 2013 July; 195 (14): 3193-200. doi: 10.1128/JB.00322-13.), and an acid-CoA ligase from Pseudomonas aeruginosa (Uniprot: Q9HX89; SEQ ID NO:57) (Hokamura A, Wakida I, Miyahara Y, Tsuge T, Shiratsuchi H, Tanaka K, Matsusaki H. Biosynthesis of poly(3-hydroxybutyrate-co-3-hydroxyalkanoates) by recombinant Escherichia coli from glucose. J Biosci Bioeng. 2015 September; 120 (3): 305-10. doi: 10.1016/j.jbiosc.2015.01.022.).

SEQ ID NO: 55:
>tr|Q2LRH0|Q2LRHO_SYNAS 4-hydroxybenzoate--CoA ligase/benzoate--CoA ligase
OS = Syntrophus aciditrophicus (strain SB) OX = 56780 GN = SYN_02896 PE = 4 SV = 1
MAKLYPEEFYNSADWFVDRHVREGRGDNICAYTDKGNYTYRDIQKMANKMANMFKDLDIR
MGDRIIMLVLDTPWFYSTFWGAVKMGAVPVPSSTMLTPADYEYYLNDSQARTLVVSSRLL
PVVNQIEELRFLKNMIVVDDDGVFSTPYQQIYASASDEFQTVFTSADDVAFWLYTSGTTG
GPKGAVHSQSDMQYSAEAYGKHILEITEKDICYSAARLFFTYGLGNAMFFPMSVGAAAVL
NPDPPAPAHVFRLIKTYKATLFFGVPTLFGQMLITQDKIDAEKGAGADPKDHDLVSLRAC
PSAGEALPPDLYHKFKARYGVEILDGPGSTEMLHIYLSNKLGDVKPGSSGKPVPGYEEKI
MDEEGKNELPDGEVGNLWIKGRSSLRYYWRKRDKTAATVIGEWVNSGDKYYKDAEGYYWP
SGRADDMLKVGGIWVSPLEVENCLREHASVMECAVVGAMDEENLVKPKAFVVLNQGFAQS
PELEKELKQWVLDRLAKFKYPRWIVFIDSLPKTATGKIQRFKLR
SEQ ID NO: 56:
>tr|Q2LRH7|Q2LRH7_SYNAS 4-hydroxybenzoate--CoA ligase/benzoate--CoA ligase
OS = Syntrophus aciditrophicus (strain SB) OX = 56780 GN = SYN_02898 PE = 4 SV = 1
MAVYTEEFYNSVNYFIDRHIEEGRGDKICAYTDKGNYTYRDMQKMVNKMANMFRKLDIRI
GDRVIMLVFDTPWFFSTEWGAVRIGAVPVPSNTMLTSDDYQYYLNDSQARTLVISEKLLP
LIKGIKGELRYLRDVIVVDDDGEFSTPYQQMYAQASEEAETAFTTKDDVAFWQYTSGTTG
APKGAVHSHSDMQYVAEAYGKHVLGMTENDVCYSAARLFFAYGIGNGMVYPLSVGAASVL
NPDPPTPERAFRLNSTYKVTLFFGIPTLFGQMLEYKVKQEKEAGITPDPKAPHELSSVRA
CPSAGEALPPDLYHREKERFGVEILDGPGSTEMLHIYLSNTLGDVKAGSSGKVVPGYEAK
IVGEEGETLPDGEIGTLWVKGDSSLRYYWRKKEKTASTIIGGWVNTGDKYYRDKDGYFWP
SGRADDMLKVGGIWVSPLEVENCLREHPAVLETAVIGAEDEKNLVKPKAFVVLKQGFAPS
PELEKELKQWVLDRLAKFKYPRWIVEMDELPKTATGKIQRFKLR
SEQ ID NO: 57:
>tr|Q9HX89|Q9HX89_PSEAE Probable medium-chain acyl-CoA ligase OS = Pseudomonas
aeruginosa (strain ATCC 15692/DSM 22644/CIP 104116/JCM 14847/LMG
12228/1C/PRS 101/PAO1) OX = 208964 GN = PA3924 PE = 4 SV = 1
MLKTRLIPAAAGAYQYPLLIKSLMLSGRRYEKSHEIVYRDQVRYSYATENERVARLANVL
SEAGVKAGDTVAVMDWDSHRYLECMFAIPMIGAVLHTINIRLSPEQILYTMNHAEDRFVL
VNSEFVPLYQAVAGQLATVERTILLTDGAEKSAELPGLVGEYESLLAAASPRYDFPDEDE
NSIATTFYTTGTTGNPKGVYFSHRQLVLHTLAMASTIGSLDSIRLIGTSDVYMPITPMFH
VHAWGTPYVATMLGVKQVYPGRYDPELLVELWKREKVTFSHCVPTILQMVMNARAAQGVD
FKGWKVIIGGSALNRSLYEAAKARGIQLTAAYGMSETCPLISCAYLNDELLAGSEDERTT
YRIKAGVPVPLVDAAIMDEQGRFLPADGESQGELVLRSPWLTQGYFREPERGEELWRGGW
MHTGDVATLDGMGFIEIRDRIKDVIKTGGEWLSSLELEDLISRHPAVREVAVVGVPDPQW
GERPFALLVVREGQQLDARGLKEHLKPFVEQGNINKWAIPSQIAVVTDIPKTSVGKLDKK
RIRIEIAQWQEAGSAFLSTV

### 2/EC 2.8.3: CoA-Transferases

This enzyme class catalyses the reversible transfer of CoA from one carboxylate to another. When acetyl-CoA is used as CoA donor the reaction is as follows:
Carboxylate+acetyl-CoA→acyl-CoA+acetate

A CoA-transferase from Cupriavidus necator (Ralstonia eutropha) (Uniprot: Q0K874; SEQ ID NO:9) was used to convert crotonic acid (CA) to crotonyl-CoA with acetyl-CoA as CoA donor (Lindenkamp N, Schürmann M, Steinbüchel A. A propionate CoA-transferase of Ralstonia eutropha H16 with broad substrate specificity catalyzing the CoA thioester formation of various carboxylic acids. Appl Microbiol Biotechnol. 2013 September; 97(17):7699-709. doi: 10.1007/s00253-012-4624-9. Epub 2012 Dec. 19. PMID: 23250223.)

SEQ ID NO: 9:
>tr|Q0K874|Q0K874_CUPNH Acetate CoA-transferase YdiF OS = Cupriavidus necator
(strain ATCC 17699/DSM 428/KCTC 22496/NCIMB 10442/H16/Stanier 337)
OX = 381666 GN = pct PE = 3 SV = 1
MKVITAREAAALVQDGWTVASAGFVGAGHAEAVTEALEQRFLQSGLPRDLTLVYSAGQGD
RGARGVNHFGNAGMTASIVGGHWRSATRLATLAMAEQCEGYNLPQGVLTHLYRAIAGGKP
GVMTKIGLHTFVDPRTAQDARYHGGAVNERARQAIAEGKACWVDAVDERGDEYLFYPSFP
IHCALIRCTAADARGNLSTHREAFHHELLAMAQAAHNSGGIVIAQVESLVDHHEILQAIH
VPGILVDYVVVCDNPANHQMTFAESYNPAYVTPWQGEAAVAEAEAAPVAAGPLDARTIVQ
RRAVMELARRAPRVVNLGVGMPAAVGMLAHQAGLDGFTLTVEAGPIGGTPADGLSFGASA
YPEAVVDQPAQFDFYEGGGIDLAILGLAELDGHGNVNVSKFGEGEGASIAGVGGFINITQ
SARAVVFMGTLTAGGLEVRAGDGGLQIVREGRVKKIVPEVSHLSENGPYVASLGIPVLYI
TERAVFEMRAGADGEARLTLVEIAPGVDLQRDVLDQCSTPIAVAQDLREMDARLFQAGPL
HL

Cloning of the Enzymes with an N-Terminal 6His Tag

The sequence of the enzymes was codon-optimized for the expression in E. coli and synthesized by GeneArt (Life Technologies).

The polynucleotide sequences were cloned alone in a pET-25b (+) (Novagen) expression vector with a polynucleotide tag in 5′ coding for a 6-His purification tag.

### Purification of the 6His-Tagged Enzymes by Affinity Chromatography (Ni-NTA)

The enzymes were purified by affinity chromatography (Ni-NTA) from bacterial cultures of BL21 (DE3) cells containing the gene cloned into a pET-25b (+) (Novagen) expression vector with a polynucleotide tag in 5′ coding for a 6-His purification tag.

### Characterization of the Acid-CoA Ligases (Example 5A; FIG. 27)

The acid-CoA ligases were tested as purified enzymes with a coupled assay using purified FabV from Treponema denticola (EC 1.3.1.44) (Uniprot Accession No: Q73Q47) (to convert crotonyl-CoA to butyryl-CoA). The reaction was carried out as follows: 50 μg/ml acid-CoA ligase and 100 μg/ml FabV with a buffered solution containing 100 mM Tris-HCl pH7.5, 20 mM NaCl, 100 mM KCl, 2 mM MgCl₂, 1 mM crotonic acid (TCI chemicals), 1 mM ATP (Sigma-Aldrich), 1 mM coenzyme A (Sigma-Aldrich) and 1 mM NADH (Sigma-Aldrich) were incubated 18 h at 34° C. in a water bath, and the reaction was stopped with acetonitrile. A positive control with 1 mM crotonyl-CoA as substrate showed that FabV could convert all of it to butyryl-CoA in these conditions. The produced butyryl-CoA (Sigma-Aldrich) and the consumed crotonic acid (TCI chemicals) were quantified by HPLC with a calibration curve. The samples were injected on a Zorbax SB-Aq column (Agilent) at 30° C. and eluted with 8.4 mM H₂SO₄ and a gradient of acetonitrile.

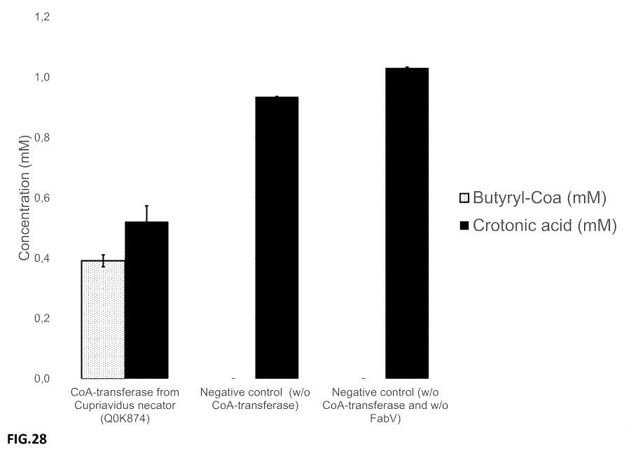

### Characterization of the CoA-Transferase (Example 5B; FIG. 28)

The CoA-transferase was tested as purified enzyme with a coupled assay using purified FabV from Treponema denticola (EC 1.3.1.44) (Uniprot Accession No: Q73Q47) (to convert crotonyl-CoA to butyryl-CoA). The reaction was carried out as follows: 50 μg/ml CoA-transferase and 100 μg/ml FabV with a buffered solution containing 100 mM Tris-HCl pH7.5, 20 mM NaCl, 100 mM KCl, 2 mM MgCl₂, 1 mM crotonic acid (TCI chemicals), 1 mM acetyl-CoA (Sigma-Aldrich) and 1 mM NADH (Sigma-Aldrich) were incubated 18 h at 34° C. in a water bath, and the reaction was stopped with acetonitrile. A positive control with 1 mM crotonyl-CoA as substrate showed that FabV could convert all of it to butyryl-CoA in these conditions. The produced butyryl-CoA (Sigma-Aldrich) and the consumed crotonic acid (TCI chemicals) were quantified by HPLC with a calibration curve. The samples were injected on a Zorbax SB-Aq column (Agilent) at 30° C. and eluted with 8.4 mM H₂SO₄ and a gradient of acetonitrile.

1. A recombinant organism or microorganism having a decreased pool of crotonic acid compared to the organism or microorganism from which it is derived due to at least one of:
(i) an increased conversion of crotonyl-CoA into butyryl-CoA; and/or an increased conversion of butyryl-CoA into butyric acid;
(ii) an increased conversion of crotonyl-CoA into 3-hydroxybutyryl-CoA; and/or an increased conversion of 3-hydroxybutyryl-CoA into 3-hydroxybutyric acid;
(iii) an increased conversion of crotonic acid into crotonyl-CoA;
(iv) an increased conversion of crotonyl-[acyl-carrier protein] into butyryl [acyl-carrier-protein];
(v) a decreased conversion of crotonyl-[acyl-carrier protein] into crotonic acid; and/or
(vi) a decreased conversion of crotonyl-CoA into crotonic acid.

2. The recombinant organism or microorganism according to claim 1, wherein said recombinant organism or microorganism is a recombinant microorganism; in particular wherein said recombinant microorganism is a fungus or a bacterium; in particular wherein said bacterium is Escherichia coli.

3. The recombinant organism or microorganism according to claim 1, wherein;
(a) the increased conversion of crotonyl-CoA into butyryl-CoA of 1(i) is due to an increased level and/or activity of at least one enzyme capable of reducing a carbon-carbon double bond (EC 1.3) in said organism or microorganism, in particular wherein the enzyme capable of reducing a carbon-carbon double bond (EC 1.3) is NADH or NADPH-dependent (EC 1.3.1) or flavin-dependent (EC 1.3.8);
(b) wherein the increased conversion of butyl-CoA into butyric acid in 1(i) is due to an increased level and/or activity of a thioester hydrolase (EC 3.1.2), a CoA-transferase (EC 2.8.3), an acid thiol ligase (EC 6.2.1), a phosphate acyltransferase (EC 2.3.1) and/or acid kinase (EC 2.7.2) in said organism or microorganism;
(c) wherein the increased conversion of crotonyl-CoA into 3-hydroxybutyryl-CoA in 1(ii) is due to an increased level and/or activity of a hydro-lyase (EC 4.2.1) in said organism or microorganism;
(d) wherein the increased conversion of 3-hydroxybutyryl-CoA into 3-hydroxybutyric acid in 1(ii) is due to an increased level and/or activity of a thioester hydrolase (EC 3.1.3) in said organism or microorganism;
(e) wherein the increased conversion of crotonic acid into crotonyl-CoA in 1(iii) is due to an increased level and/or activity of a CoA-transferase (EC 2.8.3) and/or an acid thiol ligase (EC 6.2.1) and/or an acid kinase (EC 2.7.2) and/or phosphate acyltransferase (EC 2.3.1) in said organism or microorganism;
(f) wherein the increased conversion of crotonyl-[acyl-carrier protein] into butyryl-[acyl-carrier protein] in 1(iv) is due to an increased level and/or activity of an NADH or NADPH-dependent enoyl-[acyl-carrier-protein] reductase (EC 1.3.1) in said organism or microorganism, in particular wherein the NADH or NADPH-dependent enoyl-[acyl-carrier-protein] reductase (EC 1.3.1) in an enoyl-[acyl-carrier-protein] reductase (NADH-dependent) (EC 1.3.1.9), an enoyl-[acyl-carrier-protein] reductase (NADPH-dependent) (EC 1.3.1.104), an enoyl-[acyl-carrier-protein] reductase (NADPH-dependent, Re-specific) (EC 1.3.1.39), an enoyl-[acyl-carrier-protein] reductase (NADPH-dependent, Si-specific) (EC 1.3.1.10), and/or a trans-2-enoyl-CoA reductase (NAD+) (EC 1.1.1.44);
(g) wherein the decreased conversion of crotonyl-[acyl-carrier-protein] and/or crotonyl-CoA into crotonic acid in 1(v) or 1(vi) is due to a decreased level and/or a decreased activity of a thioester hydrolase (EC 3.1.2) in said organism or microorganism;
(h) wherein the increased conversion of crotonyl-CoA into butyryl-CoA in 1(i) is due to an increased level and/or activity or a trans-2-enoyl-CoA reductase (EC 1.3.1.44) and/or an enoyl-[acyl-carrier-protein] reductase (EC 1.3.1.9); and/or
(i) wherein the decreased conversion of crotonyl-[acyl-carrier-protein] and/or crotonyl-CoA into crotonic acid in 1(v) or 1(vi) is due to a decreased level and/or a decreased activity of a thioester hydrolase (EC 3.1.2) in said organism or microorganism.

4. The recombinant organism or microorganism according to claim 3, wherein;
(a) the NADH or NADPH-dependent enzyme capable of reducing a carbon-carbon double bond (EC 1.3.1) of 3(a) is a crotonyl-CoA reductase (EC 1.3.1.86), a trans-2-enoyl-CoA reductase (EC 1.3.1.44) and/or an enoyl-[acyl-carrier-protein] reductase (EC 1.3.1.9);
(b) the flavin-dependent enzyme capable of reducing a carbon-carbon double bond (EC 1.3.8) of 3(a) is a short-chain acyl-CoA dehydrogenase (EC 1.3.8.1);
(c) the thioester hydrolase (EC 3.1.2) of 3(b) is a 1,4-dihydroxy-2-naphtoyl-CoA hydrolase (EC 3.1.2.28) and/or an acyl-CoA thioesterase 2 (E3.1.2.20);
(d) the CoA-transferase (EC 2.8.3) of 3(b) is an acetate CoA-transferase and/or a butyryl-CoA:acetate CoA-transferase (EC 2.8.3.8);
(e) the acid thiol ligase (EC 6.2.1) of 3(b) is an acetate-CoA ligase (ADP-forming) (EC 6.2.1.13);
(f) the phosphate acyltransferase (EC 2.3.1) of 3(b) is a phosphate butyryltransferase (EC 2.3.1.19) and/or the acid kinase (EC 2.7.2) of 3(b) is a butyrate kinase (EC 2.7.2.7);
(g) the hydro-lyase (EC 4.2.1) of 3(c) is a short-chain-enoyl-CoA hydratase (EC 4.2.1.150), a 3-hydroxybutyryl-CoA dehydratase (EC 4.2.1.55) and/or an enoyl-CoA hydratase (EC 4.2.1.17);
(h) the thioester hydrolase (EC 3.1.2) in 3(d) is a palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28);
(i) the CA-transferase (EC 2.8.3) of 3(e) is an acetate CoA-transferase (EC 2.8.3.8) and/or a butyryl-CoA acetate CoA-transferase (EC 2.8.3.8);
(j) the acid thiol ligase (EC 6.2.1) of 3(e) is a medium-chain acyl-CoA lease (EC 6.2.1.2), a 4-hydroxybenzoate-CoA ligase/benzoate-CoA ligase (EC 6.2.1.25 and 6.2.1.27), a 4-Hydroxybutyrate-CoA ligase (EC 6.2.1.40), and/or a methylmercaptopropionate (MMPA)-coenzyme A (CoA) ligase (EC 6.2.1.44);
(k) the phosphate acyltransferase (EC 2.3.1) of 3(e) is a phosphate butyryltransferase (EC 2.3.1.19) and/or the acid kinase (EC 2.7.2) is a butyrate kinase (EC 2.7.2.7);
(l) the NADH or NADPH-dependent enoyl-[acyl-carrier-protein] reductase of 4(i) is FabI from Escherichia coli (EC 1.3.1.9 and 1.3.1.104), FabI from Bacillus subtills (EC 1.3.1.9), FabL from Bacillus subtilis (EC 1.3.1.104), FabI from Staphylococcus aureus (EC 1.3.1.39), FabK from Porphyromonas gingivalis (EC 1.3.1.10 and EC 1.3.1.39), FabK from Streptococcus pneumoniae (EC 1.3.1.10), ETR1 from Saccharomyces cerevisiae (EC 1.3.1.104), FabV from Burkholderia mallei (EC 1.3.1.9 and 1.3.1.44), FabV from Pseudomonas aeruginosa (EC 1.3.1.9 and 1.3.1.44), FabV from Vibrio cholera (EC 1.3.1.9 and 1.3.1.44), FabV from Treponema denticola (EC 1.3.1.44), FabI from Pseudomonas aeruginosa (EC 1.3.1.9), and/or FabI from Burkholderia pseudomallei (EC 1.3.1.9); and/or
(m) wherein the thioester hydrolase (EC 3.1.2) of 3(g) is a palmitoyl-CoA hydrolase (EC 3.1.2.2), an acyl-CoA thioesterase 2 (EC 3.1.2.20) and/or a 1,4-dihydroxy-2-naphtoyl-CoA hydrolase (EC 3.1.2.28).

5. The recombinant organism or microorganism according to claim 4, wherein
(i) the trans-2-enoyl-CoA reductase (EC 1.3.1.44) of 4(a) is FabV from Treponema denticola;
(ii) the crotonyl-CoA reductase (EC 1.3.1.86) of 4(a) is Ccr from Streptomyces collinus; and/or
(iii) the enoyl-[acyl-carrier-protein] reductase (EC 1.3.1.9) of 4(a) is FabI from Escherichia coli;
(iv) the short-chain acyl-CoA dehydrogenase (EC 1.3.8.11 of 4(b) is a short-chain acyl-CoA dehydrogenase from Megasphaera elsdenii; and/or a butyryl-CoA dehydrogenase (Bcd) with the electron transferring flavoprotein (Etf) from Acidaminococcus fermentans;
(v) the 1,4-dihydroxy-2-naphtoyl-CoA hydrolase (EC 3.1.2.28) of 4(c) is MenI from Escherichia coli;
(vi) the acyl-CoA thioesterase 2 (EC 3.1.2.20) of 4(c) is TesB from Escherichia coli;
(vii) the acetate CoA-transferase (EC 2.8.3.8) of 4(d) is YdiF (Pct) from Cupriavidus necator;
(viii) the butyrate acetyl-CoA-transferase (EC 2.8.3.8) of 4(d) is encoded by SwoI 1932 or SwoI 0436 from Syntrophomonas wolfei subsp. wolfei;
(ix) the acetate-CoA ligase (ADP-forming) (EC 6.2.1.13) of 4(e) is encoded by the gene Caur 3920 from Chloroflexus aurantiacus and/or the gene EHI 178960 from Entamoeba histolytica and/or wherein the acetate-CoA ligase (ADP-forming) (EC 6.2.1.13) of 4(e) is the protein Q9Y1N2 from Giardia intestinalis (Giardia lamblia);
(x) the phosphate butyryltransferase (EC 2.3.1.19) of 4(l) is Plb from Clostridium acetobutylicum;
(xi) the butyrate kinase (EC 2.7.2.7) of 4(f) is Buk from Clostridium acetobutylicum;
(xii) the short-chain-enoyl-CoA hydratases (EC 4.2.1.150) of 4(g) is a short-chain-enoyl-CoA hydratase from Meiothermus ruber, Metallosphaera sedula or Clostridium acetobutylicum;
(xiii) the 3-hydroxybutyryl-CoA dehydratase (EC 4.2.1.55) of 4(h) is a 3-hydroxybutyryl-CoA dehydratase from Ferroglobus placidus;
(xiv) the enoyl-CoA hydratase EC 4.2.1.17) of 4(g) is an enoyl-CoA hydratase from Rattus norvegicus;
(xv) the palmitoyl-CoA hydrolase (EC 3.1.2.2) of 4(h) is a palmitoyl-CoA hydrolase from Photobacterium profundum;
(xvi) the acyl-CoA thioesterase 2 (EC 3.1.2.20) of 4(h) is TesB or YclA from Escherichia coli;
(xvii) the acetate CoA-transferase (EC 2.8.3.8) of 4(i) is YdlF (Pct) from Cupriavidus necator;
(xviii) the butyrate:acetyl-CoA-transferase (EC 2.8.3.8) of 4(j) is encoded by SwoI 1932 or SwoI 0436 from Syntrophomonas wolfei subsp. wolfei;
(xix) the medium-chain acyl-CoA ligase (EC 6.2.1.2) of 4(i) is encoded by the gene PA3924 from Pseudomonas aeruginosa;
(xx) the 4-hydroxybenzoate-CoA ligase/benzoate-CoA ligase (EC 6.2.1.25 and 6.2.1.27) of 4(j) is encoded by the gene SYN 02896 from Syntrophus aciditrophicus (strain SB) or by the gene SYN 02698 from Syntrophus aciditrophicus (strain SB);
(xxi) the 4-Hydroxybutyrate-CoA ligase (EC 6.2.1.40) of 4(j) is encoded by the gene Tneu 0420 from Pyrobaculum neutrophilum (Thermoproteus neutrophilus);
(xxii) the methylmercaptopropionate (MMPA-coenzyme A (CoA) ligase (EC 6.2.1.44) of 4(j) is encoded by the gene SAR11 0248 from Pelagibacter ubique; or by the gene SPO0677 from Ruegeria pomeroyi; or by the gene SPO2045 from Ruegeria pomeroyl; or by the gene SL1157 1815 from Ruegeria lacuscaerulensis; or by the gene SL1157 2728 from Ruegeria lacuscaerulensis or by the gene PA4198 from Pseudomonas aeruginosa; or by the gene BTH I2141 from Burkholderia thailandensis;
(xxiii) the phosphate butyryltransferase (EC 2.3.1.19) of 4(k) is Ptb from Clostridium acetobutylicum;
(xxiv) the butyrate kinase (EC 2.7.2.7) of 4(k) is Buk from Clostridium acetobutylicum;
(xXv) the thioester hydrolase (EC 3.1.2) of 3(g) is PaaY or PaaI from Escherichia coli;
(xxvi) the palmitoyl-CoA hydrolase (EC 3.1.2.2) of 4(m) is TesA, YclA EntH from Escherichia coli;
(xxvii) the acyl-CoA thioesterase 2 (EC 3.1.2.20) Of 4(m) is TesB or FadM from Escherichia coli; and/or
(xxvii) the 1,4-dihydroxy-2-naphtoyl-CoA hydrolase (EC 3.1.2.28) of 4(m) is MenI from Escherichia coli.

6-31. (canceled)

32. The recombinant organism or microorganism according to claim 3, wherein
(a) the increased level of an enzyme is achieved by expressing a gene encoding the respective enzyme from a recombinant promoter and/or from an improved ribosome binding site; and/or wherein the increased activity of an enzyme is due to one or more activating mutations in the gene encoding the respective enzyme;
(b) the wherein the decreased level of an enzyme is due to
(i) a complete or partial deletion of a gene encoding the respective enzyme in said organism or microorganism; and/or
(ii) a deletion or an inactivating mutation in a regulatory element of a gene encoding the respective enzyme in said organism or microorganism; and/or
wherein the decreased activity or an enzyme is due to
(i) an inactivating mutation in a gene encoding the respective enzyme in said organism or microorganism; and/or
(ii) the addition of an inhibitor of the respective enzyme.

33-38. (canceled)

39. The recombinant organism or microorganism according to claim 1 further encoding a ferulic acid decarboxylase; in particular wherein the ferulic acid decarboxylase catalyzes the formation of an alkene from a corresponding carboxylic acid.

40. The recombinant organism or microorganism according to claim 1, wherein the organism or microorganism is capable of producing a substrate of a ferulic acid decarboxylase.

41. The recombinant organism or microorganism according to claim 1, wherein the organism or microorganism is capable of
(i) enzymatically converting acetyl-CoA into 3-methylcrotonic acid and/or isobutene; and/or
(ii) enzymatically converting 3-methylcrotonic acid into isobutene; and/or
(iii) enzymatically converting cis, cis-muconic acid into 1-3-butadiene; and/or
(iv) enzymatically converting pentadienoic acid into 1-3-butadiene.

42. The recombinant organism or microorganism according to claim 41, wherein the conversion of acetyl-CoA into 3-methylcrotonic acid comprises the steps of:
(i) enzymatically converting acetyl-CoA into acetoacetyl-CoA,
(ii) enzymatically converting said produced acetoacetyl-CoA into 3-hydroxy-3-methylglutaryl-CoA,
(iii) enzymatically converting said produced 3-hydroxy-3-methylglutaryl-CoA into 3-methylglutaconyl-CoA,
(iv) enzymatically converting said produced 3-methylglutaconyl-CoA into 3-methylcrotonyl-CoA, and
(v) enzymatically converting said produced 3-methylcrotonyl-CoA into 3-methylcrotonic acid.

43. The recombinant organism or microorganism according to claim 42, wherein the recombinant organism or microorganism is capable of enzymatically converting the produced 3-methylcrotonic acid into isobutene.

44. The recombinant organism or microorganism according to claim 43, wherein the conversion of 3-methylcrotonic acid into isobutene is catalyzed by a ferulic acid decarboxylase.

45. A method of producing an alkene, wherein said method comprises culturing the recombinant organism or microorganism according to claim 1 in a suitable culture media under suitable conditional to produce an alkene, in particular wherein the alkene is isobutene or 1,3-butadiene.

46. The method of claim 45, wherein the alkene is produced by a ferulic acid decarboxylase.

47. A method for the production of 3-methylcrotonic acid and/or isobutene, the method comprising a step of culturing a recombinant organism or microorganism as defined in claim 42 in a suitable culture medium under suitable conditions.

48. A method for the production of isobutene, the method comprising the steps of:
a) producing 3-methylcrotonic acid by culturing a recombinant organism or microorganism as defined in claim 42 in a suitable culture medium under suitable conditions; and
b) enzymatically converting said produced 3-methylcrotonic acid into isobutene.

49. The method according to claim 48, wherein the conversion of 3-methylcrotonic acid into isobutene is catalyzed by a ferulic acid decarboxylase.
